SEQUENCE LISTING (153 sequences)

SEQ ID NO: 1 - protein, 109 aa, synthetic construct
DFGLDCDEHSTESRCCRYPLTVDFEAFGWDWIIAPKRYKANYCSGECEFVFLQKYPHTHLVHQANPRGSAGPCCTPTKMSPINMLYFNGKEQIIYGKIPAMVVDRCGCS

SEQ ID NO: 2 - protein, 31 aa, synthetic construct
HAEGTFTSDVSSYLEGQAAKEFIAWLVKGRG

SEQ ID NO: 3 - protein, 31 aa, synthetic construct
  SITE 2: note = 2-aminoisobutyric acid
  VARIANT 3: note = Glu or (S)-2-amino-3-(2H-tetrazol-5-yl)propanoic acid
  VARIANT 5: note = Ser or Thr
  VARIANT 6: note = Phenylalanine, alpha methyl phenylalanine, or 2-fluoro alpha methyl phenylalanine
  VARIANT 13: note = Tyr, alpha methyl phenylalanine, or alpha methyl tyrosine
  VARIANT 20: note = Lys, lysine derivative, Gln, Glu, His, or 2-aminoisobutyric acid
  VARIANT 22: note = Phe or alpha methyl phenylalanine
  VARIANT 24: note = Ala, Gln, or Glu
  VARIANT 25: note = Tryptophan or alpha methyl tyrosine
  VARIANT 28: note = Lys, Ala, Gln, or Glu
  VARIANT 31: note = Residue may be amidated
YXXGXXTSDYSSXLEEQAAXEXIXXLVXGGG

SEQ ID NO: 4 - protein, 120 aa, synthetic construct
EVQVLESGGDLVQPGGSLRLSCAASGFTFSAYAMTWVRQAPGKGLEWVSAISGSGGSAYYADSVKGRFTISRDNSKNTVYLQMNSLRAEDTAVYYCAKDGAWKMSGLDVWGQGTTVIVSS

SEQ ID NO: 5 - protein, 107 aa, synthetic construct
DIQMTQSPASLSASVGDRVTITCRASQDISDYLAWYQQKPGKIPRLLIYTTSTLQSGVPSRFRGSGSGTDFTLTISSLQPEDVATYYCQKYDSAPLTFGGGTKVEIK

SEQ ID NO: 6 - protein, 8 aa, synthetic construct
GFTFSAYA

SEQ ID NO: 7 - protein, 8 aa, synthetic construct
ISGSGGSA

SEQ ID NO: 8 - protein, 13 aa, synthetic construct
AKDGAWKMSGLDV

SEQ ID NO: 9 - protein, 6 aa, synthetic construct
QDISDY

SEQ ID NO: 10 - skipped

SEQ ID NO: 11 - protein, 9 aa, synthetic construct
QKYDSAPLT

SEQ ID NO: 12 - protein, 47 aa, synthetic construct
  SITE 2: note = 2-aminoisobutyric acid
  SITE 47: note = Amidated bromo acetyl modified residue
HXEGTFTSDYSSYLEEQAAKEFIAWLVKGGGGGGGSGGGGSGGGGSK

SEQ ID NO: 13 - protein, 47 aa, synthetic construct; identical to SEQ ID NO: 12
  SITE 2: note = 2-aminoisobutyric acid
  VARIANT 47: note = Amidated 2-amino-6-(2-(cyclooct-2-yn-1-yloxy)acetamido)hexanamide or 2-amino-6-(2-(cyclooct-2-yn-1-yloxy)acetamido)hexanamide

SEQ ID NO: 14 - protein, 32 aa, synthetic construct
  SITE 2: note = 2-aminoisobutyric acid
  SITE 6: note = Alpha methyl phenylalanine
  SITE 25: note = Alpha methyl tyrosine
  SITE 32: note = Amidated residue linked to another sequence via the side chain
YXEGTFTSDYSSYLEEQAAKEFIAYLVKGGGK

SEQ ID NO: 15 - protein, 47 aa, synthetic construct
  SITE 2: note = 2-aminoisobutyric acid
  SITE 6: note = Alpha methyl phenylalanine
  SITE 25: note = Alpha methyl tyrosine
  SITE 47: note = Amidated 2-amino-6-(2-(cyclooct-2-yn-1-yloxy)acetamido)hexanamide
YXEGTFTSDYSSYLEEQAAKEFIAYLVKGGGGGGGSGGGGSGGGGSK

SEQ ID NO: 16 - protein, 47 aa, synthetic construct; identical to SEQ ID NO: 15
  SITE 2: note = 2-aminoisobutyric acid
  SITE 6: note = Alpha methyl phenylalanine
  SITE 25: note = Alpha methyl tyrosine
  SITE 47: note = Amidated azide modified residue

SEQ ID NO: 17 - protein, 47 aa, synthetic construct; identical to SEQ ID NO: 15
  SITE 2: note = 2-aminoisobutyric acid
  SITE 6: note = Alpha methyl phenylalanine
  SITE 25: note = Alpha methyl tyrosine
  SITE 47: note = Amidated azide modified residue

SEQ ID NO: 18 - protein, 31 aa, synthetic construct
  SITE 2: note = 2-aminoisobutyric acid
  SITE 31: note = Amidated residue
YXEGTFTSDYSSYLEEQAAKEFIAWLVKGGG

SEQ ID NO: 19 - protein, 31 aa, synthetic construct; identical to SEQ ID NO: 18
  SITE 2: note = 2-aminoisobutyric acid
  SITE 6: note = 2-fluoro alpha methyl phenylalanine
  SITE 31: note = Amidated residue

SEQ ID NO: 20 - protein, 32 aa, synthetic construct
  SITE 2: note = 2-aminoisobutyric acid
  SITE 6: note = 2-fluoro alpha methyl phenylalanine
  SITE 32: note = Amidated azide modified residue
YXEGTFTSDYSSYLEEQAAKEFIAWLVKGGGK

SEQ ID NO: 21 - protein, 32 aa, synthetic construct; identical to SEQ ID NO: 20
  SITE 2: note = 2-aminoisobutyric acid
  SITE 6: note = 2-fluoro alpha methyl phenylalanine
  SITE 32: note = Amidated residue

SEQ ID NO: 22 - protein, 32 aa, synthetic construct; identical to SEQ ID NO: 20
  SITE 2: note = 2-aminoisobutyric acid
  SITE 6: note = 2-fluoro alpha methyl phenylalanine
  SITE 32: note = Amidated residue linked to another sequence via the side chain

SEQ ID NO: 23 - protein, 32 aa, synthetic construct; identical to SEQ ID NO: 20
  SITE 2: note = 2-aminoisobutyric acid
  SITE 6: note = 2-fluoro alpha methyl phenylalanine
  SITE 32: note = Amidated residue

SEQ ID NO: 24 - protein, 32 aa, synthetic construct
  SITE 2: note = 2-aminoisobutyric acid
  SITE 6: note = Alpha methyl phenylalanine
  SITE 13: note = Alpha methyl phenylalanine
  SITE 32: note = Amidated azide modified residue
YXEGTFTSDYSSFLEEQAAKEFIAWLVKGGGK

SEQ ID NO: 25 - protein, 32 aa, synthetic construct
  SITE 2: note = 2-aminoisobutyric acid
  SITE 6: note = Alpha methyl phenylalanine
  SITE 25: note = Alpha methyl tyrosine
  SITE 32: note = Amidated azide modified residue
YXEGSFTSDYSSYLEEQAAKEFIAYLVKGGGK

SEQ ID NO: 26 - protein, 32 aa, synthetic construct; identical to SEQ ID NO: 20
  SITE 2: note = 2-aminoisobutyric acid
  SITE 6: note = Alpha methyl phenylalanine
  SITE 22: note = Alpha methyl phenylalanine
  SITE 32: note = Amidated azide modified residue

SEQ ID NO: 27 - protein, 32 aa, synthetic construct; identical to SEQ ID NO: 20
  SITE 2: note = 2-aminoisobutyric acid
  SITE 6: note = Alpha methyl phenylalanine
  SITE 13: note = Alpha methyl tyrosine
  SITE 22: note = Alpha methyl phenylalanine
  SITE 32: note = Amidated azide modified residue

SEQ ID NO: 28 - protein, 32 aa, synthetic construct; identical to SEQ ID NO: 14
  SITE 2: note = 2-aminoisobutyric acid
  SITE 6: note = 2-fluoro alpha methyl phenylalanine
  SITE 25: note = Alpha methyl tyrosine
  SITE 32: note = Amidated azide modified residue

SEQ ID NO: 29 - protein, 32 aa, synthetic construct
  SITE 2: note = 2-aminoisobutyric acid
  SITE 6: note = Alpha methyl tyrosine
  SITE 25: note = Alpha methyl tyrosine
  SITE 32: note = Amidated azide modified residue
YXEGTYTSDYSSYLEEQAAKEFIAYLVKGGGK

SEQ ID NO: 30 - protein, 32 aa, synthetic construct
  SITE 2: note = 2-aminoisobutyric acid
  SITE 6: note = Alpha methyl tyrosine
  SITE 32: note = Amidated azide modified residue
YXEGTYTSDYSSYLEEQAAKEFIAWLVKGGGK

SEQ ID NO: 31 - protein, 32 aa, synthetic construct; identical to SEQ ID NO: 20
  SITE 2: note = 2-aminoisobutyric acid
  SITE 6: note = Alpha methyl phenylalanine
  SITE 32: note = Amidated azide modified residue

SEQ ID NO: 32 - protein, 47 aa, synthetic construct; identical to SEQ ID NO: 15
  SITE 2: note = 2-aminoisobutyric acid
  SITE 6: note = Alpha methyl phenylalanine
  SITE 25: note = Alpha methyl tyrosine
  SITE 47: note = Amidated residue

SEQ ID NO: 33 - protein, 32 aa, synthetic construct; identical to SEQ ID NO: 30
  SITE 2: note = 2-aminoisobutyric acid
  SITE 6: note = Alpha methyl tyrosine
  SITE 32: note = Amidated residue linked to another sequence via the side chain

SEQ ID NO: 34 - protein, 47 aa, synthetic construct
  SITE 2: note = 2-aminoisobutyric acid
  SITE 6: note = Alpha methyl tyrosine
  SITE 25: note = Alpha methyl tyrosine
  VARIANT 47: note = Amidated 2-amino-6-(2-(cyclooct-2-yn-1-yloxy)acetamido)hexanamide or 2-amino-6-(2-(cyclooct-2-yn-1-yloxy)acetamido)hexanamide
YXEGTYTSDYSSYLEEQAAKEFIAYLVKGGGGGGGSGGGGSGGGGSK

SEQ ID NO: 35 - protein, 47 aa, synthetic construct; identical to SEQ ID NO: 15
  SITE 2: note = 2-aminoisobutyric acid
  SITE 6: note = Alpha methyl phenylalanine
  SITE 25: note = Alpha methyl tyrosine
  SITE 47: note = Amidated Methyl 5-(11,12-didehydrodibenzo[b,f]azoci n-5(6H)-yl)-5-oxopentanoate modified residue

SEQ ID NO: 36 - protein, 32 aa, synthetic construct
  SITE 2: note = 2-aminoisobutyric acid
  SITE 6: note = Alpha methyl phenylalanine
  SITE 25: note = Alpha methyl tyrosine
  SITE 32: note = Amidated azide modified residue
YXEGTFTSDYSSYLEEQAAKEFIAYLVEGGGK

SEQ ID NO: 37 - protein, 32 aa, synthetic construct
  SITE 2: note = 2-aminoisobutyric acid
  SITE 6: note = Alpha methyl phenylalanine
  SITE 25: note = Alpha methyl tyrosine
  SITE 32: note = Amidated azide modified residue
YXEGTFTSDYSSYLEEQAAKEFIEYLVEGGGK

SEQ ID NO: 38 - protein, 32 aa, synthetic construct
  SITE 2: note = 2-aminoisobutyric acid
  SITE 6: note = Alpha methyl phenylalanine
  SITE 20: note = 2-aminoisobutyric acid
  SITE 25: note = Alpha methyl tyrosine
  SITE 32: note = Amidated azide modified residue
YXEGTFTSDYSSYLEEQAAXEFIEYLVEGGGK

SEQ ID NO: 39 - protein, 31 aa, synthetic construct
  SITE 2: note = 2-aminoisobutyric acid
  SITE 3: note = (S)-2-amino-3-(2H-tetrazol-5-yl)propanoic acid
  SITE 31: note = Amidated residue
YXXGTFTSDYSSYLEEQAAKEFIAWLVKGGG

SEQ ID NO: 40 - protein, 47 aa, synthetic construct
  SITE 2: note = 2-aminoisobutyric acid
  SITE 3: note = (S)-2-amino-3-(2H-tetrazol-5-yl)propanoic acid
  SITE 6: note = 2-fluoro alpha methyl phenylalanine
  SITE 47: note = Amidated residue
YXXGTFTSDYSSYLEEQAAKEFIAWLVKGGGGGGGSGGGGSGGGGSK

SEQ ID NO: 41 - protein, 352 aa, Homo sapiens
NENSEQKENVEKEGLCNACTWRQNTKSSRIEAIKIQILSKLRLETAPNISKDVIRQLLPKAPPLRELIDQYDVQRDDSSDGSLEDDDYHATTETIITMPTESDFLMQVDGKPKCCFFKFSSKIQYNKVVKAQLWIYLRPVETPTTVFVQILRLIKPMKDGTRYTGIRSLKLDMNPGTGIWQSIDVKTVLQNWLKQPESNLGIEIKALDENGHDLAVTFPGPGEDGLNPFLEVKVTDTPKRRRRDFGLDCDEHSTESRCCRYPLTVDFEAFGWDWIIAPKRYKANYCSGECEFVFLQKYPHTHLVHQANPRGSAGPCCTPTKMSPINMLYFNGKEQIIYGKIPAMVVDRCGCS

SEQ ID NO: 42 - protein, 447 aa, synthetic construct
EVQVLESGGDLVQPGGSLRLSCAASGFTFSAYAMTWVRQAPGKGLEWVSAISGSGGSAYYADSVKGRFTISRDNSKNTVYLQMNSLRAEDTAVYYCAKDGAWKMSGLDVWGQGTTVIVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 43 - protein, 214 aa, synthetic construct
DIQMTQSPASLSASVGDRVTITCRASQDISDYLAWYQQKPGKIPRLLIYTTSTLQSGVPSRFRGSGSGTDFTLTISSLQPEDVATYYCQKYDSAPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 44 - protein, 452 aa, synthetic construct
QVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCARDLLVRFLEWSHYYGMDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 45 - protein, 215 aa, synthetic construct
QSVLTQPPSASGTPGQRVTISCSGSSSNIGSNTVHWYQQLPGTAPKLLIYSDNQRPSGVPDRFSGSKSGTSASLAISGLQSEDEADYYCAAWDDSLNGVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 46 - protein, 445 aa, synthetic construct
QVQLVQSGAEVKKPGASVKVSCKASGYTFTSSYINWVRQAPGQGLEWMGTINPVSGSTSYAQKFQGRVTMTRDTSISTAYMELSRLRSDDTAVYYCARGGWFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 47 - protein, 217 aa, synthetic construct
QSALTQPASVSGSPGQSITISCTGTSSDVGSYNYVNWYQQHPGKAPKLMIYGVSKRPSGVSNRFSGSKSGNTASLTISGLQAEDEADYYCGTFAGGSYYGVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 48 - protein, 445 aa, synthetic construct
EVQLLESGGGLVQPGGSLRLSCAASGFTFSSYAMSWVRQAPGKGLEWVSTISSGGSYTSYPDSVKGRFTISRDNSKNTLYLQMNSLRAEDTAVYYCAKQDYAMNYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 49 - protein, 214 aa, synthetic construct
DIQMTQSPSSLSASVGDRVTITCKASQDVSTAVAWYQQKPGKAPKLLIYSASYRYTGVPSRFSGSGSGTDFTLTISSLQPEDFATYYCQQHYSTPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 50 - protein, 439 aa, synthetic construct
EVQLVESGGGLVQPGGSLRLSCAASGLTFSRYPMSWVRQAPGKGLVWVSAITSSGGSTYYSDTVKGRFTISRDNAKNTLYLQMNSLRAEDTAVYYCARLPDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 51 - protein, 215 aa, synthetic construct
EIVLTQSPGTLSLSPGERATLSCRASSSVSSSYLHWYQQKPGQAPRLLIYSTSNLVAGIPDRFSGSGSGTDFTLTISRLEPEDFAVYYCQHHSGYHFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 52 - protein, 47 aa, synthetic construct; identical to SEQ ID NO: 12
  SITE 2: note = 2-aminoisobutyric acid
  SITE 47: note = Amidated 2-amino-6-(2-(cyclooct-2-yn-1-yloxy)acetamido)hexanamide

SEQ ID NO: 53 - protein, 32 aa, synthetic construct
  SITE 2: note = 2-aminoisobutyric acid
  SITE 32: note = Amidated residue linked to another sequence via the side chain
HXEGTFTSDYSSYLEEQAAKEFIAWLVKGGGK

SEQ ID NO: 54 - protein, 31 aa, synthetic construct
  SITE 2: note = 2-aminoisobutyric acid
  SITE 6: note = Alpha methyl phenylalanine
  SITE 25: note = Alpha methyl tyrosine
YXEGTFTSDYSSYLEEQAAKEFIAYLVKGGG

SEQ ID NO: 55 - protein, 5 aa, synthetic construct
LLQGG

SEQ ID NO: 56 - protein, 4 aa, synthetic construct
LLQG

SEQ ID NO: 57 - protein, 6 aa, synthetic construct
LSLSQG

SEQ ID NO: 58 - protein, 8 aa, synthetic construct
GGGLLQGG

SEQ ID NO: 59 - protein, 5 aa, synthetic construct
GLLQG

SEQ ID NO: 60 - protein, 10 aa, synthetic construct
GSPLAQSHGG

SEQ ID NO: 61 - protein, 7 aa, synthetic construct
GLLQGGG

SEQ ID NO: 62 - protein, 6 aa, synthetic construct
GLLQGG

SEQ ID NO: 63 - protein, 4 aa, synthetic construct
GLLQ

SEQ ID NO: 64 - protein, 8 aa, synthetic construct
LLQLLQGA

SEQ ID NO: 65 - protein, 5 aa, synthetic construct
LLQGA

SEQ ID NO: 66 - protein, 7 aa, synthetic construct
LLQYQGA

SEQ ID NO: 67 - protein, 6 aa, synthetic construct
LLQGSG

SEQ ID NO: 68 - protein, 6 aa, synthetic construct
LLQYQG

SEQ ID NO: 69 - protein, 7 aa, synthetic construct
LLQLLQG

SEQ ID NO: 70 - protein, 5 aa, synthetic construct
SLLQG

SEQ ID NO: 71 - protein, 5 aa, synthetic construct
LLQLQ

SEQ ID NO: 72 - protein, 6 aa, synthetic construct
LLQLLQ

SEQ ID NO: 73 - protein, 5 aa, synthetic construct
LLQGR

SEQ ID NO: 74 - protein, 125 aa, synthetic construct
QVQLVESGPGLVKPSETLSLTCTVYGGSISSGNYYWGWIRQPPGKGLEWIGTIYYSGSAYYNPSLKSRVTMSVDTSKNQFSLKLSSVTAADTAVYYCVRDYYDSSGHYYNWFDPWGQGTTVTVSS

SEQ ID NO: 75 - protein, 108 aa, synthetic construct
AIQMTQSPSSLSASVGDRVTITCRASQDIRHDLGWYQQKPGKAPKRLIYAASSLQSGVPSRFSGSGSGTEFTLTISSLQPEDFATYYCLQHNTYPWTFGQGTKVEIKR

SEQ ID NO: 76 - protein, 118 aa, synthetic construct
QVQLVESGGGVVQPGRSLRLSCAASGFTFSRYGIHWVRQAPGKGLEWVAVISYDGSDEYYVDSVKGRFSISRDNSKNTLYLQMNSLRPADSAVYYCVKGDLELGFDYWGQGTLVTVSS

SEQ ID NO: 77 - protein, 112 aa, synthetic construct
DIVMTQAAPSIPVIPGESVSMSCRSSKSLLYSNGHTYVYWFVQRPGQSPQLLIYRMSNLASGVPDRFSGSGSGTAFTLRISRVEAEDVGVYYCMQNLEFPLTFGAGTKLELK

SEQ ID NO: 78 - protein, 122 aa, synthetic construct
EVQLLESGGGLVQPGGSLRLSCAASGFTSITYAMSWVRQAPGKGLEWVSAISVSGTNTYYADSVKGRFTISRDKSKNMLYLQMNSLRAEDTAVYYCAKDLLHNWKYGTFDIWGQGTMVTVSS

SEQ ID NO: 79 - protein, 107 aa, synthetic construct
EIVMTQSPATLSVSPGERATLSCRASQSVDSNLVWYQQKPGQVPRLLIYGASTRATGIPARFSGSGSGTEFTLTISSLQSEDFAVYYCQQYNKWPLTFGGGTKVEIK

SEQ ID NO: 80 - protein, 120 aa, synthetic construct; identical to SEQ ID NO: 4

SEQ ID NO: 81 - protein, 107 aa, synthetic construct; identical to SEQ ID NO: 5

SEQ ID NO: 82 - protein, 121 aa, synthetic construct
QVQLVESGGGVVQPGKSLRLSCAASGFTFSSFGMHWVRQAPGKGLEWVAVIGYDGGNEYYADSVKGRFTISRDNSKNTLNLQMSSLRAEDTAVYYCSTISHYDILSGMDVWGRGTTVTVSS

SEQ ID NO: 83 - protein, 108 aa, synthetic construct
DIQMTQSPSSVSASVGDRVTITCRASQGISNWLAWYQQKPGKAPKLLIFAASSLQSGVPSRFSGSASGTDFTLTINSLQPEDFATYYCQQANSFPLTFGGGTKVEIKR

SEQ ID NO: 84 - protein, 20 aa, synthetic construct
  VARIANT 1..20: note = This sequence may encompass 1-4 GGGGS repeating units
GGGGSGGGGSGGGGSGGGGS

SEQ ID NO: 85 - protein, 4 aa, synthetic construct
GGGS

SEQ ID NO: 86 - protein, 5 aa, synthetic construct
GGGGS

SEQ ID NO: 87 - protein, 8 aa, synthetic construct
GGGSGGGS

SEQ ID NO: 88 - protein, 10 aa, synthetic construct
GGGGSGGGGS

SEQ ID NO: 89 - protein, 12 aa, synthetic construct
GGGSGGGSGGGS

SEQ ID NO: 90 - protein, 15 aa, synthetic construct
GGGGSGGGGSGGGGS

SEQ ID NO: 91 - protein, 16 aa, synthetic construct
GGGSGGGSGGGSGGGS

SEQ ID NO: 92 - protein, 20 aa, synthetic construct
GGGGSGGGGSGGGGSGGGGS

SEQ ID NO: 93 - protein, 20 aa, synthetic construct
GGGSGGGSGGGSGGGSGGGS

SEQ ID NO: 94 - protein, 25 aa, synthetic construct
GGGGSGGGGSGGGGSGGGGSGGGGS

SEQ ID NO: 95 - protein, 24 aa, synthetic construct
GGGSGGGSGGGSGGGSGGGSGGGS

SEQ ID NO: 96 - protein, 30 aa, synthetic construct
GGGGSGGGGSGGGGSGGGGSGGGGSGGGGS

SEQ ID NO: 97 - protein, 25 aa, synthetic construct
  VARIANT 25: note = Lysine, azide modified lysine, 2-amino-6-(2-(cyclooct-2-yn-1-yloxy)acetamido)hexanamide, bromo acetyl modified lysine, 2'-maleimido modified lysine, or propanoyl-3'-maleimido modified lysine
  VARIANT 1..24: note = This region may encompass 1-6 GGGS repeating units
GGGSGGGSGGGSGGGSGGGSGGGSK

SEQ ID NO: 98 - protein, 31 aa, synthetic construct
  VARIANT 31: note = Lysine, azide modified lysine, 2-amino-6-(2-(cyclooct-2-yn-1-yloxy)acetamido)hexanamide, bromo acetyl modified lysine, 2'-maleimido modified lysine, or propanoyl-3'-maleimido modified lysine
  VARIANT 1..30: note = This region may encompass 1-6 GGGGS repeating units
GGGGSGGGGSGGGGSGGGGSGGGGSGGGGSK

SEQ ID NO: 99 - protein, 24 aa, synthetic construct
  VARIANT 1..24: note = This sequence may encompass 1-6 GGGS repeating units
GGGSGGGSGGGSGGGSGGGSGGGS

SEQ ID NO: 100 - protein, 30 aa, synthetic construct; identical to SEQ ID NO: 96
  VARIANT 1..30: note = This sequence may encompass 1-6 GGGGS repeating units

SEQ ID NO: 101 - protein, 21 aa, synthetic construct
  SITE 21: note = Bromo acetyl modified residue
  VARIANT 1..20: note = This region may encompass 1-4 GGGGS repeating units
GGGGSGGGGSGGGGSGGGGSK

SEQ ID NO: 102 - protein, 6 aa, synthetic construct
TSDYSS

SEQ ID NO: 103 - protein, 6 aa, synthetic construct
LEEQAA

SEQ ID NO: 104 - protein, 21 aa, synthetic construct
  SITE 21: note = (S)-2-amino-6-(4-oxo-4-(dibenzocyclooctyne)butanami do)hexanamide
  VARIANT 1..20: note = This region may encompass 1-4 GGGGS repeating units
GGGGSGGGGSGGGGSGGGGSK

SEQ ID NO: 105 - protein, 21 aa, synthetic construct
  VARIANT 21: note = (2S)-2-amino-6-(4-(2-(3-(cyclooct-2-yn-1-yloxy)prop anamido)X)-1H-1,2,3-triazol-1-yl)hexanamide, where X is methyl, ethyl, or propyl
  VARIANT 1..20: note = This region may encompass 1-4 GGGGS repeating units
GGGGSGGGGSGGGGSGGGGSK

SEQ ID NO: 106 - protein, 21 aa, synthetic construct
  VARIANT 1..20: note = This region may encompass 1-4 GGGGS repeating units
GGGGSGGGGSGGGGSGGGGSK

SEQ ID NO: 107 - protein, 21 aa, synthetic construct
  SITE 21: note = 2-amino-6-(2-(cyclooct-2-yn-1-yloxy)acetamido)hexan amide
  VARIANT 1..20: note = This region may encompass 1-4 GGGGS repeating units
GGGGSGGGGSGGGGSGGGGSK

SEQ ID NO: 108 - protein, 21 aa, synthetic construct
  SITE 21: note = 2-amino-6-(2-(cyclooct-2-yn-1-yloxy)acetamido)hexan amide
  VARIANT 1..20: note = This region may encompass 1-4 GGGGS repeating units
GGGGSGGGGSGGGGSGGGGSK

SEQ ID NO: 109 - protein, 21 aa, synthetic construct
  SITE 21: note = 2-amino-6-(2-(cyclooct-2-yn-1-yloxy)acetamido)hexan amide
  VARIANT 1..20: note = This region may encompass 1-4 GGGGS repeating units
GGGGSGGGGSGGGGSGGGGSK

SEQ ID NO: 110 - protein, 20 aa, synthetic construct
  VARIANT 1..20: note = This sequence may encompass 1-4 GGGGS repeating units
GGGGSGGGGSGGGGSGGGGS

SEQ ID NO: 111 - protein, 12 aa, synthetic construct
  SITE 12: note = Amidated residue
TFYKEFIAWLVK

SEQ ID NO: 112 - protein, 12 aa, synthetic construct
  SITE 2: note = 2-fluoro alpha methyl phenylalanine
  SITE 12: note = Amidated residue
TFYKEFIAWLVK

SEQ ID NO: 113 - protein, 13 aa, synthetic construct
  SITE 2: note = 2-fluoro alpha methyl phenylalanine
  SITE 13: note = Azide modified residue
TFYKEFIAWLVKK

SEQ ID NO: 114 - protein, 28 aa, synthetic construct
  SITE 2: note = 2-fluoro alpha methyl phenylalanine
  SITE 28: note = Azido lysine modified residue
TFYKEFIAWLVKGGGGSGGGGSGGGGSK

SEQ ID NO: 115 - protein, 13 aa, synthetic construct
  SITE 2: note = 2-fluoro alpha methyl phenylalanine
  SITE 13: note = Residue linked to another sequence via the side chain
TFYKEFIAWLVKK

SEQ ID NO: 116 - protein, 13 aa, synthetic construct
  SITE 2: note = 2-fluoro alpha methyl phenylalanine
  SITE 13: note = (2S)-2-amino-6-(4-((2-(cyclooct-2-yn-1-yloxy)acetam ido)methyl)-1H-1,2,3-triazol-1-yl)hexanamide
TFYKEFIAWLVKK

SEQ ID NO: 117 - protein, 13 aa, synthetic construct
  SITE 2: note = Alpha methyl phenylalanine
  SITE 3: note = Alpha methyl phenylalanine
  SITE 13: note = Azide modified residue
TFFKEFIAWLVKK

SEQ ID NO: 118 - protein, 13 aa, synthetic construct
  SITE 2: note = Alpha methyl phenylalanine
  SITE 9: note = Alpha methyl tyrosine
  SITE 13: note = Azide modified residue
SFYKEFIAYLVKK

SEQ ID NO: 119 - protein, 13 aa, synthetic construct
  SITE 2: note = Alpha methyl phenylalanine
  SITE 6: note = Alpha methyl phenylalanine
  SITE 13: note = Azide modified residue
TFYKEFIAWLVKK

SEQ ID NO: 120 - protein, 13 aa, synthetic construct
  SITE 2: note = Alpha methyl phenylalanine
  SITE 3: note = Alpha methyl tyrosine
  SITE 6: note = Alpha methyl phenylalanine
  SITE 13: note = Azide modified residue
TFYKEFIAWLVKK

SEQ ID NO: 121 - protein, 28 aa, synthetic construct
  SITE 2: note = Alpha methyl phenylalanine
  SITE 9: note = Alpha methyl tyrosine
  SITE 28: note = 2-amino-6-(2-(cyclooct-2-yn-1-yloxy)acetamido)hexan amide
TFYKEFIAYLVKGGGGSGGGGSGGGGSK

SEQ ID NO: 122 - protein, 13 aa, synthetic construct
  SITE 2: note = 2-fluoro alpha methyl phenylalanine
  SITE 9: note = Alpha methyl tyrosine
  SITE 13: note = Azide modified residue
TFYKEFIAYLVKK

SEQ ID NO: 123 - protein, 13 aa, synthetic construct
  SITE 2: note = Alpha methyl tyrosine
  SITE 9: note = Alpha methyl tyrosine
  SITE 13: note = Azide modified residue
TYYKEFIAYLVKK

SEQ ID NO: 124 - protein, 13 aa, synthetic construct
  SITE 2: note = Alpha methyl tyrosine
  SITE 13: note = Azide modified residue
TYYKEFIAWLVKK

SEQ ID NO: 125 - protein, 13 aa, synthetic construct
  SITE 2: note = Alpha methyl phenylalanine
  SITE 13: note = Azide modified residue
TFYKEFIAWLVKK

SEQ ID NO: 126 - protein, 13 aa, synthetic construct
  SITE 2: note = Alpha methyl phenylalanine
  SITE 9: note = Alpha methyl tyrosine
  SITE 13: note = Azide modified residue
TFYKEFIAYLVKK

SEQ ID NO: 127 - protein, 28 aa, synthetic construct
  SITE 2: note = Alpha methyl phenylalanine
  SITE 9: note = Alpha methyl tyrosine
  SITE 28: note = Bromo acetyl modified residue
TFYKEFIAYLVKGGGGSGGGGSGGGGSK

SEQ ID NO: 128 - protein, 13 aa, synthetic construct
  SITE 2: note = Alpha methyl phenylalanine
  SITE 9: note = Alpha methyl tyrosine
  SITE 13: note = Residue linked to another sequence via the side chain
TFYKEFIAYLVKK

SEQ ID NO: 129 - protein, 28 aa, synthetic construct
  SITE 2: note = Alpha methyl phenylalanine
  SITE 9: note = Alpha methyl tyrosine
TFYKEFIAYLVKGGGGSGGGGSGGGGSK

SEQ ID NO: 130 - protein, 13 aa, synthetic construct
  SITE 2: note = Alpha methyl tyrosine
  SITE 13: note = Residue linked to another sequence via the side chain
TYYKEFIAWLVKK

SEQ ID NO: 131 - protein, 13 aa, synthetic construct
  SITE 2: note = Alpha methyl tyrosine
  SITE 9: note = Alpha methyl tyrosine
  SITE 13: note = Residue linked to another sequence via the side chain
TYYKEFIAYLVKK

SEQ ID NO: 132 - protein, 28 aa, synthetic construct
  SITE 2: note = Alpha methyl phenylalanine
  SITE 9: note = Alpha methyl tyrosine
  SITE 28: note = (S)-2-amino-6-(4-oxo-4-(dibenzocyclooctyne)butanami do)hexanamide
TFYKEFIAYLVKGGGGSGGGGSGGGGSK

SEQ ID NO: 133 - protein, 12 aa, synthetic construct
  SITE 2: note = Alpha methyl phenylalanine
  SITE 9: note = Alpha methyl tyrosine
  SITE 12: note = Amidated residue
TFYKEFIAYLVK

SEQ ID NO: 134 - protein, 13 aa, synthetic construct
  SITE 2: note = Alpha methyl phenylalanine
  SITE 9: note = Alpha methyl tyrosine
  SITE 13: note = Azide modified residue
TFYKEFIAYLVEK

SEQ ID NO: 135 - protein, 13 aa, synthetic construct
  SITE 2: note = Alpha methyl phenylalanine
  SITE 9: note = Alpha methyl tyrosine
  SITE 13: note = Azide modified residue
TFYKEFIEYLVEK

SEQ ID NO: 136 - protein, 13 aa, synthetic construct
  SITE 2: note = Alpha methyl phenylalanine
  SITE 4: note = 2-aminoisobutyric acid
  SITE 9: note = Alpha methyl tyrosine
  SITE 13: note = Azide modified residue
TFYXEFIEYLVEK

SEQ ID NO: 137 - protein, 21 aa, synthetic construct
  SITE 21: note = 2-amino-6-(2-(cyclooct-2-yn-1-yloxy)acetamido)hexan amide
  VARIANT 1..20: note = This region may encompass 1-4 GGGGS repeating units
GGGGSGGGGSGGGGSGGGGSK

SEQ ID NO: 138 - protein, 21 aa, synthetic construct
  SITE 21: note = 2-amino-6-(2-(cyclooct-2-yn-1-yloxy)acetamido)hexan amide
  VARIANT 1..20: note = This region may encompass 1-4 GGGGS repeating units
GGGGSGGGGSGGGGSGGGGSK

SEQ ID NO: 139 - protein, 16 aa, synthetic construct
  SITE 1: note = 2-amino-6-(2-mercaptoacetamido)hexanamide
XGGGGSGGGGSGGGGS

SEQ ID NO: 140 - protein, 16 aa, synthetic construct
  SITE 1: note = 2-amino-6-(2-((1-(4-(aminomethyl)phenyl)-4-methyl-5 ,6,7,8,9,10-hexahydrocycloocta[d]pyridazin-5-yl)oxy)acetam ido)hexanamide
XGGGGSGGGGSGGGGS

SEQ ID NO: 141 - protein, 16 aa, synthetic construct
  SITE 1: note = 2-amino-6-(2-((1-(4-(aminomethyl)phenyl)-4-methyl-5 ,6,7,8,9,10-hexahydrocycloocta[d]pyridazin-5-yl)oxy)acetam ido)hexanamide
  SITE 16: note = S-3-(1-(5,6-diamino-6-oxohexyl)-1H-1,2,3-triazol-4- yl)propanoic acid modified residue
XGGGGSGGGGSGGGGS

SEQ ID NO: 142 - protein, 16 aa, synthetic construct
  SITE 1: note = Pent-4-ynamide modified residue
  SITE 16: note = 2-amino-6-(2-(cyclooct-2-yn-1-yloxy)acetamido)hexan amide
GGGGSGGGGSGGGGSK

SEQ ID NO: 143 - protein, 16 aa, synthetic construct
  SITE 16: note = 2-amino-6-(2-(cyclooct-2-yn-1-yloxy)acetamido)hexan amide
GGGGSGGGGSGGGGSK

SEQ ID NO: 144 - protein, 16 aa, synthetic construct
  SITE 16: note = 2-amino-6-(2-(cyclooct-2-yn-1-yloxy)acetamido)hexan amide
GGGGSGGGGSGGGGSK

SEQ ID NO: 145 - protein, 16 aa, synthetic construct
  SITE 16: note = 2-amino-6-(2-((1-(4-(aminomethyl)phenyl)-4-methyl-5 ,6,7,8,9,10-hexahydrocycloocta[d]pyridazin-5-yl)oxy)acetam ido)hexanamide
GGGGSGGGGSGGGGSX

SEQ ID NO: 146 - protein, 16 aa, synthetic construct
  SITE 1: note = 2-amino-6-(2-((1-(4-(aminomethyl)phenyl)-4-methyl-5 ,6,7,8,9,10-hexahydrocycloocta[d]pyridazin-5-yl)oxy)acetam ido)hexanamide
XGGGGSGGGGSGGGGS

SEQ ID NO: 147 - protein, 21 aa, synthetic construct
  SITE 21: note = 2-amino-6-(2-(cyclooct-2-yn-1-yloxy)acetamido)hexan amide
  VARIANT 1..20: note = This region may encompass 1-4 GGGGS repeating units
GGGGSGGGGSGGGGSGGGGSK

SEQ ID NO: 148 - protein, 16 aa, synthetic construct
  SITE 1: note = 2-amino-6-(2-((4-(4-(aminomethyl)phenyl)-1-methyl-5 ,6,7,8,9,10-hexahydrocycloocta[d]pyridazin-5-yl)oxy)acetam ido)hexanamide
XGGGGSGGGGSGGGGS

SEQ ID NO: 149 - protein, 16 aa, synthetic construct
  SITE 1: note = 2-amino-6-(2-((4-(4-(aminomethyl)phenyl)-1-methyl-5 ,6,7,8,9,10-hexahydrocycloocta[d]pyridazin-5-yl)oxy)acetam ido)hexanamide
  SITE 16: note = S-3-(1-(5,6-diamino-6-oxohexyl)-1H-1,2,3-triazol-4- yl)propanoic acid modified residue
XGGGGSGGGGSGGGGS

SEQ ID NO: 150 - protein, 16 aa, synthetic construct
  SITE 16: note = 2-amino-6-(2-(cyclooct-2-yn-1-yloxy)acetamido)hexan amide
GGGGSGGGGSGGGGSX

SEQ ID NO: 151 - protein, 16 aa, synthetic construct
  SITE 16: note = 2-amino-6-(2-(cyclooct-2-yn-1-yloxy)acetamido)hexan amide
GGGGSGGGGSGGGGSX

SEQ ID NO: 152 - protein, 16 aa, synthetic construct
  SITE 16: note = 2-amino-6-(2-(cyclooct-2-yn-1-yloxy)acetamido)hexan amide
GGGGSGGGGSGGGGSX

SEQ ID NO: 153 - protein, 16 aa, synthetic construct
  SITE 16: note = 2-amino-6-(2-(cyclooct-2-yn-1-yloxy)acetamido)hexan amide
GGGGSGGGGSGGGGSX

(19) United States
(12) Patent Application Publication
(10) Pub. No.: US 2026/0315614 A1
(43) Pub. Date: Oct. 15, 2026

(54) GLP1R AND GIPR DUAL AGONISTS AND ANTIBODY CONJUGATES THEREOF

(71) Applicant: Regeneron Pharmaceuticals, Inc., Tarrytown, NY (US)

(72) Inventors: Amy HAN, Hockessin, DE (US); Chester A. METCALF, III, Briarcliff Manor, NY (US); William OLSON, Yorktown Heights, NY (US); Jason MASTAITIS, Yorktown Heights, NY (US); Judith ALTAREJOS, Armonk, NY (US); Mark W. SLEEMAN, New York, NY (US); Andrew J. MURPHY, Croton-on-Hudson, NY (US); Xiang ZHENG, Allendale, NJ (US); Ricardo LARANJEIRO, Irvington, NY (US); Frank DELFINO, Poughquag, NY (US); George D. YANCOPOULOS, Yorktown Heights, NY (US)

(73) Assignee: Regeneron Pharmaceuticals, Inc., Tarrytown, NY (US)

(21) Appl. No.: 19/637,645

(22) Filed: Apr. 2, 2026

Related U.S. Application Data
(60) Provisional application No. 63/782,751, filed on Apr. 3, 2025.
(60) Provisional application No. 63/818,878, filed on Jun. 6, 2025.

(51) Int. Cl. A61K 47/68; A61P 3/04; C07K 1/107

(52) U.S. Cl. CPC A61K 47/6811; A61K 47/6845; A61P 3/04; C07K 1/1077

(57) ABSTRACT

Described herein are series GLP1R-GIPR dual agonist peptides, protein-peptide conjugates, and pharmaceutical compositions thereof, that are useful for the treatment of diabetes, obesity, and metabolic related disorders and diseases. Those protein-peptide conjugates simultaneously engage and target glucagon-like peptide-1 (GLP-1) receptor (GLP1R), glucose-dependent insulinotropic polypeptide (GIP) receptor (GIPR), and myostatin/growth and differentiation factor-8 (GDF8) pathways, which preserves and/or maintains muscle weight while substantially reduces body weight and fat mass. More specifically, described herein are series of GLP1R and GIPR dual agonists, their conjugates with anti-myostatin (growth and differentiation factor-8 (GDF8)) antibody, methods of uses, and methods of making the same.

### CROSS-REFERENCE TO RELATED APPLICATIONS

This application claims the priority benefit of U.S. Provisional Patent Application Ser. No. 63/818,878, filed Jun. 6, 2025, and U.S. Provisional Patent Application Ser. No. 63/782,751, filed Apr. 3, 2025, each of which is hereby incorporated by reference in its entirety.

### SEQUENCE LISTING

The instant application contains a Sequence Listing which has been submitted electronically in XML format and is hereby incorporated by reference in its entirety. Said XML copy, created on Mar. 31, 2026, is named 250298_001223_SL.xml and is 230,804 bytes in size.

### FIELD OF THE DISCLOSURE

The present disclosure relates to protein-ligand conjugates (e.g., antibody-ligand conjugates, or antibody-drug conjugates), processes of manufacturing, pharmaceutical compositions, and methods of treating diseases therewith. More specifically, the present disclosure relates to GLP1R and GIPR (GLP1R-GIPR) dual agonists and GDF8 antibody conjugates, processes of manufacturing, and methods of treating GDF8, GLP1R, and GIPR associated conditions therewith. The singular molecule entity disclosed herein targets and engages all three targets GLP1R, GIPR, and growth and differentiation factor-8 (GDF8), which substantially reduces body weight while maintaining or increasing lean body mass.

### BACKGROUND OF THE DISCLOSURE

Obesity and the closely associated type 2 diabetes are a global problem for over a third of the world population. In the United States of America, the average obesity rate is over 20%. The costs of obesity-related illness are staggering, amounting to $190.2 billion, roughly 21% of annual medical costs in the U.S. Obesity is an epidemic disease characterized by chronic low-grade inflammation associated with dysfunctional (elevated) fat mass. Obesity is an important underlying risk factor for developing other diseases such as heart disease, stroke, and diabetes. Even a modest decrease in body weight (5-10% of initial body weight) lowers the risk for developing obesity-associated diseases such as heart disease and diabetes.

Glucagon-Like Peptide 1 Receptor (GLP1R) is the receptor for glucagon-like peptide 1 (GLP1) and is expressed in the pancreatic beta cells. GLP1R is also expressed in the brain where it functions in the control of appetite, memory, and learning. GLP1R is a member of the secretin family (Class B) of G protein-coupled receptors (GPCRs). Upon binding of its ligand, GLP1, GLP1R initiates a downstream signaling cascade through Gas G-proteins that raises intracellular cyclic AMP (cAMP) levels, which leads to the transcriptional regulation of genes (Donnelly, Br J Pharmacol, 166(1):27-41 (2011)). Activation of GLP1R results in increased insulin synthesis and release of insulin. GLP1R and GLP1 are highly validated targets for obesity and type 2 diabetes. Marketed GLP1R agonists have shown a remarkable efficacy in reducing bodyweight. However, those therapeutics, while effective in reducing the bodyweight, are also accompanied with significant loss of lean muscle mass. And the lost weights might return once the medication is withdrawn. There are still unmet needs for efficient management of obesity and diabetes using GLP1R agonist.

GLP-1 analogues, fusion proteins and GLP-1 receptor agonists are disclosed, for example, in U.S. Pat. Nos. 7,452,966, 8,389,689, 8,496,149, 8,497,240, 8,557,769, 8,883,447, 8,895,694, 9,409,966, US20160194371, US20140024586, US20140073563, US20120148586, US20170114115, US20170112904, US20160361390, US20150313908, US20150259416, WO2017074715, WO2016127887, WO2015021871, WO2014113357, EP3034514, EP2470198, and EP2373681.

Another incretin, glucose-dependent insulinotropic polypeptide (GIP) is secreted from the enteroendocrine K cells in the small intestine following food ingestion. Studies have shown that GIP analogs reduce body weight (BW), and a GIP/GLP-1 co-agonist augments incretin response and BW reduction compared with GLP-1R agonist in rodents and in clinical trials (Coskun, T., et al. (2018) Mol. Metab. 18, 3-14; Frias, J. P., et al. (2018), Lancet 392, 2180-2193).

Nevertheless, a need remains for treatments of diabetes and obesity with a more favorable side effect profile, such as maintaining and preserving lean mass while providing a significant body weight reduction.

Growth and Differentiation Factor-8 (GDF8), also known as myostatin, is a member of the TGF-β superfamily of growth factors. GDF8 plays a central role in the development and maintenance of skeletal muscle, acting as a negative regulator of muscle mass. GDF8 is highly conserved across species, and the amino acid sequences of murine and human GDF8 are identical (human GDF8 nucleic acid sequence and amino acid sequence shown in SEQ ID NO:338-339) (McPherron et al. 1977 Nature 387:83-90).

A number of human diseases are associated with loss or impairment of muscle tissue, for example, muscular dystrophy, muscle atrophy, muscle wasting syndrome, sarcopenia and cachexia, and inhibitors of GDF8 are applicable to treat these diseases or disorders. While the myostatin null mouse phenotype demonstrates the importance of GDF8 in the control of muscle size during development, muscle hypertrophy can also be elicited in adult muscle through inhibition of GDF8 with neutralizing antibodies, decoy receptors, or other antagonists.

Administration of GDF8 neutralizing antibodies has been reported to result in muscle mass increases of between 10 and 30%. The increased muscle mass seen is due to increased fiber diameter as opposed to myofiber hyperplasia (fiber number). A number of studies have also reported increases in muscle strength or performance commensurate with increased size including twitch and tetanic force.

Antibodies to Growth and Differentiation Factor-8 (GDF8), also known as myostatin, are known in the art. Antibodies to GDF8 and therapeutic methods are disclosed in, e.g., U.S. Pat. No. 8,840,894. Anti-GDF8 antibodies are also mentioned in, e.g., U.S. Pat. Nos. 6,096,506; 7,320,789; 7,261,893; 7,807,159; 7,888,486; 7,635,760; 7,632,499; in US Patent Appl. Publ. Nos. 2006/0263354; 2007/0178095; 2008/0299126; 2010/0166764; 2009/0148436; and International Patent Appl. Publ. Nos. WO2004/037861; WO2006/116269; WO2012/024242; WO2014/144903; WO2007/047112; WO 2010/070094; WO2011/151432; WO2010/070094; WO2023/187022. Antibodies to GDF8 and therapeutic methods are disclosed in, e.g., U.S. Pat. Nos. 6,096,506, 7,320,789, 7,807,159, WO 2007/047112, WO 2005/094446, US 2007/0087000, U.S. Pat. No. 7,261,893, WO 2010/070094, U.S. Pat. No. 8,840,894, WO2013006437, and U.S. Pat. No. 9,260,515.

Additional examples include Abrilumab (AMG 181, WO2010107752), Apitegromab (SRK-015, WO2016073879 and WO2016073879), Domagrozumab (PF-06252616, WO2013186719), GYM329 (Chugai Pharmaceutical Co. and Hoffmann-La Roche, Inc., RO7204239), Landogrozumab (LY2495655, WO2005094446, WO2007044411, WO2007047112, and WO2008030706), Stamulumab (MYO-029, WO2003027248, WO2004037861, WO2009058346, and WO2007024535), and Trevogrumab (REGN1033, WO2011150008).

The above-noted patents and patent applications are incorporated herein by reference in their entirety.

The foregoing discussion is presented solely to provide a better understanding of the nature of the problems confronting the art and should not be construed in any way as an admission as to prior art nor should the citation of any reference herein be construed as an admission that such reference constitutes “prior art” to the instant application.

### SUMMARY OF THE DISCLOSURE

Various non-limiting aspects and embodiments of the disclosure are described below.

The current disclosure describes GLP1R agonist ligand tethered GDF8 antibody that can be used for obesity management without significant loss of lean mass and offer much improvement in clinical outcomes.

In one aspect, provided herein is a conjugate, or a pharmaceutically acceptable salt thereof, comprising an antigen-binding protein (ABP) and at least one glucagon-like peptide-1 (GLP-1) receptor (GLP1R) and glucose-dependent insulinotropic polypeptide (GIP) receptor (GIPR) dual agonist (GLP1R-GIPR dual agonist), wherein the GLP1R and GIPR dual agonist is covalently conjugated to the ABP through a linker.

In some embodiments, the ABP is an antibody, or an antigen binding fragment thereof, that specifically binds to and/or blocks the activity of growth and differentiation factor-8 (GDF8).

In some embodiments, the ABP is an antibody that specifically binds to and/or blocks the activity of growth and differentiation factor-8 (GDF8) having a sequence of SEQ ID NO: 1.

In some embodiments, the GDF8 antibody, or the antigen-binding fragment thereof, comprises three heavy chain complementarity determining regions (HCDR1, HCDR2, and HCDR3) comprising SEQ ID NO: 6, SEQ ID NO: 7, and SEQ ID NO: 8, respectively, and three light chain complementarity determining regions (LCDR1, LCDR2, and LCDR3) comprising SEQ ID NO: 9, SEQ ID NO: 10, and SEQ ID NO: 11, respectively.

In some embodiments, the GDF8 antibody, or the antigen-binding fragment thereof, comprises heavy chain complementarity determining regions (HCDRs) of a heavy chain variable region (HCVR) comprising SEQ ID NO:4, and light chain complementarity determining regions (LCDRs) of a light chain variable region (LCVR) comprising SEQ ID NO:5.

In some embodiments, the GDF8 antibody, or the antigen binding fragment thereof, comprises 1A2, 21-E5, 8D12, H4H1669P, AMG 181, AMG 745, Apitegromab (SRK-015), Bimagrumab (BYM338), Domagrozumab (PF-06252616), GYM329 (RO7204239), Landogrozumab (LY2495655), Stamulumab (MYO-029), and Trevogrumab (REGN1033).

In some embodiments, the GLP1R and GIPR dual agonist comprises a sequence of Tyr1 Aib2 XX3 Gly4 XX5 XX6 Thr Ser Asp Tyr Ser Ser XX13 Leu Glu Glu Gln Ala Ala XX20 Glu XX22 Ile XX24 XX25 Leu Val XX28 Gly Gly Gly (SEQ ID NO: 3), wherein Aib represents 2-aminoisobutyric acid (α-aminoisobutyric acid); XX3 is Glu or Atz ((S)-2-amino-3-(2H-tetrazol-5-yl)propanoic acid); XX5 is Ser or Thr; XX6 is phenylalanine, alpha methyl phenylalanine (amF) or 2-fluoro alpha methyl phenylalanine (amF(2-fluoro)); XX13 is Tyr, amF, or alpha methyl tyrosine (amY); XX20 is Lys, sidechain acylated or alkylated Lys, Gln, His, or Aib; XX22 is Phe or amF; XX24 is Ala or Glu; XX25 is tryptophan or alpha methyl tyrosine (amY); and XX28 is Lys, Ala, or Glu.

In some embodiments, the GLP1R-GIPR dual agonist comprises a sequence of: Tyr1 Aib2 XX3 Gly4 XX5 XX6 Thr Ser Asp Tyr Ser Ser XX13 Leu Glu Glu Gln Ala Ala XX20 Glu XX22 Ile XX24 XX25 Leu Val XX28 Gly Gly Gly (SEQ ID NO: 3), wherein Aib represents 2-aminoisobutyric acid (α-aminoisobutyric acid); XX3 is Glu or Atz ((S)-2-amino-3-(2H-tetrazol-5-yl)propanoic acid); XX5 is Ser or Thr; XX6 is phenylalanine, alpha methyl phenylalanine (amF) or 2-fluoro alpha methyl phenylalanine (amF(2-fluoro)); XX13 is amF or alpha methyl tyrosine (amY); XX20 is Lys, sidechain-acylated or alkylated Lys, or Aib; XX22 is Phe or amF; XX24 is Ala or Glu; XX25 is tryptophan or alpha methyl tyrosine (amY); and XX28 is Lys, Ala, Gln, or Glu.

In some embodiments, the GLP1R and GIPR dual agonist comprises a sequence of: Tyr1 Aib2 XX3 Gly4 XX5 XX6 Thr Ser Asp Tyr Ser Ser XX13 Leu Glu Glu Gln Ala Ala XX20 Glu XX22 Ile XX24 XX25 Leu Val XX28 Gly Gly Gly (SEQ ID NO: 3), wherein Aib represents 2-aminoisobutyric acid or (α-aminoisobutyric acid); XX3 is Glu or Atz ((S)-2-amino-3-(2H-tetrazol-5-yl)propanoic acid); XX5 is Ser or Thr; XX6 is phenylalanine, alpha methyl phenylalanine (amF) or 2-fluoro alpha methyl phenylalanine (amF(2-fluoro)); XX13 is Tyr, amF, or alpha methyl tyrosine (amY); XX20 is Lys, sidechain-acylated or alkylated Lys, or Aib; XX22 is amF; XX24 is Ala or Glu; XX25 is tryptophan or alpha methyl tyrosine (amY); and XX28 is Lys, Ala, Gln, or Glu.

In some embodiments, the GLP1R and GIPR dual agonist comprises a sequence of: Tyr1 Aib2 XX3 Gly4 XX5 XX6 Thr Ser Asp Tyr Ser Ser XX13 Leu Glu Glu Gln Ala Ala XX20 Glu XX22 Ile XX24 XX25 Leu Val XX28 Gly Gly Gly (SEQ ID NO: 3), wherein Aib represents 2-aminoisobutyric acid (α-aminoisobutyric acid); XX3 is Glu or Atz ((S)-2-amino-3-(2H-tetrazol-5-yl)propanoic acid); XX5 is Ser or Thr; XX6 is phenylalanine, alpha methyl phenylalanine (amF) or 2-fluoro alpha methyl phenylalanine (amF(2-fluoro)); XX13 is Tyr, amF, or alpha methyl tyrosine (amY); XX20 is Lys, sidechain-acylated or alkylated Lys, or Aib; XX22 is Phe or amF; XX24 is Glu; XX25 is tryptophan or alpha methyl tyrosine (amY); and XX28 is Lys, Ala, Gln, or Glu.

In some embodiments, the GLP1R and GIPR dual agonist comprises a sequence of: Tyr1 Aib2 XX3 Gly4 XX5 XX6 Thr Ser Asp Tyr Ser Ser XX13 Leu Glu Glu Gln Ala Ala XX20 Glu XX22 Ile XX24 XX25 Leu Val XX28 Gly Gly Gly (SEQ ID NO: 3), wherein Aib represents 2-aminoisobutyric acid (α-aminoisobutyric acid); XX3 is Glu or Atz ((S)-2-amino-3-(2H-tetrazol-5-yl)propanoic acid); XX5 is Ser or Thr; XX6 is phenylalanine, alpha methyl phenylalanine (amF) or 2-fluoro alpha methyl phenylalanine (amF(2-fluoro)); XX13 is Tyr, amF, or alpha methyl tyrosine (amY); XX20 is Lys, sidechain-acylated or alkylated Lys, or Aib; XX22 is Phe or amF; XX24 is Ala or Glu; XX25 is alpha methyl tyrosine (amY); and XX28 is Lys, Ala, Gln, or Glu.

In some embodiments, the GLP1R and GIPR dual agonist comprises a sequence of: Tyr1 Aib2 XX3 Gly4 XX5 XX6 Thr Ser Asp Tyr Ser Ser XX13 Leu Glu Glu Gln Ala Ala XX20 Glu XX22 Ile XX24 XX25 Leu Val XX28 Gly Gly Gly (SEQ ID NO: 3), wherein Aib represents 2-aminoisobutyric acid (α-aminoisobutyric acid); XX3 is Glu or Atz ((S)-2-amino-3-(2H-tetrazol-5-yl)propanoic acid); XX5 is Ser or Thr; XX6 is phenylalanine, alpha methyl phenylalanine (amF) or 2-fluoro alpha methyl phenylalanine (amF(2-fluoro)); XX13 is Tyr, amF, or alpha methyl tyrosine (amY); XX20 is Lys, sidechain-acylated or alkylated Lys, or Aib; XX22 is Phe or amF; XX24 is Ala or Glu; XX25 is tryptophan or alpha methyl tyrosine (amY); and XX28 is Ala, Gln, or Glu.

In some embodiments, XX13 is amF or alpha methyl tyrosine (amY).

In some embodiments, XX22 is amF.

In some embodiments, XX24 is Ala or Glu.

In some embodiments, the GLP1R-GIPR dual agonist has a sequence selected from the group consisting of SEQ ID NOs: 3, 14-17, and 19-54.

In some embodiments, the linker covalently linking the GLP1R and GIPR dual agonist with ABP comprises L1-L2-L3-L4, where L1 is an optional spacer; L2 comprises a first reactive moiety; L3 comprises a second reactive moiety; and L4 is an optional spacer; and where the first and the second reactive moieties form a covalent bond during the conjugation process.

In some embodiments, the first and second reactive moieties are bio-orthogonal reactive moieties compatible for conjugation in an aqueous medium/environment.

In some embodiments, the first and second reactive moieties are bio-orthogonal reactive moieties for Click reaction, Diels-Alder reaction, Inverse electron deficient Diels-Alder reaction (IEDDA), and highly selective nucleophilic reaction involving a thiol group or a primary amino group.

In some embodiments, the first and second reactive moieties comprise a primary amino group and a glutamine side chain that are compatible for conjugation catalyzed by a microbial transglutaminase.

In some embodiments, the spacer L1 or L2 comprises (PEG)n or a short peptide comprising a plurality of Gly and Ser residues, wherein n is one to 12.

In some embodiments, the spacer L1 or L2 comprises —NH-(PEG)n-CO— or a short peptide comprising multiple (GlyGlyGlyGlySer)m, wherein n is one to 12 and m is one to four (SEQ ID NO: 84).

In some embodiments, the linker comprises

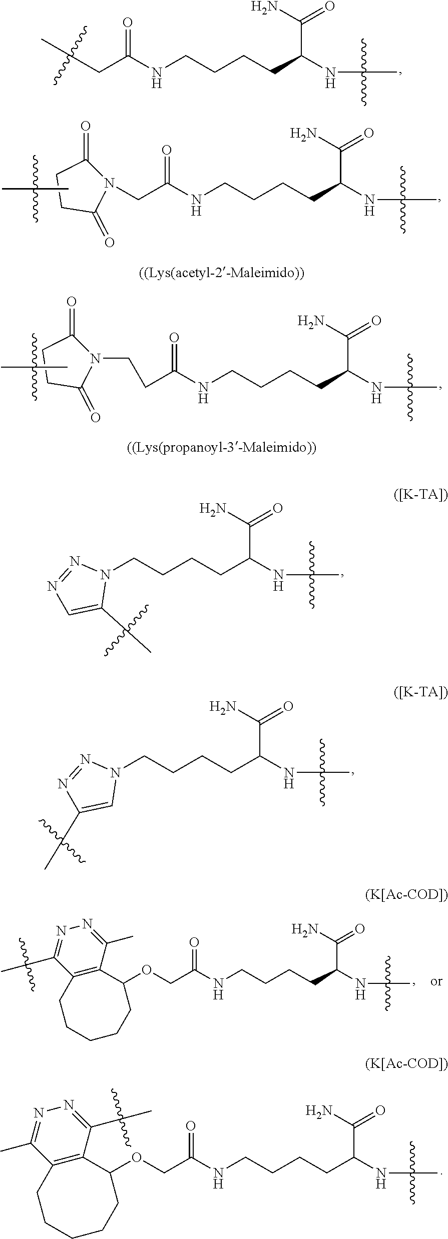

In some embodiments, the linker comprises

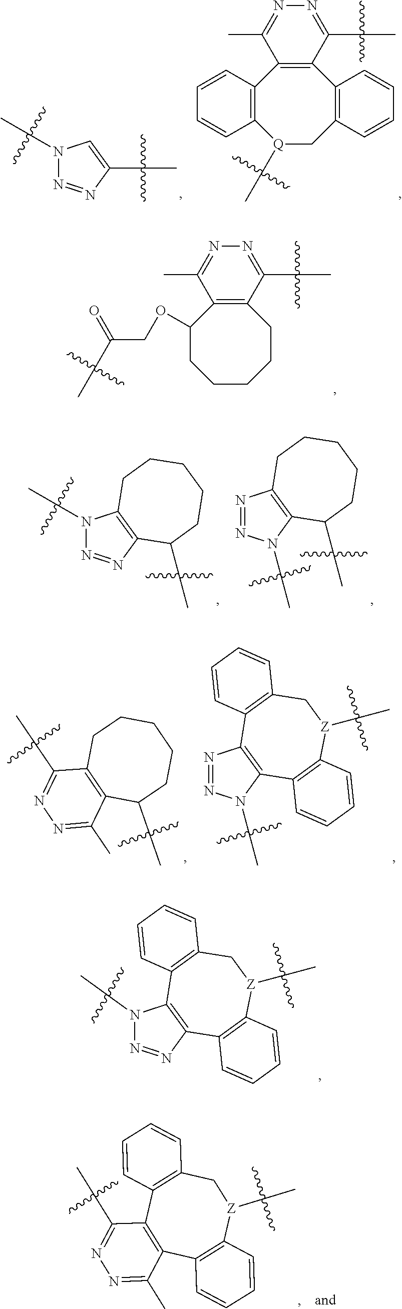

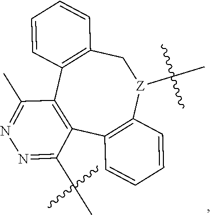

where Z is CH or N.

In some embodiments, the linker comprises a sequence selected from the group consisting of Gly₃Ser (SEQ ID NO: 85), Gly₄Ser (SEQ ID NO: 86), (Gly₃Ser)₂ (SEQ ID NO: 87), (Gly4Ser)₂ (SEQ ID NO: 88), (Gly3Ser)₃ (SEQ ID NO: 89), (Gly4Ser)₃ (SEQ ID NO: 90), (Gly3Ser)₄ (SEQ ID NO: 91), (Gly4Ser)₄ (SEQ ID NO: 92), (Gly3Ser)₅ (SEQ ID NO: 93), (Gly4Ser)₅ (SEQ ID NO: 94), (Gly3Ser)₆ (SEQ ID NO: 95), and (Gly4Ser)₆ (SEQ ID NO: 96).

In some embodiments, the linker comprises a sequence of -(Gly₃Ser)ₙ-Lys(X)- (SEQ ID NO: 97) or -(Gly₄Ser)ₙ-Lys(X)- (SEQ ID NO: 98), wherein n is an integer from one to six; and wherein X is a reactive moiety attached to the side chain of Lys for conjugation with the ABP.

In some embodiments, X is a reactive moiety comprising an azido, 2-bromo-acetyl,

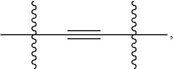 [SMILES: *C#C*]

2-(cyclooct-2-yn-lyloxy) acetyl, 2′-maleimido-acetyl, and 3′-maleimido-propanoyl.

In some embodiments, the first and second reactive moieties are bio-orthogonal reactive moieties comprising thiol, maleimido, 2-bromo acetyl, —N₃,

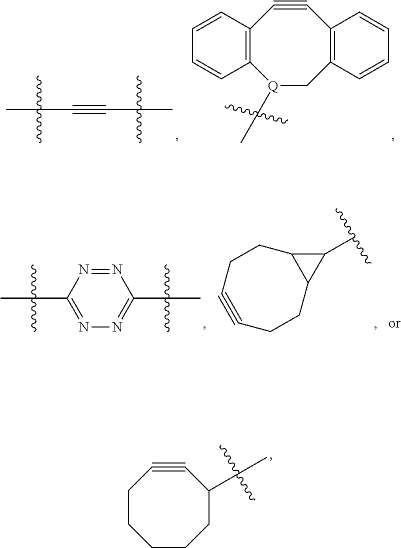

where Q is CH or N.

In some embodiments, the first and second reactive moieties are bio-orthogonal reactive moieties comprising: amino, thiol,

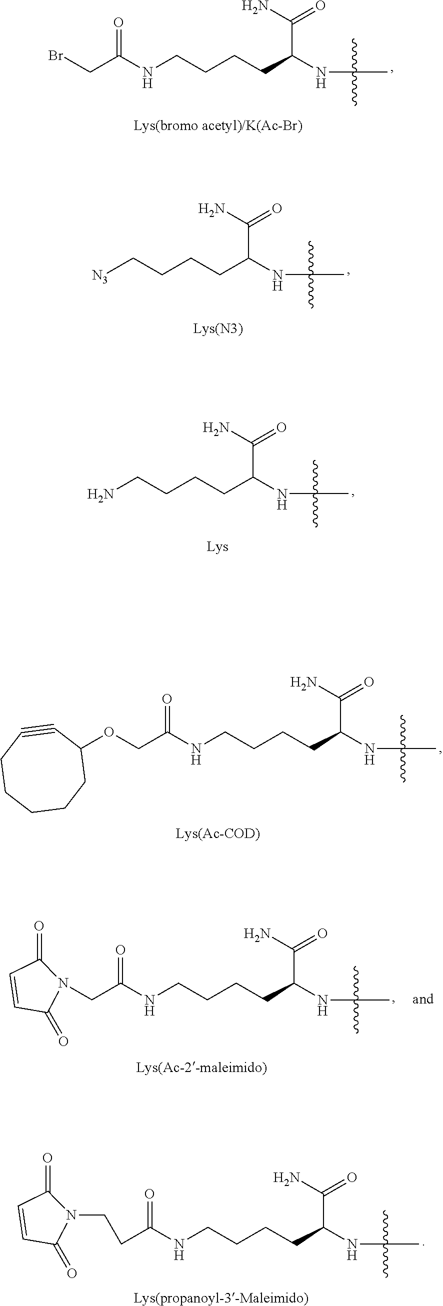

In some embodiments, the linker comprises

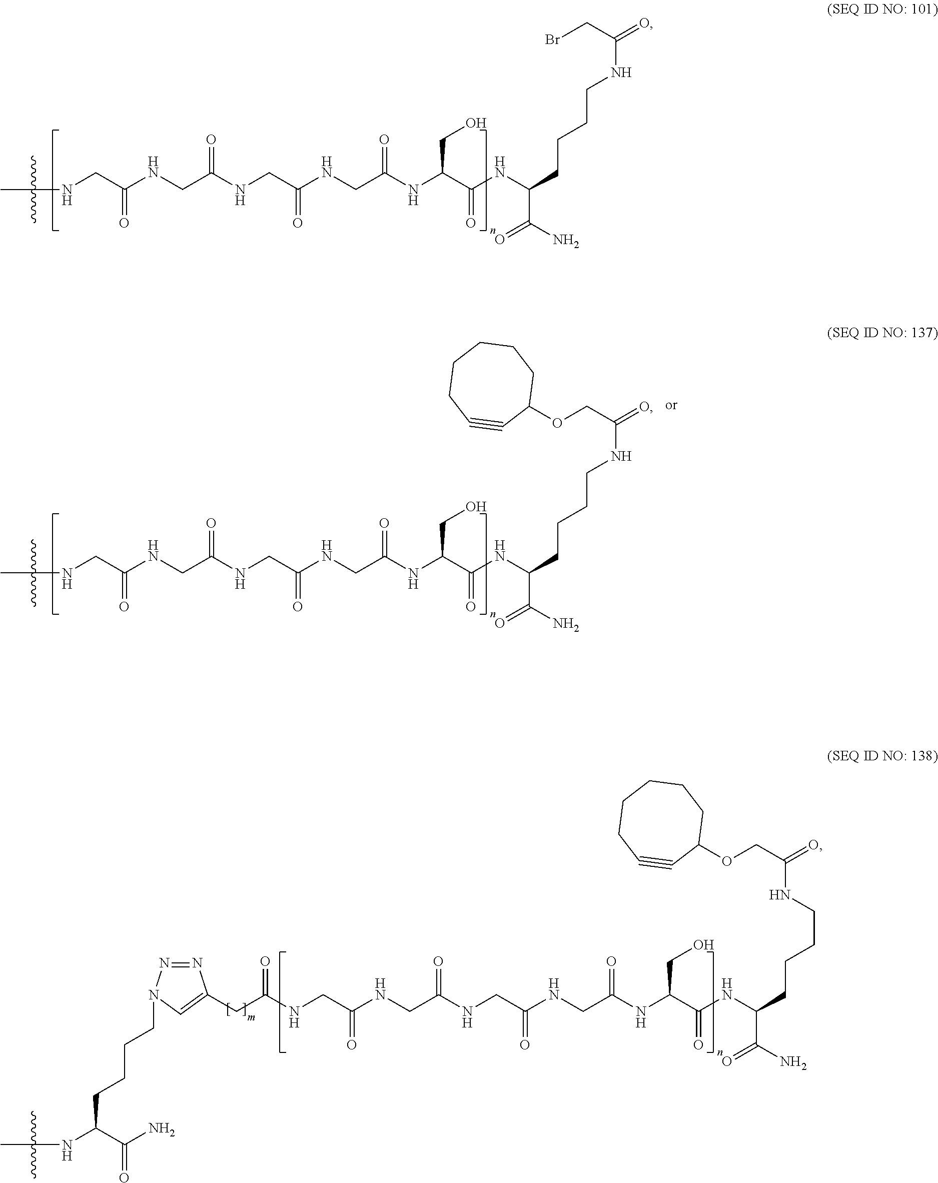

wherein m is an integer of one to three; and n is an integer of one to four.

In some embodiments, the GLP1R and GIPR dual agonist binds to and activates the signaling cascade of GLP1R and GIPR.

In some embodiments, the GLP1R and GIPR dual agonist binds to and activates the signaling cascade of GLP1R and GIPR, and wherein the GLP1R and GIPR dual agonist does not substantially bind to and/or activates the signaling cascade of glucagon receptor.

In some embodiments, the GLP1R and GIPR dual agonist binds to and activates the signaling cascade of GLP1R and GIPR, and wherein said GLP1R and GIPR dual agonist does not substantially bind to and/or activates the signaling cascade of GLP2R.

In some embodiments, the conjugate binds to and activates the signaling cascade of GLP1R and GIPR.

In some embodiments, the conjugate binds to and activates the signaling cascade of GLP1R and GIPR, and the conjugate does not substantially bind to and/or activates the signaling cascade of glucagon peptide receptor.

In some embodiments, the conjugate binds to and activates the signaling cascade of GLP1R and GIPR, and said conjugate does not substantially bind to and/or activates the signaling cascade of GLP2R.

In some embodiments, the conjugate binds to and activates the signaling cascade of GLP1R and GIPR and the conjugate binds to and/or blocks the activity of growth and differentiation factor-8 (GDF8).

In some embodiments, the conjugate binds to and activates the signaling cascade of GLP1R and GIPR and the conjugate binds to and/or blocks the activity of growth and differentiation factor-8 (GDF8) having a sequence of SEQ ID NO: 1.

In some embodiments the conjugate has a structure of

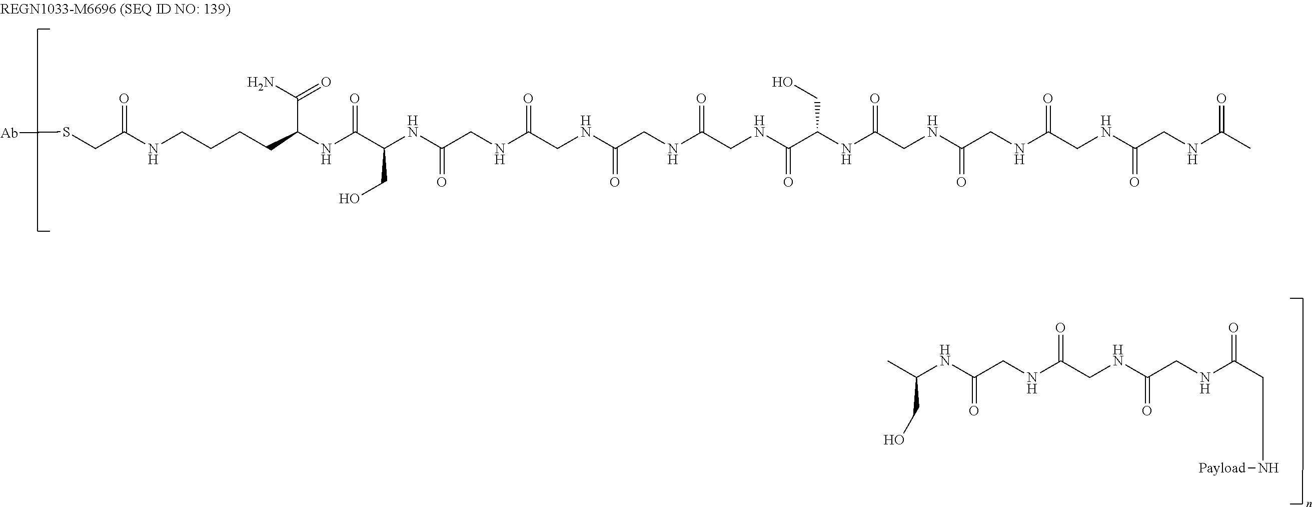

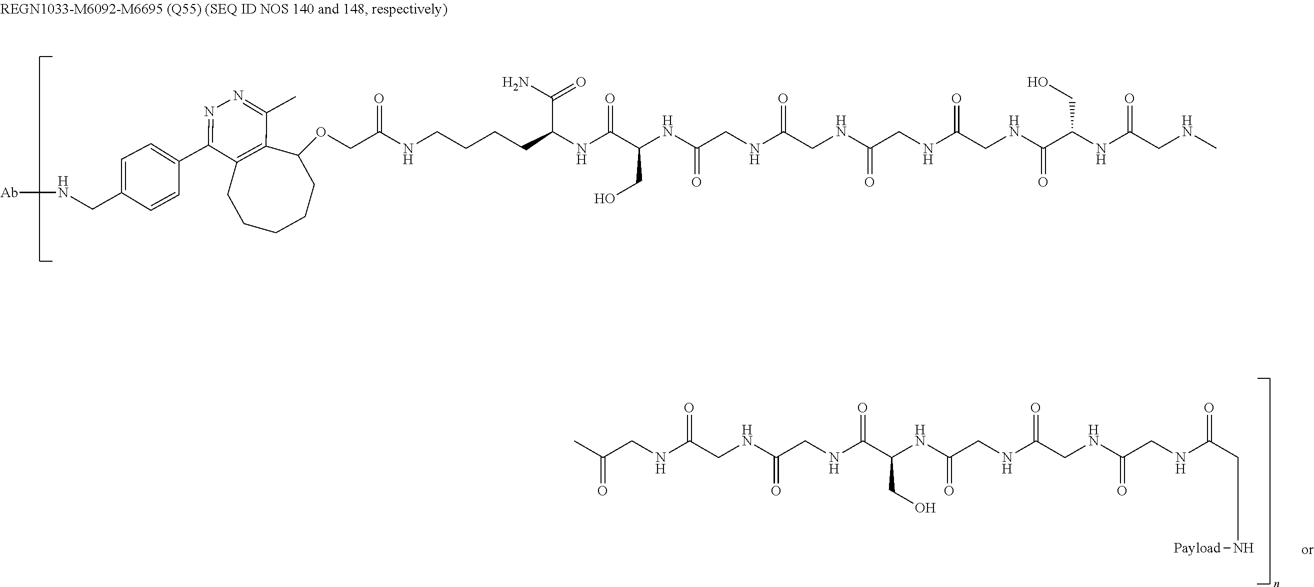

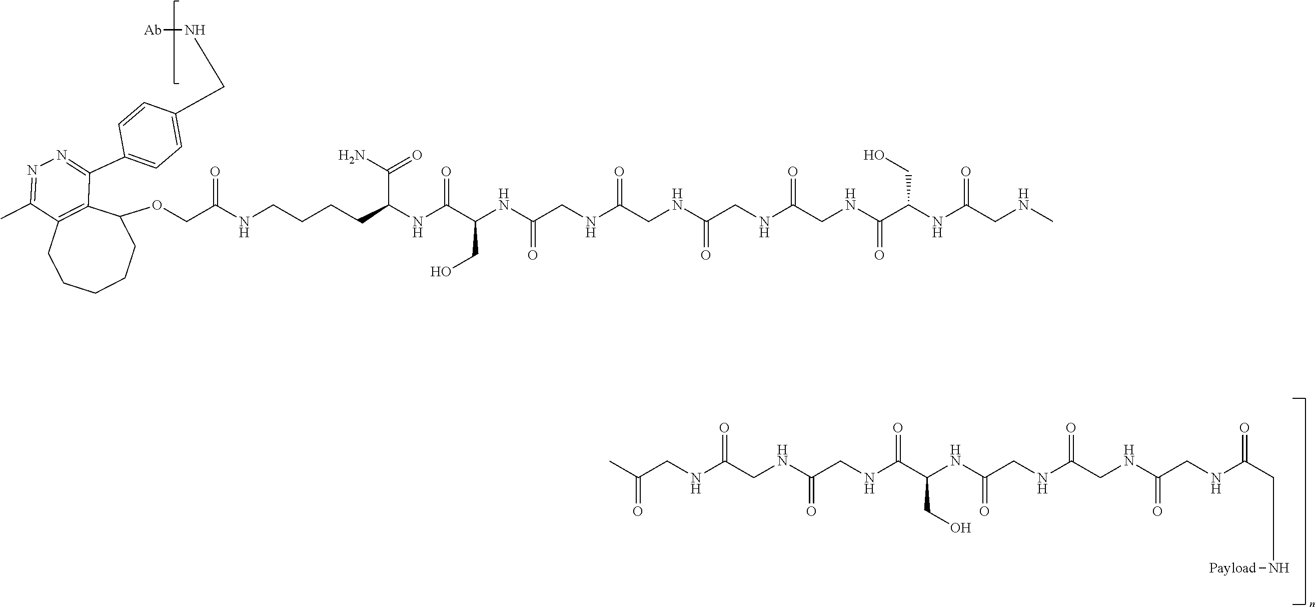

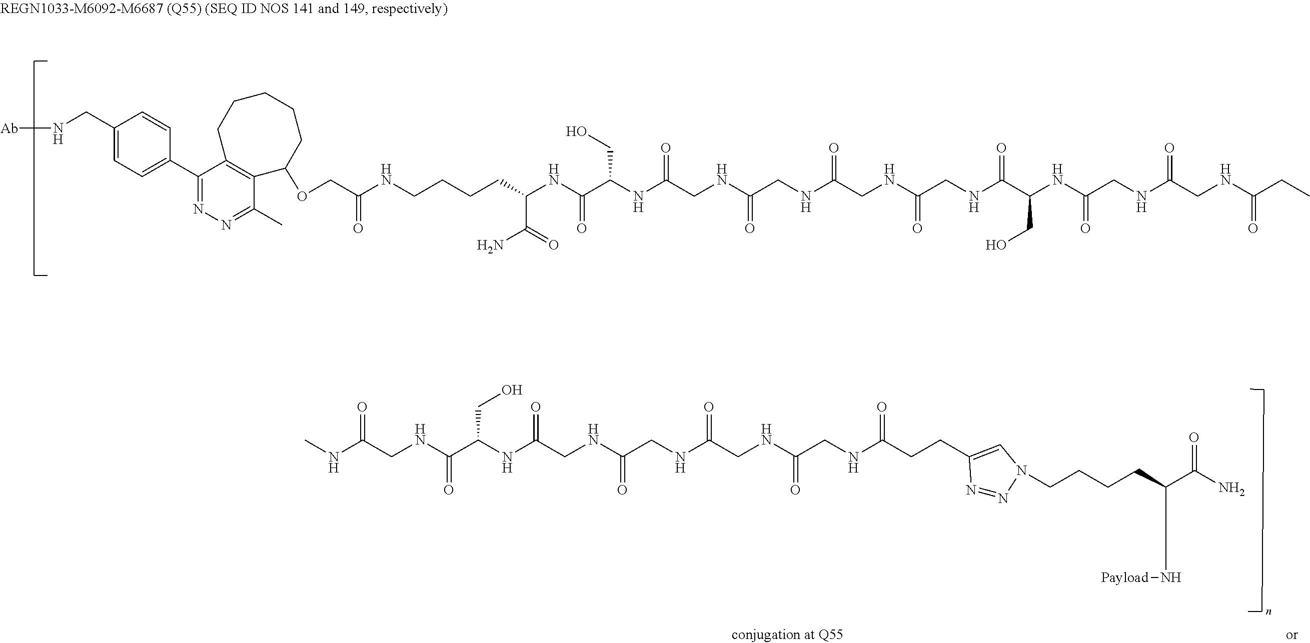

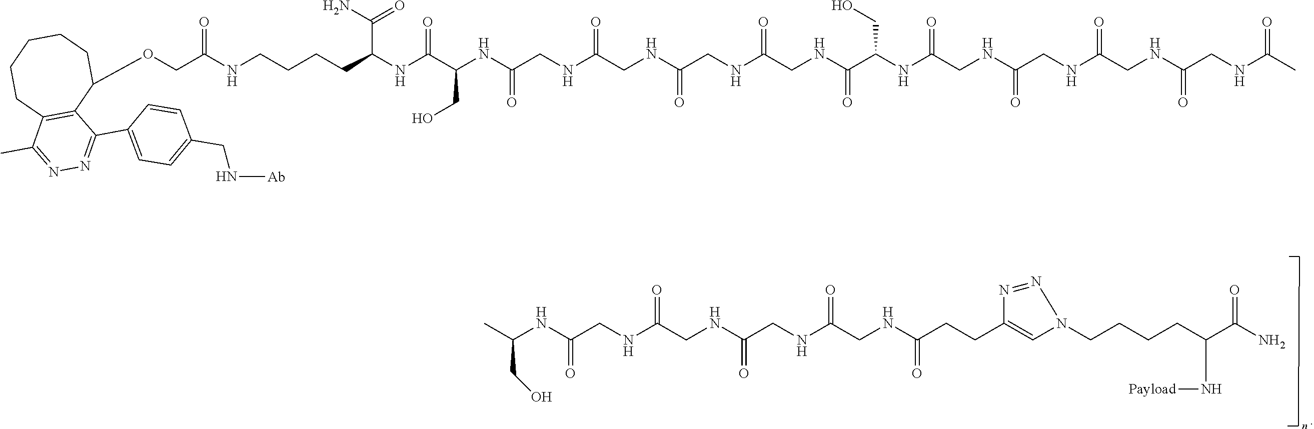

or a pharmaceutically acceptable salt thereof, where n is about 1 to 10, and Ab is a GDF8 antibody, or the antigen-binding fragment thereof, and where the Payload is a GLP1R and GIPR dual agonist having a sequence of Tyr1 Aib2 XX3 Gly4 XX5 XX6 Thr Ser Asp Tyr Ser Ser XX13 Leu Glu Glu Gln Ala Ala XX20 Glu XX22 Ile XX24 XX25 Leu Val XX28 Gly Gly Gly (SEQ ID NO: 3), wherein Aib represents 2-aminoisobutyric acid (α-aminoisobutyric acid); XX3 is Glu or Atz ((S)-2-amino-3-(2H-tetrazol-5-yl)propanoic acid); XX5 is Ser or Thr; XX6 is phenylalanine, alpha methyl phenylalanine (amF) or 2-fluoro alpha methyl phenylalanine (amF(2-fluoro)); XX13 is Tyr, amF, or alpha methyl tyrosine (amY); XX20 is Lys, sidechain-acylated or alkylated Lys, or Aib; XX22 is Phe or amF; XX24 is Ala or Glu; XX25 is tryptophan or alpha methyl tyrosine (amY); XX28 is Lys, Ala, Gln, or Glu.

In another aspect, provided herein is a pharmaceutical composition comprising the conjugate of any of the embodiments described herein, or a pharmaceutically acceptable salt thereof.

In some embodiments, the pharmaceutical composition further comprises one or more pharmaceutically accept salts, excipients, and/or diluents.

In another aspect, provided herein is a pharmaceutical composition of any of the embodiments described herein further comprising one or more other therapeutic molecules.

In another aspect, provided herein is a glucagon-like peptide-1 (GLP-1) receptor (GLP1R) and glucose-dependent insulinotropic polypeptide (GIP) receptor (GIPR) dual agonist, or a pharmaceutically acceptable salt thereof, comprising: Tyr1 Aib2 XX3 Gly4 XX5 XX6 Thr Ser Asp Tyr Ser Ser XX13 Leu Glu Glu Gln Ala Ala XX20 Glu XX22 Ile XX24 XX25 Leu Val XX28 Gly Gly Gly (SEQ ID NO: 3), where Aib represents 2-aminoisobutyric acid (α-aminoisobutyric acid); XX3 is Glu or Atz ((S)-2-amino-3-(2H-tetrazol-5-yl)propanoic acid); XX5 is Ser or Thr; XX6 is phenylalanine, alpha methyl phenylalanine (amF) or 2-fluoro alpha methyl phenylalanine (amF(2-fluoro)); XX13 is Tyr, amF, or alpha methyl tyrosine (amY); XX20 is Lys, sidechain-acylated or alkylated Lys, or Aib; XX22 is Phe or amF; XX24 is Ala or Glu; XX25 is tryptophan or alpha methyl tyrosine (amY); XX28 is Lys, Ala, Gln, or Glu; and wherein the C-terminal end is an acid or an amide.

In another aspect, provided herein is a glucagon-like peptide-1 (GLP-1) receptor (GLP1R) and glucose-dependent insulinotropic polypeptide (GIP) receptor (GIPR) dual agonist (GLP1R-GIPR dual agonist), or a pharmaceutically acceptable salt thereof, comprising: Tyr1 Aib2 XX3 Gly4 XX5 XX6 Thr Ser Asp Tyr Ser Ser XX13 Leu Glu Glu Gln Ala Ala XX20 Glu XX22 Ile XX24 XX25 Leu Val XX28 Gly Gly Gly (SEQ ID NO: 3), where Aib represents 2-aminoisobutyric acid (α-aminoisobutyric acid); XX3 is Glu or Atz ((S)-2-amino-3-(2H-tetrazol-5-yl)propanoic acid); XX5 is Ser or Thr; XX6 is phenylalanine, alpha methyl phenylalanine (amF) or 2-fluoro alpha methyl phenylalanine (amF(2-fluoro)); XX13 is amF or alpha methyl tyrosine (amY); XX20 is Lys, sidechain-acylated or alkylated Lys, or Aib; XX22 is Phe or amF; XX24 is Ala or Glu; XX25 is tryptophan or alpha methyl tyrosine (amY); XX28 is Lys, Ala, Gln, or Glu; and wherein the C-terminal end is an acid or an amide.

In another aspect, provided herein is a glucagon-like peptide-1 (GLP-1) receptor (GLP1R) and glucose-dependent insulinotropic polypeptide (GIP) receptor (GIPR) dual agonist, or a pharmaceutically acceptable salt thereof, comprising: Tyr1 Aib2 XX3 Gly4 XX5 XX6 Thr Ser Asp Tyr Ser Ser XX13 Leu Glu Glu Gln Ala Ala XX20 Glu XX22 Ile XX24 XX25 Leu Val XX28 Gly Gly Gly (SEQ ID NO: 3), where Aib represents 2-aminoisobutyric acid (α-aminoisobutyric acid); XX3 is Glu or Atz ((S)-2-amino-3-(2H-tetrazol-5-yl)propanoic acid); XX5 is Ser or Thr; XX6 is phenylalanine, alpha methyl phenylalanine (amF) or 2-fluoro alpha methyl phenylalanine (amF(2-fluoro)); XX13 is Tyr, amF, or alpha methyl tyrosine (amY); XX20 is Lys, sidechain-acylated or alkylated Lys, or Aib; XX22 is amF; XX24 is Ala or Glu; XX25 is tryptophan or alpha methyl tyrosine (amY); XX28 is Lys, Ala, Gln, or Glu; and wherein the C-terminal end is an acid or an amide.

In another aspect, provided herein is a glucagon-like peptide-1 (GLP-1) receptor (GLP1R) and glucose-dependent insulinotropic polypeptide (GIP) receptor (GIPR) dual agonist, or a pharmaceutically acceptable salt thereof, comprising: Tyr1 Aib2 XX3 Gly4 XX5 XX6 Thr Ser Asp Tyr Ser Ser XX13 Leu Glu Glu Gln Ala Ala XX20 Glu XX22 Ile XX24 XX25 Leu Val XX28 Gly Gly Gly (SEQ ID NO: 3), where Aib represents 2-aminoisobutyric acid (α-aminoisobutyric acid); XX3 is Glu or Atz ((S)-2-amino-3-(2H-tetrazol-5-yl)propanoic acid); XX5 is Ser or Thr; XX6 is phenylalanine, alpha methyl phenylalanine (amF) or 2-fluoro alpha methyl phenylalanine (amF(2-fluoro)); XX13 is Tyr, amF, or alpha methyl tyrosine (amY); XX20 is Lys, sidechain-acylated or alkylated Lys, or Aib; XX22 is Phe or amF; XX24 is Glu; XX25 is tryptophan or alpha methyl tyrosine (amY); XX28 is Lys, Ala, Gln, or Glu; and wherein the C-terminal end is an acid or an amide.

In another aspect, provided herein is a glucagon-like peptide-1 (GLP-1) receptor (GLP1R) and glucose-dependent insulinotropic polypeptide (GIP) receptor (GIPR) dual agonist, or a pharmaceutically acceptable salt thereof, comprising: Tyr1 Aib2 XX3 Gly4 XX5 XX6 Thr Ser Asp Tyr Ser Ser XX13 Leu Glu Glu Gln Ala Ala XX20 Glu XX22 Ile XX24 XX25 Leu Val XX28 Gly Gly Gly (SEQ ID NO: 3), where Aib represents 2-aminoisobutyric acid (α-aminoisobutyric acid); XX3 is Glu or Atz ((S)-2-amino-3-(2H-tetrazol-5-yl)propanoic acid); XX5 is Ser or Thr; XX6 is phenylalanine, alpha methyl phenylalanine (amF) or 2-fluoro alpha methyl phenylalanine (amF(2-fluoro)); XX13 is Tyr, amF, or alpha methyl tyrosine (amY); XX20 is Lys, sidechain-acylated or alkylated Lys, or Aib; XX22 is Phe or amF; XX24 is Ala or Glu; XX25 is alpha methyl tyrosine (amY); XX28 is Lys, Ala, Gln, or Glu; and wherein the C-terminal end is an acid or an amide.

In another aspect, provided herein is a glucagon-like peptide-1 (GLP-1) receptor (GLP1R) and glucose-dependent insulinotropic polypeptide (GIP) receptor (GIPR) dual agonist, or a pharmaceutically acceptable salt thereof, comprising: Tyr1 Aib2 XX3 Gly4 XX5 XX6 Thr Ser Asp Tyr Ser Ser XX13 Leu Glu Glu Gln Ala Ala XX20 Glu XX22 Ile XX24 XX25 Leu Val XX28 Gly Gly Gly (SEQ ID NO: 3), where Aib represents 2-aminoisobutyric acid (α-aminoisobutyric acid); XX3 is Glu or Atz ((S)-2-amino-3-(2H-tetrazol-5-yl)propanoic acid); XX5 is Ser or Thr; XX6 is phenylalanine, alpha methyl phenylalanine (amF) or 2-fluoro alpha methyl phenylalanine (amF(2-fluoro)); XX13 is Tyr, amF, or alpha methyl tyrosine (amY); XX20 is Lys, sidechain-acylated or alkylated Lys, or Aib; XX22 is Phe or amF; XX24 is Ala or Glu; XX25 is tryptophan or alpha methyl tyrosine (amY); XX28 is Ala, Gln, or Glu; and wherein the C-terminal end is an acid or an amide.

In some embodiments, XX13 is amF or alpha methyl tyrosine (amY).

In some embodiments, XX22 is alpha methyl phenylalanine (amF).

In some embodiments, XX25 is alpha methyl tyrosine (amY).

In some embodiments, the GLP1R-GIPR dual agonist has a sequence selected from the group consisting of SEQ ID NOs: 3, 14-17, and 19-54.

In some embodiments, the GLP1R and GIPR dual agonist binds to and activates the signaling cascade of GLP1R and GIPR.

In some embodiments, the GLP1R and GIPR dual agonist binds to and activates the signaling cascade of GLP1R and GIPR, and where the GLP1R and GIPR dual agonist does not substantially binds to and/or activates the signaling cascade of glucagon peptide receptor.

In another aspect, provided herein is a conjugate, or a pharmaceutically acceptable salt thereof, comprising a GLP1R and GIPR dual agonist according to any of the embodiments described herein, an antigen binding protein (ABP), and a linker covalently linking the dual agonist and ABP, where the ABP is a GDF8 antibody, or the antigen-binding fragment thereof.

In some embodiments, the GLP1R and GIPR dual agonist, or a pharmaceutically acceptable salt thereof, according to any of the embodiments described herein, further comprises a linker with a reactive moiety for conjugation with a target antibody or antigen binding fragment thereof.

In some embodiments, the linker with a reactive moiety for conjugation with a target antibody or antigen binding fragment thereof has the following structure: -(Gly₃Ser)ₙ-Lys(X)- (SEQ ID NO: 97) or -(Gly₄Ser)ₙ-Lys(X)- (SEQ ID NO: 98), where n is an integer from one to six; and where X is a reactive moiety attached to the side chain of Lys for conjugation with an antigen binding protein (ABP).

In some embodiments, X is a reactive moiety comprising an azido, 2-bromo-acetyl,

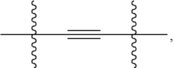 [SMILES: *C#C*]

2-(cyclooct-2-yn-lyloxy) acetyl, 2′-maleimido-acetyl, and 3′-maleimido-propanoyl, —N₃,

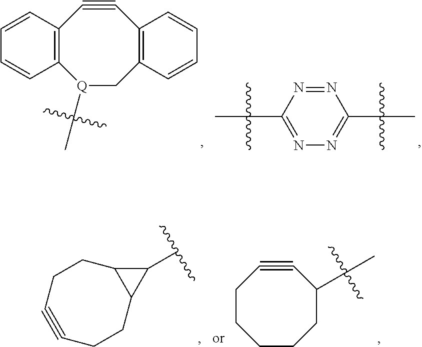

where Q is CH or N.

In some embodiments, Lys(X) has a structure of

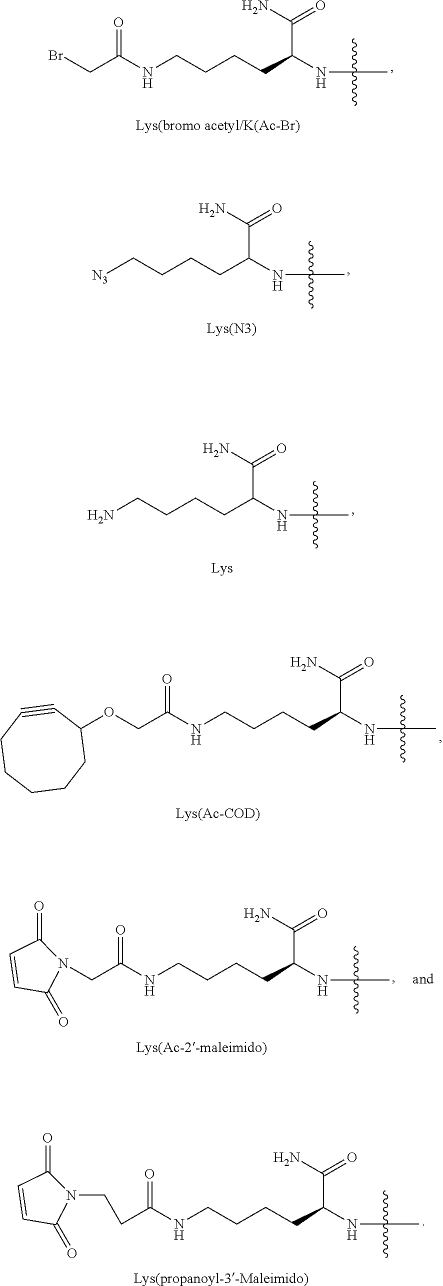

In some embodiments, the linker comprises

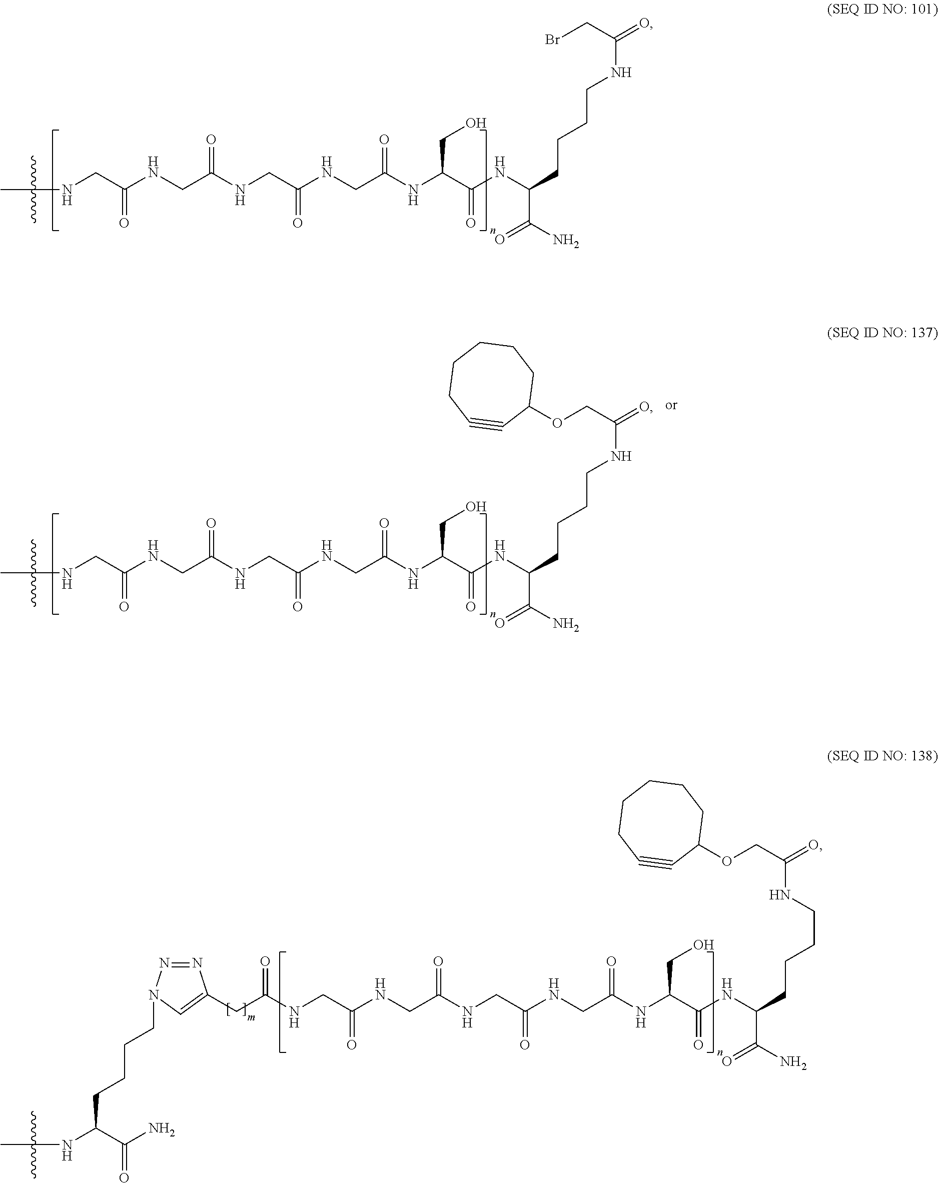

where m is an integer of one to three; and n is an integer of one to four.

In another aspect, provided herein is a pharmaceutical composition comprising the GLP1R and GIPR dual agonist according to any of the embodiments described herein, or a pharmaceutically acceptable salt thereof.

In some embodiments, the pharmaceutical composition further comprises one or more pharmaceutically acceptable salts, excipients, and/or diluents.

In another aspect, provided herein is a GLP1R-GIPR dual agonist and an antigen binding protein (ABP) conjugate, or a pharmaceutically acceptable salt thereof, having a Formula (I):

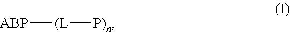

where
   ABP is an antibody, or an antigen-binding fragment thereof, that specifically binds to and/or blocks the biological activity of human Growth and Differentiation Factor-8 (GDF8);
   P is a glucagon-like peptide-1 (GLP-1) receptor (GLP1R) and glucose-dependent insulinotropic polypeptide (GIP) receptor (GIPR) dual agonist;
   L is a linker that covalently links P and ABP; and
   n ranges from about 1 to about 6.

In some embodiments, the conjugate has a formula (II):

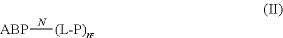

where [symbol] represents that the connection between the ABP and the L is through the side chain of [symbol] a glutamine residue (Gln) of the ABP.

In some embodiments, the Gln is a naturally occurring Gln or an engineered Gln.

In some embodiments, the Gln is Gln55 (Q55).

In some embodiments, the Gln is Gln295 (Q295) or Gln297 (Q297).

In some embodiments, ABP is first modified with a handle comprising (a) a reactive moiety for Click or Diels-Alder reaction and (b) a primary amine suitable for microbial transglutaminase-mediated conjugation.

In some embodiments, the handle is selected from the group consisting of

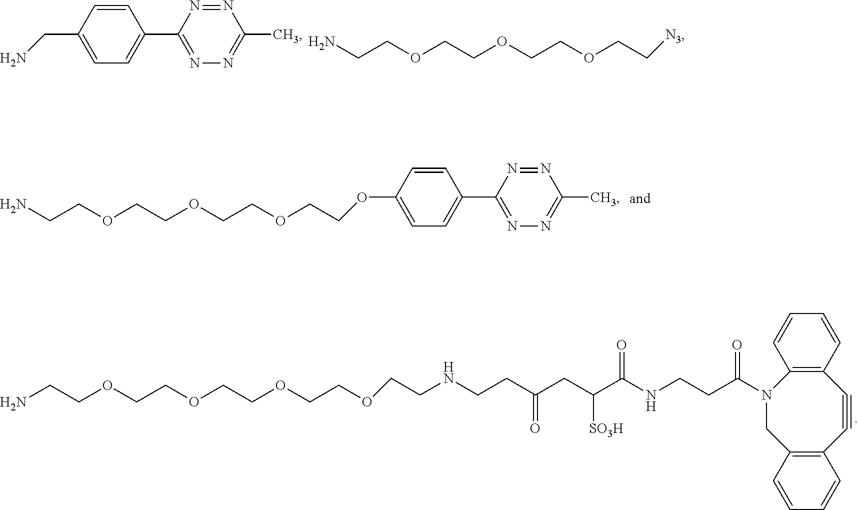 [SMILES: Cc1nnc(-c2ccc(CN)cc2)nn1 ; [N-]=[N+]=NCCOCCOCCOCCN ; Cc1nnc(-c2ccc(OCCOCCOCCOCCN)cc2)nn1 ; NCCOCCOCCOCCOCCNCCC(=O)CC(C(=O)NCCC(=O)N1Cc2ccccc2C#Cc2ccccc21)S(=O)(=O)O]

In some embodiments, the reactive moiety for Click or Diels-Alder reaction is selected from the group consisting of from

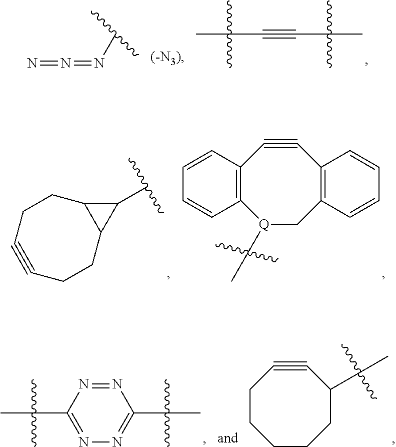

where Q is CH or N.

In some embodiments, the linker comprises a Click or Diels-Alder reaction adduct selected from the group consisting of:

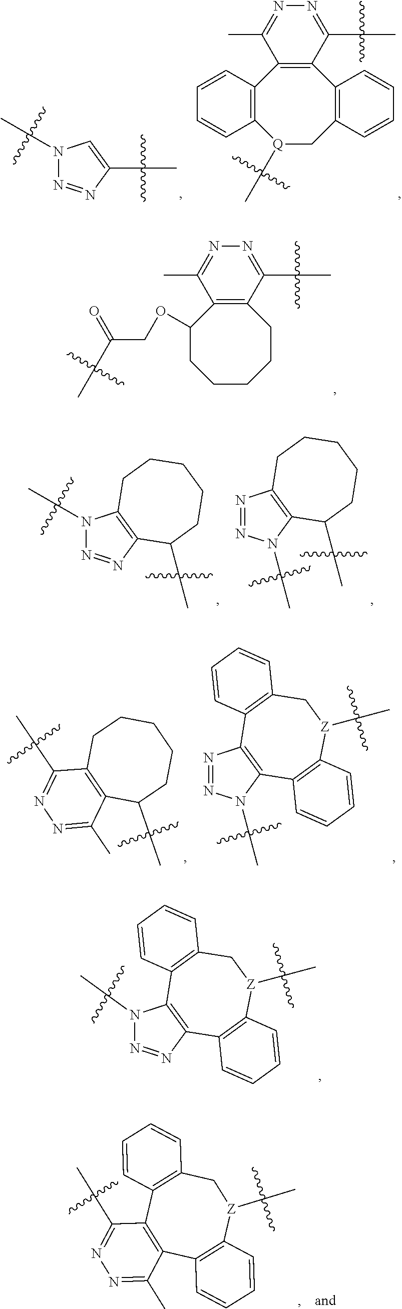

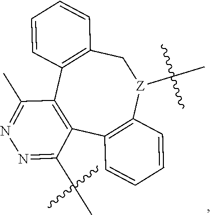

and where Z is CH or N.

In some embodiments, the linker comprises

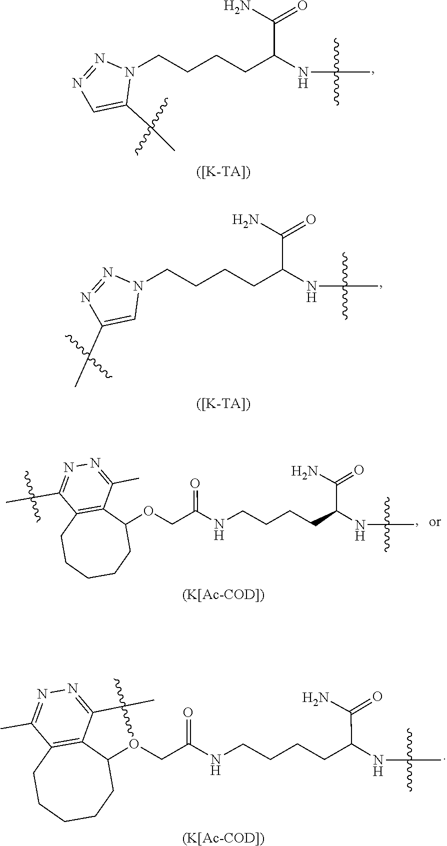

In some embodiments, the conjugate has a Formula (III):

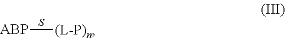

where [symbol] represents that the connection between ABP and linker L via the side chain of a cysteine residue (Cys) of the ABP.

In some embodiments, the Cys of the BA is a naturally occurring Cys or an engineered Cys.

In some embodiments, L comprises a moiety resulted from chemical conjugation of a reactive group (RG) on the L-P with the side chain of Cys of the ABP.

In some embodiments, the reactive group is a bromo acetyl or maleimide.

In some embodiments, the linker L comprises

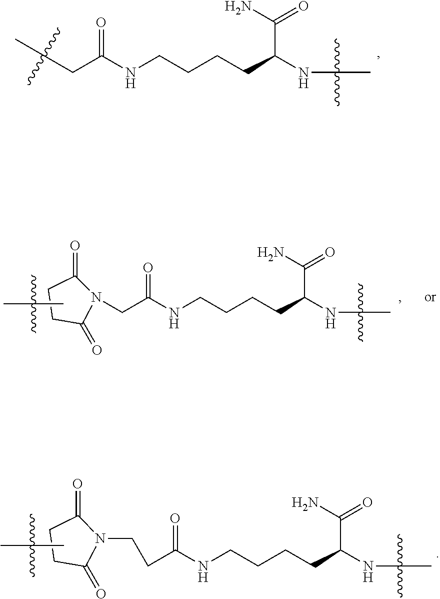

In some embodiments, the linker L further comprises a sequence selected from the group consisting of -(Gly₃Ser)ₘ- (SEQ ID NO: 99) and -(Gly₄Ser)ₘ- (SEQ ID NO: 100), wherein m is an integer from one to six.

In some embodiments, the ABP of the GDF8 antibody or the antigen binding fragment thereof comprises 1A2, 21-E5, 8D12, H4H1669P, AMG 181, AMG 745, Apitegromab (SRK-015), Bimagrumab (BYM338), Domagrozumab (PF-06252616), GYM329 (RO7204239), Landogrozumab (LY2495655), Stamulumab (MYO-029), and Trevogrumab (REGN1033).

In some embodiments, the ABP of the GDF8 antibody or the antigen binding fragment thereof binds to and/or blocks the activity of growth and differentiation factor-8 (GDF8) having a sequence of SEQ ID NO: 1.

In some embodiments, ABP of the GDF8 antibody or the antigen binding fragment thereof comprises three heavy chain complementarity determining regions (HCDR1, HCDR2, and HCDR3) comprising SEQ ID NO: 6, SEQ ID NO: 7, and SEQ ID NO: 8, respectively, and three light chain complementarity determining regions (LCDR1, LCDR2, and LCDR3) comprising SEQ ID NO: 9, SEQ ID NO: 10, and SEQ ID NO: 11, respectively.

In some embodiments, ABP of the GDF8 antibody or the antigen binding fragment thereof comprises heavy chain complementarity determining regions (HCDRs) of a heavy chain variable region (HCVR) comprising SEQ ID NO:4, and light chain complementarity determining regions (LCDRs) of a light chain variable region (LCVR) comprising SEQ ID NO:5.

In another aspect, provided herein is a method for treating obesity by reducing body weight while maintaining or increasing lean body mass in a subject, comprising administering a therapeutically effective amount of a GLP1R-GIPR dual agonist or an antigen binding protein (ABP) conjugate thereof according to any of the embodiments described herein, to the subject in need thereof.

In another aspect, provided herein is a method for treating obesity by reducing fat mass while maintaining or increasing lean body mass in a subject, comprising administering a therapeutically effective amount of a GLP1R-GIPR dual agonist or an antigen binding protein (ABP) conjugate thereof according to any of the embodiments described herein, to the subject in need thereof.

In another aspect, provided herein is a method for treating Type 2 diabetes by improving glycemic control and maintaining or increasing lean body mass while reducing fat mass in a subject, comprising administering a therapeutically effective amount of a GLP1R-GIPR dual agonist or an antigen binding protein (ABP) conjugate thereof according to any of the embodiments described herein, to the subject in need thereof.

In another aspect, provided herein is a method for treating obesity and Type 2 diabetes by improving glycemic control and maintaining or increasing lean body mass while reducing fat mass in a subject, comprising administering a therapeutically effective amount of a GLP1R-GIPR dual agonist or an antigen binding protein (ABP) conjugate thereof according to any of the embodiments described herein, to the subject in need thereof.

In another aspect, provided herein is a method for treating obesity, diabetes, and/or liver diseases associated with increased fat mass in a subject, comprising administering a therapeutically effective amount of a GLP1R-GIPR dual agonist and an antigen binding protein (ABP) conjugate according to any of the embodiments described herein, to the subject in need thereof.

In another aspect, provided herein is a method for treating obesity, diabetes, and/or liver diseases associated with increased fat mass in a subject, comprising administering a therapeutically effective amount of a GLP1R-GIPR dual agonist or an antigen binding protein (ABP) conjugate thereof according to any of the embodiments described herein, together with one or more other therapeutic agents, to the subject in need thereof.

In another aspect, provided herein is a method for treating a subject of metabolic syndrome by improving glycemic control, maintaining or increasing lean body mass while reducing fat mass in a subject, comprising administering a therapeutically effective amount of a GLP1R-GIPR dual agonist or an antigen binding protein (ABP) conjugate thereof according to any of the embodiments described herein, to the subject in need thereof.

In another aspect, provided herein is a method for treating a subject of metabolic syndrome by improving glycemic control, maintaining or increasing lean body mass while reducing fat mass in a subject, comprising administering a therapeutically effective amount of a GLP1R-GIPR dual agonist or an antigen binding protein (ABP) conjugate thereof according to any of the embodiments described herein, together with one or more other therapeutic agents, to the subject in need thereof.

In another aspect, provided herein is a process for manufacturing GLP1R-GIPR dual agonist and an antigen binding protein (ABP) conjugate, where ABP is a GDF8 antibody or antigen binding fragment thereof, comprising a) optionally attaching a handle comprising a first reactive moiety; b) exposing a GLP1R-GIPR dual agonist comprising a second reactive moiety; c) initiating conjugation reaction between the first and second reactive moieties; and d) isolating/purifying the conjugate of GLP1R-GIPR dual agonist and ABP.

In some embodiments, the reactive moieties are bio-orthogonally operable in an aqueous environment.

In some embodiments, the reactive moieties are selected from the group consisting of amino, thiol, maleimido

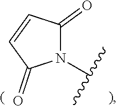 [SMILES: *N1C(=O)C=CC1=O]

succinyl ester

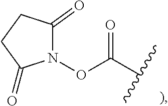 [SMILES: *C(=O)ON1C(=O)CCC1=O]

and alpha-bromo acetyl.

In some embodiments, the reactive moieties are selected from the group consisting of —N₃,

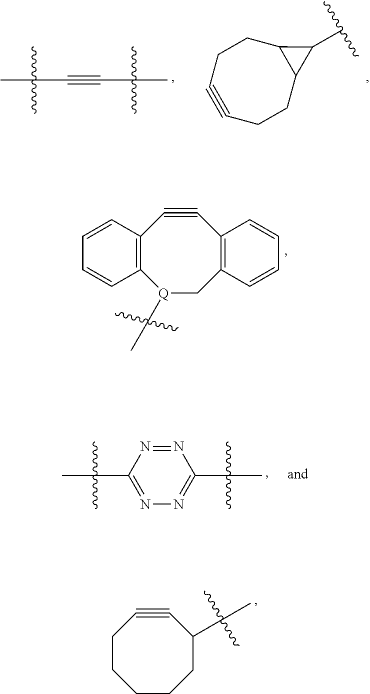

where Q is CH or N.

In some embodiments, the handle comprises a primary amino group for conjugation mediated by microbial transglutaminase and a reactive moiety suitable for Click or Diels-Alder reaction.

In some embodiments, the handle comprises

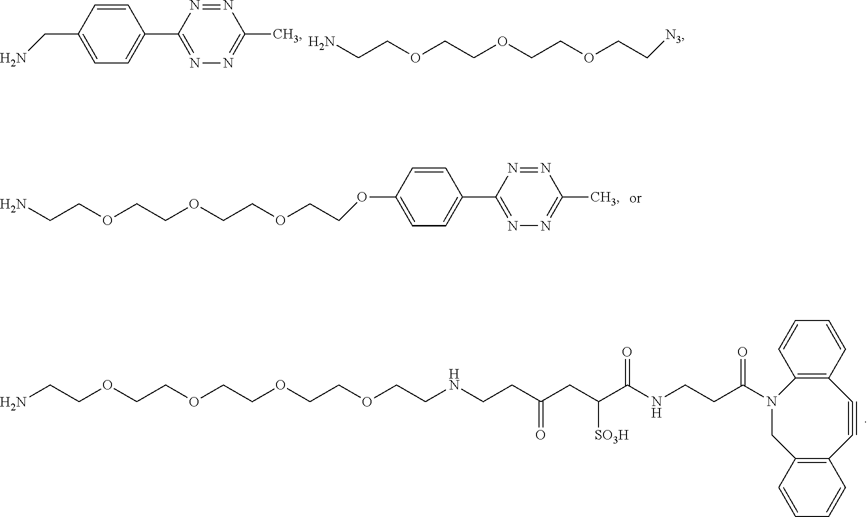 [SMILES: Cc1nnc(-c2ccc(CN)cc2)nn1 ; [N-]=[N+]=NCCOCCOCCOCCN ; Cc1nnc(-c2ccc(OCCOCCOCCOCCN)cc2)nn1 ; NCCOCCOCCOCCOCCNCCC(=O)CC(C(=O)NCCC(=O)N1Cc2ccccc2C#Cc2ccccc21)S(=O)(=O)O]

In another aspect, provided herein is a product of the process according to any of the embodiments described herein.

In another aspect, provided herein is a pharmaceutical composition comprising a GLP1R-GIPR dual agonist and an antigen binding protein (ABP) conjugate manufactured according to the process of any of the embodiments described herein, together with one or more pharmaceutically acceptable excipients.

In some embodiments, XX3 is Glu or Atz ((S)-2-amino-3-(2H-tetrazol-5-yl)propanoic acid).

In some embodiments, XX20 is Aib, Lys, Gln or Glu.

In some embodiments, XX24 is Ala, Gln or Glu.

In some embodiments, XX25 is alpha methyl tyrosine.

In some embodiments, XX28 is Lys, Gln or Glu.

In some embodiments, the GLP1R-GIPR dual agonist has a sequence of Tyr1 Aib2 XX3 Gly4 XX5 XX6 Thr Ser Asp Tyr Ser Ser XX13 Leu Glu Glu Gln Ala Ala XX20 Glu XX22 Ile XX24 XX25 Leu Val XX28 Gly Gly Gly (SEQ ID NO: 3), where Aib is 2-aminoisobutyric acid (α-aminoisobutyric acid); amF is alpha methyl phenylalanine; and amY is alpha methyl tyrosine.

In some embodiments, the conjugate has a structure of

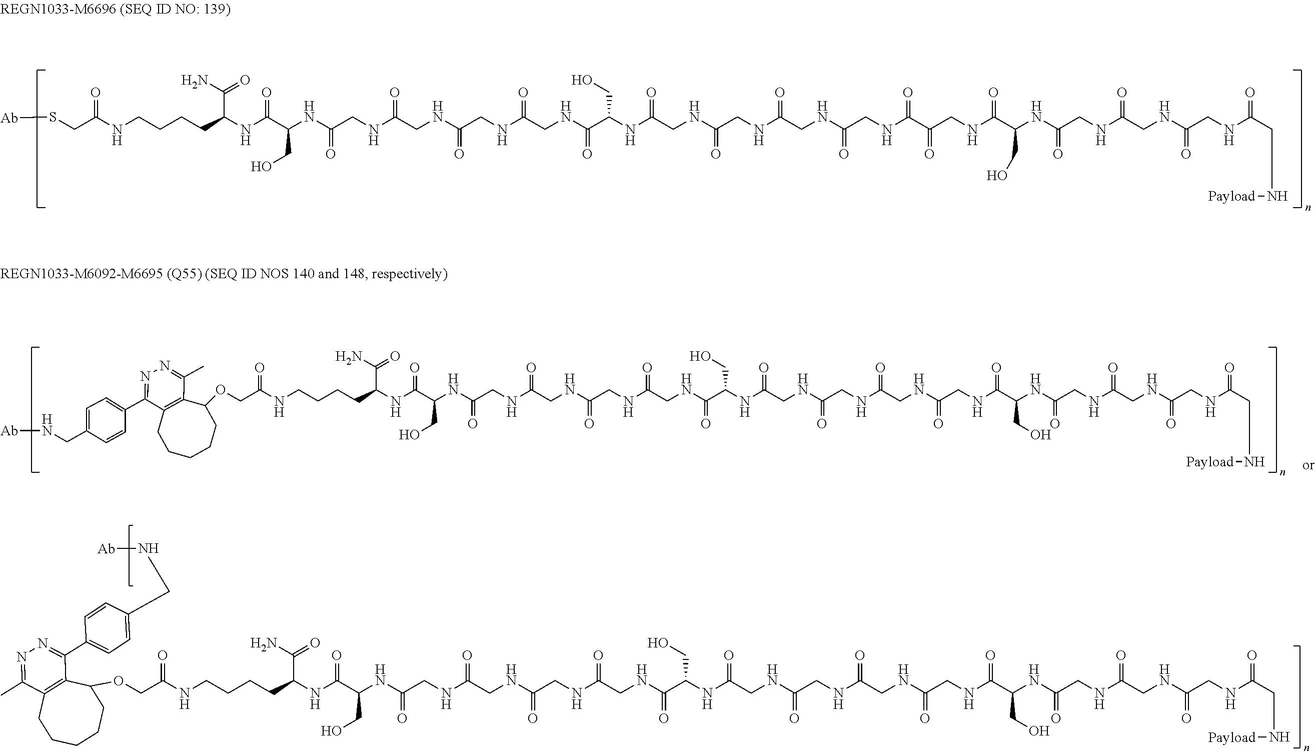

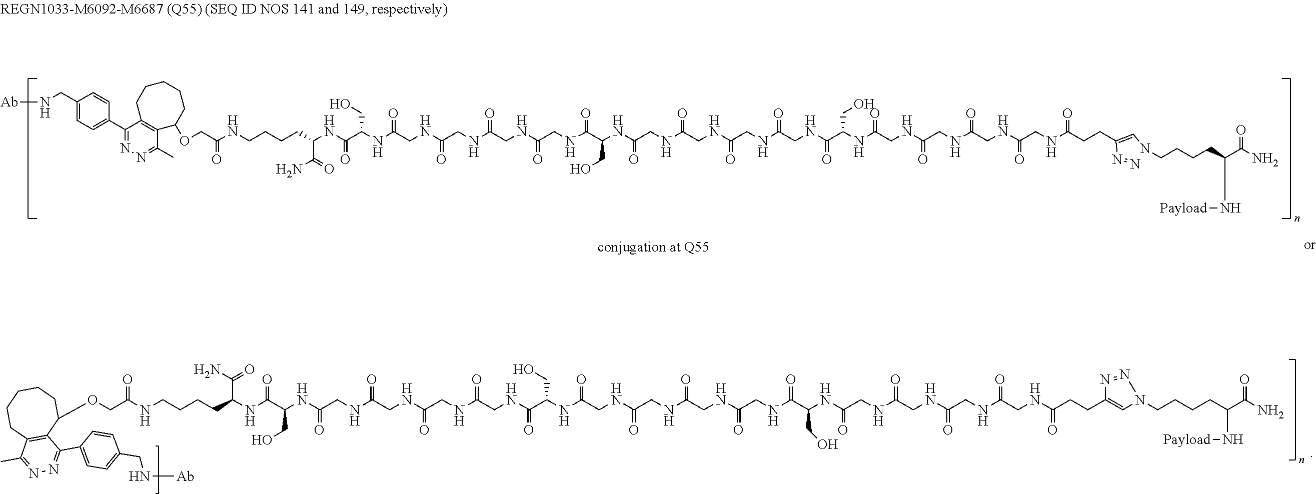

or a pharmaceutically acceptable salt thereof, where n is about 1 to 10, and Ab is a GDF8 antibody, or the antigen-binding fragment thereof, and where the Payload is a GLP1R and GIPR dual agonist having a sequence of Tyr1 Aib2 XX3 Gly4 XX5 XX6 Thr Ser Asp Tyr Ser Ser XX13 Leu Glu Glu Gln Ala Ala XX20 Glu XX22 Ile XX24 XX25 Leu Val XX28 Gly Gly Gly (SEQ ID NO: 3), wherein Aib represents 2-aminoisobutyric acid (α-aminoisobutyric acid); XX3 is Glu or Atz ((S)-2-amino-3-(2H-tetrazol-5-yl)propanoic acid); XX5 is Ser or Thr; XX6 is phenylalanine, alpha methyl phenylalanine (amF) or 2-fluoro alpha methyl phenylalanine (amF(2-fluoro)); XX13 is Tyr, amF, or alpha methyl tyrosine (amY); XX20 is Lys, sidechain-acylated or alkylated Lys, or Aib; XX22 is Phe or amF; XX24 is Ala or Glu; XX25 is tryptophan or alpha methyl tyrosine (amY); XX28 is Lys, Ala, Gln, or Glu.

In some embodiments, XX3 is Glu, Gln, or Atz ((S)-2-amino-3-(2H-tetrazol-5-yl)propanoic acid).

In some embodiments, XX20 is Lys or Aib.

In some embodiments, XX24 is Ala, Gln, or Glu.

In some embodiments, XX28 is Lys, Gln, or Glu.

These and other aspects of the present disclosure will become apparent to those skilled in the art after a reading of the following detailed description of the disclosure, including the appended claims.

### BRIEF DESCRIPTIONS OF DRAWINGS

FIG. 1A depicts general scheme for synthesis of linker-peptide using Method A (linker A) and FIG. 1B shows general scheme for conjugation with Peptide Linker A.

FIG. 2A shows general scheme for synthesis of Linker-Payload peptide with Method C (Linker C) and FIG. 2B shows general scheme for conjugation using method C. FIG. 2A discloses SEQ ID NOS 142-143, respectively, in order of appearance. FIG. 2B discloses SEQ ID NOS 144-146, respectively, in order of appearance.

FIG. 3 shows analytical SEC chromatogram of a purified anti-GDF8 conjugate.

FIG. 4 shows LC-ESI-MS spectrum of an IdeS digested anti-GDF8 antibody conjugate samples. The calculated average DAR value was 1.96.

FIG. 5 shows analytical SEC chromatogram of a purified anti-GDF8 AC.

FIG. 6 shows LC-ESI-MS spectrum of an IdeS digested anti-GDF8 antibody conjugate sample. The calculated average DAR value was 1.95.

FIGS. 7A-7B show plasma stability of M6687 and M6695 in mouse (FIG. 7A) and human plasma (FIG. 7B) over 4-hr incubation at 37° C.

FIGS. 8A-8B show GLP-1 and Tirzepatide in mouse (FIG. 8A) and human plasma (FIG. 8B) over 4-hr incubation at 37° C.

FIG. 9 shows LC-MS chromatograms of four peptide/peptidomimetics used for quantification.

FIGS. 10A-10C depict overall body weight reductions elicited after following 36 days of treatment, administration of Tirzepatide, and REGN1033-M6695. Tirzepatide treatment resulted in a net body weight loss (23%) that can be attributed to a reduction in both fat mass (40%) and lean mass (4.5%). In contrast, treatment with REGN1033-M6695 also led to a net body weight loss (20%); however, this is a result of fat mass loss (42%) and lean mass gain (5.2%).

FIGS. 11A-11C demonstrate the disparity in lean mass between Tirzepatide treatment and REGN1033-M6695, tissue weights were collected and measured after 37 days of treatment. Tissue weights are expressed as a % change from the average of the Isotype Control group±SEM. Skeletal muscle measurements revealed that treatment with REGN1033-M6695 significantly increased tibialis anterior (21%), gastrocnemius (9.3%), and quadriceps (Q15%) muscle weights compared to Isotype Control. These results are unlike Tirzepatide treatment; mice receiving Tirzepatide had slightly reduced skeletal muscle mass compared to Isotype Control. As a result, all skeletal muscle weights from REGN1033-M6695 were also significantly greater than Tirzepatide treatment.

FIG. 12 shows reduction of body weight with REGN1033-M6695 and REGN1033-M6687. DIO mice were dosed subcutaneously at 10 mg/kg weekly with the labeled treatments. Body weights were measured twice weekly throughout the study. Each data point is mean±SEM. * P<0.05 of REGN1033-M6695 vs. Control mAb, #P<0.05 of REGN1033-M6687 vs. Control mAb. Stats by two-way RM ANOVA.

FIGS. 13A-13B show reduction of body fat with REGN1033-M6695 and EGN1033-M6687. Body composition was measured by qNMR at baseline and on D14 and D23 of the study. Data expressed as percent change from the baseline reading. Each data point is mean±SEM. * P<0.05 of REGN1033-M6695 vs. Control mAb, #P<0.05 of REGN1033-M6687 vs. Control mAb. Stats by two-way RM ANOVA.

FIGS. 14A-14F show individual depot weights with REGN1033-M6695 and REGN1033-M6687. Terminal fat and skeletal muscle weights. Data expressed as percent change from Control mAb. Each data point is mean±SEM. * P<0.05, ** P<0.01, *** P<0.001 vs. designated group. Stats by one-way ANOVA.

FIGS. 15A-15B show fed glucose (FIG. 15A) and liver weight (FIG. 15B) effects with REGN1033-M6695 and REGN1033-M6687. Fed blood glucose and terminal liver and pancreas weights. Fed glucose expressed as percent change from baseline, liver and pancreas as percent change from Control mAb. Each data point is mean±SEM. Panel A: NS, Stats by two-way RM ANOVA. Panel B: * P<0.05, ** P<0.01 vs. designated group. Stats by one-way ANOVA.

### DETAILED DESCRIPTION

Detailed embodiments of the present disclosure are disclosed herein; however, it is to be understood that the disclosed embodiments are merely illustrative of the disclosure that may be embodied in various forms. In addition, each of the examples given in connection with the various embodiments of the disclosure is intended to be illustrative, and not restrictive. Therefore, specific structural and functional details disclosed herein are not to be interpreted as limiting, but merely as a representative basis for teaching one skilled in the art to variously employ the present disclosure.

### Definitions

Unless defined otherwise, all technical and scientific terms used herein have the same meaning as commonly understood by one of ordinary skills in the art to which this disclosure belongs.

As used in this specification and the appended claims, the singular forms “a”, “an”, and “the” include plural references unless the context clearly dictates otherwise. Thus, for example, a reference to “a method” includes one or more methods, and/or steps of the type described herein and/or which will become apparent to those persons skilled in the art upon reading this disclosure.

The terms “treat” or “treatment” of a state, disorder or condition include: (1) preventing, delaying, or reducing the incidence and/or likelihood of the appearance of at least one clinical or sub-clinical symptom of the state, disorder, or condition developing in a subject that may be afflicted with or predisposed to the state, disorder, or condition but does not yet experience or display clinical or subclinical symptoms of the state, disorder, or condition; or (2) inhibiting the state, disorder, or condition, i.e., arresting, reducing or delaying the development of the disease or a relapse thereof or at least one clinical or sub-clinical symptom thereof, or (3) relieving the disease, i.e., causing regression of the state, disorder, or condition or at least one of its clinical or sub-clinical symptoms. The benefit to a subject to be treated is either statistically significant or at least perceptible to the patient or to the physician. In some embodiments, treatment comprises methods wherein cells are ablated in such manner where disease is indirectly impacted. In certain embodiments, treatment comprises depleting immune cells as a hematopoietic conditioning regimen prior to therapy.

A “subject” or “patient” or “individual” or “animal”, as used herein, refers to humans, veterinary animals (e.g., cats, dogs, cows, horses, sheep, pigs, etc.) and experimental animal models of diseases (e.g., mice, rats). In a preferred embodiment, the subject is a human.

As used herein the term “effective” applied to dose or amount refers to that quantity of a compound or pharmaceutical composition that is sufficient to result in a desired activity upon administration to a subject in need thereof. Note that when a combination of active ingredients is administered, the effective amount of the combination may or may not include amounts of each ingredient that would have been effective if administered individually. The exact amount required will vary from subject to subject, depending on the species, age, and general condition of the subject, the severity of the condition being treated, the particular drug or drugs employed, the mode of administration, and the like.

The phrase “pharmaceutically acceptable salt”, as used in connection with compositions of the disclosure, refers to any salt suitable for administration to a patient. Suitable salts include, but are not limited to, those disclosed in. Berge et al., “Pharmaceutical Salts”, J. Pharm. Sci., 1977, 66:1, incorporated herein by reference. Examples of salts include, but are not limited to, acid derived, base derived, organic, inorganic, amine, and alkali or alkaline earth metal salts, including but not limited to calcium salts, magnesium salts, potassium salts, sodium salts, salts of hydrochloric acid, hydrobromic acid, sulfuric acid, nitric acid, phosphoric acid, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methane sulfonic acid, ethane sulfonic acid, para-toluene sulfonic acid, salicylic acid, and the like. In some examples, a payload described herein comprises a tertiary amine, where the nitrogen atom in the tertiary amine is the atom through which the payload is bonded to a linker or a linker-spacer. In such instances, bonding to the tertiary amine of the payload yields a quaternary amine in the linker-payload molecule. The positive charge on the quaternary amine can be balanced by a counter ion (e.g., chloro, bromo, iodo, or any other suitably charged moiety such as those described herein).

Ranges can be expressed herein as from “about” or “approximately” one particular value and/or to “about” or “approximately” another particular value. When such a range is expressed, another embodiment includes from the one particular value and/or to the other particular value.

By “comprising” or “containing” or “including” is meant that at least the named compound, element, particle, or method step is present in the composition or article or method, but does not exclude the presence of other compounds, materials, particles, or method steps, even if the other such compounds, material, particles, or method steps have the same function as what is named.

Compounds of the present disclosure include those described generally herein, and are further illustrated by the classes, subclasses, and species disclosed herein. As used herein, the following definitions shall apply unless otherwise indicated. For purposes of this disclosure, the chemical elements are identified in accordance with the Periodic Table of the Elements, CAS version, Handbook of Chemistry and Physics, 75th Ed. Additionally, general principles of organic chemistry are described in “Organic Chemistry”, Thomas Sorrell, University Science Books, Sausalito: 1999, and “March's Advanced Organic Chemistry”, 5th Ed., Ed.: Smith, M. B. and March, J., John Wiley & Sons, New York: 2001, the entire contents of which are hereby incorporated by reference.

As disclosed herein, a Click reaction, Click chemistry, or Diels-Alder reaction, is a class of bioorthogonal organic reactions that could be used to selectively and/or site-specifically modify a protein or an antibody in an aqueous environment. Diels-Alder reactions as disclosed herein comprise not only the traditional Diels-Alder reaction between a diene and a dienophile but also include those newly developed the IEDDA (inverse electron demand Diels-Alder reaction). Due to its fast, efficient, and selective nature of this class of reactions, IEDDA has also been classified as a Click reaction. For more details, please see R. Bird, et al., Bioconjugate Chem. 2021, 32, 12, 2457-2479; Knall and Slugovc, Chem. Soc. Rev., 2013, 42, 5131; Baalmann, et al., Angew. Chem. Int. Ed. 2020, 59, 12885-12893; and Deb, et al., Chem. Rev. 2021, 121, 6850-6914.

The term “adduct” of the present disclosure encompasses any moiety comprising the product of an addition reaction, independent of the synthetic steps taken to produce the moiety.

The term “covalent attachment” means formation of a covalent bond, i.e., a chemical bond that involves sharing of one or more electron pairs between two atoms. Covalent bonding may include different interactions, including but not limited to σ-bonding, π-bonding, metal-to-metal bonding, agostic interactions, bent bonds, and three-center two-electron bonds. When a first group is said to be “capable of covalently attaching” to a second group, this means that the first group is capable of forming a covalent bond with the second group, directly or indirectly, e.g., through the use of a catalyst or under specific reaction conditions. Non-limiting examples of groups capable of covalently attaching to each other may include, e.g., an amine and a carboxylic acid (forming an amide bond), a maleimide and a thiol (forming a thio-maleimide), and an azide and an alkyne (forming a triazole via a 1,3-cycloaddition reaction).

The term “stable,” as used herein, refers to compounds that are not substantially altered when subjected to conditions to allow for their production, detection, and, in certain embodiments, their recovery, purification, and use for one or more of the purposes disclosed herein.

Unless otherwise stated, structures depicted herein are also meant to include all isomeric (e.g., enantiomeric, diastereomeric, and geometric (or conformational)) forms of the structure; for example, the R and S configurations for each asymmetric center, (Z) and (E) double bond isomers, and (Z) and (E) conformational isomers. Therefore, single stereochemical isomers as well as enantiomeric, diastereomeric, and geometric (or conformational) mixtures of the present compounds are within the scope of the disclosure.

Unless otherwise stated, cyclic adducts, e.g., products of a cycloaddition reaction, e.g., an azide-acetylene cycloaddition reaction, depicted herein include all regioisomers, i.e., structural isomers that differ only in the position of a functional group or a substituent. By way of an example, the following structures represent triazole regioisomers, which differ only in the position of the substituent on the triazole ring:

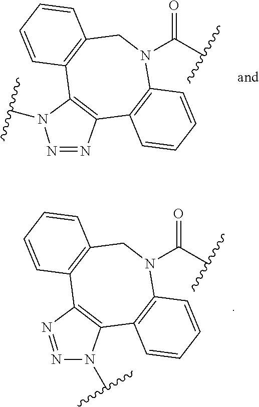 [SMILES: *C(=O)N1Cc2ccccc2-c2c(nnn2*)-c2ccccc21 ; *C(=O)N1Cc2ccccc2-c2nnn(*)c2-c2ccccc21]

Triazole regioisomers may also be represented by the following structure:

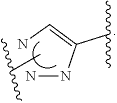

Unless otherwise stated, all tautomeric forms of the compounds of the disclosure are within the scope of the disclosure.

Additionally, unless otherwise stated, structures depicted herein are also meant to include compounds that differ only in the presence of one or more isotopically enriched atoms. For example, compounds having the present structures except for the replacement of hydrogen by deuterium or tritium, or the replacement of a carbon by a ¹¹C- or ¹³C- or ¹⁴C-enriched carbon are within the scope of this disclosure.

It is also to be understood that the mention of one or more method steps does not preclude the presence of additional method steps or intervening method steps between those steps expressly identified. Similarly, it is also to be understood that the mention of one or more components in a device or system does not preclude the presence of additional components or intervening components between those components expressly identified.

Unless otherwise stated, all crystalline forms of the compounds of the disclosure and salts thereof are also within the scope of the disclosure. The compounds of the disclosure may be isolated in various amorphous and crystalline forms, including without limitation forms which are anhydrous, hydrated, non-solvated, or solvated. Example hydrates include hemihydrates, monohydrates, dihydrates, and the like. In some embodiments, the compounds of the disclosure are anhydrous and non-solvated. By “anhydrous” is meant that the crystalline form of the compound contains essentially no bound water in the crystal lattice structure, i.e., the compound does not form a crystalline hydrate.

As used herein, “crystalline form” is meant to refer to a certain lattice configuration of a crystalline substance. Different crystalline forms of the same substance typically have different crystalline lattices (e.g., unit cells) which are attributed to different physical properties that are characteristic of each of the crystalline forms. In some instances, different lattice configurations have different water or solvent content. The different crystalline lattices can be identified by solid state characterization methods such as by X-ray powder diffraction (PXRD). Other characterization methods such as differential scanning calorimetry (DSC), thermogravimetric analysis (TGA), dynamic vapor sorption (DVS), solid state NMR, and the like further help identify the crystalline form as well as help determine stability and solvent/water content.

Crystalline forms of a substance include both solvated (e.g., hydrated) and non-solvated (e.g., anhydrous) forms. A hydrated form is a crystalline form that includes water in the crystalline lattice. Hydrated forms can be stoichiometric hydrates, where the water is present in the lattice in a certain water/molecule ratio such as for hemihydrates, monohydrates, dihydrates, etc. Hydrated forms can also be non-stoichiometric, where the water content is variable and dependent on external conditions such as humidity.

In some embodiments, the compounds of the disclosure are substantially isolated. By “substantially isolated” is meant that a particular compound is at least partially isolated from impurities. For example, in some embodiments a compound of the disclosure comprises less than about 50%, less than about 40%, less than about 30%, less than about 20%, less than about 15%, less than about 10%, less than about 5%, less than about 2.5%, less than about 1%, or less than about 0.5% of impurities. Impurities generally include anything that is not the substantially isolated compound including, for example, other crystalline forms and other substances.

Certain groups, moieties, substituents, and atoms are depicted with a wavy line. The wavy line can intersect or cap a bond or bonds. The wavy line indicates the atom through which the groups, moieties, substituents, or atoms are bonded. For example, a phenyl group that is substituted with a propyl group depicted as:

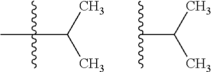

has the following structure:

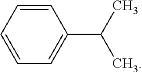

The term “GLP1R” refers to the glucagon-like peptide 1 receptor and includes recombinant GLP1R protein or a fragment thereof. GLP1R has a sequence of 463 residues. Donnelly, Br J Pharmacol, 166(1):27-41 (2011). Glucagon-like peptide 1 (GLP1) is a 31-amino acid peptide hormone released from intestinal L cells following nutrient consumption. The binding of GLP1 to GLP1R potentiates glucose-induced secretion of insulin from pancreatic beta cells, increases insulin expression, inhibits beta-cell apoptosis, promotes beta-cell neogenesis, reduces glucagon secretion, delays gastric emptying, promotes satiety, and increases peripheral glucose disposal.

The phrase “an antibody that binds GLP1R” or an “anti-GLP1R antibody” includes antibodies and antigen-binding fragments thereof that specifically recognize GLP1R.

An “agonist antibody,” as used herein (or an “antibody that increases or enhances GLP1R activity”), is intended to refer to an antibody whose binding to GLP1R results in activation of at least one biological activity of GLP1R. For example, an agonist antibody of GLP1R may elicit stimulation of the adenylate cyclase pathway resulting in increased synthesis of cyclic AMP and release of insulin if the cell is a mammalian pancreatic beta cell. An agonist antibody of GLP1R may also reduce glucose levels upon administration to a subject in need thereof.

All amino acid abbreviations used in this disclosure are those accepted by the United States Patent and Trademark Office as set forth in 37 C.F.R. § 1.822 (B)(J).

The term “protein” means any amino acid polymer having more than about 20 amino acids covalently linked via amide bonds. As used herein, “protein” includes biotherapeutic proteins, recombinant proteins used in research or therapy, trap proteins and other Fc-fusion proteins, chimeric proteins, antibodies, monoclonal antibodies, human antibodies, bispecific antibodies, antibody fragments, nanobodies, recombinant antibody chimeras, scFv fusion proteins, cytokines, chemokines, peptide hormones, and the like. Proteins can be produced using recombinant cell-based production systems, such as the insect bacculovirus system, yeast systems (e.g, Pichia sp.), mammalian systems (e.g., CHO cells and CHO derivatives like CHO-K1 cells).

All references to proteins, polypeptides and protein fragments herein are intended to refer to the human version of the respective protein, polypeptide or protein fragment unless explicitly specified as being from a non-human species. Thus, the expression “GLP1R” means human GLP1R unless specified as being from a non-human species, e.g., “mouse GLP1R,” “monkey GLP1R,” etc.

The amino acid sequence of an antibody can be numbered using any known numbering schemes, including those described by Kabat et al., (“Kabat” numbering scheme); Al-Lazikani et al., 1997, J. Mol. Biol., 273:927-948 (“Chothia” numbering scheme); MacCallum et al., 1996, J. Mol. Biol. 262:732-745 (“Contact” numbering scheme); Lefranc et al., Dev. Comp. Immunol., 2003, 27:55-77 (“IMGT” numbering scheme); and Honegge and Pluckthun, J. Mol. Biol., 2001, 309:657-70 (“AHo” numbering scheme). Unless otherwise specified, the numbering scheme used herein is the Kabat numbering scheme. However, selection of a numbering scheme is not intended to imply differences in sequences where they do not exist, and one of skill in the art can readily confirm a sequence position by examining the amino acid sequence of one or more antibodies. Unless stated otherwise, the “EU numbering scheme” is generally used when referring to a residue in an antibody heavy chain constant region (e.g., as reported in Kabat et al., supra).

The term “glutaminyl-modified antibody” refers to an antibody with at least one covalent linkage from a glutamine side chain to a primary amine compound of the present disclosure. In particular embodiments, the primary amine compound is linked through an amide linkage on the glutamine side chain. In certain embodiments, the glutamine is an endogenous glutamine. In other embodiments, the glutamine is an endogenous glutamine made reactive by polypeptide engineering (e.g., via amino acid deletion, insertion, substitution, or mutation on the polypeptide). In additional embodiments, the glutamine is polypeptide engineered with an acyl donor glutamine-containing tag (e.g., glutamine-containing peptide tags, Q-tags, or TGase recognition tag).

The term “TGase recognition tag” refers to a sequence of amino acids comprising an acceptor glutamine residue and that when incorporated into (e.g. appended to) a polypeptide sequence, under suitable conditions, is recognized by a TGase and leads to cross-linking by the TGase through a reaction between an amino acid side chain within the sequence of amino acids and a reaction partner. The recognition tag may be a peptide sequence that is not naturally present in the polypeptide comprising the TGase recognition tag. In some embodiments, the TGase recognition tag comprises at least one Gln. In some embodiments, the TGase recognition tag comprises an amino acid sequence XXQX, wherein X is any amino acid (e.g., conventional amino acid Leu, Ala, Gly, Ser, Val, Phe, Tyr, His, Arg, Asn, Glu, Asp, Cys, Gin, He, Met, Pro, Thr, Lys, or Trp or nonconventional amino acid). In some embodiments, the acyl donor glutamine-containing tag comprises an amino acid sequence selected from the group consisting of LLQGG (SEQ ID NO: 55), LLQG (SEQ ID NO: 56), LSLSQG (SEQ ID NO: 57), GGGLLQGG (SEQ ID NO: 58), GLLQG (SEQ ID NO: 59), LLQ, GSPLAQSHGG (SEQ ID NO: 60), GLLQGGG (SEQ ID NO: 61), GLLQGG (SEQ ID NO: 62), GLLQ (SEQ ID NO: 63), LLQLLQGA (SEQ ID NO: 64), LLQGA (SEQ ID NO: 65), LLQYQGA (SEQ ID NO: 66), LLQGSG (SEQ ID NO: 67), LLQYQG (SEQ ID NO: 68), LLQLLQG (SEQ ID NO: 69), SLLQG (SEQ ID NO: 70), LLQLQ (SEQ ID NO: 71), LLQLLQ (SEQ ID NO: 72), and LLQGR (SEQ ID NO: 73). See for example, WO2012059882, the entire contents of which are incorporated herein.

The term “antibody,” as used herein, means any antigen-binding molecule or molecular complex comprising at least one complementarity determining region (CDR) that specifically binds to or interacts with a particular antigen. The term “antibody” includes immunoglobulin molecules comprising four polypeptide chains, two heavy (H) chains and two light (L) chains inter-connected by disulfide bonds, as well as multimers thereof (e.g., IgM). Each heavy chain comprises a heavy chain variable region (abbreviated herein as HCVR or VH) and a heavy chain constant region. The heavy chain constant region comprises three domains, CH1, CH2, and CH3. Each light chain comprises a light chain variable region (abbreviated herein as LCVR or VL) and a light chain constant region. The light chain constant region comprises one domain (CL1). The VH and VL regions can be further subdivided into regions of hypervariability, termed complementarity determining regions (CDRs), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. In different embodiments, the FRs of the antibody (or antigen-binding portion thereof) can be identical to the human germline sequences, or can be naturally or artificially modified. An amino acid consensus sequence can be defined based on a side-by-side analysis of two or more CDRs.

The term “antibody,” as used herein, also includes antigen-binding fragments of full antibody molecules. The terms “antigen-binding portion” of an antibody, “antigen-binding fragment” of an antibody, and the like, as used herein, include any naturally occurring, enzymatically obtainable, synthetic, or genetically engineered polypeptide or glycoprotein that specifically binds an antigen to form a complex. Antigen-binding fragments of an antibody can be derived, e.g., from full antibody molecules using any suitable standard techniques such as proteolytic digestion or recombinant genetic engineering techniques involving the manipulation and expression of DNA encoding antibody variable and optionally constant domains. Such DNA is known and/or is readily available from, e.g., commercial sources, DNA libraries (including, e.g., phage-antibody libraries), or can be synthesized. The DNA can be sequenced and manipulated chemically or by using molecular biology techniques, for example, to arrange one or more variable and/or constant domains into a suitable configuration, or to introduce codons, create cysteine residues, modify, add or delete amino acids, etc.

Non-limiting examples of antigen-binding fragments include: (i) Fab fragments; (ii) F(ab′)2 fragments; (iii) Fd fragments; (iv) Fv fragments; (v) single-chain Fv (scFv) molecules; (vi) dAb fragments; and (vii) minimal recognition units consisting of the amino acid residues that mimic the hypervariable region of an antibody (e.g., an isolated complementarity determining region (CDR) such as a CDR3 peptide), or a constrained FR3-CDR3-FR4 peptide. Other engineered molecules, such as domain-specific antibodies, single domain antibodies, domain-deleted antibodies, chimeric antibodies, CDR-grafted antibodies, diabodies, triabodies, tetrabodies, minibodies, nanobodies (e.g., monovalent nanobodies, bivalent nanobodies, etc.), small modular immunopharmaceuticals (SMIPs), and shark variable IgNAR domains, are also encompassed within the expression “antigen-binding fragment,” as used herein.

An antigen-binding fragment of an antibody will typically comprise at least one variable domain. The variable domain can be of any size or amino acid composition and will generally comprise at least one CDR which is adjacent to or in frame with one or more framework sequences. In antigen-binding fragments having a VH domain associated with a VL domain, the VH and VL domains can be situated relative to one another in any suitable arrangement. For example, the variable region can be dimeric and contain VH-VH, VH-VL or VL-VL dimers.

Alternatively, the antigen-binding fragment of an antibody can contain a monomeric VH or VL domain.

In certain embodiments, an antigen-binding fragment of an antibody can contain at least one variable domain covalently linked to at least one constant domain. Non-limiting, exemplary configurations of variable and constant domains that can be found within an antigen-binding fragment of an antibody of the present description include: (i) VH-CH1; (ii) VH-CH2; (iii) VH-CH3; (iv) VH-CH1-CH2; (V) VH-CH1-CH2-CH3; (vi) VH-CH2-CH3; (vii) VH-CL; (viii) VL-CH1; (ix) VL-CH2; (x) VL-CH3; (xi) VL-CH1-CH2; (xii) VL-CH1-CH2-CH3; (xiii) VL-CH2-CH3; and (xiv) VL-CL. In any configuration of variable and constant domains, including any of the exemplary configurations listed herein, the variable and constant domains can be either directly linked to one another or can be linked by a full or partial hinge or linker region. A hinge region can consist of at least 2 (e.g., 5, 10, 15, 20, 40, 60, or more) amino acids which result in a flexible or semi-flexible linkage between adjacent variable and/or constant domains in a single polypeptide molecule.

Moreover, an antigen-binding fragment of an antibody of the present description can comprise a homo-dimer or hetero-dimer (or other multimer) of any of the variable and constant domain configurations listed herein in non-covalent association with one another and/or with one or more monomeric VH or VL domain (e.g., by disulfide bond(s)).

As with full antibody molecules, antigen-binding fragments can be monospecific or multispecific (e.g., bispecific). A multispecific antigen-binding fragment of an antibody will typically comprise at least two different variable domains, wherein each variable domain is capable of specifically binding to a separate antigen or to a different epitope on the same antigen. Any multispecific antibody format, including the exemplary bispecific antibody formats disclosed herein, can be adapted for use in the context of an antigen-binding fragment of an antibody of the present description using routine techniques available in the art.

In certain embodiments, the antibodies of the description, e.g., anti-GLP1R antibodies, are human antibodies. The term “human antibody,” as used herein, is intended to include antibodies having variable and constant regions derived from human germline immunoglobulin sequences. The human antibodies of the description can include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody,” as used herein, is not intended to include antibodies in which CDR sequences derived from the germline of another mammalian species, such as a mouse, have been grafted onto human framework sequences.

The antibodies can, in some embodiments, be recombinant human antibodies. The term “recombinant human antibody,” as used herein, is intended to include all human antibodies that are prepared, expressed, created or isolated by recombinant means, such as antibodies expressed using a recombinant expression vector transfected into a host cell (described further below), antibodies isolated from a recombinant, combinatorial human antibody library (described further below), antibodies isolated from an animal (e.g., a mouse) that is transgenic for human immunoglobulin genes (See, e.g., Taylor et al. (1992) Nucl. Acids Res. 20:6287-6295) or antibodies prepared, expressed, created or isolated by any other means that involves splicing of human immunoglobulin gene sequences to other DNA sequences. Such recombinant human antibodies have variable and constant regions derived from human germline immunoglobulin sequences. In certain embodiments, however, such recombinant human antibodies are subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo.

Human antibodies can exist in two forms that are associated with hinge heterogeneity. In one form, an immunoglobulin molecule comprises a stable four chain construct of approximately 150-160 kDa in which the dimers are held together by an interchain heavy chain disulfide bond. In a second form, the dimers are not linked via inter-chain disulfide bonds and a molecule of about 75-80 kDa is formed composed of a covalently coupled light and heavy chain (half-antibody). These forms have been extremely difficult to separate, even after affinity purification. The frequency of appearance of the second form in various intact IgG isotypes is due to, but not limited to, structural differences associated with the hinge region isotype of the antibody. A single amino acid substitution in the hinge region of the human IgG4 hinge can significantly reduce the appearance of the second form (Angal et al. (1993) Molecular Immunology 30: 105) to levels typically observed using a human IgG1 hinge. The instant description encompasses antibodies having one or more mutations in the hinge, CH2 or CH3 region which can be desirable, for example, in production, to improve the yield of the desired antibody form.

The antibodies of the description can be isolated or purified antibodies. An “isolated antibody” or “purified antibody,” as used herein, means an antibody that has been identified and separated and/or recovered from at least one component of its natural environment. For example, an antibody that has been separated or removed from at least one component of an organism, or from a tissue or cell in which the antibody naturally exists or is naturally produced, is an “isolated antibody” for purposes of the present description. For example, an antibody that has been purified from at least one component of a reaction or reaction sequence, is a “purified antibody” or results from purifying the antibody. An isolated antibody also includes an antibody in situ within a recombinant cell. Isolated antibodies are antibodies that have been subjected to at least one purification or isolation step. According to certain embodiments, an isolated antibody or purified antibody can be substantially free of other cellular material and/or chemicals.

The antibodies disclosed herein can comprise one or more amino acid substitutions, insertions and/or deletions in the framework and/or CDR regions of the heavy and light chain variable domains as compared to the corresponding germline sequences from which the antibodies were derived. Such mutations can be readily ascertained by comparing the amino acid sequences disclosed herein to germline sequences available from, for example, public antibody sequence databases. The present description includes antibodies, and antigen-binding fragments thereof, which are derived from any of the amino acid sequences disclosed herein, wherein one or more amino acids within one or more framework and/or CDR regions are mutated to the corresponding residue(s) of the germline sequence from which the antibody was derived, or to the corresponding residue(s) of another human germline sequence, or to a conservative amino acid substitution of the corresponding germline residue(s) (such sequence changes are referred to herein collectively as “germline mutations”). A person of ordinary skill in the art, starting with given heavy and light chain variable region sequences, can easily produce numerous antibodies and antigen-binding fragments which comprise one or more individual germline mutations or combinations thereof. In certain embodiments, all of the framework and/or CDR residues within the VH and/or VL domains are mutated back to the residues found in the original germline sequence from which the antibody was derived. In other embodiments, only certain residues are mutated back to the original germline sequence, e.g., only the mutated residues found within the first 8 amino acids of FR1 or within the last 8 amino acids of FR4, or only the mutated residues found within CDR1, CDR2, or CDR3. In other embodiments, one or more of the framework and/or CDR residue(s) are mutated to the corresponding residue(s) of a different germline sequence (i.e., a germline sequence that is different from the germline sequence from which the antibody was originally derived).

Furthermore, the antibodies of the present description can contain any combination of two or more germline mutations within the framework and/or CDR regions, e.g., wherein certain individual residues are mutated to the corresponding residue of a particular germline sequence while certain other residues that differ from the original germline sequence are maintained or are mutated to the corresponding residue of a different germline sequence. Once obtained, antibodies and antigen-binding fragments that contain one or more germline mutations can be easily tested for one or more desired property such as, improved binding specificity, increased binding affinity, improved or enhanced antagonistic or agonistic biological properties (as the case may be), reduced immunogenicity, improved drug-to-antibody ratio (DAR) for antibody-drug conjugates, etc. Antibodies and antigen-binding fragments obtained in this general manner are encompassed within the present description.

The term “aglycosylated antibody” refers to an antibody that does not comprise a glycosylation sequence that might interfere with a transglutamination reaction, for instance an antibody that does not have saccharide group at N297 on one or more heavy chains. In particular embodiments, an antibody heavy chain has an N297 mutation. In other words, the antibody is mutated to no longer have an asparagine residue at position 297 according to the EU numbering system as disclosed by Kabat et al. In particular embodiments, an antibody heavy chain has an N297Q or an N297D mutation. Such an antibody can be prepared by site-directed mutagenesis to remove or disable a glycosylation sequence or by site-directed mutagenesis to insert a glutamine residue at site apart from any interfering glycosylation site or any other interfering structure. Such an antibody also can be isolated from natural or artificial sources. Aglycosylated antibodies also include antibodies comprising a T299 or S298P or other mutations, or combinations of mutations that result in a lack of glycosylation.

The term “deglycosylated antibody” refers to an antibody in which a saccharide group at is removed to facilitate transglutaminase-mediated conjugation. Saccharides include, but are not limited to, N-linked oligosaccharides. In some embodiments, deglycosylation is performed at residue N297. In some embodiments, removal of saccharide groups is accomplished enzymatically, included but not limited to via PNGase.

The term “epitope” refers to an antigenic determinant that interacts with a specific antigen binding site in the variable region of an antibody molecule known as a paratope. A single antigen can have more than one epitope. Thus, different antibodies can bind to different areas on an antigen and can have different biological effects. Epitopes can be either conformational or linear. A conformational epitope is produced by spatially juxtaposed amino acids from different segments of the linear polypeptide chain. A linear epitope is one produced by adjacent amino acid residues in a polypeptide chain. In certain circumstance, an epitope can include moieties of saccharides, phosphoryl groups, or sulfonyl groups on the antigen.

The terms “conjugated protein” or “conjugated antibody” as used herein refers to a protein or an antibody covalently linked to one or more chemical moieties. The chemical moiety can include an amine compound of the present disclosure. Linkers (L) and payloads (P) suitable for use with the present disclosure are described in detail herein. In particular embodiments, a conjugated antibody comprising a therapeutic moiety is an antibody-drug conjugate (ADC), or antibody-tethered ligand (ATL), or an antibody-tethered drug conjugate (ATDC), also referred to as an antibody-payload conjugate, or an antibody-linker-payload conjugate.

The term “Drug-to-Antibody Ratio” or (DAR) is the average number of therapeutic moieties, e.g., drugs, conjugated to a binding agent of the present disclosure.

The term “Linker Antibody Ratio” or (LAR), also denoted as the lower case, in some embodiments, is the average number of reactive primary amine compounds conjugated to a binding agent of the present disclosure. Such binding agents, e.g., antibodies, can be conjugated with primary amine compounds comprising, e.g., a suitable azide or alkyne. The resulting binding agent, which is functionalized with an azide or an alkyne can subsequently react with a therapeutic moiety comprising the corresponding azide or alkyne via the 1,3-cycloaddition reaction.

The phrase “pharmaceutically acceptable amount” refers to an amount effective or sufficient in treating, reducing, alleviating, or modulating the effects or symptoms of at least one health problem in a subject in need thereof. For example, a pharmaceutically acceptable amount of an antibody or antibody-drug conjugate is an amount effective for modulating a biological target using the antibody or antibody-drug-conjugates provided herein. Suitable pharmaceutically acceptable amounts include, but are not limited to, from about 0.001% up to about 10%, and any amount in between, such as about 0.01%, about 0.02%, about 0.03%, about 0.04%, about 0.05%, about 0.06%, about 0.07%, about 0.08%, about 0.09%, about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, or about 10% of an antibody or antibody-drug-conjugate provided herein.

The phrase “reaction pH” refers to the pH of a reaction after all reaction components or reactants have been added.

The term “substantial identity” or “substantially identical,” when referring to a nucleic acid or fragment thereof, indicates that, when optimally aligned with appropriate nucleotide insertions or deletions with another nucleic acid (or its complementary strand), there is nucleotide sequence identity in at least about 95%, and more preferably at least about 96%, 97%, 98%, or 99% of the nucleotide bases, as measured by any well-known algorithm of sequence identity, such as FASTA, BLAST or Gap, as discussed below. A nucleic acid molecule having substantial identity to a reference nucleic acid molecule can, in certain instances, encode a polypeptide having the same or substantially similar amino acid sequence as the polypeptide encoded by the reference nucleic acid molecule.

As applied to polypeptides, the term “substantial similarity” or “substantially similar” means that two peptide sequences, when optimally aligned, such as by the programs GAP or BESTFIT using default gap weights, share at least 95% sequence identity, even more preferably at least 98% or 99% sequence identity. Preferably, residue positions which are not identical differ by conservative amino acid substitutions. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent sequence identity or degree of similarity can be adjusted upwards to correct for the conservative nature of the substitution. Means for making this adjustment are well-known to those of skill in the art. See, e.g., Pearson (1994) Methods Mol. Biol. 24: 307-331. Examples of groups of amino acids that have side chains with similar chemical properties include (1) aliphatic side chains: glycine, alanine, valine, leucine and isoleucine; (2) aliphatic-hydroxyl side chains: serine and threonine; (3) amide-containing side chains: asparagine and glutamine; (4) aromatic side chains: phenylalanine, tyrosine, and tryptophan; (5) basic side chains: lysine, arginine, and histidine; (6) acidic side chains: aspartate and glutamate, and (7) sulfur-containing side chains are cysteine and methionine. In some embodiments, conservative amino acids substitution groups are: valine-leucine-isoleucine, phenylalanine-tyrosine, lysine-arginine, alanine-valine, glutamate-aspartate, and asparagine-glutamine.

Alternatively, a conservative replacement is any change having a positive value in the PAM250 log-likelihood matrix disclosed in Gonnet et al. (1992) Science 256: 1443-1445. A “moderately conservative” replacement is any change having a nonnegative value in the PAM250 log-likelihood matrix.

Sequence similarity for polypeptides, which is also referred to as sequence identity, is typically measured using sequence analysis software. Protein analysis software matches similar sequences using measures of similarity assigned to various substitutions, deletions and other modifications, including conservative amino acid substitutions. For instance, GCG software contains programs such as Gap and Bestfit which can be used with default parameters to determine sequence homology or sequence identity between closely related polypeptides, such as homologous polypeptides from different species of organisms or between a wild type protein and a mutein thereof. See, e.g., GCG Version 6.1. Polypeptide sequences also can be compared using FASTA using default or recommended parameters, a program in GCG Version 6.1. FASTA (e.g., FASTA2 and FASTA3) provides alignments and percent sequence identity of the regions of the best overlap between the query and search sequences (Pearson (2000) supra). Another particular algorithm when comparing a sequence of the description to a database containing a large number of sequences from different organisms is the computer program BLAST, especially BLASTP or TBLASTN, using default parameters. See, e.g., Altschul et al. (1990) J. Mol. Biol. 215:403-410 and Altschul et al. (1997) Nucleic Acids Res. 25:3389-402.

Additional disclosures can be found in U.S. Application Ser. No. 63/744,076, filed Jan. 10, 2025, and U.S. Application Ser. No. 63/770,235, filed on Mar. 11, 2025, both of which are incorporated herein by reference in their entirety.

### Conjugates

In one aspect, provided herein is a conjugate, or a pharmaceutically acceptable salt thereof, comprising an antigen-binding protein (ABP) and at least one glucagon-like peptide-1 (GLP-1) receptor (GLP1R) and glucose-dependent insulinotropic polypeptide (GIP) receptor (GIPR) dual agonist (GLP1R-GIPR dual agonist), wherein the GLP1R and GIPR dual agonist is covalently conjugated to the ABP through a linker.

In some embodiments, the ABP is an antibody, or an antigen binding fragment thereof, that specifically binds to and/or blocks the activity of growth and differentiation factor-8 (GDF8).

In some embodiments, the ABP is an antibody that specifically binds to and/or blocks the activity of growth and differentiation factor-8 (GDF8) having a sequence of SEQ ID NO: 1.

In another aspect, provided herein is a conjugate, or a pharmaceutically acceptable salt thereof, comprising a GLP1R and GIPR dual agonist according to any of the embodiments described herein, an antigen binding protein (ABP), and a linker covalently linking the dual agonist and ABP, where the ABP is a GDF8 antibody, or the antigen-binding fragment thereof.

In another aspect, provided herein is a GLP1R-GIPR dual agonist and an antigen binding protein (ABP) conjugate, or a pharmaceutically acceptable salt thereof, having a Formula (I):

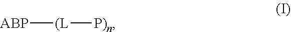

where
   ABP is an antibody, or an antigen-binding fragment thereof, that specifically binds to and/or blocks the biological activity of human Growth and Differentiation Factor-8 (GDF8);
   P is a glucagon-like peptide-1 (GLP-1) receptor (GLP1R) and glucose-dependent insulinotropic polypeptide (GIP) receptor (GIPR) dual agonist;
   L is a linker that covalently links P and ABP; and
   n ranges from about 1 to about 6.

In some embodiments, the conjugate has a formula (II):

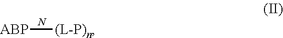

where [symbol] represents that the connection between the ABP and the L is through the side chain of a glutamine residue (Gln) of the ABP.

In some embodiments, the Gln is a naturally occurring Gln or an engineered Gln.

In some embodiments, the Gln is Gln55 (Q55).

In some embodiments, the Gln is Gln295 (Q295) or Gln297 (Q297).

In some embodiments, ABP is first modified with a handle comprising (a) a reactive moiety for Click or Diels-Alder reaction and (b) a primary amine suitable for microbial transglutaminase-mediated conjugation.

In some embodiments, the handle comprises a primary amino group for conjugation mediated by microbial transglutaminase and a reactive moiety suitable for Click or Diels-Alder reaction.

In some embodiments, the handle comprises

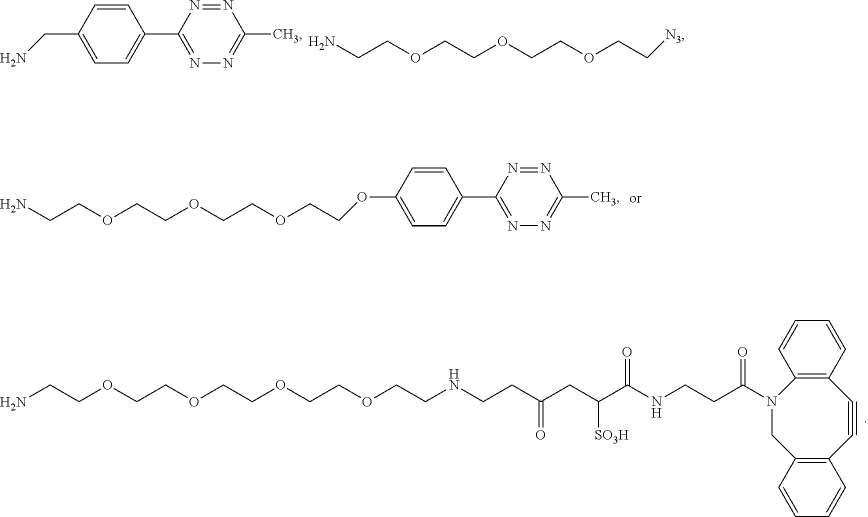 [SMILES: Cc1nnc(-c2ccc(CN)cc2)nn1 ; [N-]=[N+]=NCCOCCOCCOCCN ; Cc1nnc(-c2ccc(OCCOCCOCCOCCN)cc2)nn1 ; NCCOCCOCCOCCOCCNCCC(=O)CC(C(=O)NCCC(=O)N1Cc2ccccc2C#Cc2ccccc21)S(=O)(=O)O]

In some embodiments, the handle is selected from the group consisting of

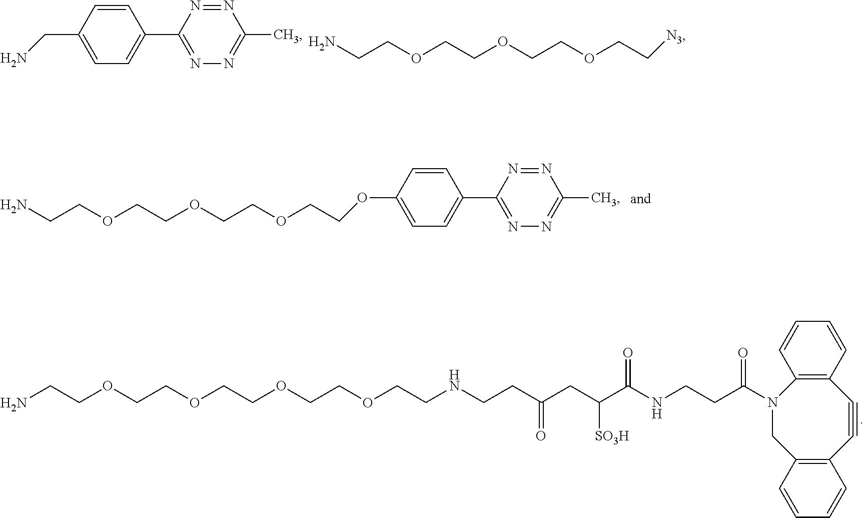 [SMILES: Cc1nnc(-c2ccc(CN)cc2)nn1 ; [N-]=[N+]=NCCOCCOCCOCCN ; Cc1nnc(-c2ccc(OCCOCCOCCOCCN)cc2)nn1 ; NCCOCCOCCOCCOCCNCCC(=O)CC(C(=O)NCCC(=O)N1Cc2ccccc2C#Cc2ccccc21)S(=O)(=O)O]

In some embodiments, the reactive moiety for Click or Diels-Alder reaction is selected from the group consisting of from

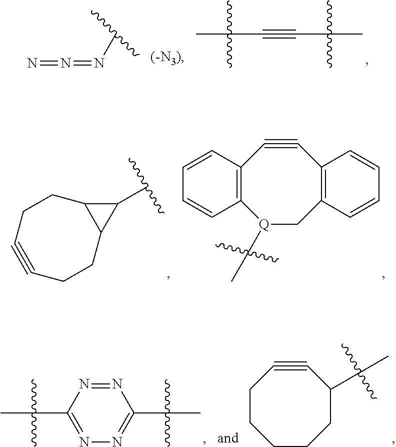

where Q is CH or N.

In some embodiments, the conjugate has a Formula (III):

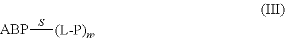

where [symbol] represents that the connection between ABP and linker L via the side chain of a cysteine residue (Cys) of the ABP.

In some embodiments, the Cys of the BA is a naturally occurring Cys or an engineered Cys.

In some embodiments, L comprises a moiety resulted from chemical conjugation of a reactive group (RG) on the L-P with the side chain of Cys of the ABP.

In some embodiments, the reactive group is a bromo acetyl or maleimide.

### Anti-GDF8 Antibodies and Antigen-Binding Fragments Thereof

Growth and Differentiation Factor-8 (GDF8), also known as myostatin, is a member of the TGF-β superfamily of growth factors. GDF8 is a negative regulator of skeletal muscle mass, highly expressed in the developing and adult skeletal muscle.

GDF8 is highly conserved across species, and the amino acid sequences of murine and human GDF8 are identical (human GDF8 nucleic acid sequence and amino acid sequence shown in SEQ ID NO:338-339) (McPherron et al. 1977 Nature 387:83-90).

Antibodies to GDF8 and therapeutic methods are disclosed in, e.g., U.S. Pat. Nos. 6,096,506, 7,320,789, 7,807,159, WO 2007/047112, WO 2005/094446, US 2007/0087000, U.S. Pat. No. 7,261,893, and WO 2010/070094.

These antibodies can be full-length (for example, an IgG1 or IgG4 antibody) or may comprise only an antigen-binding portion (for example, a Fab, F(ab′)₂ or scFv fragment), and may be modified to affect functionality, e.g., to eliminate residual effector functions (Reddy et al. (2000) J. Immunol. 164:1925-1933).

In certain specific embodiments of the present disclosure, the GDF-8 inhibitor is a GDF8-specific binding protein, and the protein, or the GDF8-specific binding domain, comprises or consists of an anti-GDF8 antibody or antigen-binding fragment thereof. Anti-GDF8 antibodies are mentioned in, e.g., U.S. Pat. Nos. 6,096,506; 7,320,789; 7,261,893; 7,807,159; 7,888,486; 7,635,760; 7,632,499; in US Patent Appl. Publ. Nos. 2007/0178095; 2010/0166764; 2009/0148436; and International Patent Appl. Publ. No. WO 2010/070094. Anti-GDF8 antibodies are also described in U.S. patent application Ser. No. 13/115,170, filed on May 25, 2011, and published as US 20110293630, and the continuation application Ser. No. 14/462,085, filed Aug. 18, 2014, published as US 20140356369, including the antibodies designated 8D12, H4H1657N2, and H4H1669P. In some embodiments, the anti-GDF8 antibodies are selected from the group consisting of AMG 181, AMG 745, Apitegromab (SRK-015), Domagrozumab (PF-06252616), GYM329 (RO7204239), Landogrozumab (LY2495655), Stamulumab (MYO-029), and Trevogrumab (REGN1033).

In one embodiment, the anti-GDF8 antibody is REGN1033, also known as trevogrumab, or H4H1657N2. Any of the anti-GDF8 antibodies mentioned and/or described in any of the foregoing patents or publications, or antigen-binding fragments thereof, can be used in the context of the present disclosure, so long as such antibodies and/or antigen-binding fragments “specifically bind” GDF8, as that expression is defined herein.

The present invention also includes anti-GDF8 antibodies comprising variants of any of the HCVR, LCVR, and/or CDR amino acid sequences disclosed herein having one or more conservative substitutions. For example, the present invention includes anti-GDF8 antibodies having HCVR, LCVR, and/or CDR amino acid sequences with, e.g., 10 or fewer, 8 or fewer, 6 or fewer, 4 or fewer, etc. conservative amino acid substitutions relative to any of the HCVR, LCVR, and/or CDR amino acid sequences disclosed herein.

Suitable GDF8 antibody or the antigen binding fragment thereof that can be used include the ones mentioned in U.S. Pat. No. 8,840,894. In some embodiments, the GDF8 antibody or the antigen binding fragment thereof comprises 1A2, 21-E5, 8D12, H4H1657N2 (REGN1033), and H4H1669P. See U.S. Pat. No. 8,840,894.

In some embodiments, the GDF8 antibody, or the antigen binding fragment thereof, comprises 1A2, 21-E5, 8D12, H4H1669P, AMG 181, AMG 745, Apitegromab (SRK-015), Bimagrumab (BYM338), Domagrozumab (PF-06252616), GYM329 (RO7204239), Landogrozumab (LY2495655), Stamulumab (MYO-029), and Trevogrumab (REGN1033).

In one embodiment, the ABP is an antibody that specifically binds to and/or blocks the activity of growth and differentiation factor-8 (GDF8) having a sequence of SEQ ID NO: 1.

In another embodiment, the anti-GDF8 antibody or antigen-binding fragment thereof comprises the heavy chain complementarity determining regions (HCDRs) of a heavy chain variable region (HCVR) comprising SEQ ID NO:4, and the light chain complementarity determining regions (LCDRs) of a light chain variable region (LCVR) comprising SEQ ID NO: 5.

In another embodiment, the anti-GDF8 antibody or antigen-binding fragment thereof comprises three heavy chain complementarity determining regions (HCDR1, HCDR2, and HCDR3) comprising SEQ ID NO: 6, SEQ ID NO: 7, and SEQ ID NO: 8, respectively, and three light chain complementarity determining regions (LCDR1, LCDR2, and LCDR3) comprising SEQ ID NO: 9, SEQ ID NO: 10, and SEQ ID NO: 11, respectively.

In some embodiments, the ABP of the GDF8 antibody or the antigen binding fragment thereof comprises 1A2, 21-E5, 8D12, H4H1669P, AMG 181, AMG 745, Apitegromab (SRK-015), Bimagrumab (BYM338), Domagrozumab (PF-06252616), GYM329 (RO7204239), Landogrozumab (LY2495655), Stamulumab (MYO-029), and Trevogrumab (REGN1033).

In some embodiments, the ABP of the GDF8 antibody or the antigen binding fragment thereof binds to and/or blocks the activity of growth and differentiation factor-8 (GDF8) having a sequence of SEQ ID NO: 1.

In some embodiments, ABP of the GDF8 antibody or the antigen binding fragment thereof comprises three heavy chain complementarity determining regions (HCDR1, HCDR2, and HCDR3) comprising SEQ ID NO: 6, SEQ ID NO: 7, and SEQ ID NO: 8, respectively, and three light chain complementarity determining regions (LCDR1, LCDR2, and LCDR3) comprising SEQ ID NO: 9, SEQ ID NO: 10, and SEQ ID NO: 11, respectively.

In some embodiments, ABP of the GDF8 antibody or the antigen binding fragment thereof comprises heavy chain complementarity determining regions (HCDRs) of a heavy chain variable region (HCVR) comprising SEQ ID NO:4, and light chain complementarity determining regions (LCDRs) of a light chain variable region (LCVR) comprising SEQ ID NO:5.

### Sequence Listing

| | SEQ | |
|---|---|---|
| | ID NO: | amino acid sequence |
| GDF8 (mature | 1 | Asp Phe Gly Leu Asp Cys Asp Glu His Ser Thr Glu Ser Arg |
| protein, myostatin) | | Cys Cys Arg Tyr Pro Leu Thr Val Asp Phe Glu Ala Phe Gly |
| | | Trp Asp Trp Ile Ile Ala Pro Lys Arg Tyr Lys Ala Asn Tyr Cys |
| | | Ser Gly Glu Cys Glu Phe Val Phe Leu Gln Lys Tyr Pro His |
| | | Thr His Leu Val His Gln Ala Asn Pro Arg Gly Ser Ala Gly |
| | | Pro Cys Cys Thr Pro Thr Lys Met Ser Pro Ile Asn Met Leu |
| | | Tyr Phe Asn Gly Lys Glu Gln Ile Ile Tyr Gly Lys Ile Pro Ala |
| | | Met Val Val Asp Arg Cys Gly Cys Ser |
| glucagon-like | 2 | His Ala Glu Gly Thr Phe Thr Ser Asp Val Ser Ser Tyr Leu |
| peptide-1(GLP-1) | | Glu Gly Gln Ala Ala Lys Glu Phe Ile Ala Trp Leu Val Lys |
| (7-37) | | Gly Arg Gly |
| Payload | 3 | Payload is a GLPIR and GIPR dual agonist having a |
| | | sequence of Tyr1 Aib2 XX3 Gly4 XX5 XX6 Thr Ser Asp Tyr |
| | | Ser Ser XX13 Leu Glu Glu Gln Ala Ala XX20 Glu XX22 Ile |
| | | XX24 XX25 Leu Val XX28 Gly Gly Gly, wherein Aib |
| | | represents 2-aminoisobutyric acid (a-aminoisobutyric acid); |
| | | XX3 is Glu or Atz ((S)-2-amino-3-(2H-tetrazol-5- |
| | | yl)propanoic acid); XX5 is Ser or Thr; XX6 is phenylalanine, |
| | | alpha methyl phenylalanine (amF) or 2-fluoro alpha methyl |
| | | phenylalanine (amF(2-fluoro)); XX13 is Tyr, amF, or alpha |
| | | methyl tyrosine (amY); XX20 is Lys, sidechain- acylated or |
| | | alkylated Lys, or Aib; XX22 is Phe or amF; XX24 is Ala or |
| | | Glu; XX25 is tryptophan or alpha methyl tyrosine (amY); |
| | | XX28 is Lys, Ala, Gln, or Glu. |
| anti-GDF8 ab | 4 | Glu Val Gln Val Leu Glu Ser Gly Gly Asp Leu Val Gln Pro |
| HCVR | | Gly Gly Ser Leu Arg Leu Ser Cys Ala Ala Ser Gly Phe Thr |
| | | Phe Ser Ala Tyr Ala Met Thr Trp Val Arg Gln Ala Pro Gly |
| | | Lys Gly Leu Glu Trp Val Ser Ala Ile Ser Gly Ser Gly Gly Ser |
| | | Ala Tyr Tyr Ala Asp Ser Val Lys Gly Arg Phe Thr Ile Ser Arg |
| | | Asp Asn Ser Lys Asn Thr Val Tyr Leu Gln Met Asn Ser Leu |
| | | Arg Ala Glu Asp Thr Ala Val Tyr Tyr Cys Ala Lys Asp Gly |
| | | Ala Trp Lys Met Ser Gly Leu Asp Val Trp Gly Gln Gly Thr |
| | | Thr Val Ile Val Ser Ser |
| anti-GDF8 ab | 5 | Asp Ile Gln Met Thr Gln Ser Pro Ala Ser Leu Ser Ala Ser Val |
| LCVR | | Gly Asp Arg Val Thr Ile Thr Cys Arg Ala Ser Gln Asp Ile Ser |
| | | Asp Tyr Leu Ala Trp Tyr Gln Gln Lys Pro Gly Lys Ile Pro |
| | | Arg Leu Leu Ile Tyr Thr Thr Ser Thr Leu Gln Ser Gly Val |
| | | Pro Ser Arg Phe Arg Gly Ser Gly Ser Gly Thr Asp Phe Thr |
| | | Leu Thr Ile Ser Ser Leu Gln Pro Glu Asp Val Ala Thr Tyr Tyr |
| | | Cys Gln Lys Tyr Asp Ser Ala Pro Leu Thr Phe Gly Gly Gly |
| | | Thr Lys Val Glu Ile Lys |
| anti-GDF8 ab | 6 | Gly Phe Thr Phe Ser Ala Tyr Ala |
| HCDR1 | | |
| anti-GDF8 ab | 7 | Ile Ser Gly Ser Gly Gly Ser Ala |
| HCDR2 | | |
| anti-GDF8 ab | 8 | Ala Lys Asp Gly Ala Trp Lys Met Ser Gly Leu Asp Val |
| HCDR3 | | |
| anti-GDF8 ab | 9 | Gln Asp Ile Ser Asp Tyr |
| LCDR1 | | |
| anti-GDF8 ab | | Thr Thr Ser |
| LCDR2 | | |
| anti-GDF8 ab | 11 | Gln Lys Tyr Asp Ser Ala Pro Leu Thr |
| LCDR3 | | |
| M6457 | 12 | H[Aib]EGTFTSDYSSYLEEQAAKEFIAWLVKGGG- |
| | | (GGGGS)3-K(Ac-Br)-amide |
| M6562 | 13 | H[Aib]EGTFTSDYSSYLEEQAAKEFIAWLVKGGG- |
| | | (GGGGS)3-K(Ac-COT/ALO)-amide |
| M6687 | 14 | 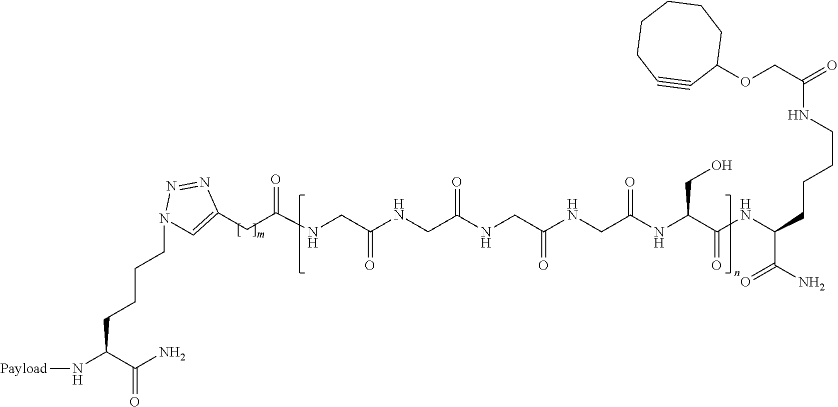 |
| M6687 | 14 | Y-Aib-EGT-amF-TSDYSSYLEEQAA KEFIA-am Y- |
| | | LVKGGG-K[T-CH₂CH₂CO-(GGGGS)3-K(ALO)] amide |
| M6695 | 15 | 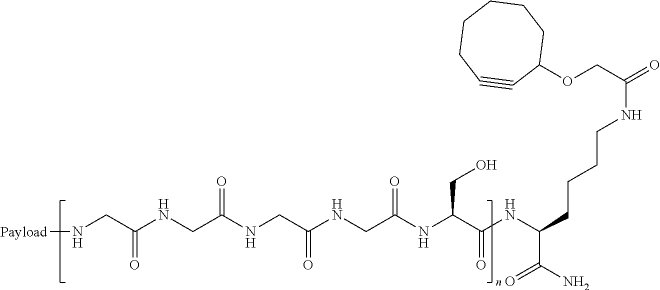 |
| M6695 | 15 | Y-Aib-EGT-amF-TSDYSSYLEEQAA KEFIA-am Y- |
| | | LVKGGG-(GGGGS)3-K(ALO) amide |
| M6696 | 16 | 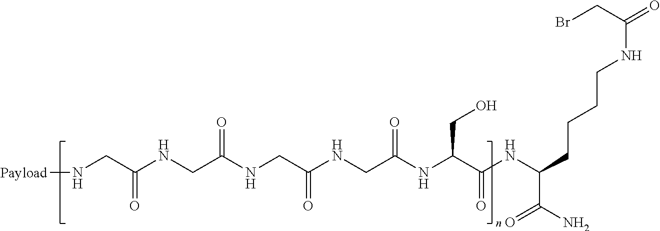 |
| M6696 | 16 | Y-Aib-EGT-amF-TSDYSSYLEEQAA KEFIA-amY- |
| | | LVKGGG-(GGGGS)3-K(N3) amide |
| M6682 | 17 | Y-Aib-EGT-amF-TSDYSSYLEEQAA KEFIA-am Y- |
| | | LVKGGG-(GGGGS)3-K(N3) amide |
| M6486 | 18 | Y-Aib-EGT-F-TSDYSSYLEEQAA KEFIA-W-LVKGGG |
| | | amide |
| M6487 | 19 | Y-Aib-EGT-amF(2-fluoro)-TSDYSSYLEEQAA KEFIA-W- |
| | | LVKGGG amide |
| M6953 | 20 | Y-Aib-EGT-amF(2-fluoro)-TSDYSSYLEEQAA KEFIA-W- |
| | | LVKGGG-K(N3) amide |
| M6954 | 21 | Y-Aib-EGT-amF(2-fluoro)-TSDYSSYLEEQAA KEFIA-W- |
| | | LVKGGG-K[K(N3)] amide |
| M6989 | 22 | Y-Aib-EGT-amF(2-fluoro)-TSDYSSYLEEQAA KEFIA-W- |
| | | LVKGGG-K[T-CH2CH2CO-(GGGGS)3-K(Ac-COT/ALO)] |
| | | amide |
| M6990 | 23 | Y-Aib-EGT-amF(2-fluoro)-TSDYSSYLEEQAA KEFIA-W- |
| | | LVKGGG -K[T-CH2NH-(ALO)] amide* |
| | | *- K[T-CH2-NH-(ALO)] amide represents: |
| | | 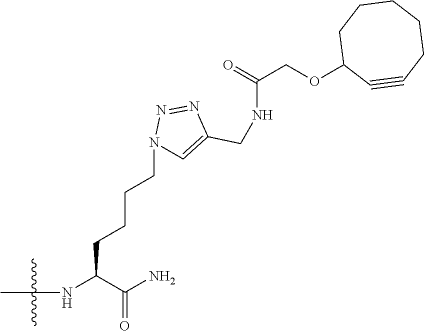 [SMILES: *N[C@@H](CCCCn1cc(CNC(=O)COC2C#CCCCCC2)nn1)C(N)=O] |
| M6956 | 24 | Y-Aib-EGT-amF-TSDYSS-amF-LEEQAA KEFIA-W- |
| | | LVKGGG-K(N3) amide |
| M6957 | 25 | Y-Aib-EGS-amF-TSDYSSYLEEQAA KEFIA-am Y- |
| | | LVKGGG-K(N3) amide |
| M6958 | 26 | Y-Aib-EGT-amF-TSDYSSYLEEQAA KE-amF-IA-W- |
| | | LVKGGG-K(N3) amide |
| M6959 | 27 | Y-Aib-EGT-amF-TSDYSS-am Y-LEEQAA KE-amF-IA-W- |
| | | LVKGGG-K(N3) amide |
| M6960 | 28 | Y-Aib-EGT-amF(2-fluoro)-TSDYSSYLEEQAA KEFIA- |
| | | am Y-LVKGGG-K(N3) amide |
| M6961 | 29 | Y-Aib-EGT-am Y-TSDYSSYLEEQAA KEFIA-am Y- |
| | | LVKGGG-K(N3) amide |
| M6697 | 30 | Y-Aib-EGT-am Y-TSDYSSYLEEQAA KEFIA-W- |
| | | LVKGGG-K(N3) amide |
| M6962 | 31 | Y-Aib-EGT-amF-TSDYSSYLEEQAA KEFIA-W-LVKGGG- |
| | | K(N3) amide |
| M6810 | 32 | Y-Aib-EGT-amF-TSDYSSYLEEQAA KEFIA-amY- |
| | | LVKGGG-(GGGGS)3-K amide |
| M6969 | 33 | Y-Aib-EGT-amY-TSDYSSYLEEQAA KEFIA-W- |
| | | LVKGGG-K[T-CH₂CH₂CO-(GGGGS)3-K(ALO)] amide |
| M6970 | 34 | Y-Aib-EGT-am Y-TSDYSSYLEEQAA KEFIA-am Y- |
| | | LVKGGG-(GGGGS)3-K(Ac-COT/ALO) amide |
| M6976 | 35 | Y-Aib-EGT-amF-TSDYSSYLEEQAA KEFIA-am Y- |
| | | LVKGGG-(GGGGS)3-K(DIBAC) amide |
| M6977 | 36 | Y-Aib-EGT-amF-TSDYSSYLEEQAA KEFIA-am Y- |
| | | LVEGGG-K(N3) amide |
| M6978 | 37 | Y-Aib-EGT-amF-TSDYSSYLEEQAA KEFIE-amY- |
| | | LVEGGG-K(N3) amide |
| M6979 | 38 | Y-Aib-EGT-amF-TSDYSSYLEEQAA -Aib-EFIE-am Y- |
| | | LVEGGG-K(N3) amide |
| M6491 | 39 | Y-Aib-Atz-GTFTSDYSSYLEEQAA KEFIA-W-LVKGGG |
| | | amide |
| M6988 | 40 | Y-Aib-Atz-GT-amF(2-fluoro)-TSDYSSYLEEQAA KEFIA- |
| | | W-LVKGGG-(GGGGS)3-K[K(N3)] amide |
| M6562 | 52 | H[Aib JEGTFTSDYSSYLEEQAAKEFIAWLVKGGG |
| | | (G4S)3-K(Ac-COT)-amide |
| M6677 | 53 | H[Aib]EGTFTSDYSSYLEEQAAKEFIAWLVKGGG K[TA- |
| | | (G4S)3-K(COT)]-amide |
| M6991 | 54 | Y-Aib-EGT-amF-TSDYSSYLEEQAA KEFIA-am Y- |
| | | LVKGGG; |
| | | or |
| | | Tyr (Aib) Glu Gly Thr (amF) Thr Ser Asp Tyr Ser Ser Tyr |
| | | Leu Glu Glu Gln Ala Ala Lys Glu Phe Ile Ala (amY) Leu Val |
| | | Lys Gly Gly Gly |
| Abbreviations | | T is triazole; Atz (2-amino-3-(2H-tetrazol-5-yl) propanoic |
| | | acid); amF: alpha methyl phenylalanine; |
| | | am Y: alpha methyl tyrosine; |
| | | amF(2F) or amF(2-fluoro): alpha methyl 2-fluoro- |
| | | phenylalanine; |
| | | ALO: 2-(cyclooct-2-yn-lyloxy) acetyl; |
| | | COT: 2-(cyclooct-2-yn-lyloxy) |

### Additional Sequences of Cited Protein and Antibodies.

Dimeric human GDF8 was expressed without a tag (human GDF8, REGN30:
NENSEQKENVEKEGLCNACTWRQNTKSSRIEAIKIQILSKLRLETAPNISKDVIRQLLPKA
PPLRELIDQYDVQRDDSSDGSLEDDDYHATTETIITMPTESDFLMQVDGKPKCCFFKFSS
KIQYNKVVKAQLWIYLRPVETPTTVFVQILRLIKPMKDGTRYTGIRSLKLDMNPGTGIW
QSIDVKTVLQNWLKQPESNLGIEIKALDENGHDLAVTFPGPGEDGLNPFLEVKVTDTPK
RRRRDFGLDCDEHSTESRCCRYPLTVDFEAFGWDWIIAPKRYKANYCSGECEFVFLQKY
PHTHLVHQANPRGSAGPCCTPTKMSPINMLYFNGKEQIIYGKIPAMVVDRCGCS, SEQ ID
NO: 41).
Trevogrumab, REGN1033 heavy chain sequence:
(SEQ ID NO: 42)
EVQVLESGGDLVQPGGSLRLSCAASGFTFSAYAMTWVRQAPGKGLEWVSAISGSGGSA
YYADSVKGRFTISRDNSKNTVYLQMNSLRAEDTAVYYCAKDGAWKMSGLDVWGQGTT
VIVSSASTKGPSVFPLAPCSRSTSESTAALGCLVKDYFPEPVTVSWNSGALTSGVHTFPAV
LQSSGLYSLSSVVTVPSSSLGTKTYTCNVDHKPSNTKVDKRVESKYGPPCPPCPAPEFLG
GPSVFLFPPKPKDTLMISRTPEVTCVVVDVSQEDPEVQFNWYVDGVEVHNAKTKPREEQ
FNSTYRVVSVLTVLHQDWLNGKEYKCKVSNKGLPSSIEKTISKAKGQPREPQVYTLPPSQ
EEMTKNQVSLTCLVKGFYPSDIAVEWESNGQPENNYKTTPPVLDSDGSFFLYSRLTVDKS
RWQEGNVFSCSVMHEALHNHYTQKSLSLSLGK
Trevogrumab, REGN1033 light chain sequence:
DIQMTQSPASLSASVGDRVTITCRASQDISDYLAWYQQKPGKIPRLLIYTTSTLQ*SGVPS
RFRGSGSGTDFTLTISSLQPEDVATYYCQKYDSAPLTFGGGTKVEIKRTVAAPSVFIFPPSD
EQLKSGTASVVCLLNNFYPREAKVQWKVDNALQSGNSQESVTEQDSKDSTYSLSSTLTL
SKADYEKHKVYACEVTHQGLSSPVTKSFNRGEC (SEQ ID NO: 43; *Q55 represents a
potential conjugation site)
Apitegromab heavy chain
(SEQ ID NO: 44)
QVQLVESGGGVVQPGRSLRLSCAASGFTFSSYGMHWVRQAPGKGLEWVAVISYDGSN
KYYADSVKGRFTISRDNSKNTLYLQMNSLRAEDTAVYYCARDL 100
LVRFLEWSHYYGMDVWGQGTTVTVSSASTKGPSVFPLAPCSRSTSESTAALGCLVKD
YFPEPVTVSWNSGALTSGVHTFPAVLQSSGLYSLSSVVTVPSS 200
SLGTKTYTCNVDHKPSNTKVDKRVESKYGPPCPPCPAPEFLGGPSVFLFPPKPKDTL
MISRTPEVTCVVVDVSQEDPEVQFNWYVDGVEVHNAKTKPREE 300
QFNSTYRVVSVLTVLHQDWLNGKEYKCKVSNKGLPSSIEKTISKAKGQPREPQVYTL
PPSQEEMTKNQVSLTCLVKGFYPSDIAVEWESNGQPENNYKTT 400
PPVLDSDGSFFLYSRLTVDKSRWQEGNVFSCSVMHEALHNHYTQKSLSLSLG
Apitegromab light chain
(SEQ ID NO: 45)
QSVLTQPPSASGTPGQRVTISCSGSSSNIGSNTVHWYQQLPGTAPKLLIYSDNQRPS
GVPDRFSGSKSGTSASLAISGLQSEDEADYYCAAWDDSLNGVF 100
GGGTKLTVLGQPKAAPSVTLFPPSSEELQANKATLVCLISDFYPGAVTVAWKADSSP
VKAGVETTTPSKQSNNKYAASSYLSLTPEQWKSHRSYSCQVTH 200
EGSTVEKTVAPTECS 215
Bimagrumab heavy chain Sequence
(SEQ ID NO: 46)
QVQLVQSGAEVKKPGASVKVSCKASGYTFTSSYINWVRQAPGQGLEWMGTINPVSGS
TSYAQKFQGRVTMTRDTSISTAYMELSRLRSDDTAVYYCARGG 100
WFDYWGQGTLVTVSSASTKGPSVFPLAPSSKSTSGGTAALGCLVKDYFPEPVTVSWN
SGALTSGVHTFPAVLQSSGLYSLSSVVTVPSSSLGTQTYICNV 200
NHKPSNTKVDKRVEPKSCDKTHTCPPCPAPEAAGGPSVFLFPPKPKDTLMISRTPEV
TCVVVDVSHEDPEVKFNWYVDGVEVHNAKTKPREEQYNSTYRV 300
VSVLTVLHQDWLNGKEYKCKVSNKALPAPIEKTISKAKGQPREPQVYTLPPSREEMT
KNQVSLTCLVKGFYPSDIAVEWESNGQPENNYKTTPPVLDSDG 400
SFFLYSKLTVDKSRWQQGNVFSCSVMHEALHNHYTQKSLSLSPGK
Bimagrumab Light chain sequence
(SEQ ID NO: 47)
QSALTQPASVSGSPGQSITISCTGTSSDVGSYNYVNWYQQHPGKAPKLMIYGVSKRP
SGVSNRFSGSKSGNTASLTISGLQAEDEADYYCGTFAGGSYYG 100
VFGGGTKLTVLGQPKAAPSVTLFPPSSEELQANKATLVCLISDFYPGAVTVAWKADS
SPVKAGVETTTPSKQSNNKYAASSYLSLTPEQWKSHRSYSCQV 200
THEGSTVEKTVAPTECS
Domagrozumab heavy chain
(SEQ ID NO: 48)
EVQLLESGGGLVQPGGSLRLSCAASGFTFSSYAMSWVRQAPGKGLEWVSTISSGGSY
TSYPDSVKGRFTISRDNSKNTLYLQMNSLRAEDTAVYYCAKQD 100
YAMNYWGQGTLVTVSSASTKGPSVFPLAPSSKSTSGGTAALGCLVKDYFPEPVTVSW
NSGALTSGVHTFPAVLQSSGLYSLSSVVTVPSSSLGTQTYICN 200
VNHKPSNTKVDKKVEPKSCDKTHTCPPCPAPEAAGAPSVFLFPPKPKDTLMISRTPE
VTCVVVDVSHEDPEVKFNWYVDGVEVHNAKTKPREEQYNSTYR 300
VVSVLTVLHQDWLNGKEYKCKVSNKALPAPIEKTISKAKGQPREPQVYTLPPSREEM
TKNQVSLTCLVKGFYPSDIAVEWESNGQPENNYKTTPPVLDSD 400
GSFFLYSKLTVDKSRWQQGNVFSCSVMHEALHNHYTQKSLSLSPG
Domagrozumab light chain
(SEQ ID NO: 49)
DIQMTQSPSSLSASVGDRVTITCKASQDVSTAVAWYQQKPGKAPKLLIYSASYRYTG
VPSRFSGSGSGTDFTLTISSLQPEDFATYYCQQHYSTPWTFGG 100
GTKVEIKRTVAAPSVFIFPPSDEQLKSGTASVVCLLNNFYPREAKVQWKVDNALQSG
NSQESVTEQDSKDSTYSLSSTLTLSKADYEKHKVYACEVTHQG 200
LSSPVTKSFNRGEC
Landogrozumab heavy chain
(SEQ ID NO: 50)
EVQLVESGGGLVQPGGSLRLSCAASGLTFSRYPMSWVRQAPGKGLVWVSAITSSGGS
TYYSDTVKGRFTISRDNAKNTLYLQMNSLRAEDTAVYYCARLP 100
DYWGQGTLVTVSSASTKGPSVFPLAPCSRSTSESTAALGCLVKDYFPEPVTVSWNSG
ALTSGVHTFPAVLQSSGLYSLSSVVTVPSSSLGTKTYTCNVDH 200
KPSNTKVDKRVESKYGPPCPPCPAPEFLGGPSVFLFPPKPKDTLMISRTPEVTCVVV
DVSQEDPEVQFNWYVDGVEVHNAKTKPREEQFNSTYRVVSVLT 300
VLHQDWLNGKEYKCKVSNKGLPSSIEKTISKAKGQPREPQVYTLPPSQEEMTKNQVS
LTCLVKGFYPSDIAVEWESNGQPENNYKTTPPVLDSDGSFFLY 400
SRLTVDKSRWQEGNVFSCSVMHEALHNHYTQKSLSLSLG
Landogrozumab light chain
(SEQ ID NO: 51)
EIVLTQSPGTLSLSPGERATLSCRASSSVSSSYLHWYQQKPGQAPRLLIYSTSNLVA
GIPDRFSGSGSGTDFTLTISRLEPEDFAVYYCQHHSGYHFTFG 100
GGTKVEIKRTVAAPSVFIFPPSDEQLKSGTASVVCLLNNFYPREAKVQWKVDNALQS
GNSQESVTEQDSKDSTYSLSSTLTLSKADYEKHKVYACEVTHQ 200
GLSSPVTKSFNRGEC.

Antibody identifier (HCVR/LCVR): 21-E5 (SEQ ID NO:74/75); 1A2 (SEQ ID NO:78/79); 8D12-1 (SEQ ID NO:76/77); H4H1657N2 (SEQ ID NO:80/81); H4H1669P (SEQ ID NO:82/83).

```
<210> 74
<211> 125
<212> PRT
<213> Artificial Sequence
<220>
<223> Synthetic
<400> 74
Gln Val Gln Leu Val Glu Ser Gly Pro Gly Leu Val Lys Pro Ser Glu
1 5 10 15
Thr Leu Ser Leu Thr Cys Thr Val Tyr Gly Gly Ser Ile Ser Ser Gly
                        20 25 30
Asn Tyr Tyr Trp Gly Trp Ile Arg Gln Pro Pro Gly Lys Gly Leu Glu
                35 40 45
Trp Ile Gly Thr Ile Tyr Tyr Ser Gly Ser Ala Tyr Tyr Asn Pro Ser
        50 55 60
Leu Lys Ser Arg Val Thr Met Ser Val Asp Thr Ser Lys Asn Gln Phe
65 70 75 80
Ser Leu Lys Leu Ser Ser Val Thr Ala Ala Asp Thr Ala Val Tyr Tyr
                                85 90 95
Cys Val Arg Asp Tyr Tyr Asp Ser Ser Gly His Tyr Tyr Asn Trp Phe
                        100 105 110
Asp Pro Trp Gly Gln Gly Thr Thr Val Thr Val Ser Ser
                115 120 125
<210> 75
<211> 108
<212> PRT
<213> Artificial Sequence
<220>
<223> Synthetic
<400> 75
Ala Ile Gln Met Thr Gln Ser Pro Ser Ser Leu Ser Ala Ser Val Gly
1 5 10 15
Asp Arg Val Thr Ile Thr Cys Arg Ala Ser Gln Asp Ile Arg His Asp
                        20 25 30
Leu Gly Trp Tyr Gln Gln Lys Pro Gly Lys Ala Pro Lys Arg Leu Ile
                35 40 45
Tyr Ala Ala Ser Ser Leu Gln Ser Gly Val Pro Ser Arg Phe Ser Gly
        50 55 60
Ser Gly Ser Gly Thr Glu Phe Thr Leu Thr Ile Ser Ser Leu Gln Pro
65 70 75 80
Glu Asp Phe Ala Thr Tyr Tyr Cys Leu Gln His Asn Thr Tyr Pro Trp
                                85 90 95
Thr Phe Gly Gln Gly Thr Lys Val Glu Ile Lys Arg
                        100 105
<210> 76
<211> 118
<212> PRT
<213> Artificial Sequence
<220>
<223> Synthetic
<400> 76
Gln Val Gln Leu Val Glu Ser Gly Gly Gly Val Val Gln Pro Gly Arg
1 5 10 15
Ser Leu Arg Leu Ser Cys Ala Ala Ser Gly Phe Thr Phe Ser Arg Tyr
                        20 25 30
Gly Ile His Trp Val Arg Gln Ala Pro Gly Lys Gly Leu Glu Trp Val
                35 40 45
Ala Val Ile Ser Tyr Asp Gly Ser Asp Glu Tyr Tyr Val Asp Ser Val
        50 55 60
Lys Gly Arg Phe Ser Ile Ser Arg Asp Asn Ser Lys Asn Thr Leu Tyr
65 70 75 80
Leu Gln Met Asn Ser Leu Arg Pro Ala Asp Ser Ala Val Tyr Tyr Cys
                                85 90 95
Val Lys Gly Asp Leu Glu Leu Gly Phe Asp Tyr Trp Gly Gln Gly Thr
                        100 105 110
Leu Val Thr Val Ser Ser
                115
<210> 77
<211> 112
<212> PRT
<213> Artificial Sequence
<220>
<223> Synthetic
<400> 77
Asp Ile Val Met Thr Gln Ala Ala Pro Ser Ile Pro Val Ile Pro Gly
1 5 10 15
Glu Ser Val Ser Met Ser Cys Arg Ser Ser Lys Ser Leu Leu Tyr Ser
                        20 25 30
Asn Gly His Thr Tyr Val Tyr Trp Phe Val Gln Arg Pro Gly Gln Ser
                35 40 45
Pro Gln Leu Leu Ile Tyr Arg Met Ser Asn Leu Ala Ser Gly Val Pro
        50 55 60
Asp Arg Phe Ser Gly Ser Gly Ser Gly Thr Ala Phe Thr Leu Arg Ile
65 70 75 80
Ser Arg Val Glu Ala Glu Asp Val Gly Val Tyr Tyr Cys Met Gln Asn
                                85 90 95
Leu Glu Phe Pro Leu Thr Phe Gly Ala Gly Thr Lys Leu Glu Leu Lys
                        100 105 110
<210> 78
<211> 122
<212> PRT
<213> Artificial Sequence
<220>
<223> Synthetic
<400> 78
Glu Val Gln Leu Leu Glu Ser Gly Gly Gly Leu Val Gln Pro Gly Gly
1 5 10 15
Ser Leu Arg Leu Ser Cys Ala Ala Ser Gly Phe Thr Ser Ile Thr Tyr
                        20 25 30
Ala Met Ser Trp Val Arg Gln Ala Pro Gly Lys Gly Leu Glu Trp Val
                35 40 45
Ser Ala Ile Ser Val Ser Gly Thr Asn Thr Tyr Tyr Ala Asp Ser Val
        50 55 60
Lys Gly Arg Phe Thr Ile Ser Arg Asp Lys Ser Lys Asn Met Leu Tyr
65 70 75 80
Leu Gln Met Asn Ser Leu Arg Ala Glu Asp Thr Ala Val Tyr Tyr Cys
                                85 90 95
Ala Lys Asp Leu Leu His Asn Trp Lys Tyr Gly Thr Phe Asp Ile Trp
                        100 105 110
Gly Gln Gly Thr Met Val Thr Val Ser Ser
                115 120
<210> 79
<211> 107
<212> PRT
<213> Artificial Sequence
<220>
<223> Synthetic
<400> 79
Glu Ile Val Met Thr Gln Ser Pro Ala Thr Leu Ser Val Ser Pro Gly
1 5 10 15
Glu Arg Ala Thr Leu Ser Cys Arg Ala Ser Gln Ser Val Asp Ser Asn
                        20 25 30
Leu Val Trp Tyr Gln Gln Lys Pro Gly Gln Val Pro Arg Leu Leu Ile
                35 40 45
Tyr Gly Ala Ser Thr Arg Ala Thr Gly Ile Pro Ala Arg Phe Ser Gly
        50 55 60
Ser Gly Ser Gly Thr Glu Phe Thr Leu Thr Ile Ser Ser Leu Gln Ser
65 70 75 80
Glu Asp Phe Ala Val Tyr Tyr Cys Gln Gln Tyr Asn Lys Trp Pro Leu
                                85 90 95
Thr Phe Gly Gly Gly Thr Lys Val Glu Ile Lys
                        100 105
<210> 80
<211> 120
<212> PRT
<213> Artificial Sequence
<220>
<223> Synthetic
<400> 80
Glu Val Gln Val Leu Glu Ser Gly Gly Asp Leu Val Gln Pro Gly Gly
1 5 10 15
Ser Leu Arg Leu Ser Cys Ala Ala Ser Gly Phe Thr Phe Ser Ala Tyr
                        20 25 30
Ala Met Thr Trp Val Arg Gln Ala Pro Gly Lys Gly Leu Glu Trp Val
                35 40 45
Ser Ala Ile Ser Gly Ser Gly Gly Ser Ala Tyr Tyr Ala Asp Ser Val
        50 55 60
Lys Gly Arg Phe Thr Ile Ser Arg Asp Asn Ser Lys Asn Thr Val Tyr
65 70 75 80
Leu Gln Met Asn Ser Leu Arg Ala Glu Asp Thr Ala Val Tyr Tyr Cys
                                85 90 95
Ala Lys Asp Gly Ala Trp Lys Met Ser Gly Leu Asp Val Trp Gly Gln
                        100 105 110
Gly Thr Thr Val Ile Val Ser Ser
                115 120
<210> 81
<211> 107
<212> PRT
<213> Artificial Sequence
<220>
<223> Synthetic
<400> 81
Asp Ile Gln Met Thr Gln Ser Pro Ala Ser Leu Ser Ala Ser Val Gly
1 5 10 15
Asp Arg Val Thr Ile Thr Cys Arg Ala Ser Gln Asp Ile Ser Asp Tyr
                        20 25 30
Leu Ala Trp Tyr Gln Gln Lys Pro Gly Lys Ile Pro Arg Leu Leu Ile
                35 40 45
Tyr Thr Thr Ser Thr Leu Gln Ser Gly Val Pro Ser Arg Phe Arg Gly
        50 55 60
Ser Gly Ser Gly Thr Asp Phe Thr Leu Thr Ile Ser Ser Leu Gln Pro
65 70 75 80
Glu Asp Val Ala Thr Tyr Tyr Cys Gln Lys Tyr Asp Ser Ala Pro Leu
                                85 90 95
Thr Phe Gly Gly Gly Thr Lys Val Glu Ile Lys
                        100 105
<210> 82
<211> 121
<212> PRT
<213> Artificial Sequence
<220>
<223> Synthetic
<400> 82
Gln Val Gln Leu Val Glu Ser Gly Gly Gly Val Val Gln Pro Gly Lys
1 5 10 15
Ser Leu Arg Leu Ser Cys Ala Ala Ser Gly Phe Thr Phe Ser Ser Phe
                        20 25 30
Gly Met His Trp Val Arg Gln Ala Pro Gly Lys Gly Leu Glu Trp Val
                35 40 45
Ala Val Ile Gly Tyr Asp Gly Gly Asn Glu Tyr Tyr Ala Asp Ser Val
        50 55 60
Lys Gly Arg Phe Thr Ile Ser Arg Asp Asn Ser Lys Asn Thr Leu Asn
65 70 75 80
Leu Gln Met Ser Ser Leu Arg Ala Glu Asp Thr Ala Val Tyr Tyr Cys
                                85 90 95
Ser Thr Ile Ser His Tyr Asp Ile Leu Ser Gly Met Asp Val Trp Gly
                        100 105 110
Arg Gly Thr Thr Val Thr Val Ser Ser
                115 120
<210> 83
<211> 108
<212> PRT
<213> Artificial Sequence
<220>
<223> Synthetic
<400> 83
Asp Ile Gln Met Thr Gln Ser Pro Ser Ser Val Ser Ala Ser Val Gly
1 5 10 15
Asp Arg Val Thr Ile Thr Cys Arg Ala Ser Gln Gly Ile Ser Asn Trp
                        20 25 30
Leu Ala Trp Tyr Gln Gln Lys Pro Gly Lys Ala Pro Lys Leu Leu Ile
                35 40 45
Phe Ala Ala Ser Ser Leu Gln Ser Gly Val Pro Ser Arg Phe Ser Gly
        50 55 60
Ser Ala Ser Gly Thr Asp Phe Thr Leu Thr Ile Asn Ser Leu Gln Pro
65 70 75 80
Glu Asp Phe Ala Thr Tyr Tyr Cys Gln Gln Ala Asn Ser Phe Pro Leu
                                85 90 95
Thr Phe Gly Gly Gly Thr Lys Val Glu Ile Lys Arg
                        100 105
```

### Glucagon-Like Peptide-1 (GLP-1) Receptor (GLP1R) and Glucose-Dependent Insulinotropic Polypeptide (GIP) Receptor (GIPR) Dual Agonist (GLP1R-GIPR Dual Agonist)

In some embodiments, the GLP1R and GIPR dual agonist comprises a sequence of Tyr1 Aib2 XX3 Gly4 XX5 XX6 Thr Ser Asp Tyr Ser Ser XX13 Leu Glu Glu Gln Ala Ala XX20 Glu XX22 Ile XX24 XX25 Leu Val XX28 Gly Gly Gly (SEQ ID NO: 3), wherein Aib represents 2-aminoisobutyric acid (α-aminoisobutyric acid); XX3 is Glu or Atz ((S)-2-amino-3-(2H-tetrazol-5-yl)propanoic acid); XX5 is Ser or Thr; XX6 is phenylalanine, alpha methyl phenylalanine (amF) or 2-fluoro alpha methyl phenylalanine (amF(2-fluoro)); XX13 is Tyr, amF, or alpha methyl tyrosine (amY); XX20 is Lys, Lys derivative, Gin, His, or Aib; XX22 is Phe or amF; XX24 is Ala or Glu; XX25 is tryptophan or alpha methyl tyrosine (amY); and XX28 is Lys, Ala, or Glu.

In some embodiments, the GLP1R-GIPR dual agonist comprises a sequence of: Tyr1 Aib2 XX3 Gly4 XX5 XX6 Thr Ser Asp Tyr Ser Ser XX13 Leu Glu Glu Gln Ala Ala XX20 Glu XX22 Ile XX24 XX25 Leu Val XX28 Gly Gly Gly (SEQ ID NO: 3), wherein Aib represents 2-aminoisobutyric acid (α-aminoisobutyric acid); XX3 is Glu or Atz ((S)-2-amino-3-(2H-tetrazol-5-yl)propanoic acid); XX5 is Ser or Thr; XX6 is phenylalanine, alpha methyl phenylalanine (amF) or 2-fluoro alpha methyl phenylalanine (amF(2-fluoro)); XX13 is amF or alpha methyl tyrosine (amY); XX20 is Lys, sidechain-acylated or alkylated Lys, or Aib; XX22 is Phe or amF; XX24 is Ala or Glu; XX25 is tryptophan or alpha methyl tyrosine (amY); and XX28 is Lys, Ala, Gln, or Glu.

In some embodiments, the GLP1R and GIPR dual agonist comprises a sequence of: Tyr1 Aib2 XX3 Gly4 XX5 XX6 Thr Ser Asp Tyr Ser Ser XX13 Leu Glu Glu Gln Ala Ala XX20 Glu XX22 Ile XX24 XX25 Leu Val XX28 Gly Gly Gly (SEQ ID NO: 3), wherein Aib represents 2-aminoisobutyric acid or (α-aminoisobutyric acid); XX3 is Glu or Atz ((S)-2-amino-3-(2H-tetrazol-5-yl)propanoic acid); XX5 is Ser or Thr; XX6 is phenylalanine, alpha methyl phenylalanine (amF) or 2-fluoro alpha methyl phenylalanine (amF(2-fluoro)); XX13 is Tyr, amF, or alpha methyl tyrosine (amY); XX20 is Lys, sidechain-acylated or alkylated Lys, or Aib; XX22 is amF; XX24 is Ala or Glu; XX25 is tryptophan or alpha methyl tyrosine (amY); and XX28 is Lys, Ala, Gln, or Glu.

In some embodiments, the GLP1R and GIPR dual agonist comprises a sequence of: Tyr1 Aib2 XX3 Gly4 XX5 XX6 Thr Ser Asp Tyr Ser Ser XX13 Leu Glu Glu Gln Ala Ala XX20 Glu XX22 Ile XX24 XX25 Leu Val XX28 Gly Gly Gly (SEQ ID NO: 3), wherein Aib represents 2-aminoisobutyric acid (α-aminoisobutyric acid); XX3 is Glu or Atz ((S)-2-amino-3-(2H-tetrazol-5-yl)propanoic acid); XX5 is Ser or Thr; XX6 is phenylalanine, alpha methyl phenylalanine (amF) or 2-fluoro alpha methyl phenylalanine (amF(2-fluoro)); XX13 is Tyr, amF, or alpha methyl tyrosine (amY); XX20 is Lys, sidechain-acylated or alkylated Lys, or Aib; XX22 is Phe or amF; XX24 is Glu; XX25 is tryptophan or alpha methyl tyrosine (amY); and XX28 is Lys, Ala, Gln, or Glu.

In some embodiments, the GLP1R and GIPR dual agonist comprises a sequence of: Tyr1 Aib2 XX3 Gly4 XX5 XX6 Thr Ser Asp Tyr Ser Ser XX13 Leu Glu Glu Gln Ala Ala XX20 Glu XX22 Ile XX24 XX25 Leu Val XX28 Gly Gly Gly (SEQ ID NO: 3), wherein Aib represents 2-aminoisobutyric acid (α-aminoisobutyric acid); XX3 is Glu or Atz ((S)-2-amino-3-(2H-tetrazol-5-yl)propanoic acid); XX5 is Ser or Thr; XX6 is phenylalanine, alpha methyl phenylalanine (amF) or 2-fluoro alpha methyl phenylalanine (amF(2-fluoro)); XX13 is Tyr, amF, or alpha methyl tyrosine (amY); XX20 is Lys, sidechain-acylated or alkylated Lys, or Aib; XX22 is Phe or amF; XX24 is Ala or Glu; XX25 is alpha methyl tyrosine (amY); and XX28 is Lys, Ala, Gln, or Glu.

In some embodiments, the GLP1R and GIPR dual agonist comprises a sequence of: Tyr1 Aib2 XX3 Gly4 XX5 XX6 Thr Ser Asp Tyr Ser Ser XX13 Leu Glu Glu Gln Ala Ala XX20 Glu XX22 Ile XX24 XX25 Leu Val XX28 Gly Gly Gly (SEQ ID NO: 3), wherein Aib represents 2-aminoisobutyric acid (α-aminoisobutyric acid); XX3 is Glu or Atz ((S)-2-amino-3-(2H-tetrazol-5-yl)propanoic acid); XX5 is Ser or Thr; XX6 is phenylalanine, alpha methyl phenylalanine (amF) or 2-fluoro alpha methyl phenylalanine (amF(2-fluoro)); XX13 is Tyr, amF, or alpha methyl tyrosine (amY); XX20 is Lys, sidechain-acylated or alkylated Lys, or Aib; XX22 is Phe or amF; XX24 is Ala or Glu; XX25 is tryptophan or alpha methyl tyrosine (amY); and XX28 is Ala, Gln, or Glu.

In some embodiments, XX3 is Glu, Gln, or Atz ((S)-2-amino-3-(2H-tetrazol-5-yl)propanoic acid). In some embodiments, XX3 is Glu or Atz ((S)-2-amino-3-(2H-tetrazol-5-yl)propanoic acid).

In some embodiments, XX13 is amF or alpha methyl tyrosine (amY).

In some embodiments, XX20 is Aib, Lys, Gln or Glu. In some embodiments, XX20 is Lys or Aib.

In some embodiments, XX22 is amF.

In some embodiments, XX24 is Ala, Gln, or Glu. In some embodiments, XX24 is Ala or Glu.

In some embodiments, XX25 is alpha methyl tyrosine.

In some embodiments, XX28 is Lys, Gln, or Glu.

In some embodiments, the GLP1R-GIPR dual agonist has a sequence of Tyr (Aib) Glu Gly Thr (amF) Thr Ser Asp Tyr Ser Ser Tyr Leu Glu Glu Gln Ala Ala Lys Glu Phe Ile Ala (amY) Leu Val Lys Gly Gly Gly (SEQ ID NO: 54), where Aib is 2-aminoisobutyric acid (α-aminoisobutyric acid); amF is alpha methyl phenylalanine; and amY is alpha methyl tyrosine.

In some embodiments, the GLP1R-GIPR dual agonist has a sequence selected from the group consisting of SEQ ID NOs: 3, 14-17, and 19-54.

In some embodiments, the GLP1R and GIPR dual agonist binds to and activates the signaling cascade of GLP1R and GIPR.

In some embodiments, the GLP1R and GIPR dual agonist binds to and activates the signaling cascade of GLP1R and GIPR, and wherein the GLP1R and GIPR dual agonist does not substantially bind to and/or activates the signaling cascade of glucagon receptor.

In some embodiments, the GLP1R and GIPR dual agonist binds to and activates the signaling cascade of GLP1R and GIPR, and wherein said GLP1R and GIPR dual agonist does not substantially bind to and/or activates the signaling cascade of GLP2R.

In some embodiments, the conjugate binds to and activates the signaling cascade of GLP1R and GIPR.

In some embodiments, the conjugate binds to and activates the signaling cascade of GLP1R and GIPR, and the conjugate does not substantially bind to and/or activates the signaling cascade of glucagon peptide receptor.

In some embodiments, the conjugate binds to and activates the signaling cascade of GLP1R and GIPR, and said conjugate does not substantially bind to and/or activates the signaling cascade of GLP2R.

In some embodiments, the conjugate binds to and activates the signaling cascade of GLP1R and GIPR and the conjugate binds to and/or blocks the activity of growth and differentiation factor-8 (GDF8).

In some embodiments, the conjugate binds to and activates the signaling cascade of GLP1R and GIPR and the conjugate binds to and/or blocks the activity of growth and differentiation factor-8 (GDF8) having a sequence of SEQ ID NO: 1.

In another aspect, provided herein is a glucagon-like peptide-1 (GLP-1) receptor (GLP1R) and glucose-dependent insulinotropic polypeptide (GIP) receptor (GIPR) dual agonist, or a pharmaceutically acceptable salt thereof, comprising: Tyr1 Aib2 XX3 Gly4 XX5 XX6 Thr Ser Asp Tyr Ser Ser XX13 Leu Glu Glu Gln Ala Ala XX20 Glu XX22 Ile XX24 XX25 Leu Val XX28 Gly Gly Gly (SEQ ID NO: 3), where Aib represents 2-aminoisobutyric acid (α-aminoisobutyric acid); XX3 is Glu or Atz ((S)-2-amino-3-(2H-tetrazol-5-yl)propanoic acid); XX5 is Ser or Thr; XX6 is phenylalanine, alpha methyl phenylalanine (amF) or 2-fluoro alpha methyl phenylalanine (amF(2-fluoro)); XX13 is Tyr, amF, or alpha methyl tyrosine (amY); XX20 is Lys, sidechain-acylated or alkylated Lys, or Aib; XX22 is Phe or amF; XX24 is Ala or Glu; XX25 is tryptophan or alpha methyl tyrosine (amY); XX28 is Lys, Ala, Gln, or Glu; and wherein the C-terminal end is an acid or an amide.

In another aspect, provided herein is a glucagon-like peptide-1 (GLP-1) receptor (GLP1R) and glucose-dependent insulinotropic polypeptide (GIP) receptor (GIPR) dual agonist (GLP1R-GIPR dual agonist), or a pharmaceutically acceptable salt thereof, comprising: Tyr1 Aib2 XX3 Gly4 XX5 XX6 Thr Ser Asp Tyr Ser Ser XX13 Leu Glu Glu Gln Ala Ala XX20 Glu XX22 Ile XX24 XX25 Leu Val XX28 Gly Gly Gly (SEQ ID NO: 3), where Aib represents 2-aminoisobutyric acid (α-aminoisobutyric acid); XX3 is Glu or Atz ((S)-2-amino-3-(2H-tetrazol-5-yl)propanoic acid); XX5 is Ser or Thr; XX6 is phenylalanine, alpha methyl phenylalanine (amF) or 2-fluoro alpha methyl phenylalanine (amF(2-fluoro)); XX13 is amF or alpha methyl tyrosine (amY); XX20 is Lys, sidechain-acylated or alkylated Lys, or Aib; XX22 is Phe or amF; XX24 is Ala or Glu; XX25 is tryptophan or alpha methyl tyrosine (amY); XX28 is Lys, Ala, Gln, or Glu; and wherein the C-terminal end is an acid or an amide.

In another aspect, provided herein is a glucagon-like peptide-1 (GLP-1) receptor (GLP1R) and glucose-dependent insulinotropic polypeptide (GIP) receptor (GIPR) dual agonist, or a pharmaceutically acceptable salt thereof, comprising: Tyr1 Aib2 XX3 Gly4 XX5 XX6 Thr Ser Asp Tyr Ser Ser XX13 Leu Glu Glu Gln Ala Ala XX20 Glu XX22 Ile XX24 XX25 Leu Val XX28 Gly Gly Gly (SEQ ID NO: 3), where Aib represents 2-aminoisobutyric acid (α-aminoisobutyric acid); XX3 is Glu or Atz ((S)-2-amino-3-(2H-tetrazol-5-yl)propanoic acid); XX5 is Ser or Thr; XX6 is phenylalanine, alpha methyl phenylalanine (amF) or 2-fluoro alpha methyl phenylalanine (amF(2-fluoro)); XX13 is Tyr, amF, or alpha methyl tyrosine (amY); XX20 is Lys, sidechain-acylated or alkylated Lys, or Aib; XX22 is amF; XX24 is Ala or Glu; XX25 is tryptophan or alpha methyl tyrosine (amY); XX28 is Lys, Ala, Gln, or Glu; and wherein the C-terminal end is an acid or an amide.

In another aspect, provided herein is a glucagon-like peptide-1 (GLP-1) receptor (GLP1R) and glucose-dependent insulinotropic polypeptide (GIP) receptor (GIPR) dual agonist, or a pharmaceutically acceptable salt thereof, comprising: Tyr1 Aib2 XX3 Gly4 XX5 XX6 Thr Ser Asp Tyr Ser Ser XX13 Leu Glu Glu Gln Ala Ala XX20 Glu XX22 Ile XX24 XX25 Leu Val XX28 Gly Gly Gly (SEQ ID NO: 3), where Aib represents 2-aminoisobutyric acid (α-aminoisobutyric acid); XX3 is Glu or Atz ((S)-2-amino-3-(2H-tetrazol-5-yl)propanoic acid); XX5 is Ser or Thr; XX6 is phenylalanine, alpha methyl phenylalanine (amF) or 2-fluoro alpha methyl phenylalanine (amF(2-fluoro)); XX13 is Tyr, amF, or alpha methyl tyrosine (amY); XX20 is Lys, sidechain-acylated or alkylated Lys, or Aib; XX22 is Phe or amF; XX24 is Glu; XX25 is tryptophan or alpha methyl tyrosine (amY); XX28 is Lys, Ala, Gln, or Glu; and wherein the C-terminal end is an acid or an amide.

In another aspect, provided herein is a glucagon-like peptide-1 (GLP-1) receptor (GLP1R) and glucose-dependent insulinotropic polypeptide (GIP) receptor (GIPR) dual agonist, or a pharmaceutically acceptable salt thereof, comprising: Tyr1 Aib2 XX3 Gly4 XX5 XX6 Thr Ser Asp Tyr Ser Ser XX13 Leu Glu Glu Gln Ala Ala XX20 Glu XX22 Ile XX24 XX25 Leu Val XX28 Gly Gly Gly (SEQ ID NO: 3), where Aib represents 2-aminoisobutyric acid (α-aminoisobutyric acid); XX3 is Glu or Atz ((S)-2-amino-3-(2H-tetrazol-5-yl)propanoic acid); XX5 is Ser or Thr; XX6 is phenylalanine, alpha methyl phenylalanine (amF) or 2-fluoro alpha methyl phenylalanine (amF(2-fluoro)); XX13 is Tyr, amF, or alpha methyl tyrosine (amY); XX20 is Lys, sidechain-acylated or alkylated Lys, or Aib; XX22 is Phe or amF; XX24 is Ala or Glu; XX25 is alpha methyl tyrosine (amY); XX28 is Lys, Ala, Gln, or Glu; and wherein the C-terminal end is an acid or an amide.

In another aspect, provided herein is a glucagon-like peptide-1 (GLP-1) receptor (GLP1R) and glucose-dependent insulinotropic polypeptide (GIP) receptor (GIPR) dual agonist, or a pharmaceutically acceptable salt thereof, comprising: Tyr1 Aib2 XX3 Gly4 XX5 XX6 Thr Ser Asp Tyr Ser Ser XX13 Leu Glu Glu Gln Ala Ala XX20 Glu XX22 Ile XX24 XX25 Leu Val XX28 Gly Gly Gly (SEQ ID NO: 3), where Aib represents 2-aminoisobutyric acid (α-aminoisobutyric acid); XX3 is Glu or Atz ((S)-2-amino-3-(2H-tetrazol-5-yl)propanoic acid); XX5 is Ser or Thr; XX6 is phenylalanine, alpha methyl phenylalanine (amF) or 2-fluoro alpha methyl phenylalanine (amF(2-fluoro)); XX13 is Tyr, amF, or alpha methyl tyrosine (amY); XX20 is Lys, sidechain-acylated or alkylated Lys, or Aib; XX22 is Phe or amF; XX24 is Ala or Glu; XX25 is tryptophan or alpha methyl tyrosine (amY); XX28 is Ala, Gln, or Glu; and wherein the C-terminal end is an acid or an amide.

In some embodiments, XX13 is amF or alpha methyl tyrosine (amY).

In some embodiments, XX22 is alpha methyl phenylalanine (amF).

In some embodiments, XX25 is alpha methyl tyrosine (amY).

In some embodiments, the GLP1R-GIPR dual agonist has a sequence selected from the group consisting of SEQ ID NOs: 3, 14-17, and 19-54.

In some embodiments, the GLP1R and GIPR dual agonist binds to and activates the signaling cascade of GLP1R and GIPR.

In some embodiments, the GLP1R and GIPR dual agonist binds to and activates the signaling cascade of GLP1R and GIPR, and where the GLP1R and GIPR dual agonist does not substantially binds to and/or activates the signaling cascade of glucagon peptide receptor.

In some embodiments, the GLP1R and GIPR dual agonist, or a pharmaceutically acceptable salt thereof, according to any of the embodiments described herein, further comprises a linker with a reactive moiety for conjugation with a target antibody or antigen binding fragment thereof.

### Linkers

In some embodiments, the linker covalently linking the GLP1R and GIPR dual agonist with ABP comprises L1-L2-L3-L4, where L1 is an optional spacer; L2 comprises a first reactive moiety; L3 comprises a second reactive moiety; and L4 is an optional spacer; and where the first and the second reactive moieties form a covalent bond during the conjugation process.

In some embodiments, the first and second reactive moieties are bio-orthogonal reactive moieties compatible for conjugation in an aqueous medium/environment.

In some embodiments, the first and second reactive moieties are bio-orthogonal reactive moieties for Click reaction, Diels-Alder reaction, Inverse electron deficient Diels-Alder reaction (IEDDA), and highly selective nucleophilic reaction involving a thiol group or a primary amino group.

In some embodiments, the first and second reactive moieties comprise a primary amino group and a glutamine side chain that are compatible for conjugation catalyzed by a microbial transglutaminase.

In some embodiments, the reactive moieties are bio-orthogonally operable in an aqueous environment.

In some embodiments, the reactive moieties are selected from the group consisting of amino, thiol, maleimido

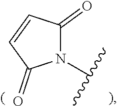 [SMILES: *N1C(=O)C=CC1=O]

succinyl ester

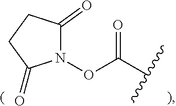 [SMILES: *C(=O)ON1C(=O)CCC1=O]

and alpha-bromo acetyl.

In some embodiments, the reactive moieties are selected from the group consisting of —N₃,

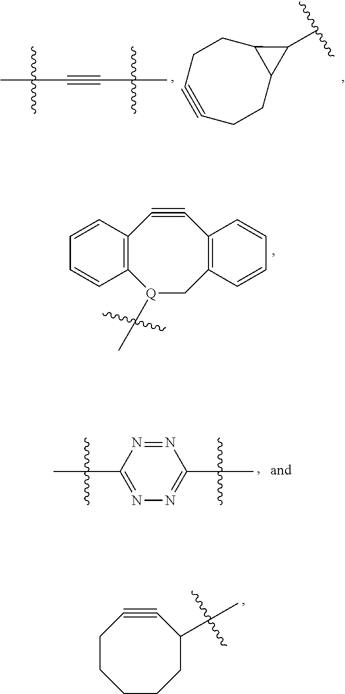

where Q is CH or N.

In some embodiments, the spacer L1 or L2 comprises (PEG)ₙ or a short peptide comprising a plurality of Gly and Ser residues, wherein n is one to 12.

In some embodiments, the spacer L1 or L2 comprises —NH-(PEG)ₙ-CO— or a short peptide comprising multiple (GlyGlyGlyGlySer)m, wherein n is one to 12 and m is one to four (SEQ ID NO: 84).

In some embodiments, the linker comprises

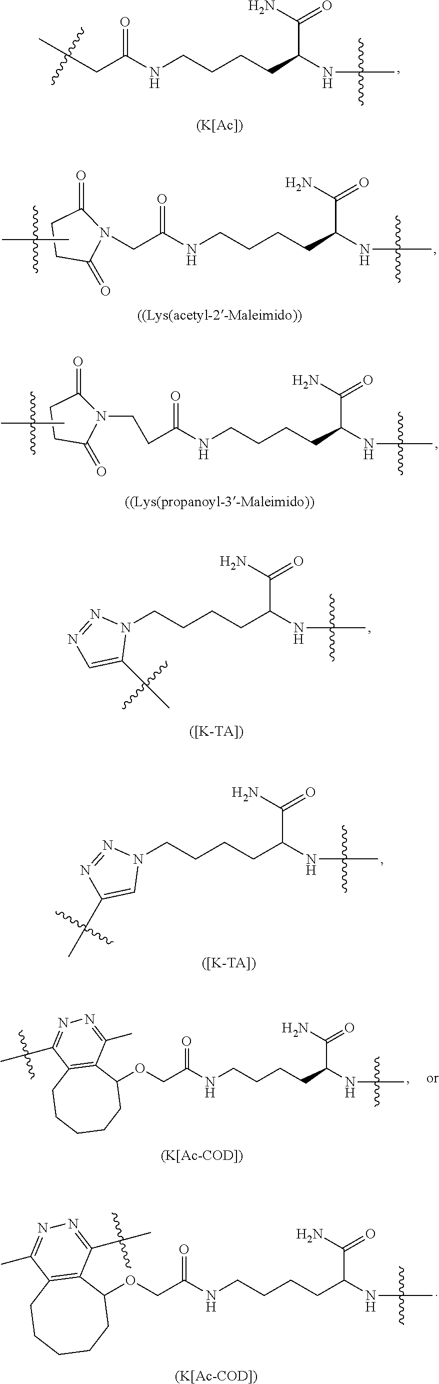

In some embodiments, the linker L comprises

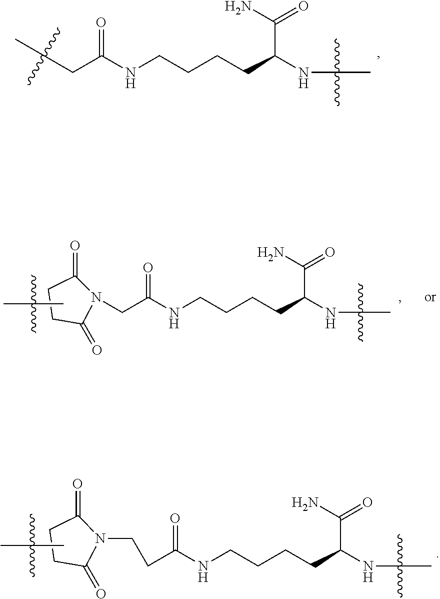

In some embodiments, the linker L comprises

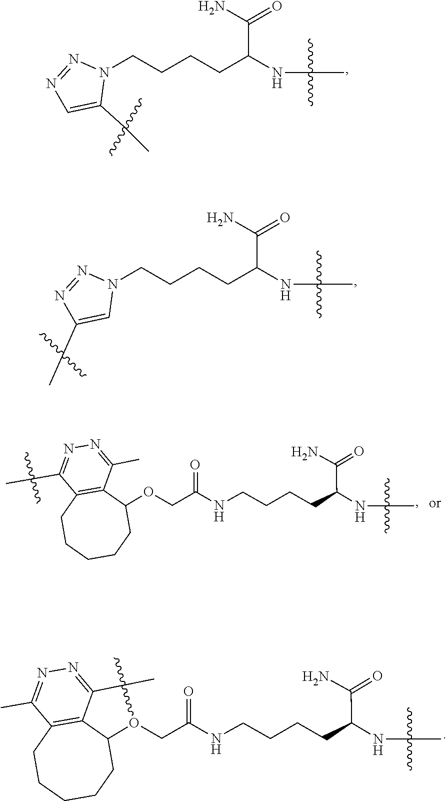 [SMILES: *NC(CCCCn1nncc1*)C(N)=O ; *NC(CCCCn1cc(*)nn1)C(N)=O ; *N[C@@H](CCCCNC(=O)COC1CCCCCc2c(*)nnc(C)c21)C(N)=O ; *N[C@@H](CCCCNC(=O)COC1CCCCCc2c(C)nnc(*)c21)C(N)=O]

In some embodiments, the linker comprises a Click or Diels-Alder reaction adduct selected from the group consisting of:

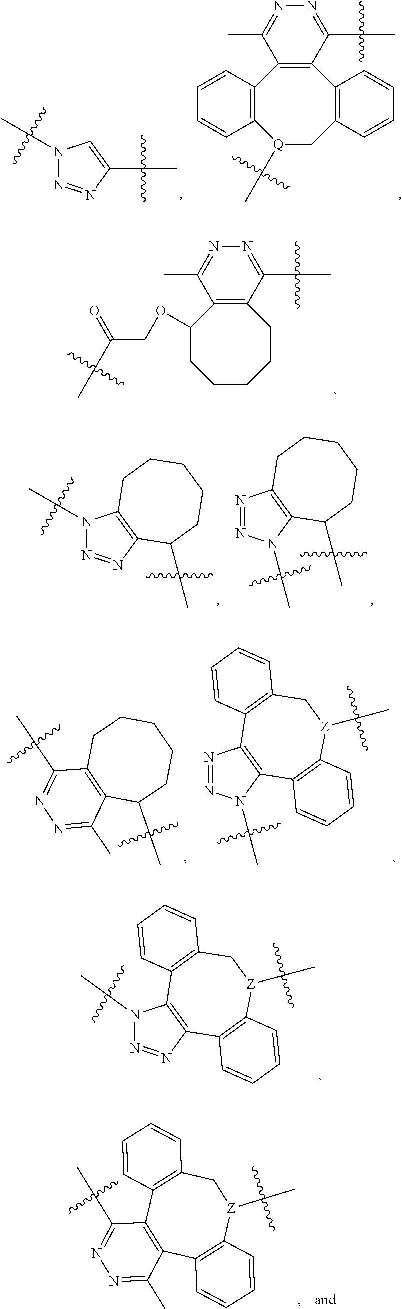

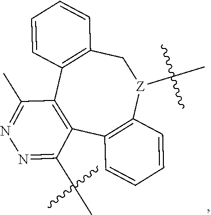

where Z is CH or N.

In some embodiments, the linker L further comprises a sequence selected from the group consisting of -(Gly₃Ser)ₘ- (SEQ ID NO: 99) and -(Gly₄Ser)ₘ- (SEQ ID NO: 100), wherein m is an integer from one to six.

In some embodiments, the linker comprises a sequence selected from the group consisting of Gly₃Ser (SEQ ID NO: 85), Gly₄Ser (SEQ ID NO: 86), (Gly₃Ser)₂ (SEQ ID NO: 87), (Gly₄Ser)₂ (SEQ ID NO: 88), (Gly₃Ser)₃ (SEQ ID NO: 89), (Gly₄Ser)₃ (SEQ ID NO: 90), (Gly3Ser)₄ (SEQ ID NO: 91), (Gly4Ser)₄ (SEQ ID NO: 92), (Gly3Ser)₅ (SEQ ID NO: 93), (Gly₄Ser)₅ (SEQ ID NO: 94), (Gly₃Ser)₆ (SEQ ID NO: 95), and (Gly₄Ser)₆ (SEQ ID NO: 96).

In some embodiments, the linker comprises a sequence of -(Gly₃Ser)ₙ-Lys(X)- (SEQ ID NO: 97) or -(Gly₄Ser)ₙ-Lys(X)- (SEQ ID NO: 98), wherein n is an integer from one to six; and wherein X is a reactive moiety attached to the side chain of Lys for conjugation with the ABP.

In some embodiments, X is a reactive moiety comprising an azido, 2-bromo-acetyl,

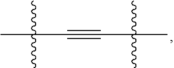 [SMILES: *C#C*]

2-(cyclooct-2-yn-lyloxy) acetyl, 2′-maleimido-acetyl, and 3′-maleimido-propanoyl.

In some embodiments, the first and second reactive moieties are bio-orthogonal reactive moieties comprising thiol, maleimido, 2-bromo acetyl, —N₃,

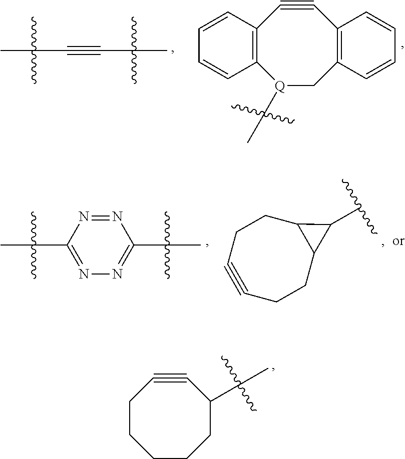

where Q is CH or N.

In some embodiments, the first and second reactive moieties are bio-orthogonal reactive moieties comprising: amino, thiol,

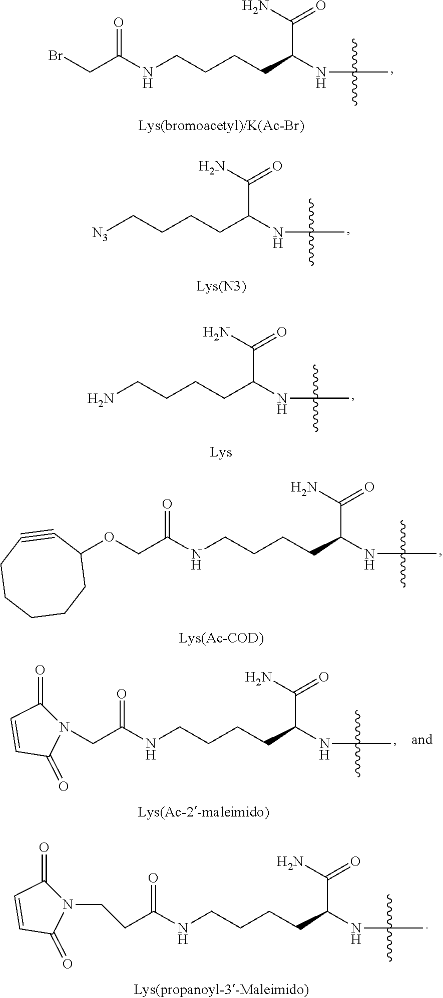 [SMILES: *N[C@@H](CCCCNC(=O)CBr)C(N)=O ; *NC(CCCCN=[N+]=[N-])C(N)=O ; *NC(CCCCN)C(N)=O ; *N[C@@H](CCCCNC(=O)COC1C#CCCCCC1)C(N)=O ; *N[C@@H](CCCCNC(=O)CN1C(=O)C=CC1=O)C(N)=O ; *N[C@@H](CCCCNC(=O)CCN1C(=O)C=CC1=O)C(N)=O]

In some embodiments, the linker comprises

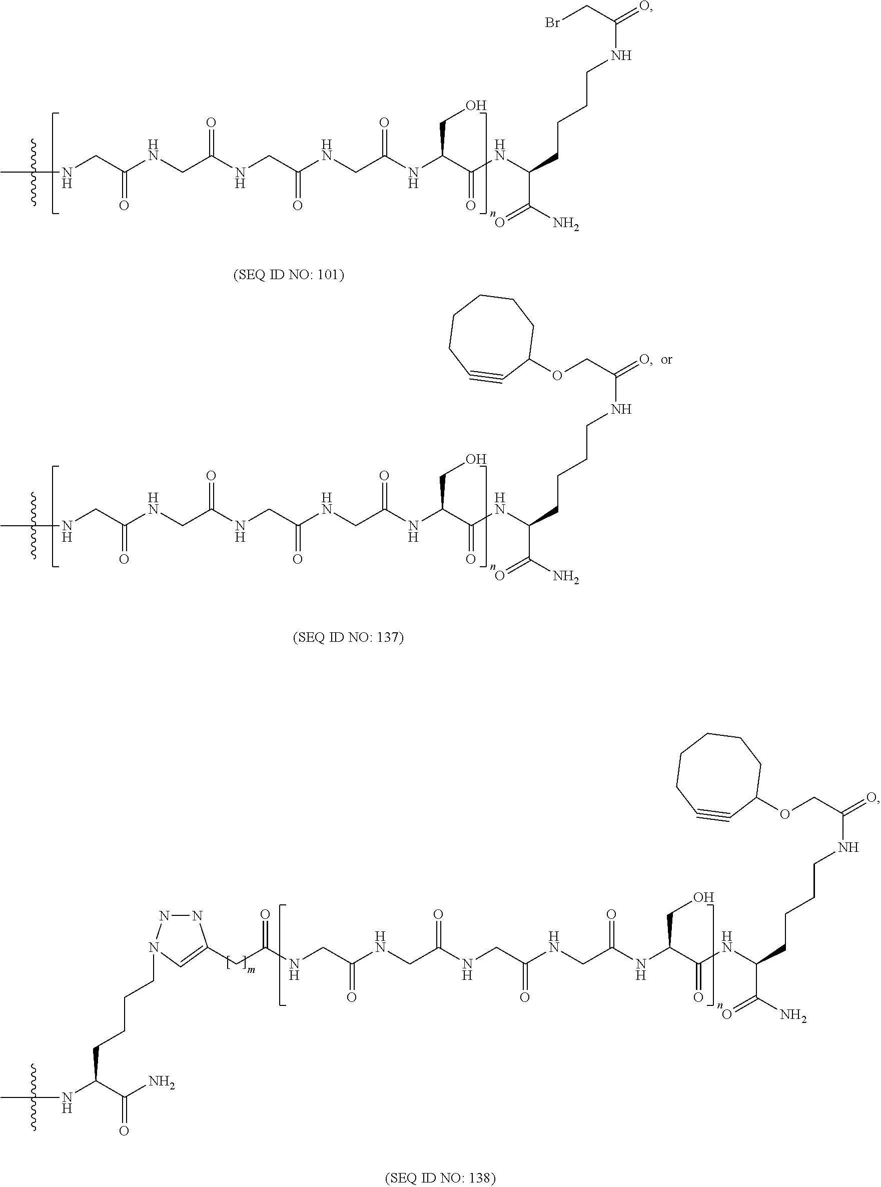

wherein m is an integer of one to three; and n is an integer of one to four.

In some embodiments, the conjugate has a structure of

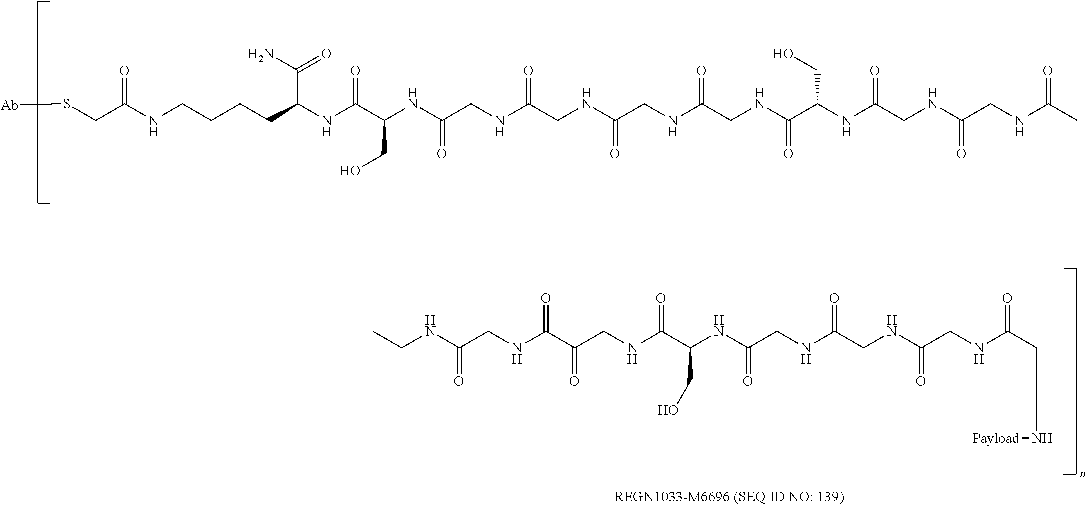

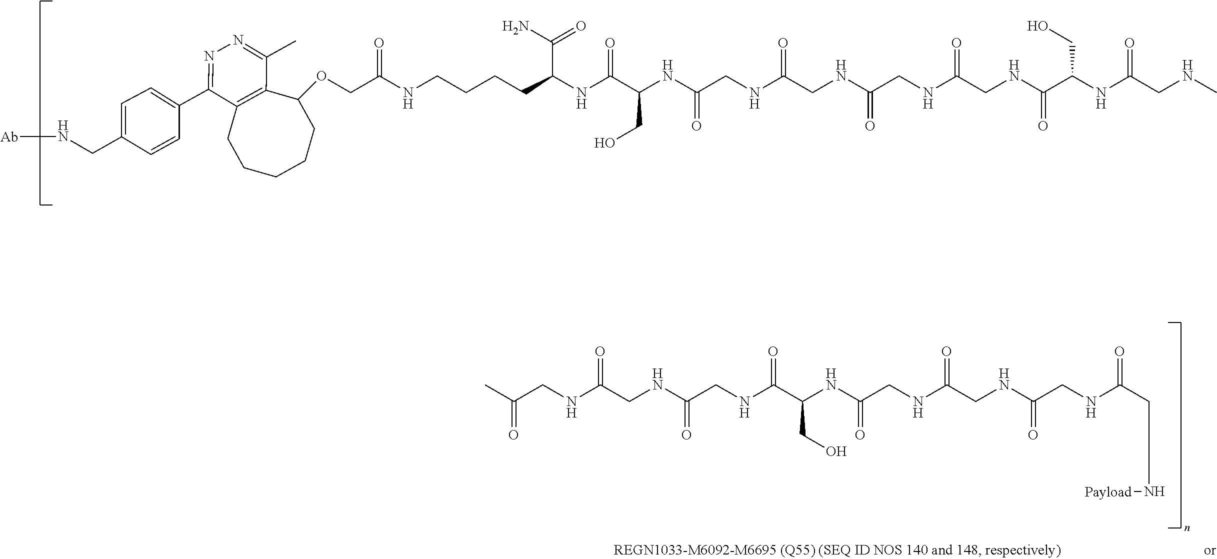

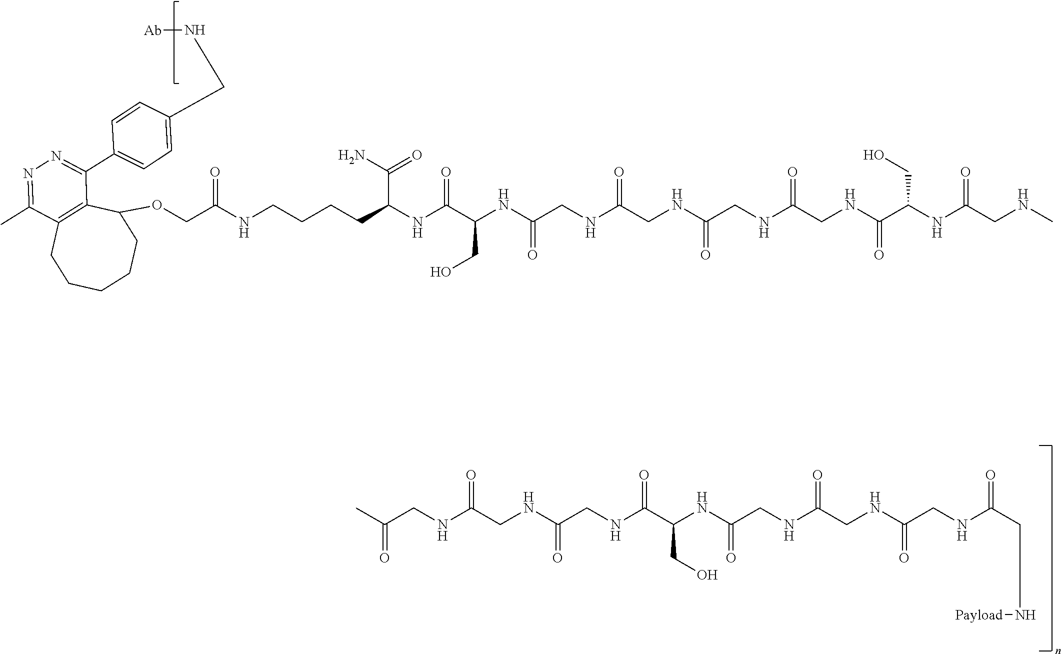

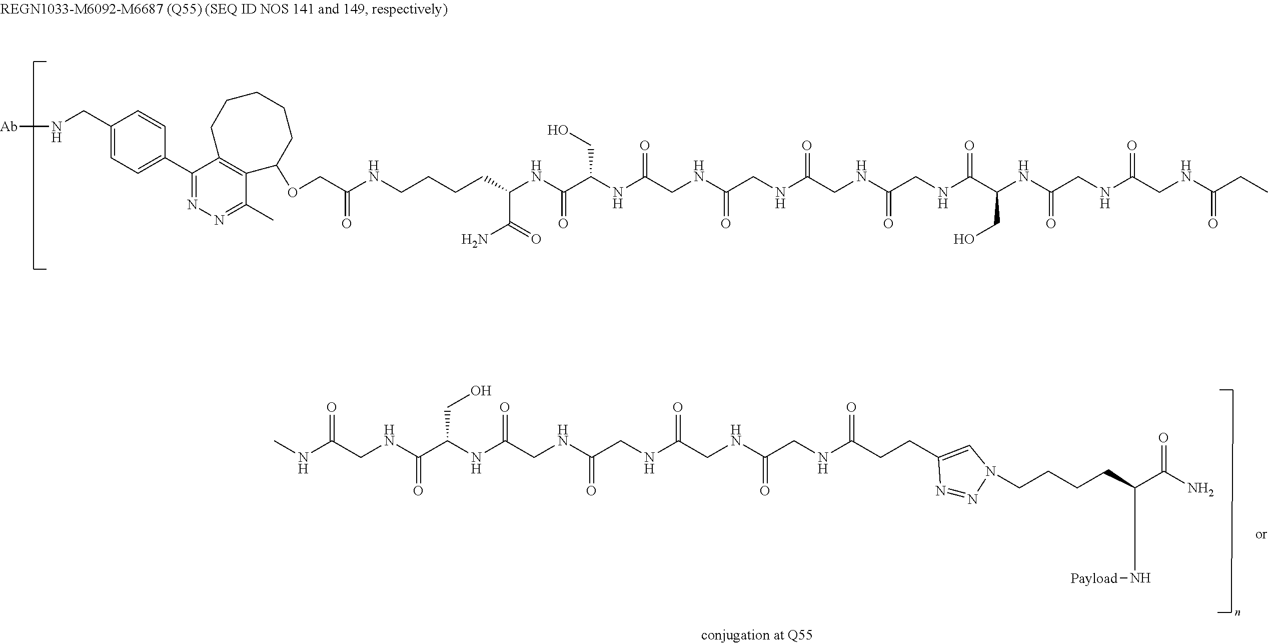

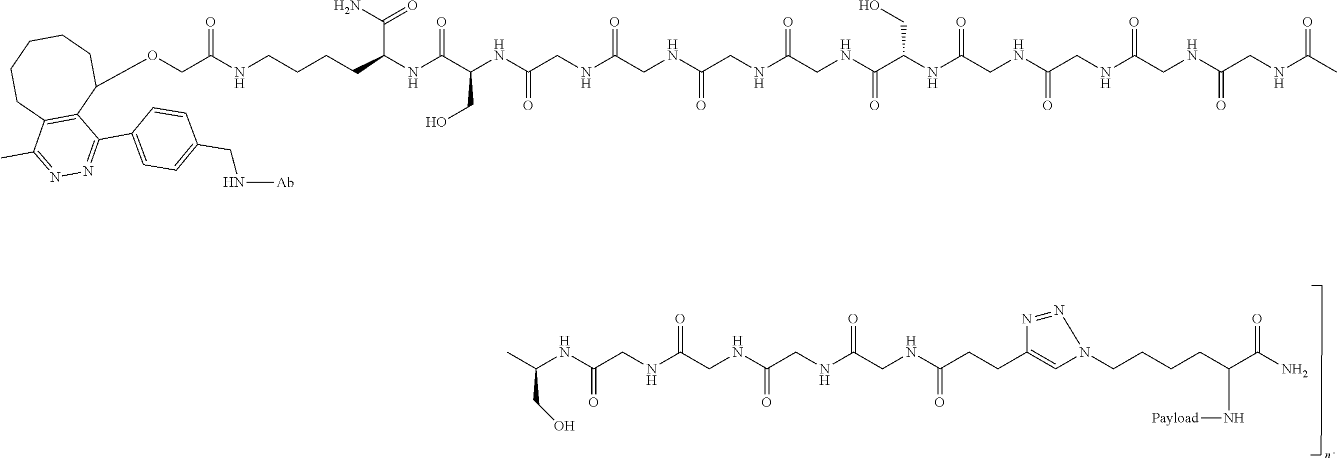

or a pharmaceutically acceptable salt thereof, where n is about 1 to 10, and Ab is a GDF8 antibody, or the antigen-binding fragment thereof, and where the Payload is a GLP1R and GIPR dual agonist having a sequence of Tyr1 Aib2 XX3 Gly4 XX5 XX6 Thr Ser Asp Tyr Ser Ser XX13 Leu Glu Glu Gln Ala Ala XX20 Glu XX22 Ile XX24 XX25 Leu Val XX28 Gly Gly Gly (SEQ ID NO: 3), wherein Aib represents 2-aminoisobutyric acid (α-aminoisobutyric acid); XX3 is Glu or Atz ((S)-2-amino-3-(2H-tetrazol-5-yl)propanoic acid); XX5 is Ser or Thr; XX6 is phenylalanine, alpha methyl phenylalanine (amF) or 2-fluoro alpha methyl phenylalanine (amF(2-fluoro)); XX13 is Tyr, amF, or alpha methyl tyrosine (amY); XX20 is Lys, sidechain-acylated or alkylated Lys, or Aib; XX22 is Phe or amF; XX24 is Ala or Glu; XX25 is tryptophan or alpha methyl tyrosine (amY); XX28 is Lys, Ala, Gln, or Glu.

In some embodiments the conjugate has a structure of

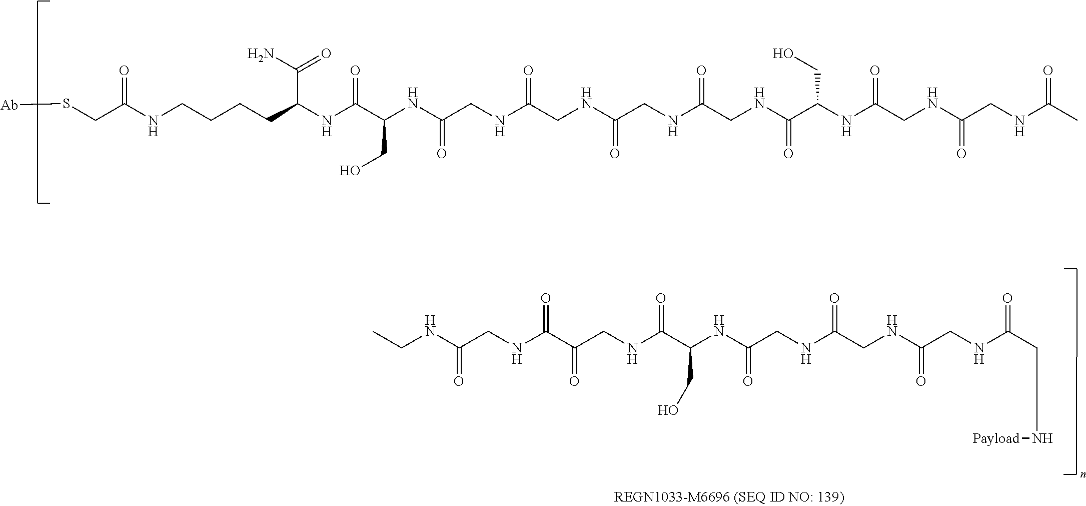

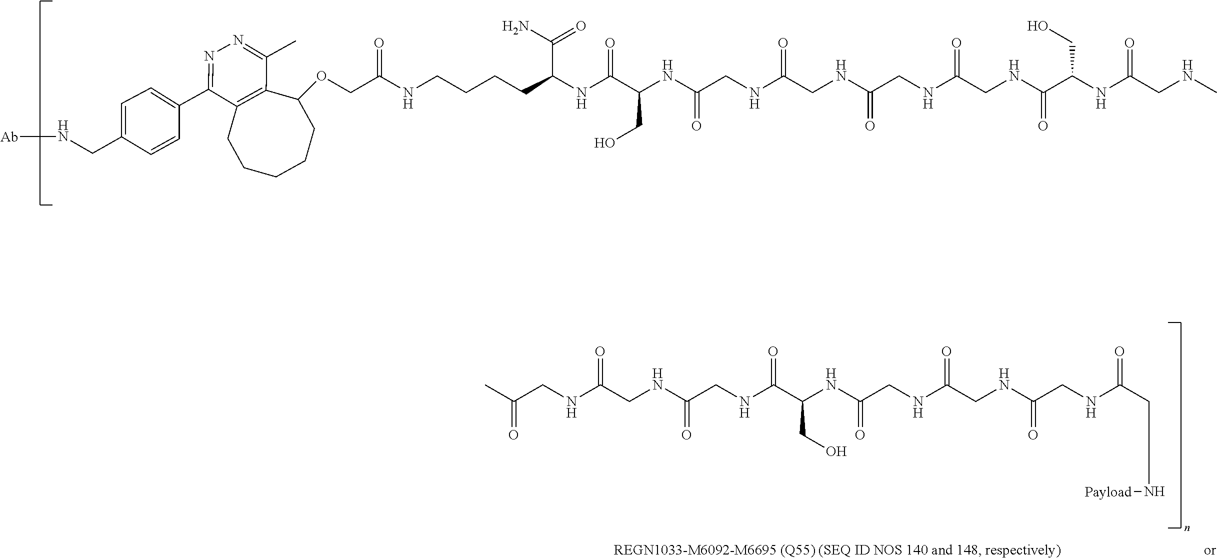

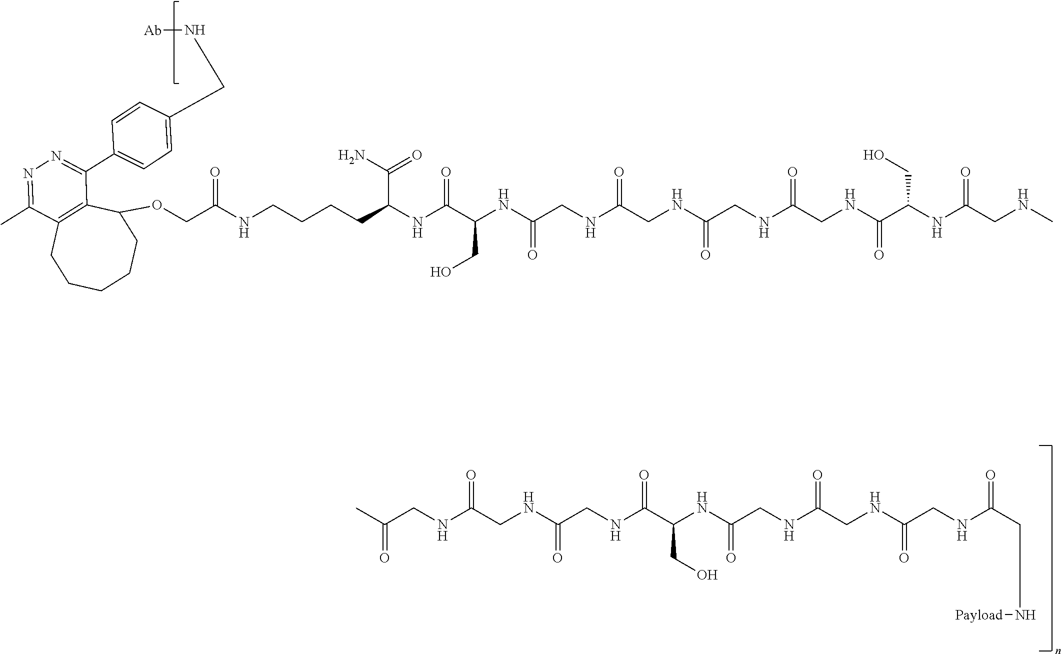

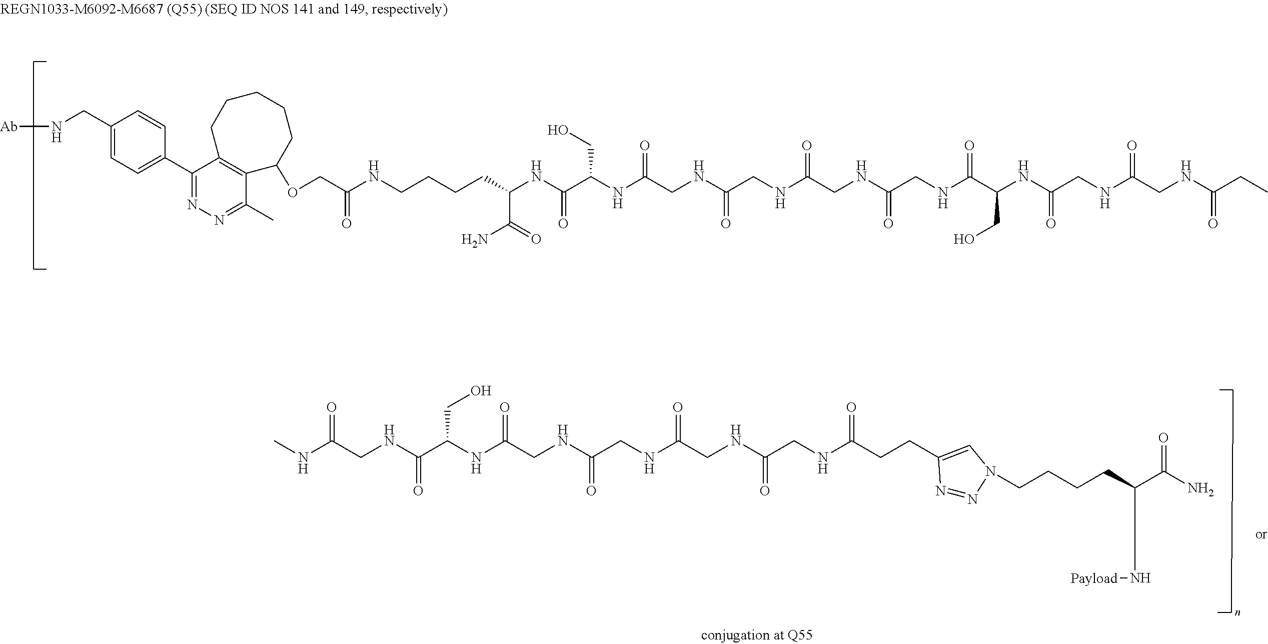

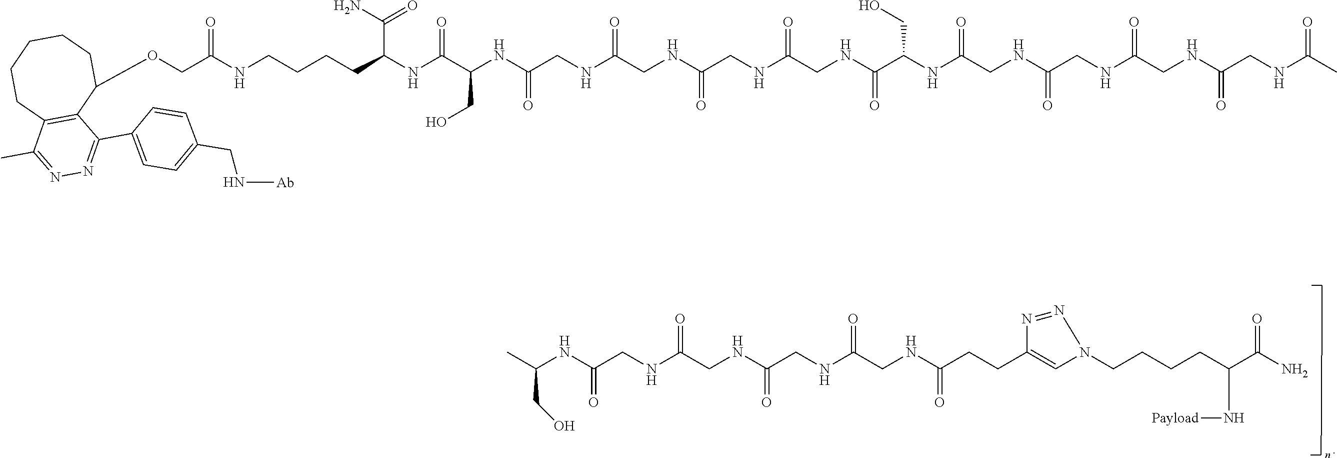

or a pharmaceutically acceptable salt thereof, where n is about 1 to 10, and Ab is a GDF8 antibody, or the antigen-binding fragment thereof, and where the Payload is a GLP1R and GIPR dual agonist having a sequence of Tyr1 Aib2 XX3 Gly4 XX5 XX6 Thr Ser Asp Tyr Ser Ser XX13 Leu Glu Glu Gln Ala Ala XX20 Glu XX22 Ile XX24 XX25 Leu Val XX28 Gly Gly Gly (SEQ ID NO: 3); wherein Aib represents 2-aminoisobutyric acid (α-aminoisobutyric acid); XX3 is Glu or Atz ((S)-2-amino-3-(2H-tetrazol-5-yl)propanoic acid); XX5 is Ser or Thr; XX6 is phenylalanine, alpha methyl phenylalanine (amF) or 2-fluoro alpha methyl phenylalanine (amF(2-fluoro)); XX13 is Tyr, amF, or alpha methyl tyrosine (amY); XX20 is Lys, sidechain-acylated or alkylated Lys, or Aib; XX22 is Phe or amF; XX24 is Ala or Glu; XX25 is tryptophan or alpha methyl tyrosine (amY); XX28 is Lys, Ala, Gln, or Glu.

In some embodiments, the linker with a reactive moiety for conjugation with a target antibody or antigen binding fragment thereof has the following structure: -(Gly₃Ser)ₙ-Lys(X)- (SEQ ID NO: 97) or -(Gly₄Ser)ₙ-Lys(X)- (SEQ ID NO: 98), where n is an integer from one to six; and where X is a reactive moiety attached to the side chain of Lys for conjugation with an antigen binding protein (ABP).

In some embodiments, X is a reactive moiety comprising an azido, 2-bromo-acetyl,

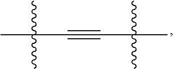 [SMILES: *C#C*]

2-(cyclooct-2-yn-lyloxy) acetyl, 2′-maleimido-acetyl, and 3′-maleimido-propanoyl, —N₃,

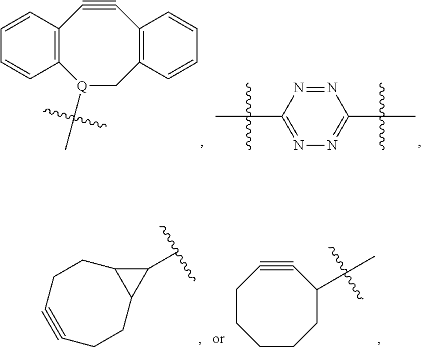

where Q is CH or N.

In some embodiments, Lys(X) has a structure of

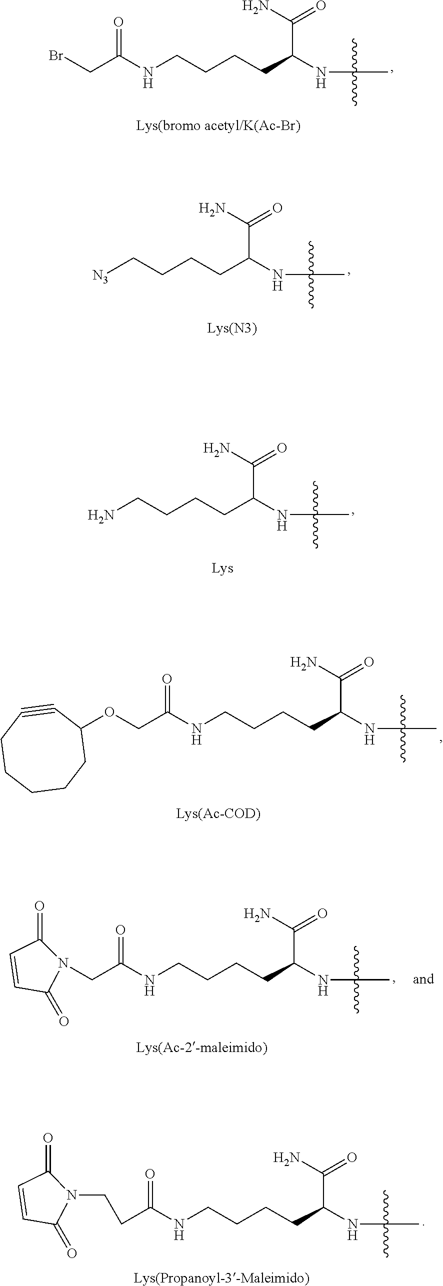

In some embodiments, the linker comprises

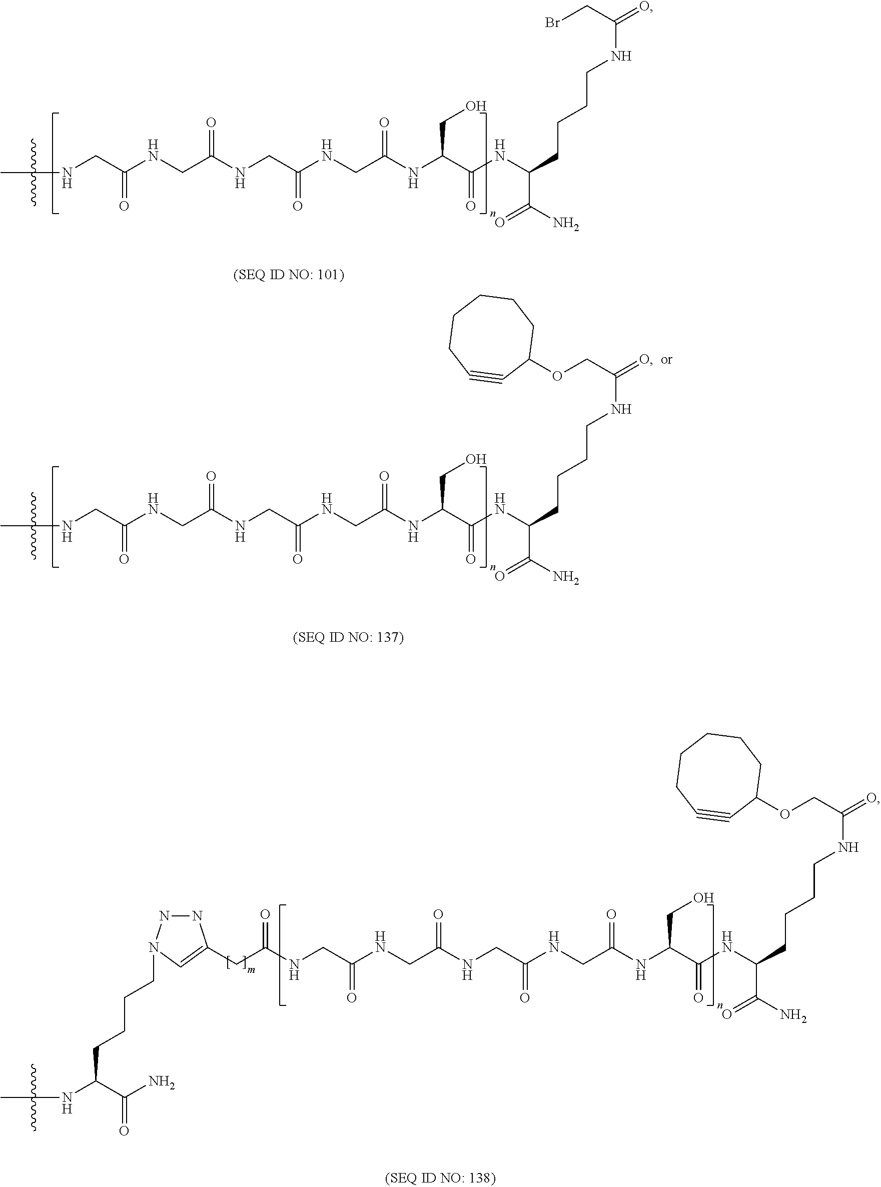

where m is an integer of one to three; and n is an integer of one to four.

### Antibody Conjugation

In another aspect, the present disclosure provides a process for manufacturing GLP1R-GIPR dual agonist and an antigen binding protein (ABP) conjugate, where ABP is a GDF8 antibody or antigen binding fragment thereof, comprising a) optionally attaching a handle comprising a first reactive moiety; b) exposing a GLP1R-GIPR dual agonist comprising a second reactive moiety; c) initiating conjugation reaction between the first and second reactive moieties; and d) isolating/purifying the conjugate of GLP1R-GIPR dual agonist and ABP.

Techniques and linkers for conjugating to residues of an antibody or antigen binding fragment are known in the art. Exemplary amino acid attachments that can be used in the context of this aspect, e.g., lysine (see, e.g., U.S. Pat. No. 5,208,020; US 2010/0129314; Hollander et al., Bioconjugate Chem., 2008, 19:358-361; WO 2005/089808; U.S. Pat. No. 5,714,586; US 2013/0101546; and US 2012/0585592), cysteine (see, e.g., US 2007/0258987; WO 2013/055993; WO 2013/055990; WO 2013/053873; WO 2013/053872; WO 2011/130598; US 2013/0101546; and U.S. Pat. No. 7,750,116), selenocysteine (see, e.g., WO 2008/122039; and Hofer et al., Proc. Natl. Acad. Sci., USA, 2008, 105:12451-12456), formyl glycine (see, e.g., Carrico et al., Nat. Chem. Biol., 2007, 3:321-322; Agarwal et al., Proc. Natl. Acad. Sci., USA, 2013, 110:46-51, and Rabuka et al., Nat. Protocols, 2012, 10:1052-1067), non-natural amino acids (see, e.g., WO 2013/068874, and WO 2012/166559), and acidic amino acids (see, e.g., WO 2012/05982). Lysine conjugation can also proceed through NHS (N-hydroxy succinimide). Linkers can also be conjugated to cysteine residues, including cysteine residues of a cleaved interchain disulfide bond, by forming a carbon bridge between thiols (see, e.g., U.S. Pat. Nos. 9,951,141, and 9,950,076). Linkers can also be conjugated to an antigen-binding protein via attachment to carbohydrates (see, e.g., US 2008/0305497, WO 2014/065661, and Ryan et al., Food & Agriculture Immunol., 2001, 13:127-130) and disulfide linkers (see, e.g., WO 2013/085925, WO 2010/010324, WO 2011/018611, and Shaunak et al., Nat. Chem. Biol., 2006, 2:312-313). Site specific conjugation techniques can also be employed to direct conjugation to particular residues of the antibody or antigen binding protein (see, e.g., Schumacher et al. J Clin Immunol (2016) 36 (Suppl 1): 100). In specific embodiments discussed in more detail below, Site specific conjugation techniques, include glutamine conjugation via transglutaminase (see e.g., Schibli, Angew Chemie Inter Ed. 2010, 49:9995).

GLP1R and GIPR dual agonists according to the disclosure linked through lysine and/or cysteine, e.g., via a maleimide or amide conjugation, are included within the scope of the present disclosure.

In some embodiments, the conjugates of the present disclosure are produced according to a two-step process, where Step 1 is lysine-based linker conjugation, e.g., with an NHS-ester linker, and Step 2 is a GLP1R and GIPR dual agonist conjugation reaction (e.g., a 1,3-cycloaddition reaction).

In some embodiments, the conjugates of the present disclosure are produced according to a two-step process, where Step 1 is cysteine-based linker conjugation, e.g., with a maleimide linker, and Step 2 is a GLP1R and GIPR dual agonist conjugation reaction (e.g., a 1,3-cycloaddition reaction).

In some embodiments, the conjugates of the present disclosure are produced according to a two-step process, where Step 1 is transglutaminase-mediated site specific conjugation and Step 2 is a GLP1R and GIPR dual agonist conjugation reaction (e.g., a 1,3-cycloaddition reaction).

### Step 1: Transglutaminase Mediated Site Specific Conjugation

In some embodiments, proteins (e.g., antibodies) may be modified in accordance with known methods to provide glutaminyl modified proteins. Techniques for conjugating antibodies and primary amine compounds are known in the art. Site specific conjugation techniques are employed herein to direct conjugation to glutamine using glutamine conjugation via transglutaminase (see e.g., Schibli, Angew Chemie Inter Ed. 2010, 49, 9995).

Primary amine-comprising compounds (e.g., linkers L) of the present disclosure can be conjugated to one or more glutamine residues of a binding agent (e.g., a protein, e.g., an antibody, e.g., an anti-GLP1R antibody) via transglutaminase-based chemo-enzymatic conjugation (see, e.g., Dennler et al., Protein Conjugate Chem. 2014, 25, 569-578, and WO 2017/147542). For example, in the presence of transglutaminase, one or more glutamine residues of an antibody can be coupled to a primary amine linker compound. Briefly, in some embodiments, a binding agent having a glutamine residue (e.g., a Gln295, i.e. Q295 residue) is treated with a primary amine-containing linker L in the presence of the enzyme transglutaminase. In certain embodiments, the binding agent is aglycosylated. In certain embodiments, the binding agent is deglycosylated.

In certain embodiments, the binding agent (e.g., a protein, e.g., an antibody) comprises at least one glutamine residue in at least one polypeptide chain sequence. In certain embodiments, the binding agent comprises two heavy chain polypeptides, each with one Gln295 residue. In further embodiments, the binding agent comprises one or more glutamine residues at a site other than a heavy chain 295.

In some embodiments, a binding agent, such as an antibody, can be prepared by site-directed mutagenesis to insert a glutamine residue at a site without resulting in disabled antibody function or binding. For example, included herein are antibodies bearing Asn297Gln (N297Q) mutation(s) as described herein. In some embodiments, an antibody having a Gln295 residue and/or an N297Q mutation contains one or more additional naturally occurring glutamine residues in their variable regions, which can be accessible to transglutaminase and therefore capable of conjugation to a linker or a linker-payload. An exemplary naturally occurring glutamine residue can be found, e.g., at Q55 of the light chain. In such instances, the binding agent, e.g., antibody, conjugated via transglutaminase can have a higher than expected LAR value (e.g., a LAR higher than 4). Any such antibodies can be isolated from natural or artificial sources.

### Step 2: GLP1R and GIPR Dual Agonist Conjugation Reaction

In certain embodiments, linkers L according to the present disclosure comprise at least one first reactive group capable of further reaction after transglutamination. In these embodiments, the glutaminyl-modified protein (e.g., antibody) is capable of further reaction with a GLP1R and GIPR dual agonist or a GLP1R and GIPR dual agonist attached to a reactive linker (L-P) to form a conjugate. More specifically, the reactive linker-GLP1R and GIPR dual agonist may comprise a second reactive group that is capable of reacting with the first reactive group of the linker L. In certain embodiments, a first or second reactive group according to the present disclosure comprises a moiety that is capable of undergoing a 1,3-cycloaddition reaction. In certain embodiments, the reactive group is an azide. In certain embodiments, the reactive group comprises an alkyne (e.g., a terminal alkyne, or an internal strained alkyne). In certain embodiments of the present disclosure the reactive group is compatible with the binding agent and transglutamination reaction conditions.

In certain embodiments of the disclosure, linker L molecules comprise a first reactive group. In certain embodiments of the disclosure, linker L molecules comprise more than one reactive group.

In certain embodiments, the reactive linker-GLP1R and GIPR dual agonist comprises one GLP1R and GIPR dual agonist molecule. In certain other embodiments, the reactive linker-GLP1R and GIPR dual agonist comprises two or more GLP1R and GIPR dual agonist molecules.

In certain embodiments of the disclosure, the drug-antibody ratio or DAR is from about 1 to about 30, or from about 1 to about 24, or from about 1 to about 20, or from about 1 to about 16, or from about 1 to about 12, or from about 1 to about 10, or from about 1 to about 8, or about 1, 2, 3, 4, 5, 6, 7, or 8 payload molecules per antibody. In some embodiments, the DAR is from 1 to 30. In some embodiments, the DAR is from 1 to 16. In some embodiments, the DAR is from 1 to 8. In some embodiments, the DAR is from 1 to 6. In certain embodiments, the DAR is from 2 to 4. In some cases, the DAR is from 2 to 3. In certain cases, the DAR is from 0.5 to 3.5. In some embodiments, the DAR is about 1, or about 1.5, or about 2, or about 2.5, or about 3, or about 3.5. In some embodiments, the DAR is 2. In some embodiments, the DAR is 4. In some embodiments, the DAR is 8.

Further details on the antibody preparation and their bioconjugation with the GLP1 peptide mimetics to manufacture antibody-tethered ligands can be found in U.S. Patent Application Publication No. 2022/0096648 and U.S. Patent Application Publication No. 2023/0330254, both of which are incorporated herein by reference in their entirety.

In another aspect, provided herein is a product of the process according to any of the embodiments described herein.

### Therapeutic Formulation and Administration

The present disclosure provides pharmaceutical compositions comprising the conjugates of the present disclosure.

The present disclosure provides pharmaceutical compositions comprising the GLP1R and GIPR dual agonists of the present disclosure or a pharmaceutically acceptable salt thereof.

In one aspect, the present disclosure provides compositions comprising a population of conjugates according to the present disclosure having a drug-antibody ratio (DAR) of about 0.5 to about 30.0.

In one embodiment, the composition has a DAR of about 1.0 to about 2.5.

In one embodiment, the composition has a DAR of about 2.

In one embodiment, the composition has a DAR of about 3.0 to about 4.5.

In one embodiment, the composition has a DAR of about 4.

In one embodiment, the composition has a DAR of about 6.5 to about 8.5.

In one embodiment, the composition has a DAR of about 8.

The compositions of the disclosure are formulated with one or more pharmaceutically accept salts, excipients, diluents, suitable carriers, and other agents that provide improved transfer, delivery, tolerance, and the like. A multitude of appropriate formulations can be found in the formulary known to all pharmaceutical chemists: Remington's Pharmaceutical Sciences, Mack Publishing Company, Easton, PA. These formulations include, for example, powders, pastes, ointments, jellies, waxes, oils, lipids, lipid (cationic or anionic) containing vesicles (such as LIPOFECTIN™, Life Technologies, Carlsbad, CA), DNA conjugates, anhydrous absorption pastes, oil-in-water and water-in-oil emulsions, emulsions carbowax (polyethylene glycols of various molecular weights), semi-solid gels, and semi-solid mixtures containing carbowax. See also Powell et al. “Compendium of excipients for parenteral formulations” PDA (1998) J Pharm Sci Technol 52:238-311.

In some embodiments, the pharmaceutical composition comprises one or more other therapeutic molecules.

The dose of a conjugate administered to a patient may vary depending upon the age and the size of the patient, target disease, conditions, route of administration, and the like. The suitable dose is typically calculated according to body weight or body surface area. When a conjugate of the present disclosure is used for therapeutic purposes in an adult patient, it may be advantageous to intravenously administer the conjugate of the present disclosure normally at a single dose of about 0.01 to about 20 mg/kg body weight, more preferably about 0.02 to about 7 mg/kg body weight, about 0.03 to about 5 mg/kg body weight, or about 0.05 to about 3 mg/kg body weight. Depending on the severity of the condition, the frequency and the duration of the treatment can be adjusted. Effective dosages and schedules for administering a conjugate may be determined empirically; for example, patient progress can be monitored by periodic assessment, and the dose adjusted accordingly. Moreover, interspecies scaling of dosages can be performed using well-known methods in the art (e.g., Mordenti et al., 1991, Pharmaceut. Res. 8:1351).

Various delivery systems are known and can be used to administer the pharmaceutical composition of the disclosure, e.g., encapsulation in liposomes, microparticles, microcapsules, recombinant cells capable of expressing the mutant viruses, receptor mediated endocytosis (see, e.g., Wu et al., 1987, J. Biol. Chem. 262:4429-4432). Methods of introduction include, but are not limited to, intradermal, intramuscular, intraperitoneal, intravenous, subcutaneous, intranasal, epidural, and oral routes. The composition may be administered by any convenient route, for example by infusion or bolus injection, by absorption through epithelial or mucocutaneous linings (e.g., oral mucosa, rectal, and intestinal mucosa, etc.) and may be administered together with other biologically active agents. Administration can be systemic or local.

A pharmaceutical composition of the present disclosure can be delivered subcutaneously or intravenously with a standard needle and syringe. In addition, with respect to subcutaneous delivery, a pen delivery device readily has applications in delivering a pharmaceutical composition of the present disclosure. Such a pen delivery device can be reusable or disposable. A reusable pen delivery device generally utilizes a replaceable cartridge that contains a pharmaceutical composition. Once all of the pharmaceutical composition within the cartridge has been administered and the cartridge is empty, the empty cartridge can readily be discarded and replaced with a new cartridge that contains the pharmaceutical composition. The pen delivery device can then be reused. In a disposable pen delivery device, there is no replaceable cartridge. Rather, the disposable pen delivery device comes prefilled with the pharmaceutical composition held in a reservoir within the device. Once the reservoir is emptied of the pharmaceutical composition, the entire device is discarded.

Numerous reusable pen and autoinjector delivery devices have applications in the subcutaneous delivery of a pharmaceutical composition of the present disclosure. Examples include, but are not limited to AUTOPEN™ (Owen Mumford, Inc., Woodstock, UK), DISETRONIC™ pen (Disetronic Medical Systems, Bergdorf, Switzerland), HUMALOG MIX 75/25™ pen, HUMALOG™ pen, HUMALIN 70/30™ pen (Eli Lilly and Co., Indianapolis, IN), NOVOPEN™ I, II and III (Novo Nordisk, Copenhagen, Denmark), NOVOPEN JUNIOR™ (Novo Nordisk, Copenhagen, Denmark), BD™ pen (Becton Dickinson, Franklin Lakes, NJ), OPTIPEN™, OPTIPEN PRO™, OPTIPEN STARLET™, and OPTICLIK™ (sanofi-aventis, Frankfurt, Germany), to name only a few. Examples of disposable pen delivery devices having applications in subcutaneous delivery of a pharmaceutical composition of the present disclosure include, but are not limited to the SOLOSTAR™ pen (sanofi-aventis), the FLEXPEN™ (Novo Nordisk), and the KWIKPEN™ (Eli Lilly), the SURECLICK™ Autoinjector (Amgen, Thousand Oaks, CA), the PENLET™ (Haselmeier, Stuttgart, Germany), the EPIPEN (Dey, L. P.), and the HUMIRA™ Pen (Abbott Labs, Abbott Park IL), to name only a few.

In certain situations, the pharmaceutical composition can be delivered in a controlled release system. In one embodiment, a pump may be used (see Langer, supra; Sefton, 1987, CRC Crit. Ref. Biomed. Eng. 14:201). In another embodiment, polymeric materials can be used; see, Medical Applications of Controlled Release, Langer and Wise (eds.), 1974, CRC Pres., Boca Raton, Florida. In yet another embodiment, a controlled release system can be placed in proximity of the composition's target, thus requiring only a fraction of the systemic dose (see, e.g., Goodson, 1984, in Medical Applications of Controlled Release, supra, vol. 2, pp. 115-138). Other controlled release systems are discussed in the review by Langer, 1990, Science 249:1527-1533.

The injectable preparations may include dosage forms for intravenous, subcutaneous, intracutaneous and intramuscular injections, drip infusions, etc. These injectable preparations may be prepared by methods publicly known. For example, the injectable preparations may be prepared, e.g., by dissolving, suspending, or emulsifying the antibody or its salt described above in a sterile aqueous medium or an oily medium conventionally used for injections. As the aqueous medium for injections, there are, for example, physiological saline, an isotonic solution containing glucose and other auxiliary agents, etc., which may be used in combination with an appropriate solubilizing agent such as an alcohol (e.g., ethanol), a polyalcohol (e.g., propylene glycol, polyethylene glycol), a nonionic surfactant [e.g., polysorbate 80, HCO-50 (polyoxyethylene (50 mol) adduct of hydrogenated castor oil)], etc. As the oily medium, there are employed, e.g., sesame oil, soybean oil, etc., which may be used in combination with a solubilizing agent such as benzyl benzoate, benzyl alcohol, etc. The injection thus prepared is preferably filled in an appropriate ampoule.

Advantageously, the pharmaceutical compositions for oral or parenteral use described above are prepared into dosage forms in a unit dose suited to fit a dose of the active ingredients. Such dosage forms in a unit dose include, for example, tablets, pills, capsules, injections (ampoules), suppositories, etc. The amount of the aforesaid antibody contained is generally about 5 to about 500 mg per dosage form in a unit dose; especially in the form of injection, it is preferred that the aforesaid antibody is contained in about 5 to about 100 mg and in about 10 to about 250 mg for the other dosage forms.

In another aspect, provided herein is a pharmaceutical composition comprising a GLP1R-GIPR dual agonist and an antigen binding protein (ABP) conjugate manufactured according to the process of any of the embodiments described herein, together with one or more pharmaceutically acceptable excipients.

### Therapeutic Uses of the Conjugates and GLP1R-GIPR Dual Agonists

In another aspect, the compounds (e.g., a conjugate and/or a GLP1R-GIPR dual agonist), disclosed herein are useful, inter alia, for the treatment, prevention and/or amelioration of a disease, disorder or condition in need of such treatment.

In one aspect, the present disclosure provides a method of treating a condition in a subject in need thereof comprising administering to the subject a therapeutically effective amount of a compound (e.g., a conjugate and/or a GLP1R-GIPR dual agonist) according to the disclosure, or the composition comprising any compound according to the present disclosure.

In some embodiments, the compounds (e.g., a conjugate and/or a GLP1R-GIPR dual agonist) disclosed herein are useful for treating any disease or disorder in which stimulation, activation, and/or targeting of GLP1R would be beneficial. In particular, the anti-GLP1R antibody-drug conjugates of the present disclosure can be used for the treatment, prevention, and/or amelioration of any disease or disorder associated with or mediated by GLP1R expression or activity.

In some embodiments, the compounds (e.g., a conjugate and/or a GLP1R-GIPR dual agonist) disclosed herein are useful for treating a GLP1R-associated condition. In some embodiments, the GLP1R-associated condition is Type 1 or Type 2 diabetes mellitus. The administered compound (e.g., a conjugate and/or a GLP1R-GIPR dual agonist) may cause at least one of the following results: induction of insulin secretion, suppression of glucagon release, reduction of blood sugar, improvement of glycemic control, promotion of islet neogenesis, and delay of gastric emptying or potentiation of glucose resistant islets.

In some embodiments, the GLP1R-associated condition is a neurodegenerative disorder, a cognitive disorder, memory disorder, or learning disorder. The neurodegenerative disorder may be, for example, dementia, senile dementia, mild cognitive impairment, Alzheimer-related dementia, Huntington's chores, tardive dyskinesia, hyperkinesias, mania, Morbus Parkinson, steel-Richard syndrome, Down's syndrome, myasthenia gravis, nerve trauma, brain trauma, vascular amyloidosis, cerebral hemorrhage I with amyloidosis, brain inflammation, Friedrich's ataxia, acute confusion disorder, amyotrophic lateral sclerosis, glaucoma, and Alzheimer's disease.

In some embodiments, the GLP1R-associated condition is a liver disease. The liver disease may be, for example, non-alcoholic fatty liver disease (NAFLD), fatty liver, non-alcoholic steatohepatitis (NASH), and cirrhosis.

In some embodiments, the GLP1R-associated condition is a coronary artery disease. The coronary artery disease may be, for example, cardiomyopathy and myocardial infarction.

In some embodiments, the GLP1R-associated condition is an obesity.

In some embodiments, the GLP1R-associated condition is a kidney disease. The kidney disease may be, for example, hypertension or chronic kidney failure.

In some embodiments, the GLP1R-associated condition is an eating disorder. The eating disorder may be, for example, binge eating.

Without wishing to be bound by theory, the compounds (e e.g., a conjugate and/or a GLP1R-GIPR dual agonist) disclosed herein may be employed to attenuate the effects of apoptosis-mediated degenerative diseases of the central nervous system such as Alzheimer's Disease, Creutzfeld-Jakob Disease and bovine spongiform encephalopathy, chronic wasting syndrome and other prion mediated apoptotic neural diseases (see, e.g., Perry and Grieg (2004) Current Drug Targets 6:565-571). Administration of a compound (e.g., a conjugate and/or a GLP1R-GIPR dual agonist) disclosed herein may also lead to down-modulation of PAPP and thereby ameliorate Aβ mono- or oligomer-mediated pathologies associated with Alzheimer's Disease (see, e.g., Perry et al. (2003) Journal of Neuroscience Research 72:603-612).

It is also contemplated that the compounds (e.g., a conjugate and/or a GLP1R-GIPR dual agonist) disclosed herein may be used to improve learning and memory, for example, by enhancing neuronal plasticity and facilitation of cellular differentiation (see, During et al. (2003) Nature Medicine 9:1173-1179). Further, the compounds (e.g., a conjugate and/or a GLP1R-GIPR dual agonist) disclosed herein may also be used to preserve dopamine neurons and motor function in Morbus Parkinson (see, e.g., Greig et al. (2005) Abstract 897.6, Society for Neuroscience, Washington, D.C.).

In some embodiments, the compounds (e.g., a conjugate and/or a GLP1R-GIPR dual agonist) disclosed herein may also be used to treat a metabolic disorder. The metabolic disorder may be, for example, obesity, dyslipidemia, metabolic syndrome X, and pathologies emanating from islet insufficiency.

Additional diseases that may be treated by a compound (e.g., a conjugate and/or a GLP1R-GIPR dual agonist) of the present disclosure include autoimmune diseases, in particular, those associated with inflammation, including, but not limited to, autoimmune diabetes, adult onset diabetes, morbid obesity, Metabolic Syndrome X, and dyslipidemia. For example, the anti-GLP1R antibody-drug conjugate can be employed as a growth factor for the promotion of islet growth in persons with autoimmune diabetes. The compounds (e.g., a conjugate and/or a GLP1R-GIPR dual agonist) described herein may also be useful in the treatment of congestive heart failure.

In one aspect, the present disclosure provides a method for treating obesity by reducing body weight while maintaining or increasing lean body mass in a subject, comprising administering a therapeutically effective amount of a GLP1R-GIPR dual agonist or an antigen binding protein (ABP) conjugate thereof according to any of the embodiments described herein, to the subject in need thereof.

In another aspect, the present disclosure provides a method for treating obesity by reducing fat mass while maintaining or increasing lean body mass in a subject, comprising administering a therapeutically effective amount of a GLP1R-GIPR dual agonist or an antigen binding protein (ABP) conjugate thereof according to any of the embodiments described herein, to the subject in need thereof.

In another aspect, the present disclosure provides a method for treating Type 2 diabetes by improving glycemic control and maintaining or increasing lean body mass while reducing fat mass in a subject, comprising administering a therapeutically effective amount of a GLP1R-GIPR dual agonist or an antigen binding protein (ABP) conjugate thereof according to any of the embodiments described herein, to the subject in need thereof.

In yet another aspect, the present disclosure provides a method for treating obesity and Type 2 diabetes by improving glycemic control and maintaining or increasing lean body mass while reducing fat mass in a subject, comprising administering a therapeutically effective amount of a GLP1R-GIPR dual agonist or an antigen binding protein (ABP) conjugate thereof according to any of the embodiments described herein, to the subject in need thereof.

In a further aspect, the present disclosure provides a method for treating obesity, diabetes, and/or liver diseases associated with increased fat mass in a subject, comprising administering a therapeutically effective amount of a GLP1R-GIPR dual agonist and an antigen binding protein (ABP) conjugate according to any of the embodiments described herein, to the subject in need thereof.

In yet another aspect, the present disclosure provides a method for treating a subject of metabolic syndrome by improving glycemic control, maintaining or increasing lean body mass while reducing fat mass in a subject, comprising administering a therapeutically effective amount of a GLP1R-GIPR dual agonist or an antigen binding protein (ABP) conjugate thereof according to any of the embodiments described herein, to the subject in need thereof.

### Combination Therapies and Formulations

The present disclosure provides methods which comprise administering a pharmaceutical composition comprising any of the exemplary conjugates and/or a GLP1R-GIPR dual agonists described herein in combination with one or more additional therapeutic agents.

In another aspect, the present disclosure provides a method for treating obesity, diabetes, and/or liver diseases associated with increased fat mass in a subject, comprising administering a therapeutically effective amount of a GLP1R-GIPR dual agonist or an antigen binding protein (ABP) conjugate thereof according to any of the embodiments described herein, together with one or more other therapeutic agents, to the subject in need thereof.

In a further aspect, the present disclosure provides a method for treating a subject of metabolic syndrome by improving glycemic control, maintaining or increasing lean body mass while reducing fat mass in a subject, comprising administering a therapeutically effective amount of a GLP1R-GIPR dual agonist or an antigen binding protein (ABP) conjugate thereof according to any of the embodiments described herein, together with one or more other therapeutic agents, to the subject in need thereof.

Exemplary additional therapeutic agents that may be combined with or administered in combination with conjugates and/or a GLP1R-GIPR dual agonists of the present disclosure include, other GLP1R agonists (e.g., an anti-GLP1R antibody or a small molecule agonist of GLP1R or an anti-GLP1R antibody-drug conjugate). Non-limiting examples of GLP1R agonists include exenatide (Byetta, Bydureon), liraglutide (Victoza, Saxenda), lixisenatide (Lyxumia in Europe, Adlyxin in the United States), albiglutide (Tanzeum), dulaglutide (Trulicity), semaglutide (Ozempic), and taspoglutide.

Exemplary additional therapeutic agents may include dual or triple-agonists, including GLP1R/GIPR dual agonists, such as GLP1R/GCGR dual agonists, GLP1R/GIPR/GCGR triple-agonists.

Other agents that may be beneficially administered in combination with the conjugates and/or a GLP1R-GIPR dual agonists of the disclosure include those that are useful in the treatment of diabetes (e.g., type II diabetes), obesity, and/or other related metabolic diseases.

In some embodiments, the additional therapeutic agent is an antidiabetic agent. Any suitable antidiabetic agents can be used. Non-limiting examples of antidiabetic agents include insulin, insulin analogs (including insulin lispro, insulin aspart, insulin glulisine, isophane insulin, insulin zinc, insulin glargine, and insulin detemir), biguanides (including metformin, phenformin, and buformin), thiazolidinediones or TZDs (including rosiglitazone, pioglitazone, and troglitazone), sulfonylureas (including tolbutamide, acetohexamide, tolazamide, chlorpropamide, glipizide, glibenclamide, glimepiride, gliclazide, glyclopyramide, and gliquidone), meglitinides (including repaglinide and nateglinide), alpha-glucosidase inhibitors (including miglitol, acarbose, and voglibose), glucagon-like peptide analogs and agonists (including exenatide, liraglutide, semaglutide, taspoglutide, lixisenatide, albuglutide, and dulaglutide), gastric inhibitory peptide analogs, dipeptidyl peptidase-4 (DPP-4) inhibitors (including vildagliptin, sitagliptin, saxagliptin, linagliptin, alogliptin, septagliptin, teneligliptin, and gemigliptin), amylin agonist analogs, sodium/glucose cotransporter 2 (SGLT2) inhibitors, glucokinase activators, squalene synthase inhibitors, other lipid lowering agents and aspirin. In some such embodiments, the antidiabetic agent is an oral antidiabetic agents (OAA) such as metformin, acarbose, or TZDs. In some such embodiments, the antidiabetic agent is metformin.

In some embodiments, the GLP1R agonist and one or more antidiabetic agents may be formulated into the same dosage form, such as a solution or suspension for parenteral administration.

The additional therapeutically active component(s) may be administered just prior to, concurrent with, or shortly after the administration of conjugates and/or a GLP1R-GIPR dual agonists of the present disclosure; (for purposes of the present disclosure, such administration regimens are considered the administration of an antigen-binding molecule “in combination with” an additional therapeutically active component).

The present disclosure includes pharmaceutical compositions in which conjugates and/or a GLP1R-GIPR dual agonists of the present disclosure are co-formulated with one or more of the additional therapeutically active component(s) as described elsewhere herein.

### Administration Regimens

According to certain embodiments of the present disclosure, multiple doses of a conjugate and/or a GLP1R-GIPR dual agonist may be administered to a subject over a defined time course. The methods according to this aspect of the disclosure comprise sequentially administering to a subject multiple doses of a conjugate and/or a GLP1R-GIPR dual agonist of the disclosure. As used herein, “sequentially administering” means that each dose of a conjugate and/or a GLP1R-GIPR dual agonist is administered to the subject at a different point in time, e.g., on different days separated by a predetermined interval (e.g., hours, days, weeks, or months). The present disclosure includes methods which comprise sequentially administering to the patient a single initial dose of a conjugate and/or a GLP1R-GIPR dual agonist, followed by one or more secondary doses of the conjugate and/or a GLP1R-GIPR dual agonist, and optionally followed by one or more tertiary doses of the conjugate and/or a GLP1R-GIPR dual agonist.

The terms “initial dose,” “secondary doses,” and “tertiary doses,” refer to the temporal sequence of administration of the conjugate and/or a GLP1R-GIPR dual agonist of the disclosure. Thus, the “initial dose” is the dose which is administered at the beginning of the treatment regimen (also referred to as the “baseline dose”); the “secondary doses” are the doses which are administered after the initial dose; and the “tertiary doses” are the doses which are administered after the secondary doses. The initial, secondary, and tertiary doses may all contain the same amount of the conjugate and/or a GLP1R-GIPR dual agonist, but generally may differ from one another in terms of frequency of administration. In certain embodiments, however, the amount of the conjugate and/or a GLP1R-GIPR dual agonist contained in the initial, secondary and/or tertiary doses varies from one another (e.g., adjusted up or down as appropriate) during the course of treatment. In certain embodiments, two or more (e.g., 2, 3, 4, or 5) doses are administered at the beginning of the treatment regimen as “loading doses” followed by subsequent doses that are administered on a less frequent basis (e.g., “maintenance doses”).

In one exemplary embodiment of the present disclosure, each secondary and/or tertiary dose is administered 1 to 26 (e.g., 1, 1½, 2, 2½, 3, 3½, 4, 4½, 5, 5½, 6, 6½, 7, 7½, 8, 8½, 9, 9½, 10, 10½, 11, 11½, 12, 12½, 13, 13½, 14, 14½, 15, 15½, 16, 16½, 17, 17½, 18, 18½, 19, 19½, 20, 20½, 21, 21½, 22, 22½, 23, 23½, 24, 24½, 25, 25½, 26, 26½, or more) weeks after the immediately preceding dose. The phrase “the immediately preceding dose,” as used herein, means, in a sequence of multiple administrations, the dose of a conjugate and/or a GLP1R-GIPR dual agonist which is administered to a patient prior to the administration of the very next dose in the sequence with no intervening doses.

The methods according to this aspect of the disclosure may comprise administering to a patient any number of secondary and/or tertiary doses of a conjugate and/or a GLP1R-GIPR dual agonist. For example, in certain embodiments, only a single secondary dose is administered to the patient. In other embodiments, two or more (e.g., 2, 3, 4, 5, 6, 7, 8, or more) secondary doses are administered to the patient. Likewise, in certain embodiments, only a single tertiary dose is administered to the patient. In other embodiments, two or more (e.g., 2, 3, 4, 5, 6, 7, 8, or more) tertiary doses are administered to the patient.

In embodiments involving multiple secondary doses, each secondary dose may be administered at the same frequency as the other secondary doses. For example, each secondary dose may be administered to the patient 1 to 2 weeks after the immediately preceding dose. Similarly, in embodiments involving multiple tertiary doses, each tertiary dose may be administered at the same frequency as the other tertiary doses. For example, each tertiary dose may be administered to the patient 2 to 4 weeks after the immediately preceding dose. Alternatively, the frequency at which the secondary and/or tertiary doses are administered to a patient can vary over the course of the treatment regimen. The frequency of administration may also be adjusted during the course of treatment by a physician depending on the needs of the individual patient following clinical examination.

### EXAMPLES

The following examples illustrate specific aspects of the instant description. The examples should not be construed as limiting, as the examples merely provide specific understanding and practice of the embodiments and their various aspects.

The abbreviations used in the Examples and throughout the specification are shown in Table 1.

TABLE 1
Abbreviations

| | |
|---|---|
| AA | Amino acid |
| ACN | Acetonitrile |
| amAA | α-Methylated amino acid |
| Boc | tert-Butyloxycarbonyl |
| DCM | Dichlormethane |
| Dde | 1-(4,4-diMethyl-2,6-dioxocyclohexylidene)ethyl |
| DIPEA | Diisopropylethylamine |
| DIC | N,N′-Diisopropylcarbodiimide |
| DMF | N,N-Dimethylformamide |
| Fmoc | 9-Fluorenylmethyloxycarbonyl |
| MeOH | Methanol |
| Mc-COOH | 2-maleimido acetic acid or 3-Maleimidopropionic acid |
| HATU | 2-(7-Azabenzotriazol-1-yl)-N,N,N′,N′-tetramethyluronium |
| | hexafluorophosphate |
| HBTU | O-Benzotriazole-N,N,N′,N′-tetramethyl-uronium- |
| | hexafluorophosphate |
| HOBT | 1-Hydroxybenzotriazole |
| HPLC | High-performance liquid chromatography |
| MPA | 3-Mercaptopropionicacid |
| MTBE | Methyl tert-Butyl Ether |
| t-Bu | tert-Butyl |
| TEA | Triethylamine |
| TFA | Trifluoroacetic acid |
| TIS | Triisopropylsilane |
| Trt | Trityl |
| Pip | Piperidine |
| Pydec | 2-pyridyldithioethyloxycarbonyl |
| Atz | (αS)-α-Amino-1H-tetrazole-5-propanoic acid |

TABLE 2
Sequence Information of GLPIR-GIPR Dual Agonist Peptides #1-28*

| | SEQ | | | | | | | | | | | | | | C-terminal |
|---|---|---|---|---|---|---|---|---|---|---|---|---|---|---|---|
| NO | ID NO: | M# | 5 | 6 | 13 | 20 | 21 | 22 | 23 | 24 | 25 | 26 | 27 | 28 | amide** |
| 1 | 111 | M6486 | T | F | Y | K | E | F | I | A | W | L | V | K | amide |
| 2 | 112 | M6487 | T | amF(2F) | Y | K | E | F | I | A | W | L | V | K | amide |
| 3 | 113 | M6953 | T | amF(2F) | Y | K | E | F | I | A | W | L | V | K | K(N3) |
| 4 | 114 | M6954 | T | amF(2F) | Y | K | E | F | I | A | W | L | V | K | (G4S)3-K[K(N3)] |
| 5 | 115 & | M6989 | T | amF(2F) | Y | K | E | F | I | A | W | L | V | K | K[T-(CH2)2CO- |
| | 150 | | | | | | | | | | | | | | (G4S)3-K(ALO)] |
| 6 | 116 | M6990 | T | amF(2F) | Y | K | E | F | I | A | W | L | V | K | K(T-CH2N-ALO) |
| 7 | 117 | M6956 | T | amF | amF | K | E | F | I | A | W | L | V | K | K(N3) |
| 8 | 118 | M6957 | S | amF | Y | K | E | F | I | A | amY | L | V | K | K(N3) |
| 9 | 119 | M6958 | T | amF | Y | K | E | amF | I | A | W | L | V | K | K(N3) |
| 10 | 120 | M6959 | T | amF | amY | K | E | amF | I | A | W | L | V | K | K(N3) |
| 11 | 121 | M6695 | T | amF | Y | K | E | F | I | A | amY | L | V | K | (G4S)3-K(ALO) |
| 12 | 122 | M6960 | T | amF(2F) | Y | K | E | F | I | A | amY | L | V | K | K(N3) |
| 13 | 123 | M6961 | T | amY | Y | K | E | F | I | A | amY | L | V | K | K(N3) |
| 14 | 124 | M6697 | T | amY | Y | K | E | F | I | A | W | L | V | K | K(N3) |
| 15 | 125 | M6962 | T | amF | Y | K | E | F | I | A | W | L | V | K | K(N3) |
| 16 | 126 | M6682 | T | amF | Y | K | E | F | I | A | amY | L | V | K | K(N3) |
| 17 | 127 | M6696 | T | amF | Y | K | E | F | I | A | amY | L | V | K | (G4S)3-K(AcBr) |
| 18 | 128 & | M6687 | T | amF | Y | K | E | F | I | A | amY | L | V | K | K[T-(CH2)2CO- |
| | 151 | | | | | | | | | | | | | | (G4S)3-K(ALO)] |
| 19 | 129 | M6810 | T | amF | Y | K | E | F | I | A | amY | L | V | K | (G4S)3-K |
| 20 | 130 & | M6969 | T | amY | Y | K | E | F | I | A | W | L | V | K | K[T-(CH2)2CO- |
| | 152 | | | | | | | | | | | | | | (G4S)3-K(ALO)] |
| 21 | 131 & | M6970 | T | amY | Y | K | E | F | I | A | amY | L | V | K | K[T-(CH2)2CO- |
| | 153 | | | | | | | | | | | | | | (G4S)3-K(ALO)] |
| 22 | 132 | M6976 | T | amF | Y | K | E | F | I | A | amY | L | V | K | (G4S)3- |
| | | | | | | | | | | | | | | | K(DIBAC) |
| 23 | 133 | M6991 | T | aml | Y | K | E | F | I | A | amY | L | V | K | amide |
| 24 | 134 | M6977 | T | amF | Y | K | E | F | I | A | amY | L | V | E | K(N3) |
| 25 | 135 | M6978 | T | amF | Y | K | E | F | I | E | amY | L | V | E | K(N3) |
| 26 | 136 | M6979 | T | amF | Y | Aib | E | F | I | E | amY | L | V | E | K(N3) |
| 27 | 111 | M6491 | T | F | Y | K | E | F | I | A | W | L | V | K | amide |
| 28 | 114 | M6988 | T | amF(2F) | Y | K | E | F | I | A | W | L | V | K | (G4S)3-K[K(N3)] |

*Except for peptides 27 and 28, wherein the third residue is Atz (2-amino-3-(2H-tetrazol-5-yl) propanoic acid); for Peptides NO#1-26, residues 1-4 are Tyr-Aib-Glu-Gly; residues 7-12 are Thr-Ser-Asp-Tyr-Ser-Ser (SEQ ID NO: 102); residues 14-19 are Leu-Glu-Glu-Gln-Ala-Ala (SEQ ID NO: 103); residues 26-27 are Leu-Val; and residue 29-31 are Gly-Gly-Gly. Aib is 2-aminoisobutyric acid (α-aminoisobutyric acid); amF: alpha methyl phenylalanine; am Y: alpha methyl tyrosine; amF(2F): 2-fluoro-alpha methyl phenylalanine; ALO: 2-(cyclooct-2-yn-lyloxy) acetyl; and T represent triazole moiety.
**All peptides have a C-terminal amide unless otherwise specified.

TABLE 3
Summary of Chemical Characterization of Synthesized Peptides 1-30.

| | | | | | | | | SEQ |
|---|---|---|---|---|---|---|---|---|
| | | | | Purity | RT | | | ID |
| NO | M code | MF | FW* | (%) | (min) | Mass | salt | NO |
| 1 | M6486 | C₁₅₈H₂₂₈N₃₆O₅₀ | 3773.82 | 90 | 10.66 | 1144.8 [M + 3H]/3 | TFA | 18 |
| 2 | M6487 | C₁₅₉H₂₂₉FN₃₆O₅₀ | 3805.84 | 90.4 | 10.09 | 1155.4 [M + 3H]/3 | TFA | 19 |
| 3 | M6953 | C₁₆₅H₂₃₉FN₄₀O₅₁ | 3960.1 | 95.4 | 11.25 | 905.3 [M + 4H]/4 | TFA | 20 |
| 4 | M6954 | C₂₀₄H₃₀₂FN₅₇O₇₀ | 5376.11 | 95.9 | 17.32 | 1173.8 [M + 4H]/4 | TFA | 21 |
| 5 | M6989 | C₂₁₉H₃₂₁FN₅₈O₇₃ | 5409.39 | 95.1 | 10.53 | 1238.9 [M + 4H]/4 | TFA | 22 |
| 6 | M6990 | C₁₇₈H₂₅₆FN₄₁O₅₃ | 4065.28 | 94.6 | 11.91 | 960.1 [M + 4H]/4 | TFA | 23 |
| 7 | M6956 | C₁₆₆H₂₄₂N₄₀O₅₀ | 3940.05 | 91.8 | 11.76 | 900.3 [M + 4H]/4 | TFA | 24 |
| 8 | M6957 | C₁₆₄H₂₄₁N₃₉O₅₁ | 3917.01 | 96.5 | 10.87 | 895.0 [M + 4H]/4 | TFA | 25 |
| 9 | M6958 | C₁₆₆H₂₄₂N₄₀O₅₁ | 3956.05 | 93 | 11.83 | 904.2 [M + 4H]/4 | TFA | 26 |
| 10 | M6959 | C₁₆₇H₂₄₄N₄₀O₅₁ | 3970.08 | 89.8 | 11.89 | 907.8 [M + 4H]/4 | TFA | 27 |
| 11 | M6695 | C₂₀₇H₃₀₆N₅₂O₇₂ | 5017.08 | 98.2 | 10.16 | 1169.4 [M + 4H]/4 | TFA | 15 |
| 12 | M6960 | C₁₆₄H₂₄₀FN₃₉O₅₂ | 3951 | 97.7 | 10.79 | 722.7 [M + 5H]/5 | TFA | 28 |
| 13 | M6961 | C₁₆₄H₂₄₁N₃₉O₅₃ | 3949.01 | 95.3 | 10.65 | 902.7 [M + 4H]/4 | TFA | 29 |
| 14 | M6697 | C₁₆₅H₂₄₀N₄₀O₅₂ | 3958.02 | 94.4 | 11.3 | 904.7 [M + 4H]/4 | TFA | 30 |
| 15 | M6962 | C₁₆₅H₂₄₀N₄₀O₅₁ | 3942.02 | 93.9 | 11.6 | 904.7 [M + 4H]/4 | TFA | 31 |
| 16 | M6682 | C₁₆₄H₂₄₁N₃₉O₅₂ | 3933.01 | 97.7 | 13.53 | 898.5 [M + 4H]/4 | TFA | 17 |
| 17 | M6696 | C₁₉₉H₂₉₅BrN₅₂O₇₁ | 4973.75 | 96.1 | 11.94 | 1158.6 [M + 4H]/4 | TFA | 16 |
| 18 | M6687 | C₂₁₈H₃₂₃N₅₇O₇₄ | 5268.31 | 98.8 | 9.8 | 1232.3 [M + 4H]/4 | TFA | 14 |
| 19 | M6810 | C₁₉₇H₂₉₄N₅₂O₇₀ | 4966.89 | 93.4 | 11.7 | 1128.4 [M + 4H]/4 | TFA | 32 |
| 20 | M6969 | C₂₁₉H₃₂₂N₅₈O₇₄ | 5293.38 | 97.4 | 10.12 | 1238.6 [M + 4H]/4 | TFA | 33 |
| 21 | M6970 | C₂₁₈H₃₂₃N₅₇O₇₅ | 5284.37 | 99 | 12.17 | 1236.2 [M + 4H]/4 | TFA | 34 |
| 22 | M6976 | C₂₁₆H₃₀₇N₅₃O₇₂ | 4866.25 | 94.8 | 10.45 | 1200.2 [M + 4H]/4 | NH3 | 35 |
| 23 | M6991 | C₁₅₈H₂₃₁N₃₅O₅₁ | 3436.78 | 97.7 | 10.33 | 1146.2 [M + 3H]/3 | TFA | 54 |
| 24 | M6977 | C₁₆₃H₂₃₆N₃₈O₅₄ | 3819.93 | 99.5 | 11.57 | 898.8 [M + 4H]/4 | TFA | 36 |
| 25 | M6978 | C₁₆₅H₂₃₈N₃₈O₅₆ | 3877.97 | 99.5 | 11.59 | 913.3 [M + 4H]/4 | TFA | 37 |
| 26 | M6979 | C₁₆₃H₂₃₃N₃₇O₅₆ | 3720.88 | 99.5 | 13 | 722.7 [M + 5H]/5 | TFA | 38 |
| 27 | M6491 | C₁₅₇H₂₂₆N₄₀O₄₈ | 3897.85 | 93.3 | 12.89 | 1148.1 [M + 3H]/3 | TFA | 39 |
| 28 | M6988 | C₂₀₃H₃₀₀N₆₁O₆₈ | 5272.09 | 93.8 | 15.02 | 1567.9 [M + 3H]/3 | TFA | 40 |
| 29 | M6562 | C204H301N55O70 | 5100.05 | 98.3 | 10.73 | 1548.7 [M + 3H]3+ | TFA | 52 |
| 30 | M6677 | C215H318N60O72 | 5351.34 | 95.3 | 9.78 | 1632.3 [M + 3H]3+ | TFA | 53 |

*FW (formular weight) refers to the molecular weight plus the counters (TFA salt in most cases).
All peptides have a C-terminal amide unless otherwise specified.

TABLE 4
Definition, Abbreviation, and Structural Diagram

| Name | Structure | Name | Structure |
|---|---|---|---|
| Amino- tetrazine handle or Amino-azide handle | 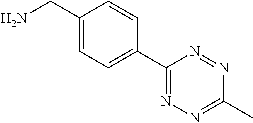 | K(N3) | 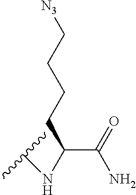 [SMILES: *N[C@@H](CCCCN=[N+]=[N-])C(N)=O] |
| | (M6092) | | |
| | or | | |
| | 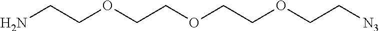 [SMILES: [N-]=[N+]=NCCOCCOCCOCCN] | | |
| | (M404) | | |
| DIBAC-Suc | 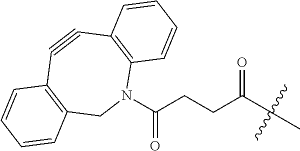 [SMILES: *C(=O)CCC(=O)N1Cc2ccccc2C#Cc2ccccc21] | ALO or Ac-COT | 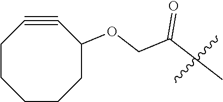 [SMILES: *C(=O)COC1C#CCCCCC1] |
| TzCOT or TCOT | 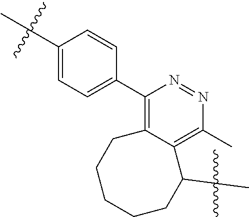 [SMILES: *c1ccc(-c2nnc(C)c3c2CCCCCC3*)cc1] | K(T) | 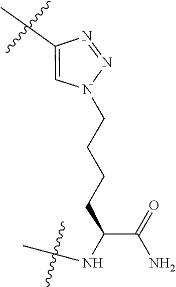 [SMILES: *N[C@@H](CCCCn1cc(*)nn1)C(N)=O] |
| | 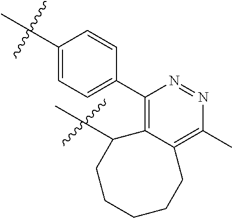 [SMILES: *c1ccc(-c2nnc(C)c3c2C(*)CCCCC3)cc1] | | |
| | or | | |
| | 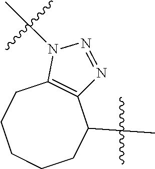 [SMILES: *C1CCCCCc2c1nnn2*] | | |
| | 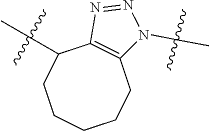 [SMILES: *C1CCCCCc2c1nnn2*] | | |
| K(ALO) | 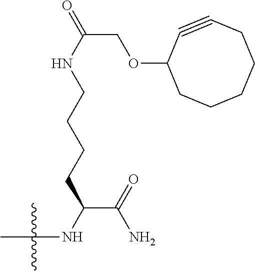 [SMILES: *N[C@@H](CCCCNC(=O)COC1C#CCCCCC1)C(N)=O] | (G4S)nK (DIBAC) (SEQ ID NO: 104) (C- terminus of M6976) n = 1-4 | 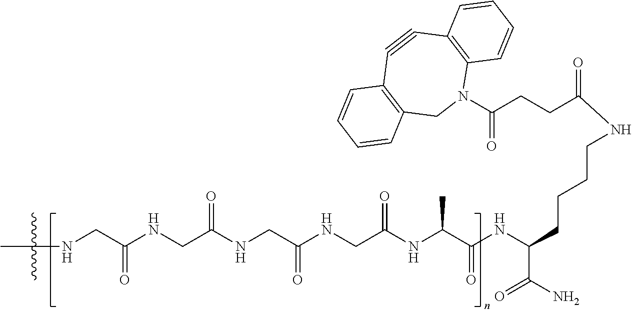 |
| amF | 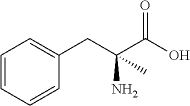 | amF(2- fluoro)/ amF(2F) | 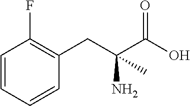 |
| amY | 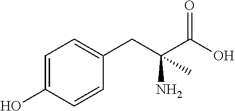 | Atz (2- amino-3- (2H- tetrazol-5- yl) propanoic acid) | 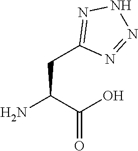 [SMILES: N[C@@H](Cc1nn[nH]n1)C(=O)O] |
| (G4S)nK(T- X-ALO) (SEQ ID NO: 105) X = (CH2)m, m = 1,2,3; n = 1,2,3,4 | 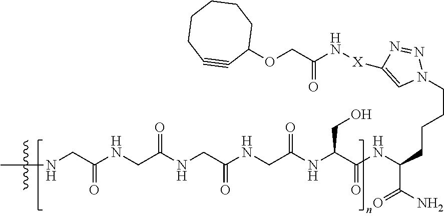 | C-terminus of M6990 | 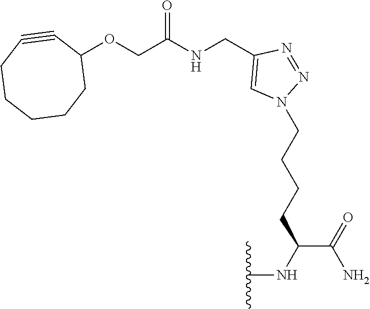 [SMILES: *N[C@@H](CCCCn1cc(CNC(=O)COC2C#CCCCCC2)nn1)C(N)=O] |

| | |
|---|---|
| Linker- Payload A (SEQ ID NO: 138) | 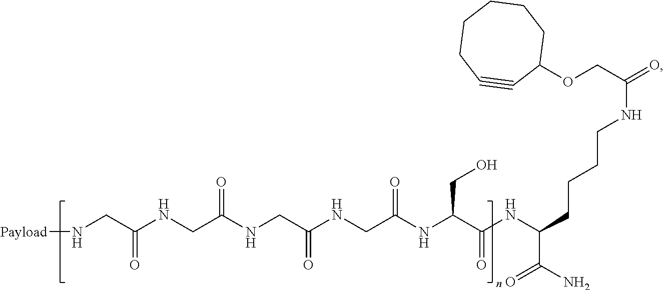 |
| | wherein the Payload has a SEQ ID NO: 3. |
| Linker- Payload C (SEQ ID NO: 147) | 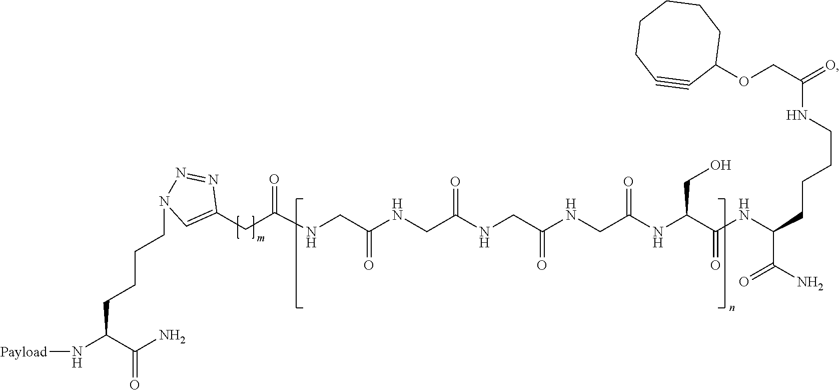 |
| | wherein the Payload has a SEQ ID NO: 3 |
| (G4S)nK n = 1-4 (SEQ ID NO: 106) | 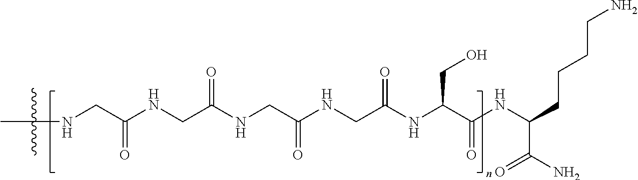 |
| (G4S)nK(ALO) n = 1-4 (SEQ ID NO: 107) (Linker A) | 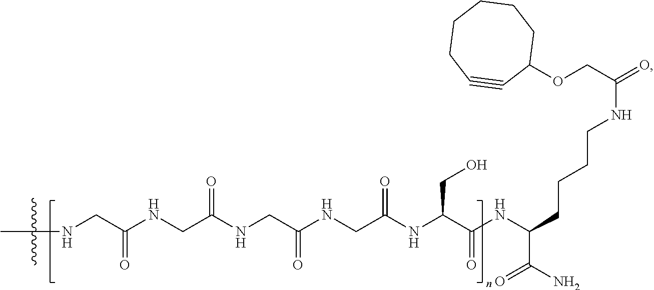 |
| K[T- (CH₂)₂CO- (G4S)n- K(ALO)], n = 1-4 (SEQ ID NO: 108) Linker C | 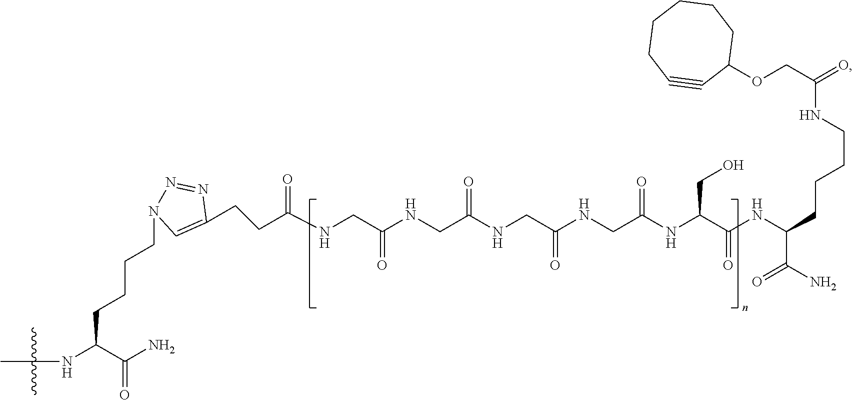 |
| K[T-(G4S)n- K(ALO)] n = 1-4 (SEQ ID NO: 109) Linker C | 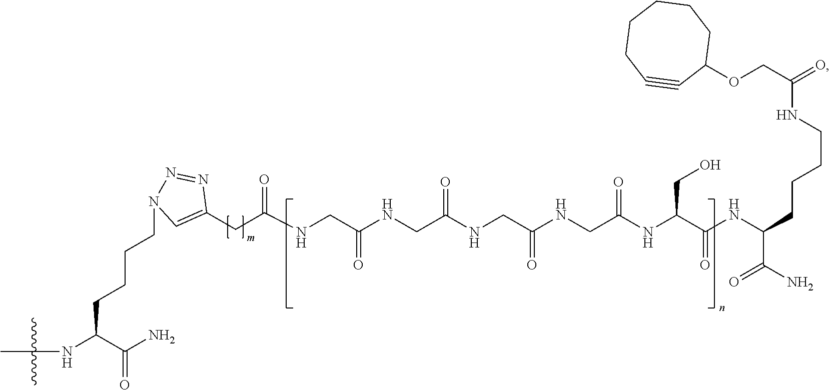 |
| | m = 1,2,3; n = 1,2,3,4 |

TABLE 5
Structures of Handles for Conjugation to an Antibody with a Gln

| Code | MW | Structure |
|---|---|---|
| M404 | 218.14 | 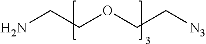 |
| M6092 | 201.23 | 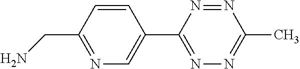 |
| M6093 | 363.42 | 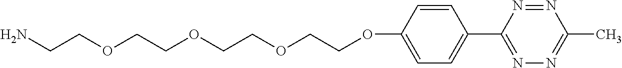 [SMILES: Cc1nnc(-c2ccc(OCCOCCOCCOCCN)cc2)nn1] |
| M2153 | 674.77 | 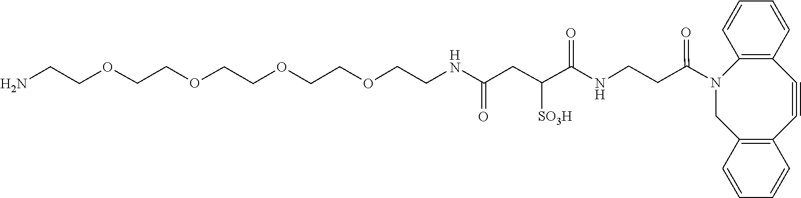 [SMILES: NCCOCCOCCOCCOCCNC(=O)CC(C(=O)NCCC(=O)N1Cc2ccccc2C#Cc2ccccc21)S(=O)(=O)O] |

### Experimental Section for Peptide Synthesis and Purification

### Example 1. General Procedures for the Synthesis of Peptides

### Peptide Synthesis

The peptide was synthesized using standard Fmoc chemistry. Most peptides were synthesized with a scale of 0.05-0.5 mmol based on the initial loading of the first amino acid.

### Resin Preparation

A solution of Rink Amide MBHA Resin (1.0 eq, Sub 0.33 mmol/g) in DMF (50.0 mL/mmol of Resin) was agitated with N₂ at 25° C. for 2 hrs. Then 20% piperidine in DMF (50.0 mL mmol of Resin) was added and agitated with N₂ at 25° C. for 15 min. The resin was washed with DMF (50.0 mL/mmol of Resin for 5 times).
Alternative method is swelling the Rink-amide MBHA resin (initial resin loading 0.3 mmol/g) with DCM (10 mL/gram of Resin×4 times). 20% piperidine in DMF solution was added into the resin. The mixture was agitated for 2×20 min. After the reaction was completed, the resin was washed with DMF (2×5 mL).

### Amide Coupling Reaction

Coupling the 1^{st} AA (Lys as an example): A solution of Oxyma (3.0 eq) and Fmoc-Lys(Dde)-OH (3.0 eq) in DMF (25.0 mL/mmol of Resin) was added to the resin, then DIC (3.0 eq) was added, and the mixture was agitated with N₂ at 25° C. for 30 min. The resin was washed with DMF (50.0 mL/mmol of Resin×5).
Coupling of native amino acid: Fmoc-AA-OH (2 eq.)/HBTU (2 eq.)/DIPEA (4 eq.) was dissolved in DMF with a final concentration of 0.2 mM. The mixture was agitated for 60-90 min at room temperature. After the reaction was completed, the resin was washed with DMF (2×5 mL).
Coupling of alpha-methylated AA (amAA): Fmoc-amAA-OH (2 eq.)/HATU (2 eq.)/DIPEA (4 eq.) was dissolved in DMF with a final concentration of 0.2 mM. The mixture was agitated for 16 h at room temperature. After the reaction was completed, the resin was washed with DMF (2×5 mL).
Coupling of the amino acid after Aib: Fmoc-AA-OH (3 eq.)/HATU (3 eq.)/DIPEA (6 eq.) was dissolved in DMF with a final concentration of 0.2 mM. The mixture was agitated for 2×45 min at room temperature. After the reaction was completed, the resin was washed with DMF (2×5 mL).
Coupling of amino acid after amAA: Fmoc-AA-OH (20 eq.)/HATU (20 eqv.)/DIPEA (30 eqv.) was dissolved in DMF with a final concentration of 0.5 mM. The mixture was agitated for 16 h at room temperature. Mini-cleavage LCMS was necessary to monitor the progress of the reaction. After the reaction was completed, the resin was washed with DMF (2×5 mL).
In some cases, after de-Fmoc of N-terminal amino acid, the free amine was protected with Boc-group.

### Deprotections

Fmoc deprotection: 20% piperidine in DMF (100.0 mL) was added and agitated the resin with N₂ at 25° C. for 15 min. The resin was washed with DMF (100.0 mL×5) and filtered to get the resin.
Dde deprotection: The resin-bound peptide was treated with hydrazine monohydrate (3% N₂H₄·H₂O/DMF): for 2 mmol scale of resin bond peptide, the reaction was run in 50 mL of 5% N₂H₄·H₂O/DMF for 20 min twice; for 0.05 mmol scale of resin bond peptide, the reaction was run in 10 mL of 5% N₂H₄·H₂O/DMF for 30 min. Once the reaction was completed, drain and wash the mixture with DMF (100 mL×5 times for large scale or wash with DMF (10 mL), MeOH (10 mL), DCM (2×10 mL) and DMF (2×10 mL).

### Cleavage

The resin bound peptide was cleaved using TFA/TIS/H₂O/MPA (17:1:1:1) for 1.5-2 hrs or TFA/TIS/H₂O/DTT (16:1:1:2) at RT for 1.5-2 hrs. For peptide containing Atz, 5 vol % of ethanedithiol was added into the cleavage cocktail. Once completed, the resin was filtered, washed with small volume of TFA twice.

### Precipitation

Precipitated the peptide with cold isopropyl ether (1.50 L per gram of peptide). Filter and collect the filter cake. The filter cake was washed with isopropyl ether (1.50 L*2) and dried under vacuum for 1 hr to get the crude peptide.
Alternative method: The peptide was precipitated by addition of ten-fold volume of cold MTBE to the TFA solution and mixed thoroughly. The mixture was centrifuged. The supernatant was decanted. The solid containing crude peptide was further washed with MTBE twice, centrifuged, and then dried under reduced pressure.

### Purification Method

The crude peptide was purified by prep-HPLC (0.0100 TFA/CH3CN/H2O) to afford the target peptide after lyophilization. Purification conditions are as follows.

| | |
|---|---|
| Dissolution condition | DMSO |
| Mobile Phase | A: H₂O |
| | B: CH₃CN |
| Gradient | 10-90-50 min., Retention time: 30 min |
| Column | Spherical, c18, 20-45 um, 100 A |
| Flow Rate | 100 mL/Min |
| Wavelength | 220/254 nm |
| Oven Tem. | 30° C. |

| | |
|---|---|
| Dissolution condition | Dissolve in 30% ACN-H2O |
| Instrument | Gilson GX-281 |
| Mobile Phase | A: H₂O (0.075% TFA in H₂O) |
| | B: CH₃CN |
| Gradient | 25-45-50 min, Retention time: 28 min |
| Column | luna, 10 um, c18, 100 A, 25 mm + Gemin, |
| | 5 um, c18, 110 A |
| Flow Rate | 20 mL/Min |
| Wavelength | 220/254 nm |
| Oven Temperature | 30° C. |

### System Information of Analytical Instruments

Analytical HPLC spectra were recorded with Agilent 1200 series system; LCMS spectra were recorded with Agilent 1200 series system coupled with Agilent 6130 Quadrupole MS detector.

### Alternative HPLC Conditions

| | |
|---|---|
| Dissolution condition | Dissolve in 30% ACN-H2O |
| Instrument | Gilson GX-281 |
| Mobile Phase | A: H₂O (0.075% TFA in H₂O) |
| | B: CH₃CN |
| Gradient | 25-45-50 min, Retention time: 28 min |
| Column | luna, 10 um, c18, 100 A, 25 mm + Gemin, |
| | 5 um, c18, 110 A |
| Flow Rate | 20 mL/Min |
| Wavelength | 220/254 nm |
| Oven Tem. | 30° C. |

### Example 2. Synthesis of Peptide M6696 Using General SPPS Procedure

The peptide M6696 (Linker-Payload for Ab-Cys conjugation) was synthesized by standard solid phase peptide synthesis strategy, including resin preparation and loading; amide coupling and de-Fmoc reaction; cleavage and purification.

| | Resin: Rink Amide MBHA (loading: 0.33 mmol/g) (1 g, 0.3 mmol). Temperature: 25° C. | | | |
|---|---|---|---|---|
| Step | | | | Reacting |
| # | Materials | Coupling reagents | solvent | time |
| 1 | Fmoc-Lys(Dde)-OH | HBTU (2.0 eq.), DIPEA (4.0 eq.) | DMF (15 mL) | 60 min |
| 2 | Fmoc-Ser(tBu)-OH | HBTU (2.0 eq.), DIPEA (4.0 eq.) | DMF (15 mL) | 60 min |
| 3 | Fmoc-Gly2-OH | HBTU (2.0 eq.), DIPEA (4.0 eq.) | DMF (15 mL) | 60 min |
| 4 | Fmoc-Gly2-OH | HBTU (2.0 eq.), DIPEA (4.0 eq.) | DMF (15 mL) | 60 min |
| 5 | Fmoc-Gly-Ser(ψMe,Me pro)- | HBTU (2.0 eq.), DIPEA (4.0 eq.) | DMF (15 mL) | 60 min |
| | OH | | | |
| 6 | Fmoc-Gly3-OH | HBTU (2.0 eq.), DIPEA (4.0 eq.) | DMF (15 mL) | 60 min |
| 7 | Fmoc-Gly-Ser(ψMe,Me pro)- | HBTU (2.0 eq.), DIPEA (4.0 eq.) | DMF (15 mL) | 60 min |
| | OH | | | |
| 8 | Fmoc-Gly3-OH | HBTU (2.0 eq.), DIPEA (4.0 eq.) | DMF (15 mL) | 60 min |
| 9 | Fmoc-Gly3-OH | HBTU (2.0 eq.), DIPEA (4.0 eq.) | DMF (15 mL) | 60 min |
| 10 | Fmoc-Lys(Boc)-OH | HBTU (2.0 eq.), DIPEA (4.0 eq.) | DMF (15 mL) | 60 min |
| 11 | Fmoc-Val-OH | HBTU (2.0 eq.), DIPEA (4.0 eq.) | DMF (15 mL) | 60 min |
| 12 | Fmoc-Leu-OH | HBTU (2.0 eq.), DIPEA (4.0 eq.) | DMF (15 mL) | 60 min |
| 13 | Fmoc-amY(OtBu)-OH | HBTU (2.0 eq.), DIPEA (4.0 eq.) | DMF (15 mL) | 60 min |
| 14 | Fmoc-Ala-OH | HATU (20. eq.), DIPEA (30 eq.) | DMF (15 mL) | 16 h |
| 15 | Fmoc-Ile-OH | HBTU (2.0 eq.), DIPEA (4.0 eq.) | DMF (15 mL) | 60 min |
| 16 | Fmoc-Phe-OH | HBTU (2.0 eq.), DIPEA (4.0 eq.) | DMF (15 mL) | 60 min |
| 17 | Fmoc-Glu(OtBu)-OH | HBTU (2.0 eq.), DIPEA (4.0 eq.) | DMF (15 mL) | 60 min |
| 18 | Fmoc-Lys(Boc)-OH | HBTU (2.0 eq.), DIPEA (4.0 eq.) | DMF (15 mL) | 60 min |
| 19 | Fmoc-Ala-OH | HBTU (2.0 eq.), DIPEA (4.0 eq.) | DMF (15 mL) | 60 min |
| 20 | Fmoc-Ala-OH | HBTU (2.0 eq.), DIPEA (4.0 eq.) | DMF (15 mL) | 60 min |
| 21 | Fmoc-Gln(Trt)-OH | HBTU (2.0 eq.), DIPEA (4.0 eq.) | DMF (15 mL) | 60 min |
| 22 | Fmoc-Glu(OtBu)-OH | HBTU (2.0 eq.), DIPEA (4.0 eq.) | DMF (15 mL) | 60 min |
| 23 | Fmoc-Glu(OtBu)-OH | HBTU (2.0 eq.), DIPEA (4.0 eq.) | DMF (15 mL) | 60 min |
| 24 | Fmoc-Leu-OH | HBTU (2.0 eq.), DIPEA (4.0 eq.) | DMF (15 mL) | 60 min |
| 25 | Fmoc-Tyr(tBu)-OH | HBTU (2.0 eq.), DIPEA (4.0 eq.) | DMF (15 mL) | 60 min |
| 26 | Fmoc-Ser(tBu)-OH | HBTU (2.0 eq.), DIPEA (4.0 eq.) | DMF (15 mL) | 60 min |
| 27 | Fmoc-Tyr(tBu)-Ser(ψMe,Me | HBTU (2.0 eq.), DIPEA (4.0 eq.) | DMF (15 mL) | 60 min |
| | pro)-OH | | | |
| 28 | Fmoc-Asp(OtBu)-OH | HBTU (2.0 eq.), DIPEA (4.0 eq.) | DMF (15 mL) | 60 min |
| 29 | Fmoc-Thr(tBu)-Ser(ψMe,Me | HBTU (2.0 eq.), DIPEA (4.0 eq.) | DMF (15 mL) | 60 min |
| | pro)-OH | | | |
| 30 | Fmoc-amF-OH | HBTU (2.0 eq.), DIPEA (4.0 eq.) | DMF (15 mL) | 60 min |
| 31 | Fmoc-Thr(tBu)-OH | HATU (20. eq.), DIPEA (30 eq.) | DMF (15 mL) | 16 h |
| 32 | Fmoc-Gly-OH | HBTU (2.0 eq.), DIPEA (4.0 eq.) | DMF (15 mL) | 60 min |
| 33 | Fmoc-Glu(OtBu)-OH | HBTU (2.0 eq.), DIPEA (4.0 eq.) | DMF (15 mL) | 60 min |
| 34 | Fmoc-Aib-OH | HBTU (2.0 eq.), DIPEA (4.0 eq.) | DMF (15 mL) | 60 min |
| 35 | Boc-Tyr(tBu)-OH | HBTU (2.0 eq.), DIPEA (4.0 eq.) | DMF (15 mL) | 90 min |
| 36 | Dde removal | HBTU (2.0 eq.), DIPEA (4.0 eq.) | DMF (15 mL) | 60 min |
| 37 | Bromoacetic acid (2 eq.) | DIC (2.0 eq.) | DMF (15 mL) | 60 min |
| | Cleavage | TFA (90%), TIS (5%), H2O (5%) | | 90 min |
| | Crude amount | 1400 mg | | |
| | Isolated amount | 40.5 mg | | |
| | Purity | 96.1% | | |
| | Yield | 2.8% | | |

### Cleavage Conditions

The resin bound peptide was cleaved using the cleavage cocktail (TFA (90%), TIS (5%), H₂O (5%)) for 90 min at 25° C. The mixture was filtered, and the solution was collected. The residual resin was washed with TFA (10 mL) twice. The combined solutions were poured into MTBE (pre-cooled to −10° C., 150 mL) and shaken to precipitate the crude peptide. The suspension was centrifuged, and the supernatant was decanted. The residual solid was further washed with MTBE (50 mL×2) and centrifuged. After decanting the supernatant, the residual solid was then dried under reduced pressure overnight to give crude peptide as an off-white solid.

Once the peptide was fully elongated, the peptide was then cleaved and purified with RP-HPLC to afford M6696 as white solid. ESI m/z: 1544.4 [M+3H]/3⁺, 1158.6 [M+4H]/4⁺, 927.0 [M+5H]/5⁺, 772.8 [M+6H]/6⁺, 662.7 [M+7H]/⁷⁺.

### Example 3. Synthesis of Peptide M6695 Using General SPPS Procedure

### Method A

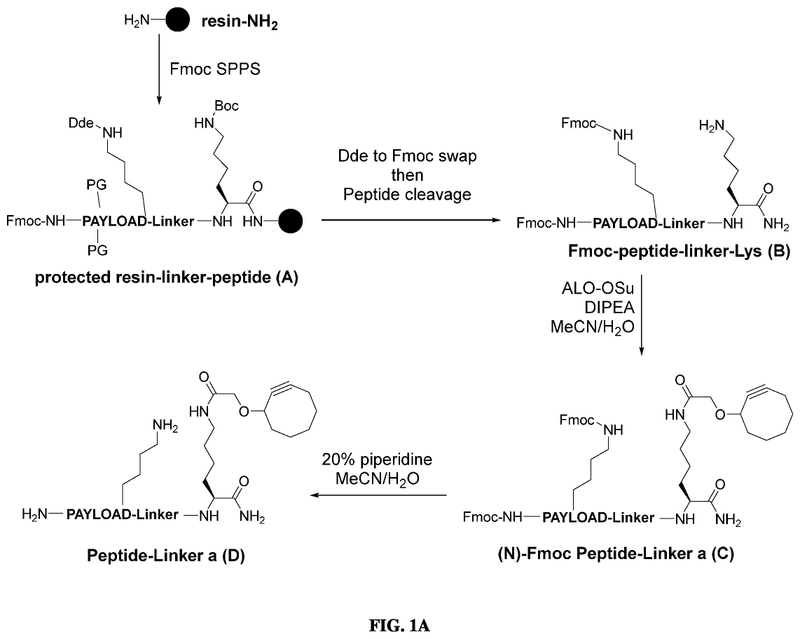

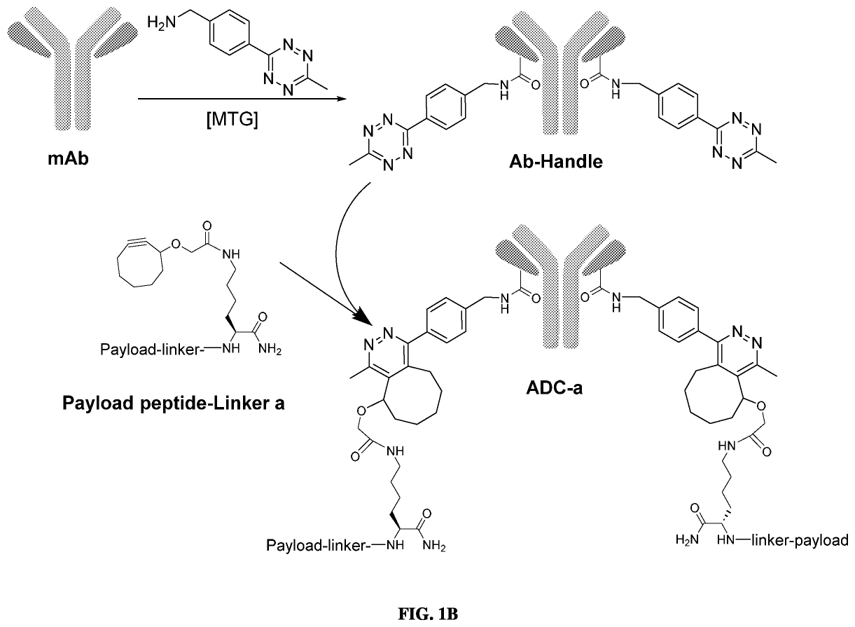

In method A shown in FIG. 1A: Peptide-linker was synthesized directly on SPPS. Here, linker includes proteogenic peptide (GGGGS)n, where n=1, 2, 3, or 4 (SEQ ID NO: 110); polysarcosine linker: (Sar)n, where n=1 to 20; PEG linker: NH2-(PEG)n-COOH, which includes aeea, aeep, aeeea, aeeep and other PEG linker with 1-20 PEG repeats. Once the linker peptide (A) was fully elongated in SPPS, modification of suitable protecting group was conducted for orthogonal protection of multiple amine groups on the peptide. Suitable protecting groups involved Fmoc, Alloc, azido, Pydec groups, etc. The Peptide-Linker was cleaved from resin (B), and the newly formed C-terminal Lys-amine was used to react with ALO-OSu to form the designed Peptide-Linker a (C), following de-Fmoc (D), for antibody conjugation.

In the conjugation shown in FIG. 1B, amino-tetrazine handle or amino-azide handle (see the structures in Table 2) was conjugated to the Gln site on antibody via MTG based site-specific conjugation. The resulting antibody bearing tetrazine or azide (Ab-Handle) was clicked with the ALO linked Linker-Peptide (D) to form the Antibody-Peptide conjugate (ATL-a).

### Synthetic Examples of Linker-Peptide-A Using Method A

The synthesis of M6695 is illustrated as an example of Linker-Payload a. There are two approaches to make M6695 using 1) K(Dde) protection and 2) using K(Pydec) protection for peptide elongations.

### Approach 1: Synthesis of M6695 Using Fmoc Protection

### SPPS of M6695-Int1

The synthesis of peptide was performed with initial loading 1.5 mmol (0.3 mmol/g, 5 g). Fmoc-Lys(Boc) was coupled at C-terminus. Fmoc-Lys(Dde)-OH was coupled at Lys20 and Lys28. Once the peptide was fully elongated, the Dde group was removed by 5% NH₂NH₂ according to general procedure, which elaborated free amine groups on the positions of Lys20 and Lys28 on the resin-bound peptide. The peptide on resin with amine-groups were subsequently carried in Fmoc protection by treating the peptidyl resin with Fmoc-OSu (2 eq.), DIPEA (4 eq.) in DMF, repeating several runs (2-3 h per run) until all free amines were fully Fmoc-protected (monitored by ninhydrin rest). The peptide was then cleaved and purified with prep-HPLC to afford M6695-Int1 as white solid. ESI m/z: 1726.5 [M+3H]/3⁺, 1295.1 [M+4H]/4⁺, 1036.3 [M+5H]/5⁺, 863.8 [M+6H]/6⁺, 740.4 [M+7H]/7⁺.

| | Resin: Rink Amide MBHA (loading: 0.33 mmol/g) (5.0 g, 1.5 mmol). Temperature: 25° C. | | | |
|---|---|---|---|---|
| Step | | | | Reacting |
| # | Materials | Coupling reagents | solvent | time |
| 1 | Fmoc-Lys(Boc)-OH | HBTU (2.0 eq.), DIPEA (4.0 eq.) | DMF (15 mL) | 60 min |
| 2 | Fmoc-Ser(tBu)-OH | HBTU (2.0 eq.), DIPEA (4.0 eq.) | DMF (15 mL) | 60 min |
| 3 | Fmoc-Gly2-OH | HBTU (2.0 eq.), DIPEA (4.0 eq.) | DMF (15 mL) | 60 min |
| 4 | Fmoc-Gly2-OH | HBTU (2.0 eq.), DIPEA (4.0 eq.) | DMF (15 mL) | 60 min |
| 5 | Fmoc-Gly-Ser(ψMe,Me | HBTU (2.0 eq.), DIPEA (4.0 eq.) | DMF (15 mL) | 60 min |
| | pro)-OH | | | |
| 6 | Fmoc-Gly3-OH | HBTU (2.0 eq.), DIPEA (4.0 eq.) | DMF (15 mL) | 60 min |
| 7 | Fmoc-Gly-Ser(ψMe,Me | HBTU (2.0 eq.), DIPEA (4.0 eq.) | DMF (15 mL) | 60 min |
| | pro)-OH | | | |
| 8 | Fmoc-Gly3-OH | HBTU (2.0 eq.), DIPEA (4.0 eq.) | DMF (15 mL) | 60 min |
| 9 | Fmoc-Gly3-OH | HBTU (2.0 eq.), DIPEA (4.0 eq.) | DMF (15 mL) | 60 min |
| 10 | Fmoc-Lys(Dde)-OH | HBTU (2.0 eq.), DIPEA (4.0 eq.) | DMF (15 mL) | 60 min |
| 11 | Fmoc-Val-OH | HBTU (2.0 eq.), DIPEA (4.0 eq.) | DMF (15 mL) | 60 min |
| 12 | Fmoc-Leu-OH | HBTU (2.0 eq.), DIPEA (4.0 eq.) | DMF (15 mL) | 60 min |
| 13 | Fmoc-amY(OtBu)-OH | HBTU (2.0 eq.), DIPEA (4.0 eq.) | DMF (15 mL) | 60 min |
| 14 | Fmoc-Ala-OH | HATU (20. eq.), DIPEA (30 eq.) | DMF (15 mL) | 16 h |
| 15 | Fmoc-Ile-OH | HBTU (2.0 eq.), DIPEA (4.0 eq.) | DMF (15 mL) | 60 min |
| 16 | Fmoc-Phe-OH | HBTU (2.0 eq.), DIPEA (4.0 eq.) | DMF (15 mL) | 60 min |
| 17 | Fmoc-Glu(OtBu)-OH | HBTU (2.0 eq.), DIPEA (4.0 eq.) | DMF (15 mL) | 60 min |
| 18 | Fmoc-Lys(Dde)-OH | HBTU (2.0 eq.), DIPEA (4.0 eq.) | DMF (15 mL) | 60 min |
| 19 | Fmoc-Ala-OH | HBTU (2.0 eq.), DIPEA (4.0 eq.) | DMF (15 mL) | 60 min |
| 20 | Fmoc-Ala-OH | HBTU (2.0 eq.), DIPEA (4.0 eq.) | DMF (15 mL) | 60 min |
| 21 | Fmoc-Gln(Trt)-OH | HBTU (2.0 eq.), DIPEA (4.0 eq.) | DMF (15 mL) | 60 min |
| 22 | Fmoc-Glu(OtBu)-OH | HBTU (2.0 eq.), DIPEA (4.0 eq.) | DMF (15 mL) | 60 min |
| 23 | Fmoc-Glu(OtBu)-OH | HBTU (2.0 eq.), DIPEA (4.0 eq.) | DMF (15 mL) | 60 min |
| 24 | Fmoc-Leu-OH | HBTU (2.0 eq.), DIPEA (4.0 eq.) | DMF (15 mL) | 60 min |
| 25 | Fmoc-Tyr(tBu)-OH | HBTU (2.0 eq.), DIPEA (4.0 eq.) | DMF (15 mL) | 60 min |
| 26 | Fmoc-Ser(tBu)-OH | HBTU (2.0 eq.), DIPEA (4.0 eq.) | DMF (15 mL) | 60 min |
| 27 | Fmoc—Y—S—OH | HBTU (2.0 eq.), DIPEA (4.0 eq.) | DMF (15 mL) | 60 min |
| 28 | Fmoc-Asp(OtBu)-OH | HBTU (2.0 eq.), DIPEA (4.0 eq.) | DMF (15 mL) | 60 min |
| 29 | Fmoc-T-S—OH | HBTU (2.0 eq.), DIPEA (4.0 eq.) | DMF (15 mL) | 60 min |
| 30 | Fmoc-amF-OH | HBTU (2.0 eq.), DIPEA (4.0 eq.) | DMF (15 mL) | 60 min |
| 31 | Fmoc-Thr(tBu)-OH | HATU (20. eq.), DIPEA (30 eq.) | DMF (15 mL) | 16 h |
| 32 | Fmoc-Gly-OH | HBTU (2.0 eq.), DIPEA (4.0 eq.) | DMF (15 mL) | 60 min |
| 33 | Fmoc-Glu(OtBu)-OH | HBTU (2.0 eq.), DIPEA (4.0 eq.) | DMF (15 mL) | 60 min |
| 34 | Fmoc-Aib-OH | HBTU (2.0 eq.), DIPEA (4.0 eq.) | DMF (15 mL) | 60 min |
| 35 | Fmoc-Tyr(Dde)-OH | HBTU (2.0 eq.), DIPEA (4.0 eq.) | DMF (15 mL) | 90 min |
| 36 | Dde removal | HBTU (2.0 eq.), DIPEA (4.0 eq.) | DMF (15 mL) | 60 min |
| 37 | Fmoc—OSu (2 eq.) | DIPEA (4.0 eq.) | DMF (15 mL) | 60 min × n |
| | Cleavage | TFA (90%), TIS (5%), H2O (5%) | | 120 min |
| | Crude amount | 4000 mg | | |
| | Isolated amount | 265 mg | | |
| | Purity | 74.86% | | |
| | Yield | 3.4% | | |

### Synthesis of M6695

To a mixture of M6695-Int1 (0.20 g, 38 mol) in MeCN/H₂O (v/v=1, 10 mL) was added DIPEA (1 mL) in one portion. Once the reaction mixture was clear, ALO-OSu (63 mg, 0.23 mmol) was added to the mixture. The solution was then incubated at 25° C. for 30 minutes until the reaction was totally completed and M6695-Int2 (ESI m/z: 1781.1 [M+3H]/3⁺1331.8 [M+4H]/4+) was formed, which was monitored by LCMS. To the mixture was added piperidine (1 mL), and the reaction mixture was incubated at 25° C. for an hour until Fmoc group was totally removed, which was monitored by LCMS. The resulting mixture was acidified by addition of acetic acid (2.5 mL) and was then separated by prep-HPLC to give M6695 (22 mg, 11.6% yield) as a white solid. ESI m/z: 1559.0 [M+3H]/3⁺, 1169.4 [M+4H]/4⁺, 780.1 [M+6H]/6⁺.

### Example 4. Alternative Process for the Synthesis of Peptide M6695 Using Pydec Protection Step 1: Preparation of M6976

To a mixture of M6695-Int1 (50 mg, 10 mol) in MeCN/H₂O (v/v=1, 5 mL) was added DIPEA (0.5 mL) in one portion. Once the reaction mixture was clear, DBCO-OSu (32 mg, 80 umol) was added to the mixture. The solution was then incubated at 25° C. for 30 minutes until the reaction was totally completed and CP6501-1 (ESI m/z: 1366.7 [M+3H]/3⁺, 1331.8 [M+4H]/4⁺) was formed, which was monitored by LCMS. To the mixture was added piperidine (1 mL), and the reaction mixture was incubated at 25° C. for an hour until Fmoc group was totally removed, which was monitored by LCMS. The resulting mixture was separated by prep-HPLC with 0.1 M NH₄HCO₃ buffer to give M6976 (8.6 mg, 17.2% yield) as a white solid. ESI m/z: 1599.9 [M+3H]/3⁺, 1200.2 [M+4H]/4⁺.

### Step 2: SPPS of M6809

The synthesis of peptide was performed with initial loading 0.15 mmol (0.3 mmol/g, 0.5 g). Fmoc-Lys(Boc) was coupled at C-terminus. Fmoc-Lys(Dde)-OH was coupled at Lys20 and Lys28. Once the peptide was fully elongated, the Dde group was removed by 5% NH₂NH₂ according to general procedure, which elaborated free amine groups on the positions of Lys20 and Lys28 on the resin-bound peptide. The peptidyl resin was subsequently carried in Pydec protection by treating the peptidyl resin with Pydec-PNP (6 eq.), DIPEA (12 eq.) in DMF, repeating several runs (60 min per run, 2 runs). All free amines were fully protected (monitored by ninhydrin rest). The peptide was then cleaved and purified with RP-HPLC to afford M6809 as white solid. ESI m/z: 1288.4 [M+4H]/4⁺, 1030.7 [M+5H]/5⁺, 859.2 [M+6H]/6⁺, 736.6 [M+7H]/7⁺, 644.6 [M+8H]/8⁺.

| | Resin: Rink Amide MBHA (loading: 0.33 mmol/g) (0.5 g, 0.17 mmol). Temperature: 25° C. | | | |
|---|---|---|---|---|
| | | | | Reacting |
| Step | Materials | Coupling reagents | solvent | time |
| 1 | Fmoc-Lys(Boc)-OH | HBTU (2.0 eq.), DIPEA (4.0 eq.) | DMF (15 mL) | 60 min |
| 2 | Fmoc-Ser(tBu)-OH | HBTU (2.0 eq.), DIPEA (4.0 eq.) | DMF (15 mL) | 60 min |
| 3 | Fmoc-Gly2-OH | HBTU (2.0 eq.), DIPEA (4.0 eq.) | DMF (15 mL) | 60 min |
| 4 | Fmoc-Gly2-OH | HBTU (2.0 eq.), DIPEA (4.0 eq.) | DMF (15 mL) | 60 min |
| 5 | Fmoc-Gly-Ser(ψMe,Me pro)- | HBTU (2.0 eq.), DIPEA (4.0 eq.) | DMF (15 mL) | 60 min |
| | OH | | | |
| 6 | Fmoc-Gly3-OH | HBTU (2.0 eq.), DIPEA (4.0 eq.) | DMF (15 mL) | 60 min |
| 7 | Fmoc-Gly-Ser(ψMe,Me pro)- | HBTU (2.0 eq.), DIPEA (4.0 eq.) | DMF (15 mL) | 60 min |
| | OH | | | |
| 8 | Fmoc-Gly3-OH | HBTU (2.0 eq.), DIPEA (4.0 eq.) | DMF (15 mL) | 60 min |
| 9 | Fmoc-Gly3-OH | HBTU (2.0 eq.), DIPEA (4.0 eq.) | DMF (15 mL) | 60 min |
| 10 | Fmoc-Lys(Dde)-OH | HBTU (2.0 eq.), DIPEA (4.0 eq.) | DMF (15 mL) | 60 min |
| 11 | Fmoc-Val-OH | HBTU (2.0 eq.), DIPEA (4.0 eq.) | DMF (15 mL) | 60 min |
| 12 | Fmoc-Leu-OH | HBTU (2.0 eq.), DIPEA (4.0 eq.) | DMF (15 mL) | 60 min |
| 13 | Fmoc-amY(OtBu)-OH | HBTU (2.0 eq.), DIPEA (4.0 eq.) | DMF (15 mL) | 60 min |
| 14 | Fmoc-Ala-OH | HATU (20. eq.), DIPEA (30 eq.) | DMF (15 mL) | 16 h |
| 15 | Fmoc-Ile-OH | HBTU (2.0 eq.), DIPEA (4.0 eq.) | DMF (15 mL) | 60 min |
| 16 | Fmoc-Phe-OH | HBTU (2.0 eq.), DIPEA (4.0 eq.) | DMF (15 mL) | 60 min |
| 17 | Fmoc-Glu(OtBu)-OH | HBTU (2.0 eq.), DIPEA (4.0 eq.) | DMF (15 mL) | 60 min |
| 18 | Fmoc-Lys(Dde)-OH | HBTU (2.0 eq.), DIPEA (4.0 eq.) | DMF (15 mL) | 60 min |
| 19 | Fmoc-Ala-OH | HBTU (2.0 eq.), DIPEA (4.0 eq.) | DMF (15 mL) | 60 min |
| 20 | Fmoc-Ala-OH | HBTU (2.0 eq.), DIPEA (4.0 eq.) | DMF (15 mL) | 60 min |
| 21 | Fmoc-Gln(Trt)-OH | HBTU (2.0 eq.), DIPEA (4.0 eq.) | DMF (15 mL) | 60 min |
| 22 | Fmoc-Glu(OtBu)-OH | HBTU (2.0 eq.), DIPEA (4.0 eq.) | DMF (15 mL) | 60 min |
| 23 | Fmoc-Glu(OtBu)-OH | HBTU (2.0 eq.), DIPEA (4.0 eq.) | DMF (15 mL) | 60 min |
| 24 | Fmoc-Leu-OH | HBTU (2.0 eq.), DIPEA (4.0 eq.) | DMF (15 mL) | 60 min |
| 25 | Fmoc-Tyr(tBu)-OH | HBTU (2.0 eq.), DIPEA (4.0 eq.) | DMF (15 mL) | 60 min |
| 26 | Fmoc-Ser(tBu)-OH | HBTU (2.0 eq.), DIPEA (4.0 eq.) | DMF (15 mL) | 60 min |
| 27 | Fmoc-Tyr(tBu)-Ser(ψMe,Me | HBTU (2.0 eq.), DIPEA (4.0 eq.) | DMF (15 mL) | 60 min |
| | pro)-OH | | | |
| 28 | Fmoc-Asp(OtBu)-OH | HBTU (2.0 eq.), DIPEA (4.0 eq.) | DMF (15 mL) | 60 min |
| 29 | Fmoc-Thr(tBu)-Ser(ψMe,Me | HBTU (2.0 eq.), DIPEA (4.0 eq.) | DMF (15 mL) | 60 min |
| | pro)-OH | | | |
| 30 | Fmoc-amF-OH | HBTU (2.0 eq.), DIPEA (4.0 eq.) | DMF (15 mL) | 60 min |
| 31 | Fmoc-Thr(tBu)-OH | HATU (20. eq.), DIPEA (30 eq.) | DMF (15 mL) | 16 h |
| 32 | Fmoc-Gly-OH | HBTU (2.0 eq.), DIPEA (4.0 eq.) | DMF (15 mL) | 60 min |
| 33 | Fmoc-Glu(OtBu)-OH | HBTU (2.0 eq.), DIPEA (4.0 eq.) | DMF (15 mL) | 60 min |
| 34 | Fmoc-Aib-OH | HBTU (2.0 eq.), DIPEA (4.0 eq.) | DMF (15 mL) | 60 min |
| 35 | Fmoc-Tyr(Dde)-OH | HBTU (2.0 eq.), DIPEA (4.0 eq.) | DMF (15 mL) | 90 min |
| 36 | Dde removal | HBTU (2.0 eq.), DIPEA (4.0 eq.) | DMF (15 mL) | 60 min |
| 37 | PyDec-PNP (6.0 eq.) | DIPEA (12.0 eq.) | DMF (15 mL) | 60 min × 2 |
| | Cleavage | 90.0% TFA/5% H2O/5% TIS, | | 90 min × 3 |
| | Crude amount | 800 mg | | |
| | Isolated amount | 180 mg | | |
| | Purity | 95% | | |
| | Yield | 21% | | |

### Step 3: Solution Phase Synthesis of M6695

To a solution of M6809 (0.50 g 94 mol) in MeCN/H₂O (1:1, v/v, 20 mL) were added DIPEA (0.4 mL) and ALO-OSu (55 mg, 0.12 mmol, 2.0 eq) successively, and the reaction mixture turned clear. The solution was incubated at 25° C. for 30 min LCMS monitoring indicated that the reaction was completed. To the solution was added piperidine (4 mL), and the mixture was incubated at 25° C. for an hour. TCEP (0.56 g) was then added to the reaction mixture and the solution was incubated for another an hour. LCMS indicated the reaction was completed. The resulting mixture was acidified by addition of 1 mL of acetic acid at 0° C., and the mixture was directly separated by prep-HPLC (Column: Gemini C18 21.2×250 mm, 10 μm, 110 Å; Mobile Phase: A: water (containing 0.050% TFA), B: ACN; Gradient: 29-39% B in A within 30 min) to provide M6695 (0.11 g, 240% yield) as a white solid after lyophilization. ESI m/z: 1559.0 [M+3H]/3⁺, 1169.4 [M+4H]/4⁺, 780.1 [M+6H]/6⁺.

### Example 5. Process for the Synthesis of Linker-Payload Peptide M6687

Overall Procedure for Peptides with a Linker C (Method C)

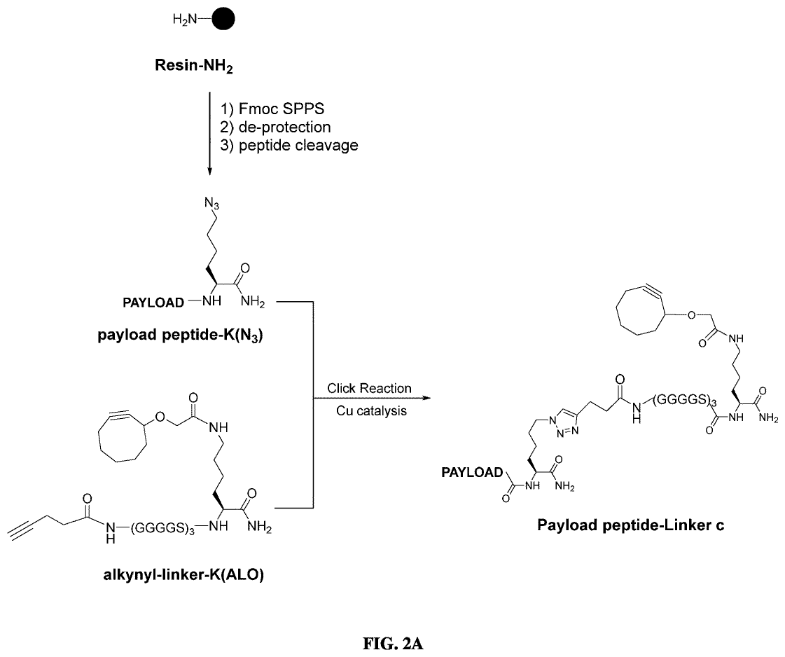

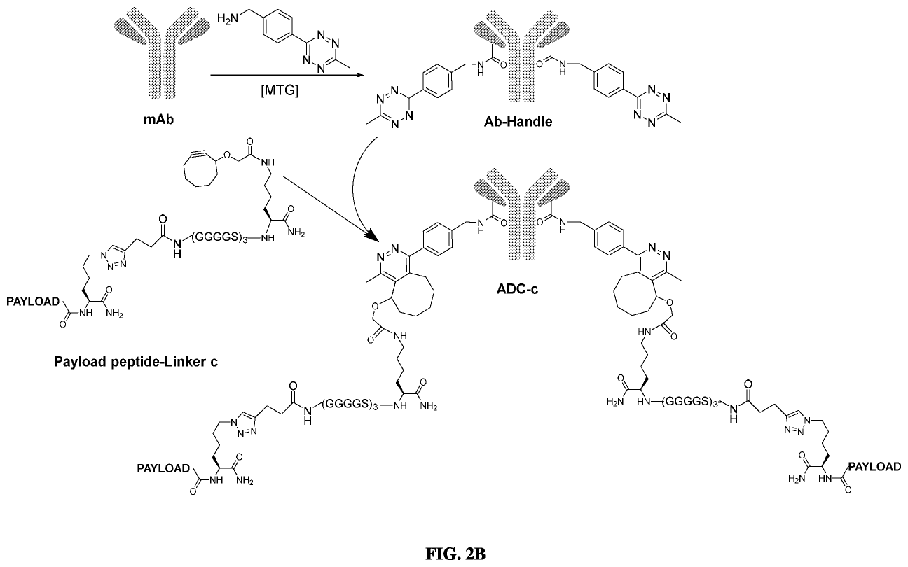

In method C shown in FIG. 2A, the Payload peptide bearing C-terminal Lys(N₃) was synthesized directly on SPPS. The alkyne-Linker-ALO was synthesized using hybrid SPPS/LPPS. The linker includes proteogenic peptide (GGGGS)n, where n=1, 2, 3, or 4 (SEQ ID NO: 110); polysarcosine linker: (Sar)n, where n=1 to 20; PEG linker: NH2-(PEG)n-COOH, which includes aeea, aeep, aeeea, aeeep and other PEG linker with 1-20 PEG repeats. The payload peptide and linker peptide were coupled catalyzed click reaction to afford the designed Peptide-Linker c. Here, the peptide-K(N3) reacted exclusively with the single alkyne instead of the cyclooctyne.

In the conjugation shown in FIG. 2B, amino-tetrazine handle or amino-azide handle (see the structures in Table 2) was conjugated to the Gln site on antibody via MTG based site-specific conjugation. The resulting antibody bearing tetrazine or azide (Ab-handle) is clicked with the cyclooctyne in Peptide-Linker c via IEDDA reaction to generate the conjugate ATL-c. Synthetic examples of Linker-Peptide-C (aka method C)

The synthesis of M6687 is illustrated as an example of Linker-Payload C (Linker C).

M6687 was synthesized from a Cu(I)-catalyzed azide-alkyne 1,3-dipolar cycloaddition (CuAAC) between M6682 (peptide azide) and M6694 (alkyne linker) in solution phase reaction. Both M6682 and M6694 were obtained from SPPS and LPPS, separately.

The bifunctional linker M6694 was synthesized by amide coupling from M6694-1 and activated ester COT-OSu (M6694-2, CAS: 1425803-45-7) in LPPS. The peptide M6694-1 was synthesized by standard SPPL (solid phase peptide synthesis) strategy, including resin preparation and loading; amide coupling and de-Fmoc reaction; cleavage and purification.

There are two approaches of SPPS for M6694-1 starting from Rink Amide AM Resin and MBHA Resin. M6694-1 obtained using AM resin doesn't need additional purification by reversed-phase column chromatography, while using MBHA resin will introduce an impurity of peptide+163 (from MBHA) and crude product M6694-1 needs additional purification by reversed-phase column chromatography.

### SPPS of M6694-1

### Synthesis of M6694-1 Using Rink Amide AM Resin

| | Resin: Rink Amide AM (loading, 0.6-0.8 mmol/g, 4.3 g, 3 mmol), 25° C. | | | |
|---|---|---|---|---|
| Step | Materials (2.0 eq. ea) | Coupling reagents | solvent | time |
| 1 | Fmoc-Lys(Boc)-OH | DIC (2.0 eq.), Oxyma (3.0 eq.) | DMF (100 mL) | 90 min. |
| 2 | Fmoc-Gly- | DIC (2.0 eq.), Oxyma (3.0 eq.) | DMF (100 mL) | 90 min. |
| | Ser(Psi(Me,Me)pro)-OH | | | |
| 3 | Fmoc-Gly-Gly-Gly-OH | DIC (2.0 eq.), Oxyma (3.0 eq.) | DMF (100 mL) | 90 min. |
| 4 | Fmoc-Gly- | DIC (2.0 eq.), Oxyma (3.0 eq.) | DMF (100 mL) | 90 min. |
| | Ser(Psi(Me,Me)pro)-OH | | | |
| 5 | Fmoc-Gly-Gly-Gly-OH | DIC (2.0 eq.), Oxyma (3.0 eq.) | DMF (100 mL) | 90 min. |
| 6 | Fmoc-Gly- | DIC (2.0 eq.), Oxyma (3.0 eq.) | DMF (100 mL) | 90 min. |
| | Ser(Psi(Me,Me)pro)-OH | | | |
| 7 | Fmoc-Gly-Gly-Gly-OH (2.0 | DIC (2.0 eq.), Oxyma (3.0 eq.) | DMF (100 mL) | 90 min. |
| | eq.) | | | |
| 8 | PYA-OH | DIC (2.0 eq.), Oxyma (3.0 eq.) | DMF (100 mL) | 90 min. |
| | Cleavage | 95.0% TFA/2.5% H2O/ | | 90 min. |
| | | 2.5% TIS | | |

| | |
|---|---|
| Crude amount | 2.7 g |
| Isolated amount | 2.7 g (without purification) |
| Yield | 67% |

### Resin Preparation

After swelling the Rink Amide AM resin (4.3 g, 3.0 mmol, 1.0 eq., Sub 0.6-0.8 mmol/g) with DMF (60 mL) under nitrogen agitating at 25° C. for 0.5 hours, 2000 piperidine in DMF solution (60 mL) was added into the resin. The mixture was agitated at 25° C. for 15 min. The procedure was repeated twice. After the reaction was completed, the resin was washed with DMF (60 mL×3).

### Amino-Acid Loading to Resin

A solution of Fmoc-Lys(Boc)-OH (2.8 g, 6.0 mmol, 2.0 eq.), DIC (1 mL, 6.0 mmol, 2.0 eq.) and Oxyma (1.3 g, 9.0 mmol, 3.0 eq.) in DMF (100 mL) was added to the resin prepared above, and the mixture was agitated with N₂ at 25° C. for 90 min. The resin was washed with DMF (60 mL×6).

### Amide Coupling Reaction

A solution of N-Fmoc-amino acid (2.0 eq.), DIC (2.0 eq.) and Oxyma (3.0 eq.) in DMF (100 mL) was added to the resin. The reaction mixture was agitated with N₂ for 90 minutes at room temperature. After the reaction completed, the resin was washed with DMF (20 mL/g of Resin×5).

### Deprotections

Fmoc deprotection (after each coupling reaction of native Fmoc-amino acid): 20% piperidine in DMF (60 mL) was added and agitated the resin with N₂ at 25° C. for 20 min. The procedure was repeated 2 times, and then the resin was washed with DMF (100 mL×6) and filtered to get the resin-peptide with amine at N-terminal, which was used directly for next coupling.

### Cleavage

The resin bound peptide was cleaved using TFA/H₂O/TIS (v/v/v=95.0:2.5:2.5, 300 mL) for 90 min. The mixture was filtered, and the solution was collected. The residual resin was washed with TFA (20 mL×2). The combined solutions were poured into MTBE (pre-cooled to −10° C., 1700 mL) and shaken to precipitate the crude peptide M6694-1. The suspension was centrifuged, and the supernatant was decanted. The residual solid was further washed with MTBE (1000 mL×2) and centrifuged. After decanting the supernatant, the residual solid was then dried under reduced pressure overnight to give crude peptide M6694-1 (2.7 g, 2.3 mmol, 70% yield, TFA Salt) as a white solid, which was used for the next step without further purification. ESI m/z: 1171.3 [M+H]⁺, 586.3 [M+2H]/2⁺.

### LPPS Synthesis of M6694 (a Linker)

To a stirred mixture of peptide M6694-1 (1.0 g, 0.85 mmol, 1.0 eq.) in a mixed solvent of acetonitrile and water (v/v=1:1, 120 mL) were added DIPEA (0.3 mL, 1.71 mmol, 2.0 eq.) and ALO-OSu (286 mg, 1.03 mmol, 1.2 eq.) subsequently at room temperature, and the reaction mixture was stirred at room temperature for 16 hours, which was monitored by LCMS until the materials were consumed. A white viscous solid was then precipitated from the solution. The resulting mixture was centrifuged, and the supernatant was decanted. To the residue was added MTBE (150 mL), and the mixture was stirred at room temperature for 10 minutes. The resulting suspension was centrifuged, and the supernatant was decanted. The residual solid was slurried in water (150 mL) and stirred for 10 minutes, filtered and lyophilized to provide M6694 (600 mg, 0.45 mmol, 5800 yield) as a white solid. ESI m/z: 1335.4 [M+H]⁺, 668.4 [M+2H]/2⁺.

### Solubility of M6694

| | | | | aq. guanidine |
|---|---|---|---|---|
| Solvent | H₂O | DMF | Methanol | hydrochloride* |
| Solubility (mg/mL) | 0.02 | 0.2 | 0.02 | 40 |

*Preparation of aq. guanidine hydrochloride solution of M6694: 6 g of guanidine hydrochloride was dissolved in 10 g of water. To the solution (10 μL) was added M6694 (0.4 mg) until the solid precipitated.

In order to avoid analytical errors due to poor solubility which was introduced by peptide-hydrogen-bonding, aq. guanidine hydrochloride was added to totally dissolve the sample of M6694 before the LCMS test.

### SPPS Synthesis of M6682

The peptide M6682 was synthesized by standard solid phase peptide synthesis strategy, including resin preparation and loading; amide coupling and de-Fmoc reaction; cleavage and purification.

| | Sieber amide Resin (loading: 0.30 mmol/g) (13 g, 7.2 mmol). Temperature: 25° C. | | | |
|---|---|---|---|---|
| Step | Materials | Coupling reagents | solvent | time |
| 1 | Fmoc—K(N3)—OH (2.0 eq.) | DIC (2.0 eq.), Oxyma (2.0 eq.) | DMF (150 mL) | 100 min |
| 2 | Fmoc-Gly3-OH (2.0 eq.) | DIC (2.0 eq.), Oxyma (2.0 eq.) | DMF (150 mL) | 100 min |
| 3 | Fmoc-Lys(Boc)-OH (2.0 eq.) | DIC (2.0 eq.), Oxyma (2.0 eq.) | DMF (150 mL) | 100 min |
| 4 | Fmoc-Val-OH (2.0 eq.) | DIC (2.0 eq.), Oxyma (2.0 eq.) | DMF (150 mL) | 100 min |
| 5 | Fmoc-Leu-OH (2.0 eq.) | DIC (2.0 eq.), Oxyma (2.0 eq.) | DMF (150 mL) | 100 min |
| 6 | Fmoc-amY(tBu)-OH (2.0 eq.) | DIC (2.0 eq.), Oxyma (2.0 eq.) | DMF (150 mL) | 100 min |
| 7 | Fmoc-Ala-OH (3.0 eq.) | DIC (3.0 eq.), Oxyma (6.0 eq.) | DMF (150 mL) | 16 h |
| 8 | Fmoc-Ile-OH (2.0 eq.) | DIC (2.0 eq.), Oxyma (2.0 eq.) | DMF (150 mL) | 100 min |
| 9 | Fmoc-Phe-OH (2.0 eq.) | DIC (2.0 eq.), Oxyma (2.0 eq.) | DMF (150 mL) | 100 min |
| 10 | Fmoc-Glu(OtBu)-OH (2.0 eq.) | DIC (2.0 eq.), Oxyma (2.0 eq.) | DMF (150 mL) | 100 min |
| 11 | Fmoc-Lys(Boc)-OH (2.0 eq.) | DIC (2.0 eq.), Oxyma (2.0 eq.) | DMF (150 mL) | 100 min |
| 12 | Fmoc-Ala-OH (2.0 eq.) | DIC (2.0 eq.), Oxyma (2.0 eq.) | DMF (150 mL) | 100 min |
| 13 | Fmoc-Ala-OH (2.0 eq.) | DIC (2.0 eq.), Oxyma (2.0 eq.) | DMF (150 mL) | 100 min |
| 14 | Fmoc-Gln(Trt)-OH (2.0 eq.) | DIC (2.0 eq.), Oxyma (2.0 eq.) | DMF (150 mL) | 100 min |
| 15 | Fmoc-Glu(OtBu)-OH (2.0 eq.) | DIC (2.0 eq.), Oxyma (2.0 eq.) | DMF (150 mL) | 100 min |
| 16 | Fmoc-Glu(OtBu)-OH (2.0 eq.) | DIC (2.0 eq.), Oxyma (2.0 eq.) | DMF (150 mL) | 100 min |
| 17 | Fmoc-Leu-OH (2.0 eq.) | DIC (2.0 eq.), Oxyma (2.0 eq.) | DMF (150 mL) | 100 min |
| 18 | Fmoc-Tyr(OtBu)-OH (2.0 eq.) | DIC (2.0 eq.), Oxyma (2.0 eq.) | DMF (150 mL) | 100 min |
| 19 | Fmoc-Ser(tBu)-OH (2.0 eq.) | DIC (2.0 eq.), Oxyma (2.0 eq.) | DMF (150 mL) | 100 min |
| 20 | Fmoc-Ser(tBu)-OH (2.0 eq.) | DIC (2.0 eq.), Oxyma (2.0 eq.) | DMF (150 mL) | 100 min |
| 21 | Fmoc-Tyr(OtBu)-OH (2.0 eq.) | DIC (2.0 eq.), Oxyma (2.0 eq.) | DMF (150 mL) | 100 min |
| 22 | Fmoc-Asp(OtBu)-OH (2.0 eq.) | DIC (2.0 eq.), Oxyma (2.0 eq.) | DMF (150 mL) | 100 min |
| 23 | Fmoc-Ser(tBu)-OH (2.0 eq.) | DIC (2.0 eq.), Oxyma (2.0 eq.) | DMF (150 mL) | 100 min |
| 24 | Fmoc-Thr(tBu)-OH (2.0 eq.) | DIC (2.0 eq.), Oxyma (2.0 eq.) | DMF (150 mL) | 100 min |
| 25 | Fmoc-amF-OH (2.0 eq.) | DIC (2.0 eq.), Oxyma (2.0 eq.) | DMF (150 mL) | 100 min |
| 26 | Fmoc-Thr(tBu)-OH (3.0 eq.) | DIC (3.0 eq.), Oxyma (6.0 eq.) | DMF (150 mL) | 16 h |
| 27 | Fmoc-Gly-OH (2.0 eq.) | DIC (2.0 eq.), Oxyma (2.0 eq.) | DMF (150 mL) | 100 min |
| 28 | Fmoc-Glu(OtBu)-OH (2.0 eq.) | DIC (2.0 eq.), Oxyma (2.0 eq.) | DMF (150 mL) | 100 min |
| 29 | Fmoc-Aib-OH (2.0 eq.) | DIC (2.0 eq.), Oxyma (2.0 eq.) | DMF (150 mL) | 100 min |
| 30 | Fmoc-Tyr(OtBu)-OH (3.0 eq.) | DIC (3.0 eq.), Oxyma (6.0 eq.) | DMF (150 mL) | 16 h |
| | Cleavage | TFA (90%), TIS (5%), H₂O (5%) | | 90 min |
| | Crude amount | 15 g | | |
| | Isolated amount | 7 g | | |
| | Purity | 97.7% | | |
| | Yield | 46% | | |

### Resin Preparation

To a solution of Sieber amide Resin (loading: 0.30 mmol/g) (13 g, 4.0 mmol) in DMF (150 mL) was agitated with N₂ at 25° C. for 2-5 hours. Then 20% piperidine in DMF (150 mL) was added and agitated with N₂ at 25° C. for 15-30 min. This procedure was repeated twice. The resin was washed with DMF (150 mL) for 3 times.

### Amino Acid Loading to Resin

A solution of DIC (2.0 eq.), DIPEA (4.0 eq.), and Fmoc-K(N₃)—OH (2.0 eq.) in DMF (150 mL) was added to the resin obtained above DMF (150 mL), and the mixture was agitated with N₂ at 25° C. for 60 min. The resin was washed with DMF (150 mL) for 3 times.

### Amide Coupling Reaction

General coupling condition A: Coupling of native amino acid: Fmoc-AA-OH (2.0 eq.)/DIC (2.0 eq.)/Oxyma (2.0 eq.) was dissolved in DMF (150 mL). The mixture was agitated for 60-90 min at room temperature. After the reaction was completed, the resin was washed with DMF (10-20 mL/g of Resin) for 3 times.

Coupling of the next AA after amAA: AA (3.0 eq.)/DIC (3.0 eq.)/Oxyma (6.0 eq.) was dissolved DMF (150 mL). The mixture was agitated for 16 hours at room temperature. After the reaction was completed, the resin was washed with DMF (150 mL) for 3 times.

### Fmoc Deprotection

Fmoc deprotection (after each coupling reaction of native Fmoc-amino acid): 20% piperidine in DMF (150 mL) was added and agitated the resin with N₂ at 25° C. for 15-30 min, this procedure was repeated twice. The residue was washed with DMF (150 mL) for 3 times and filtered to get the resin-peptide with amine at N-terminus, which was used directly for next coupling.

### Cleavage Conditions

The resin bound peptide was cleaved using the cleavage cocktail (TFA (90%), TIS (5%), H₂O (5%)) for 90 min at 25° C. The mixture was filtered, and the solution was collected. The residual resin was washed with TFA (70 mL) twice. The combined solutions were poured into MTBE (pre-cooled to −10° C., 1.5 L) and shaken to precipitate the crude peptide. The suspension was centrifuged, and the supernatant was decanted. The residual solid was further washed with MTBE (1.0 L×2) and centrifuged. After decanting the supernatant, the residual solid was then dried under reduced pressure overnight to give crude peptide as an off-white solid.

Once the peptide was fully elongated, the peptide was then cleaved and purified with RP-HPLC to afford M6682 as white solid. ESI m/z: 1795.8 [M+2H]/2⁺, 1197.8 [M+3H]/3⁺, 898.5 [M+4H]/4⁺.

M6682, with a functional reactive group of azide, could be used as a linker-payload directly for conjugation to an antibody.

### Synthesis of M6687 (Linker-Payload)

To a solution of the linker M6694 (0.11 g, 82 μmol) in tert-butanol (6 mL) were added aq. ascorbic acid (1.7 mL, 100 mg/mL) and aq. copper sulfate (1.5 mL, 100 mg/mL). The mixture was added into a solution of the peptide M6682 (0.20 g, 51 μmol) in a mixed solvent tert-butanol and water (v/v=1:1, 20 mL). The resulting mixture was incubated at room temperature for 150 minutes, which was monitored by LCMS until the peptide M6682 was totally consumed. aq. disodium edetate dihydrate (5.4 mL, 112 mg/mL) was added to the mixture, and then a mixed solvent acetonitrile and water (v/v=1:1, 20 mL) was added, the resulting mixture was centrifuged, the superannuate was directly purified by prep-HPLC (Column: XBridge OBD C18 19 mm×250 mm, 10 μm, 130 Å; Mobile Phase: A: water (containing 0.05% TFA), B: ACN; Gradient: 20-80% B in A within 30 min) to provide M6687 (0.20 g, 60% yield) as a white solid after lyophilization. ESI m/z: 1642.7 [M+3H]/3⁺, 1232.3 [M+4H]/⁴⁺.

Detailed characterization of those peptide products prepared are shown in the Table 3.

Additional synthesis examples can be found in the patent applications U.S. Application Ser. No. 63/744,076, filed Jan. 10, 2025, and U.S. Application Ser. No. 63/770,235, filed on Mar. 11, 2025, both of which are incorporated herein by reference in their entirety.

### Example 6. Antibody-Ligand/Linker-Payload Conjugations

TABLE 6
List of Antibody-Ligand Conjugates

| | | Ab | | Conj. | | Linker- | ADC | Purity |
|---|---|---|---|---|---|---|---|---|
| | ADC name | Target | Ab # | site | Handle | payload | DAR | (%) |
| 1 | REGN1033-M6092- | Anti- | REGN1033 | LC | M6092 | M6687 | 1.96 | 95.6 |
| | M6687 | GDF8 | | Q55 | | | | |
| 2 | REGN1033-M6092- | Anti- | REGN1033 | LC | M6092 | M6695 | 1.95 | 96.9 |
| | M6695 | GDF8 | | Q55 | | | | |

### Generation of Antibody-Conjugate (AC) Via Site-Specific Conjugation on Light Chain Q-Site of an Anti-GDF8 Antibody (FIG. 3)

Anti-GDF8 human IgG4 antibody REGN1033 containing a Gln-site on light chain (Q55) was mixed with 30-50 molar equivalents of the conjugation handle (M6092). The resulting solution was mixed with microbial transglutaminase (mTG; 0.5-1 units per mg of antibody, wild-type mTG from Zedira) resulting in a final concentration of the antibody at 10-20 mg/mL. The reaction mixture was incubated at 25-37° C. for 0.5-2 hours with gentle shaking. The reaction was monitored by ESI-MS and upon completion excess linker-payload, protein aggregates and MTG were removed by Protein A affinity chromatography (ProA) (FIG. 3).

The purified conjugation intermediate (REGN1033-M6092) was mixed with 5-10 molar equivalent of linker payloads. The reaction mixture was incubated at 25-37° C. for 2-48 hours with gentle shaking to generate antibody conjugate REGN1033-M6092-M6695, also refers as REGN1033-M6695 antibody conjugate for simplicity. The reaction was monitored by ESI-MS and upon completion excess linker-payload and protein aggregates were removed by Protein A affinity chromatography (ProA), buffer exchanged into the formulation buffer and sterile filtered. The protein concentration was measured by a UV-vis spectrophotometer. Overall protein recovery was 70-90% and the monomer of the antibody conjugate purity (>90%) was determined by analytical SEC. ADCs were further characterized by liquid chromatography electrospray ionization mass spectrometry (LC-ESI-MS) to calculate the drug-to-antibody ratio (DAR>1.8).

### Preparation of REGN1033-M6092-M6687: a Conjugation Method for Generating GLP1R/GIPR Agonist Peptidomimetic Anti-GDF8 Conjugate Via Site-Specific Conjugation at the Q-Site on the Light Chain Using M6092 and M6687

A specific example for the site-specific conjugation: A 10 g anti-GDF8 antibody was mixed with 30 molar equivalents of conjugation handle (M6092), followed by the addition of wild-type microbial transglutaminase (Zedira, 0.5 unit mTG per mg of antibody), resulting in a final antibody concentration of 19 mg/mL. The reaction mixture was incubated at 25° C. for an hour with gentle shaking. The reaction was monitored by ESI-MS and upon completion excess handle and MTG were removed by proA column (AKTA avant, MabSelect SurRe, 429 mL CV, flowrate 17-25 mL/min). The eluted sample was immediately neutralized to pH 7.0, sterile filtered, and stored at 4 C or −20 C. Then, 1.2 g of the purified conjugation intermediate was mixed with 5 molar equivalents of linker payload (M6687). The reaction mixture was incubated at 37° C. for 28 h with gentle shaking. The reaction was monitored by ESI-MS and upon completion aggregate and excess amount of linker payload were removed by proA column (AKTA avant, MabSelect Sure, 51.5 mL CV, flowrate 4-6 mL/min). The eluted sample was immediately neutralized to pH 7.0, formulated into PBS buffer with 5% glycerol and sterile filtered. The protein concentration was measured by a UV-vis spectrophotometer. Overall protein recovery was >76% and antibody conjugate monomer purity was determined by analytical SEC (FIG. 3). Antibody conjugates were further characterized by liquid chromatography electrospray ionization mass spectrometry (LC-ESI-MS) to calculate the drug-to-antibody ratio (FIG. 4).

Liquid chromatography electrospray ionization mass spectrometry (LC-ESI-MS) analysis was performed to determine the drug distribution profile and to calculate the average drug-to-antibody ratio (DAR). Each sample (20 μL at 1 mg/mL) was digested by IdeS (30 minutes at 37° C.) then loaded onto an ACQUITY UPLC Protein BEH C4 column (10K psi, 300 A, 1.7 um, 75 um×100 mm). Mass spectrum was acquired on a Waters Synapt G2-Si mass spectrometer. As shown in FIG. 4, the major peak is DAR 2 species.

### REGN1033-M6092-M6695: a Conjugation Method for Generating GLP1R/GIPR Agonist Peptidomimetic Anti-GDF8 Conjugate Via Site-Specific Conjugation at the Q-Site on the Light Chain Using M6092 and M6695

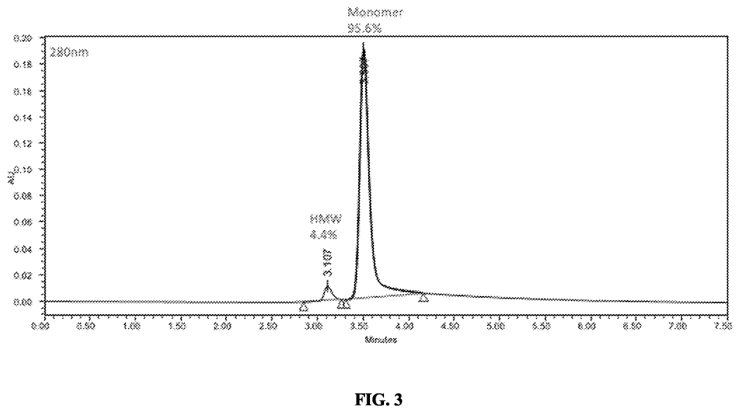

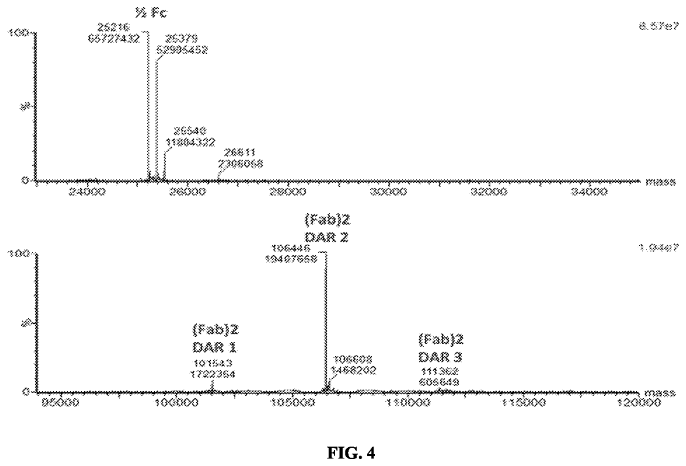

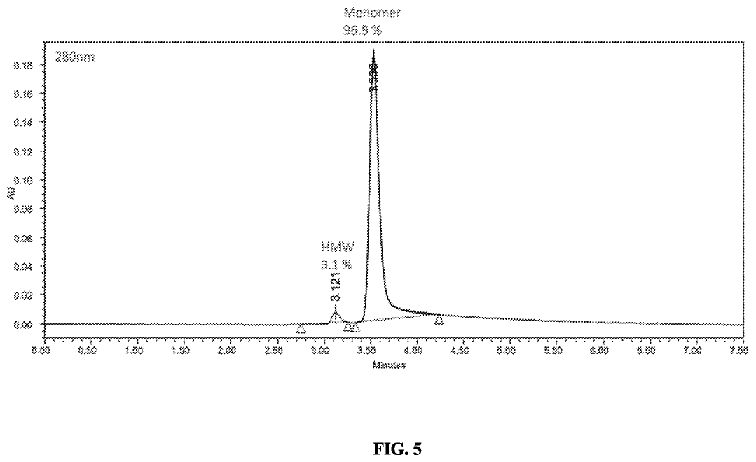

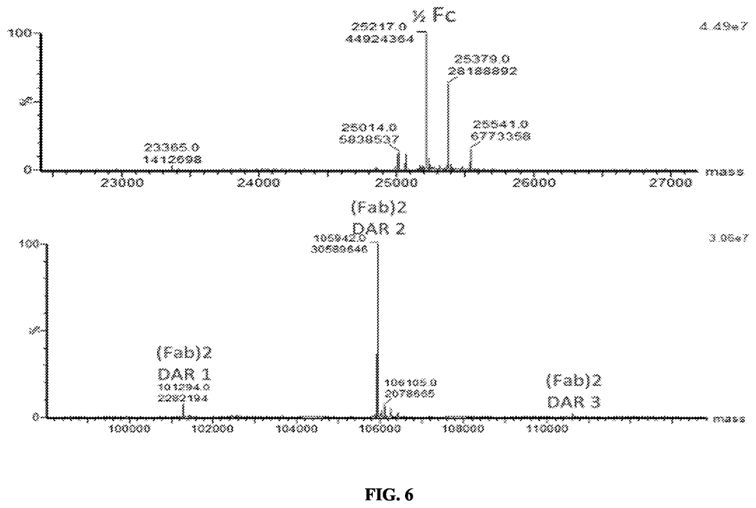

A specific example for the site-specific conjugation: A 10 g anti-GDF8 antibody was mixed with 30 molar equivalents of conjugation handle (M6092), followed by the addition of wild-type microbial transglutaminase (Zedira, 0.5 unit mTG per mg of antibody), resulting in a final antibody concentration of 19 mg/mL. The reaction mixture was incubated at 25° C. for an hour with gentle shaking. The reaction was monitored by ESI-MS and upon completion excess handle and MTG were removed by proA column (AKTA avant, MabSelect SurRe, 429 mL CV, flowrate 17-25 mL/min). The eluted sample was immediately neutralized to pH 7.0, sterile filtered, and stored at 4 C or −20 C. The amount of 250 mg of the purified conjugation intermediate was thawed at ambient temperature and mixed with 5 molar equivalents of linker payload (M6695). The reaction mixture was incubated at 37° C. for 25 hrs with gentle shaking. The reaction was monitored by ESI-MS and upon completion aggregate and excess amount of linker payload were removed by proA column (AKTA avant, HiTrap MabSelectSure 5 ml+5 ml in tandem, 10 mL CV, flowrate 3-5 mL/min). The eluted sample was immediately neutralized to pH 7.0, formulated into PBS buffer with 5% glycerol and sterile filtered. The protein concentration was measured by a UV-vis spectrophotometer. Overall protein recovery was >84% and antibody conjugate monomer purity was determined by analytical SEC (FIG. 5). Antibody conjugates were further characterized by liquid chromatography electrospray ionization mass spectrometry (LC-ESI-MS) to calculate the drug-to-antibody ratio (FIG. 6).

Liquid chromatography electrospray ionization mass spectrometry (LC-ESI-MS) analysis was performed to determine the drug distribution profile and to calculate the average drug-to-antibody ratio (DAR). Each sample (20 μL at 1 mg/mL) was digested by IdeS (30 minutes at 37° C.) then loaded onto an ACQUITY UPLC Protein BEH C4 column (10K psi, 300 A, 1.7 um, 75 um×100 mm). Mass spectrum was acquired on a Waters Synapt G2-Si mass spectrometer. As shown in FIG. 6, the major peak is DAR 2 species.

TABLE 7
Mass Species (m/z) of IdeS Digested Ab and Site-
Specific Conjugates (i.e., (Fab)₂ Species)

| | | | Ab-Handle | Ab-Handle-linker | |
|---|---|---|---|---|---|
| No. | Name | Ab (Da) | (Da) | payload (ADC/ATL) (Da) | DAR |
| ADC1 | REGN1033-M6687 | 96282 | 96648 | 106446 | 1.96 |
| ADC2 | REGN1033-M6695 | 96282 | 96648 | 105942 | 1.95 |

Additional conjugation and characterization examples were reported in U.S. Patent Application Ser. No. 63/744,076, filed Jan. 10, 2025, and U.S. Patent Application Ser. No. 63/770,235, filed on Mar. 11, 2025, both of which are incorporated herein by reference in their entirety.

### Example 7. In Vitro Evaluations of GLP1R-GIPR Dual Agonists and their GDF8 Antibody Conjugates Toward GLP1R and GIPR Activation and the Blocking of GDF8 Activities Using CRE-Luc Assay and CAGA-Luc Assay

### Experimental Procedure (Include Description of Relevant Cell Lines, Proteins, Reagents, and Instrument Type and Model):

To test GLP1R and GIPR agonist activities of test articles, cell-based cAMP responsive luciferase reporter assays were developed. To generate the assay cell lines, the firefly luciferase gene was placed under the control of four copies of a cAMP response element (4xCRE) located upstream of a minimal promoter and transfected into HEK293 HZ cells (a highly transfectable subclone that was authenticated via STR profiling to be 81% identical to HEK293) and referred to herein as HEK293/CRE-Luc cells. HEK293/CRE-Luc cells were then engineered to express full-length human GLP1R (GenBank: AAI12127.1) (HEK293/CRE-Luc/hGLP1R) or full-length human GIPR (accession number NP_000155.1) (HEK293/CRE-Luc/hGIPR).

To test for agonistic specificity of test articles, additional members of the secretin family of GPCRs were engineered into HEK293/CRE-Luc cells: human glucagon receptor (GCGR) to generate HEK293/CRE-Luc/hGCGR cells and myc-tagged human glucagon-like peptide 2 receptor (GLP2R) to generate HEK293/CRE-Luc/hGLP2R cells.

To assess the ability of anti-GDF8 antibody and antibody conjugates (ADCs) to block GDF8-induced signaling, a luciferase reporter assay using the cell line A204 (a human rhabdomyosarcoma cell line that endogenously expresses GDF8 receptors) was developed. The A204 cell line (ATCC, #HTB-82) was stably transfected with a Smad2/3-luciferase reporter plasmid (CAGAx12-Luc), and a clonal cell line that displayed robust GDF8-mediated luminescent signal was isolated (referred to herein as A204/CAGA-Luc cells).

For all assays, cells were seeded into 384-well plates (Falcon #353988) at 6,000 cells/well in assay media (Opti-MEM, 1% FBS, 1× Penicillin-Streptomycin) and incubated overnight at 37° C. Serial dilutions of Regeneron peptidomimetics (M #reagents) were first made in DMSO and subsequently diluted 1:500 in assay media (constant 0.2% DMSO in assay wells). All other test article serial dilutions were made in assay media. Test articles were added to cells using VIAFLO 384 (Integra) with the last well in each serial dilution series serving as a blank control containing only assay media or assay media with 0.2% DMSO. Constant concentration of GDF8 (diluted in assay media) was added to cells using VIAFLO 384 for GDF8 blocking assay. After a 5-hour incubation at 37° C., luciferase activity was determined by addition of ONE-Glo reagent (Promega, #E6130) followed by measurement of relative light units (RLUs) on an EnVision Plate Reader (Revvity). For CRE-Luc assays, the antibody conjugate concentration was adjusted based on the drug-to-antibody ratio (DAR) of each test article by multiplying antibody concentration by DAR. For GDF8 blocking assay (CAGA-Luc assay), antibody conjugate's concentration was based on antibody concentration.

EC/IC₅₀ values were determined using a four-parameter logistic equation over a 12-point dose response curve (Dotmatics). The blank was plotted as a continuation of the serial dilution.

The maximum signal relative to endogenous ligand (Emax (% endogenous ligand)) in CRE-Luc assays was calculated using the following equation:

$${E}_{max} ( \% endogenous ligand ) = ( {RLU}_{max test article} - {RLU}_{blank} ) / ( {RLU}_{max endogenous ligand} - {RLU}_{blank} ) \times 100$$

The maximum blocking percentage (Max blocking (%)) in CAGA-Luc assay was calculated using the following equation:

$$Max blocking ( \% ) = ( {RLU}_{max test article} - {RLU}_{min test article} ) / ( {RLU}_{max test article} - {RLU}_{blank} ) \times 100$$

### Results Summary and Conclusions:

As shown in Table 8, the endogenous GLP1R ligand, GLP-1 (7-36) amide (Phoenix Pharmaceuticals #028-11) (referred to as GLP-1), increased CRE-dependent luciferase reporter activity in HEK293/CRE-Luc/hGLP1R cells with EC₅₀ value of 210 pM and showed very weak activation of HEK293/CRE-Luc/hGIPR cells up to 1 pM. The endogenous GIPR ligand, GIP (Tocris #2084), increased CRE-dependent luciferase reporter activity in HEK293/CRE-Luc/hGIPR cells with EC₅₀ value of 9.53 pM and showed no activation of HEK293/CRE-Luc/hGLP1R cells up to 1 pM. Semaglutide induced CRE-dependent luciferase reporter activity in HEK293/CRE-Luc/hGLP1R cells with EC₅₀ value of 49.7 pM and very weak activation of HEK293/CRE-Luc/hGIPR cells up to 1 pM. Tirzepatide increased CRE-dependent luciferase reporter activity in both HEK293/CRE-Luc/hGLP1R and HEK293/CRE-Luc/hGIPR cells with EC₅₀ values of 229 pM and 6.87 pM, respectively.

A list of 28 compounds (i.e., M6486 to M6991 listed in Table 2) was tested in CRE-dependent luciferase reporter activity assays and found to activate both HEK293/CRE-Luc/hGLP1R and HEK293/CRE-Luc/hGIPR cells with EC₅₀ values ranging from 0.227 to 68.9 pM for hGLP1R cells and from 0.107 to 158 pM for hGIPR cells (Table 8).

Anti-GDF8 monoclonal antibody (mAb) REGN1033 was conjugated to dual agonist (GLP1R/GIPR) linker-payloads (LPs) M6687 and M6695 via light chain glutamine at position 55 (Q55) to generate anti-GDF8 Antibody conjugates. As shown in Table 8, anti-GDF8 mAb REGN1033 did not induce CRE-dependent luciferase reporter activity in either HEK293/CRE-Luc/hGLP1R or HEK293/CRE-Luc/hGIPR cells up to 0.5 pM. In contrast, REGN1033-M6687 and REGN1033-M6695 (anti-GDF8 Antibody conjugates) increased CRE-dependent luciferase reporter activity in HEK293/CRE-Luc/hGLP1R cells with EC₅₀ values of 46.4 pM and 44.7 pM, respectively, and increased CRE-dependent luciferase reporter activity in HEK293/CRE-Luc/hGIPR cells with EC₅₀ values of 6.77 pM and 5.31 pM, respectively.

TABLE 8
CRE-Dependent Reporter Activity Induced by Test Articles
in HEK293/CRE-Luc/hGLP1R and HEK293/CRE-Luc/hGIPR Cells

| | HEK293/CRE- | | | |
|---|---|---|---|---|
| | Luc/hGLP1R | | HEK293/CRE-Luc/hGIPR | |
| | | Eₘₐₓ | | Eₘₐₓ |
| Test article | EC₅₀ (M) | (% GLP-1) | EC₅₀ (M) | (% GIP) |
| GLP-1 (7-36) | 2.10E−10 | 100.0 | >1.00E−06 | 33.1 |
| amide | | | | |
| GIP | >1.00E−06 | 1.1 | 9.53E−12 | 100.0 |
| Semaglutide | 4.97E−11 | 98.1 | >1.00E−06 | 30.9 |
| Tirzepatide | 2.29E−10 | 96.8 | 6.87E−12 | 96.0 |
| M6486 | 9.47E−13 | 82.1 | 7.00E−12 | 70.9 |
| M6487 | 7.85E−13 | 76.4 | 5.51E−13 | 65.2 |
| M6491 | 1.03E−12 | 79.6 | 1.58E−10 | 76.0 |
| M6682 | 2.70E−12 | 81.1 | 2.55E−12 | 72.9 |
| M6687 | 1.45E−12 | 80.6 | 5.89E−13 | 69.9 |
| M6695 | 1.53E−12 | 82.0 | 1.38E−12 | 73.0 |
| M6696 | 1.19E−12 | 81.2 | 5.63E−13 | 73.3 |
| M6697 | 2.48E−12 | 88.5 | 6.01E−12 | 75.8 |
| M6810 | 5.34E−13 | 81.9 | 4.69E−13 | 68.4 |
| M6953 | 5.34E−12 | 73.3 | 2.92E−12 | 67.5 |
| M6954 | 2.46E−12 | 74.6 | 6.19E−13 | 70.9 |
| M6956 | 1.01E−11 | 72.0 | 7.70E−12 | 67.2 |
| M6957 | 2.10E−12 | 73.3 | 5.93E−12 | 66.7 |
| M6958 | 7.89E−12 | 73.0 | 4.17E−12 | 69.7 |
| M6959 | 4.85E−12 | 73.2 | 2.63E−12 | 70.5 |
| M6960 | 1.59E−12 | 75.9 | 5.66E−13 | 68.1 |
| M6961 | 7.45E−13 | 74.9 | 2.64E−12 | 69.3 |
| M6962 | 2.71E−12 | 76.8 | 2.58E−12 | 74.9 |
| M6969 | 9.10E−13 | 81.6 | 2.62E−12 | 73.4 |
| M6970 | 5.51E−13 | 82.8 | 2.58E−12 | 74.4 |
| M6976 | 1.34E−12 | 86.0 | 7.82E−13 | 75.3 |
| M6977 | 2.27E−13 | 83.9 | 1.69E−13 | 72.0 |
| M6978 | 3.21E−13 | 86.8 | 1.89E−13 | 72.7 |
| M6979 | 2.36E−13 | 84.9 | 1.07E−13 | 81.1 |
| M6988 | 5.65E−13 | 85.3 | 1.53E−12 | 93.8 |
| M6989 | 2.10E−12 | 93.7 | 1.10E−12 | 90.4 |
| M6990 | 6.89E−11 | 83.4 | 6.53E−11 | 77.5 |
| M6991 | 5.09E−13 | 81.1 | 3.54E−13 | 72.1 |
| REGN1033 | >5.00E−07 | 0.2 | >5.00E−07 | 0.7 |
| (anti-GDF8 | | | | |
| mAb) | | | | |
| REGN1033- | 4.64E−11 | 98.0 | 6.77E−12 | 95.1 |
| M6687 | | | | |
| REGN1033- | 4.47E−11 | 98.9 | 5.31E−12 | 96.6 |
| M6695 | | | | |

>=EC₅₀ values could not be determined with accuracy because luminescence signal did not reach saturation within the tested concentration range. EC₅₀ is reported as greater than the highest tested concentration.

To test specificity of compounds to activate GLP1R and GIPR, CRE-dependent luciferase reporter activity was assessed in reporter cell lines expressing additional members of the secretin family of GPCRs, namely GCGR and GLP2R, for a subset of compounds. As shown in Table 9, only glucagon induced CRE-dependent luciferase reporter activity in HEK/CRE-Luc/hGCGR cells (EC₅₀ value of 1.33 nM) and only GLP-2 induced CRE-dependent luciferase reporter activity in HEK/CRE-Luc/hGLP2R cells (EC₅₀ value of 2.23 nM), whereas GLP-1, semaglutide, tirzepatide, M6687, M6695, REGN1033-M6687, and REGN1033-M6695 did not induce significant reporter activity in either GCGR or GLP2R cell lines.

TABLE 9
CRE-Dependent Reporter Activity Induced by Test Articles
in HEK293/CRE-Luc/hGCGR and HEK293/CRE-Luc/hGLP2R Cells

| | HEK293/CRE-Luc/hGCGR | | HEK293/CRE-Luc/hGLP2R | |
|---|---|---|---|---|
| | | Eₘₐₓ | | Eₘₐₓ |
| Test article | EC₅₀ (M) | (% Glucagon) | EC₅₀ (M) | (% GLP-2) |
| Glucagon | 1.33E−09 | 100.0 | >1.00E−06 | 2.9 |
| GLP-2 | >1.00E−06 | 6.9 | 2.23E−09 | 100.0 |
| GLP-1 (7-36) amide | >1.00E−06 | 2.0 | >1.00E−06 | 1.0 |
| Semaglutide | >1.00E−06 | 0.9 | >1.00E−06 | 0.6 |
| Tirzepatide | >1.00E−06 | 1.7 | >1.00E−06 | 31.6 |
| M6687 | >1.00E−06 | 6.0 | >1.00E−06 | 55.0 |
| M6695 | >1.00E−06 | 0.2 | >1.00E−06 | 34.2 |
| REGN1033-M6687 | >1.00E−06 | 1.7 | >1.00E−06 | 6.8 |
| (anti-GDF8 antibody | | | | |
| conjugate) | | | | |
| REGN1033-M6695 | >1.00E−06 | 1.0 | >1.00E−06 | 2.0 |
| (anti-GDF8 antibody | | | | |
| conjugate) | | | | |

>=EC₅₀ values could not be determined with accuracy because luminescence signal did not reach saturation within the tested concentration range. EC₅₀ is reported as greater than the highest tested concentration.

GDF8 induces a robust luminescent signal in A204/CAGA-Luc cells with EC₅₀ value of 335 pM. As shown in Table 10, REGN1033, an anti-GDF8 mAb, and REGN1033 antibody conjugates (REGN1033-M6687 and REGN1033-M6695) blocked GDF8-dependent reporter activity (1 nM of GDF8) in A204/CAGA-Luc cells, with IC₅₀ values ranging from 481 pM to 870 pM and maximum blocking percentages ranging from 98.9 to 100.0%. Isotype control antibody conjugate (i.e., non-GDF8 binding antibody conjugated to M6562 via light chain Q55; M6562 is a GLP1R agonist with minimal GIPR activities) did not promote significant blocking of GDF8-dependent signaling in A204/CAGA-Luc cells.

These results show that REGN1033-M6687 and REGN1033-M6695 antibody conjugates activate both GLP1R and GIPR (i.e., dual agonist ability) in cell-based bioassays while maintaining similar GDF8 blocking activity as the unconjugated REGN1033 mAb.

TABLE 10
Blocking of GDF8-Dependent Reporter Activity in
A204/CAGA-Luc Cells by mAb/Antibody conjugates

| | A204/CAGA-Luc | |
|---|---|---|
| | With 1 nM GDF8 | |
| Test article | IC₅₀ (M) | Max blocking (%) |
| REGN1033 | 8.70E−10 | 99.4 |
| (anti-GDF8 mAb) | | |
| REGN1033-M6687 | 4.85E−10 | 100.0 |
| (anti-GDF8 antibody conjugate) | | |
| REGN1033-M6695 | 4.81E−10 | 98.9 |
| (anti-GDF8 antibody conjugate) | | |
| Isotype control-M6562 | >1.00E−07 | 13.7 |
| (isotype antibody conjugate) | | |

>=IC₅₀ values could not be determined with accuracy because luminescence signal did not reach the lower plateau within the tested concentration range. IC₅₀ is reported as greater than the highest tested concentration.

### Example 8. Biacore Kinetics of Anti-GDF8 Antibody Conjugates Binding to Dimeric Human GDF8 in an Antibody Capture Format at 25° C.

Equilibrium dissociation constants (K_{D} values) of anti-GDF8 antibody conjugates binding to dimeric human GDF8 were determined using real-time surface plasmon resonance biosensor technology on a Biacore T-200 instrument. Dimeric human GDF8 was expressed without a tag (human GDF8, REGN30: NENSEQKENVEKEGLCNACTWRQNTKSSRIEAIKIQILSKLRLETAPNISKDVIRQLLPKA PPLRELIDQYDVQRDDSSDGSLEDDDYHATTETIITMPTESDFLMQVDGKPKCCFFKFSS KIQYNKVVKAQLWIYLRPVETPTTVFVQILRLIKPMKDGTRYTGIRSLKLDMNPGTGIW QSIDVKTVLQNWLKQPESNLGIEIKALDENGHDLAVTFPGPGEDGLNPFLEVKVTDTPK RRRRDFGLDCDEHSTESRCCRYPLTVDFEAFGWDWIIAPKRYKANYCSGECEFVFLQKY PHTHLVHQANPRGSAGPCCTPTKMSPINMLYFNGKEQIIYGKIPAMVVDRCGCS, SEQ ID NO: 41). Briefly, the CM5 Biacore sensor surface was derivatized by amine coupling a monoclonal mouse anti-human Fc monoclonal antibody. All Biacore binding studies were performed in a buffer composed of 10 mM HEPES pH 7.4, 150 mM NaCl, 0.05% v/v Surfactant P20 (HBS-EP running buffer). Anti-GDF8 antibody conjugates were captured onto the anti-hFc surface, and different concentrations of dimeric human GDF8, ranging from 0.6 nM to 48.5 nM in 3-fold serial dilutions, were injected at a flow rate of 30 μL/minute. Antibody-ligand association was monitored for 5 minutes, and dissociation was monitored for 10 minutes. At the end of each cycle, the hIgG capture surface was regenerated using a 10 second injection of 20 mM phosphoric acid. All binding kinetics experiments were performed at 25° C.

The specific SPR-Biacore sensorgrams were obtained by a double referencing procedure. The double referencing was performed by first subtracting the signal of each injection over a reference surface (anti-hFc) from the signal over the experimental surface (anti-hFc-captured anti-GDF8-antibody conjugates) thereby removing contributions from refractive index changes. In addition, running buffer injections were performed to allow subtraction of the signal changes resulting from the dissociation of captured antibodies from the coupled anti-hFc surface. Kinetic association (kₐ) and dissociation (k_{d}) rate constants were determined by fitting the real-time sensorgrams to a 1:1 binding model using Scrubber v2.0c curve fitting software. Binding dissociation equilibrium constants (K_{D}) and dissociative half-lives (t½) were calculated from the kinetic rate constants as:

$${K}_{D} ( M ) = \frac{k d}{k a} , and t \frac{1}{2} ( min ) = \frac{ln ( 2 )}{60 * k d}$$

Human GDF8 binding kinetics results to anti-GDF8-antibody conjugates obtained at 25° C. are summarized in Table 11. The unconjugated anti-GDF8 mAb had similar or weaker binding affinity to dimeric human GDF8 as the anti-GDF8-antibody conjugates.

Results: Binding kinetics of human GDF8 binding to anti-GDF8 antibody conjugates obtained at 25° C. are summarized in Table 11.

TABLE 11
Kinetic and Equilibrium Binding Parameters of Human GDF8 binding to
Surface-captured anti-GDF8 antibody conjugates

| | | 6 nM | | | | |
|---|---|---|---|---|---|---|
| | | hGDF8 | | | | |
| | | (REGN30) | | | | |
| | mAb Capture | Bound | ka | kd | KD | t½ |
| Test Article | (RU) | (RU) | (1/Ms) | (1/s) | (M) | (min) |
| REGN1033 | 382.2 ± 2.4 | 76.7 | 3.10E+06 | 3.20E−05 | 1.03E−11 | 361.0 |
| (anti-GDF8 mAb) | | | | | | |
| REGN1033-M6562 | 301.9 ± 1.7 | 68.5 | 5.89E+06 | 3.31E−05 | 5.61E−12 | 349.3 |
| REGN1033-M6677 | 310.4 ± 1.7 | 68.4 | 4.33E+06 | 3.47E−05 | 8.01E−12 | 332.9 |
| REGN1033-M6687 | 191.6 ± 1.0 | 46.8 | 8.30E+06 | 4.25E−05 | 5.11E−12 | 272.1 |
| REGN1033-M6689 | 320.1 ± 2.1 | 70.6 | 4.95E+06 | 3.32E−05 | 6.70E−12 | 348.3 |
| REGN1033-M6690 | 301.6 ± 2.2 | 64.2 | 3.21E+06 | 3.25E−05 | 1.01E−11 | 355.5 |
| REGN1033-M6695 | 305.0 ± 1.7 | 66.8 | 5.09E+06 | 4.60E−05 | 9.04E−12 | 251.1 |

### Example 9. In Vitro Plasma Stability of Dual Agonist Linker Payload M6687 and M6695 by LC-MS

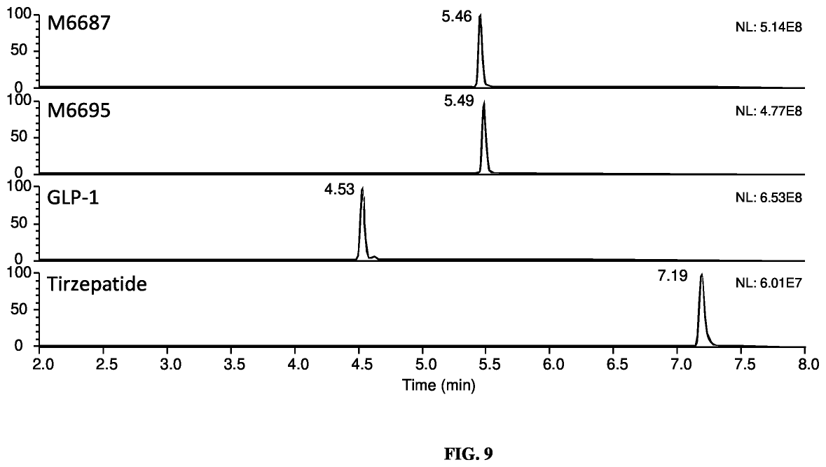

M6687, M6695, GLP-1 or Tirzepatide (1 mg/mL in DMSO) was spiked into mouse and human plasma to achieve a final concentration of 15 μg/mL. The resulting mixture was incubated at 37° C. with gentle shaking, and 15 μL of plasma was collected at 0, 20, 40, 60, 120, 180, and 240 minutes, followed by immediate quenching with 60 μL of cold methanol containing 0.1% formic acid in a 96-well plate. An internal standard solution containing 250 ng/mL each of M6324, M6447, and M3190 in 50% methanol was added to each well before mixing at 1250 rpm for 2 minutes. The 96-well plate was then centrifuged at 3500 rpm for 10 minutes at 5° C. After the centrifugation, 37.5 μL of the supernatant was transferred to a new low binding 96-well plate containing 22.5 μL of water with 0.1% formic acid. The plate was then vortexed at 1250 rpm for 2 min and briefly centrifuged before LC-MS analysis on an Orbitrap Exploris 480 Mass Spectrometer (Thermo Scientific) coupled with Vanquish UHPLC. The separation was carried on Waters ACQUITY Peptide CSH C18 column (130 Å 1.7 m 2.1 mm×50 mm) with mobile phase A: 0.1% formic acid in water, mobile phase B: 0.1% formic acid in acetonitrile. LC gradient was from 15% mobile phase B to 60% mobile phase B over 7.2 min at a flow rate of 0.30 ml/min with column temperature at 40° C. Eluted peptide or peptidomimetics were analyzed by Orbitrap MS analyzer equipped with heated-ESI probe operating in positive mode at 3.5 kV spray voltage. MS analyzer was set at the following parameters: MS resolution: 60,000; MS scan range: 400-2000 m z, MS AGC target: 300%; RF lens: 50%. Raw data was analyzed by Quan Browser integrated in Thermo Scientific Xcalibur to quantify peptides based on sum of three most abundant ions with mass accuracy setting<20 ppm (Table 12, FIG. 9).

### Results Summary and Conclusions:

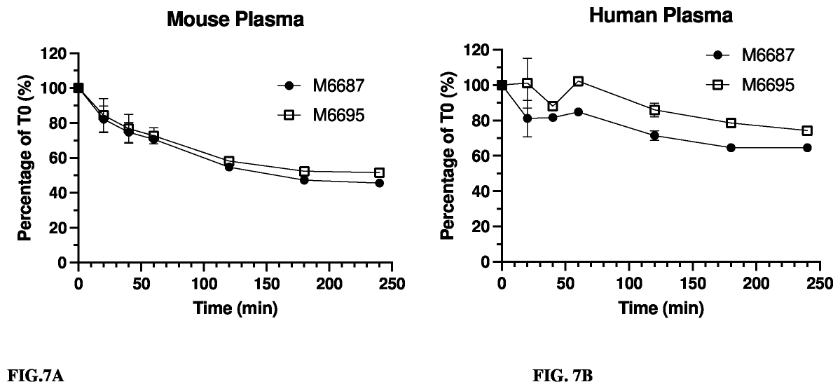

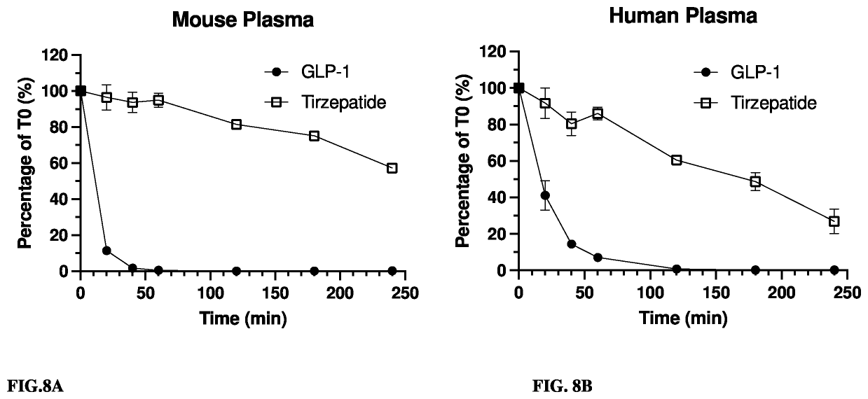

After a 4-hour incubation at 37° C., approximately 50% of M6687 and M6695 remained intact in mouse plasma, while about 75% M6687 and M6695 remained intact in human plasma (FIGS. 7A-7B and FIGS. 8A-8B). Both M6687 and M6695 demonstrated improved plasma stability over endogenous GLP1 peptide and showed similar or improved stability as Tirzepatide in mouse and human plasma, respectively.

TABLE 12
Summary of m/z Values for Peptide/Peptidomimetics
Quantification

| | | Charge | Linear range |
|---|---|---|---|
| Sample ID | m/z of ions for quantification | State | (μg/mL) |
| M6687 | 1232.0897, 1232.3405, 1232.5914 | 4+ | 0.3-20 |
| M6695 | 1169.3052, 1169.5560, 1169.8068 | 4+ | 0.3-20 |
| Tirzepatide | 1203.8893, 1204.1402, 1204.3910 | 4+ | 1-20 |
| GLP-1 | 824.9299, 825.1738, 825.4246 | 4+ | 0.3-20 |

### Example 10. First In Vivo Efficacy Study: A GDF8 Antibody Conjugated GLP1R-GIPR Dual Agonist REGN1033-M6695

The effects of a GDF8 antibody conjugated GLP1R agonist/GIPR agonist peptidomimetic drug of the invention, REGN1033-M6695, on body weight and body composition were determined in 60% high fat diet-fed (HFD) obese female 75% C57BL/6NTac 25% 129S6/SvEvTac mice carrying the human version of the GIPR gene in place of the mouse version of the Gipr gene (Gipr^{hu/hu}) aged between 39 to 59 weeks old.

Gipr^{hu/hu} mice were placed on 60% HFD for 31 weeks prior to the start of the study. At the start of the study, body weight and body composition were measured by qNMR for the purpose of randomization into 3 treatment groups of 8 to 9 mice. The treatment groups were as follows:
1. Isotype Control antibody
2. Tirzepatide
3. REGN1033-M6695 antibody conjugate

All treatments were administered via subcutaneous injection. A dose escalation was performed over the 37-day study. Between days 0 to 13, Tirzepatide was administered twice weekly at 0.14 mg/kg, and Isotype Control and REGN1033-M6695 were administered once weekly at 2.3 mg/kg. Between days 14 to 37, Tirzepatide was administered twice weekly at 0.48 mg/kg, and Isotype Control and REGN1033-M6695 were administered once weekly at 7.7 mg/kg.

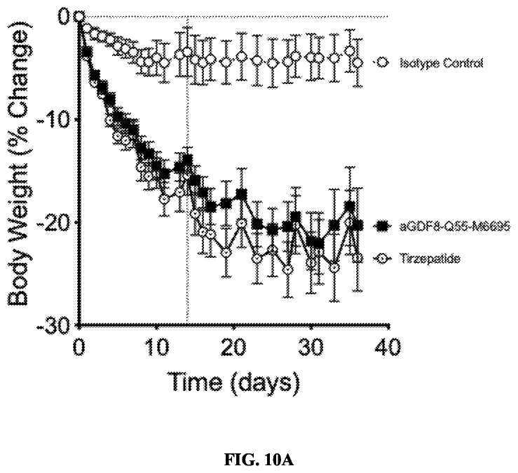

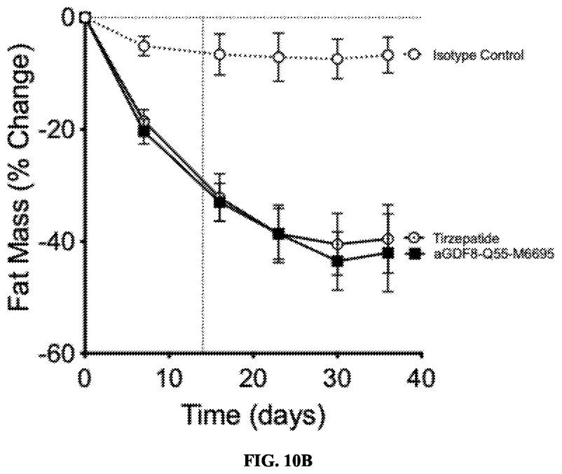

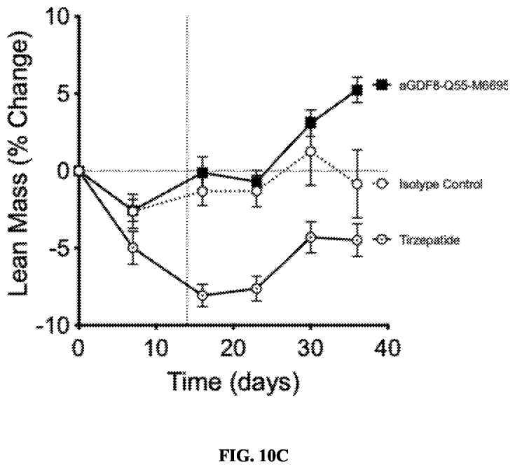

Body weights were measured at least 3 days per week, and body composition was quantified by qNMR once weekly. FIGS. 10A-10C summarizes body weight and body composition over the course of the study. All results are expressed as average % change from baseline±SEM.

As shown in FIGS. 10A-10C, following 36 days of treatment, administration of Tirzepatide and REGN1033-M6695 conjugate elicited significant reductions in overall body weight. Tirzepatide treatment resulted in a net body weight loss (23%) that can be attributed to a reduction in both fat mass (40%) and lean mass (4.5%). In contrast, treatment with REGN1033-M6695 conjugate also led to a net body weight loss (20%); however, this is a result of fat mass loss (42%) and lean mass gain (5.2%).

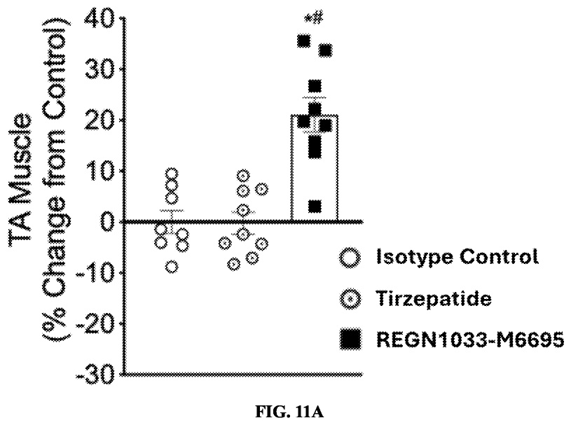

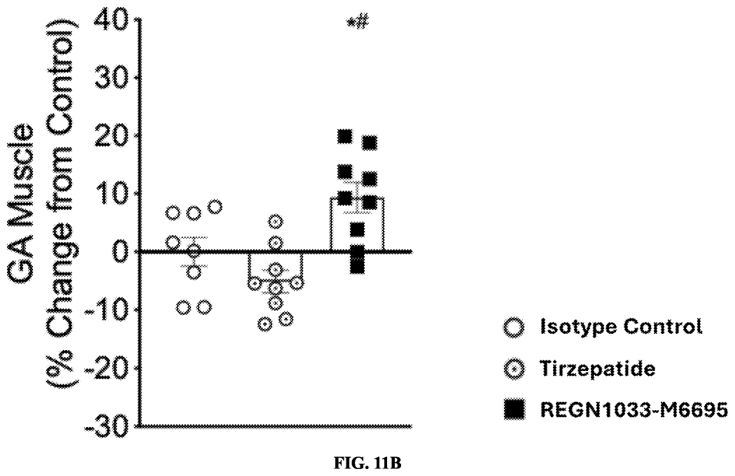

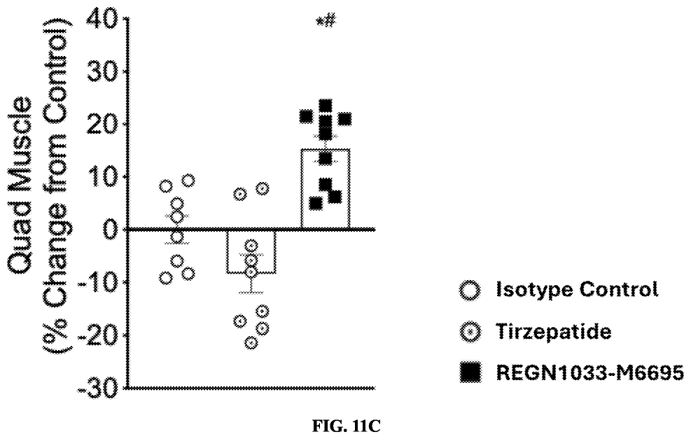

To further investigate the disparity in lean mass between Tirzepatide treatment and REGN1033-M6695, tissue weights were collected and measured after 37 days of treatment (FIGS. 11A-11C). Tissue weights are expressed as a % change from the average of the Isotype Control group±SEM.

As shown in FIGS. 11A-11C, skeletal muscle measurements revealed that treatment with REGN1033-M6695 significantly increased tibialis anterior (21%), gastrocnemius (9.3%), and quadriceps (Q15%) muscle weights compared to Isotype Control. The tibialis anterior (TA) is a muscle located in the front part of your lower leg, just beside the shin bone (tibia). It plays a crucial role in foot and ankle movement. The gastrocnemius (GA) is one of the major muscles in the calf region of the lower leg. It is a superficial muscle, meaning it is located close to the skin, and is one of the most prominent muscles in the calf. The gastrocnemius is responsible for several important functions, particularly in movement and posture. The quadriceps (Quad), commonly referred to as the “quads,” are a group of four muscles located in the front of the thigh. These muscles are critical for movement, stability, and strength in the lower body. Together, they are responsible for extending the knee and play a key role in walking, running, jumping, and squatting. These results are unlike Tirzepatide treatment; mice receiving Tirzepatide had slightly reduced skeletal muscle mass compared to Isotype Control. As a result, all skeletal muscle weights from REGN1033-M6695 antibody conjugate treatment were also significantly greater than Tirzepatide treatment.

### Example 11. Second In Vivo Efficacy Study: Comparison of GDF8 Antibody Conjugated GLP1R-GIPR Dual Agonist REGN1033-M6695 and REGN1033-M6687 for Inducing Fat Mass Loss while Preserving Lean Mass

An antibody conjugate of REGN1033-GLP1R-GIPR dual agonist was prepared where the linker payload was generated through a different synthetic route (REGN1033-M6687). For the study, 50-week-old male humanized GIPR mice, who were on a 60% high-fat diet for 41 weeks, were separated into three groups of 10 mice each based on their baseline fat mass and body weight. The three treatment groups were as follows: 1) Control mAb: isotype control dosed using step-up dosing; 2.3 mg/kg for the first two weeks followed by 7.7 mg/kg for the next two weeks; 2) REGN1033-M6695 also at 2.3-7.7 mg/kg; and 3) REGN1033-M6687 also at 2.3-7.7 mg/kg. Antibody conjugates of GLP1R agonist peptides M6562 and M6677 were included in the study for comparison. Those two peptides have no measurable GIPR affinity. Each mouse was given 4 treatment doses over a four-week period (dosing schedule and groups are listed in Table 13).

TABLE 13
Study Groups and Dosing

| Group | (N) | Treatment (dose) | Group | Frequency | Route |
|---|---|---|---|---|---|
| 1 | 10 | Isotype Control, 2.3 | Control mAb | Weekly | S.C. |
| | | mg/kg doses 1-2; 7.7 | | | |
| | | mg/kg, doses 3-4 | | | |
| 2 | 10 | 2.3 mg/kg doses 1-2; 7.7 | REGN1033- | Weekly | S.C. |
| | | mg/kg, doses 3-4 | M6695 | | |
| 3 | 10 | 2.3 mg/kg doses 1-2; 7.7 | REGN1033- | Weekly | S.C. |
| | | mg/kg, doses 3-4 | M6687 | | |

Isotype Control Antibody Used in the Efficacy Study is H4H30045P, Target Antigen(s): FGFR3 [Homo sapiens].

Body weights were taken throughout the study and body composition was measured by EchoMRI at baseline and on study days 10 and 25. Fed glucose was measured by glucometer from the tail prior to dosing every week. On day 26, the mice were euthanized in a staggered fashion and the following organ weights were measured: tibialis anterior (TA) muscle, quadriceps muscle (Quad), gastrocnemius muscle (GA; dissection also included the soleus), subcutaneous white adipose (scWAT), gonadal white adipose tissue (gWAT), mesenteric adipose tissue (mWAT) and liver.

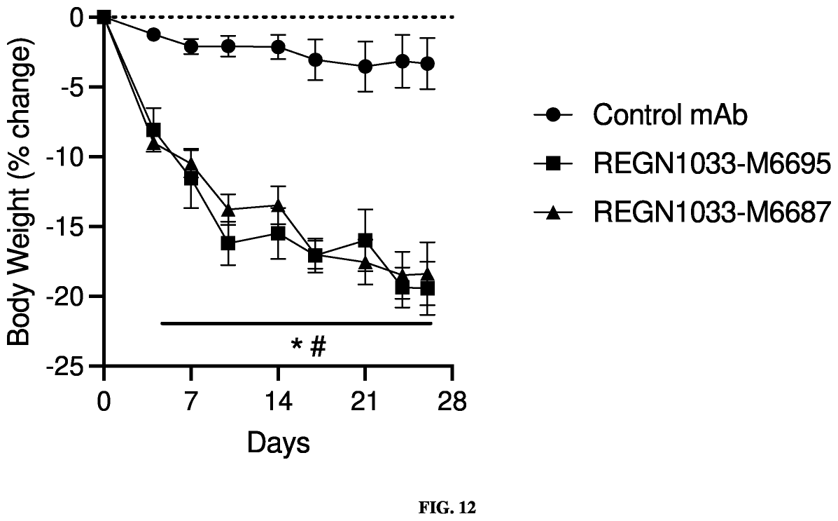

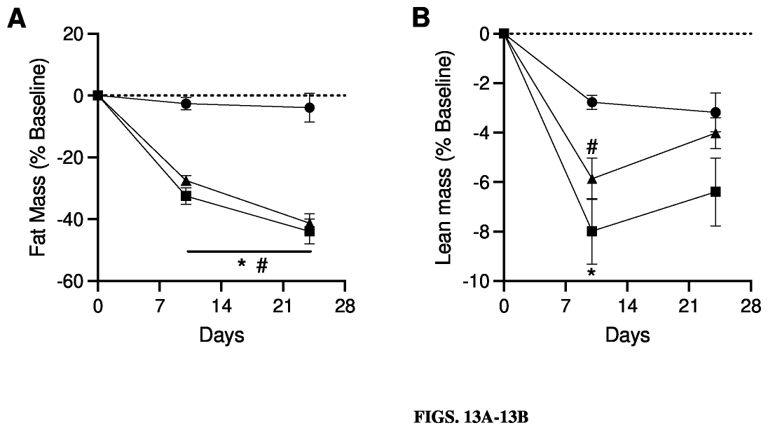

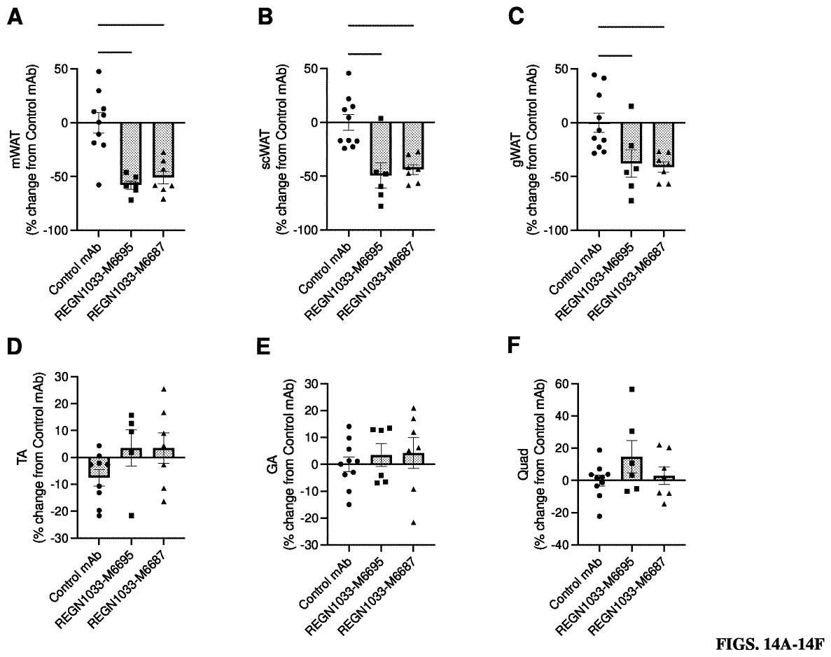

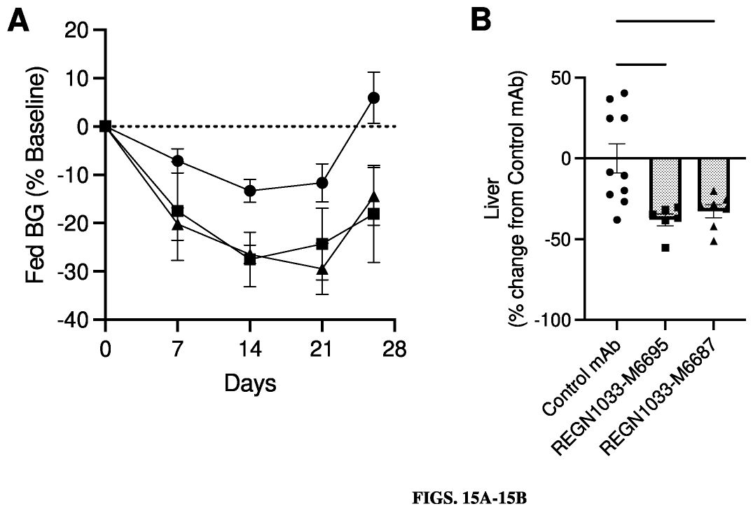

Change in body weight, total body fat and total lean mass were calculated as the percent difference from each time point compared to each baseline reading. Body weights are shown in FIG. 12 and body composition is shown in FIGS. 13A-13B. Terminal muscle and fat weights are shown in FIGS. 14A-14F, while terminal liver weights and fed glucose are shown in FIGS. 15A-15B, all expressed as percent change from baseline. Changes over time were assessed by two-way ANOVA while the final results were compared by one-way ANOVA.

### Results

Body Weight—Percent change in body weight is shown in FIG. 12. All the groups which received a GLP1R-GIPR agonist conjugated to REGN1033 lost 18-19% of their body weight over the four-week study.

Body Composition—Body composition was assessed at baseline, D10 of the study (following the first two doses) and at D25 (following all four doses). Change from baseline is plotted in FIGS. 13A-13B for % change in fat mass (FIG. 13A) and % change in lean mass (FIG. 13B) from baseline.

Fat mass—All groups which received a GLP1R-GIPR agonist peptidomimetic conjugate lost fat mass with the REGN1033-M6562 group losing nearly 26% and the REGN-M6677 group losing nearly 22%. Peptides M6562 and M6677 are a GLP1R agonist with minimal GIPR activities.

Lean mass—REGN1033-M6695 had a −6.4% change in lean mass while the REGN1033-M6687 had a −4% change in lean mass at the end of the study vs. −3.2% for the control. None of these significantly different from the control at the end of the study.

The terminal fat and muscle weights showed similar effects (FIGS. 14A-14F). GLP1R-GIPR agonist significantly reduced mesenteric, subcutaneous and gonadal fat (FIGS. 14A-14C) while muscle groups showed no significant change at the end of four weeks (FIGS. 14D-14F).

Blood Glucose and Liver Weight—Percent change in fed glucose and liver weight is shown in FIGS. 15A-15B. All the groups which received a GLP1R-GIPR agonist conjugated to REGN1033 trended to reduce blood glucose and significantly reduced liver weight over the four-week study (FIGS. 15A-15B).

Results Summary—REGN1033-M6695 and REGN1033-M6687 reduced fat mass and body weight similarly, while preserving lean mass to a similar extent.

As various changes can be made in the above-described subject matter without departing from the scope and spirit of the present disclosure, it is intended that all subject matter contained in the above description, or defined in the appended claims, be interpreted as descriptive and illustrative of the present disclosure. Many modifications and variations of the present disclosure are possible in light of the above teachings. Accordingly, the present description is intended to embrace all such alternatives, modifications, and variances which fall within the scope of the appended claims.

All patents, applications, publications, test methods, literature, and other materials cited herein are hereby incorporated by reference in their entirety as if physically present in this specification.

1. A conjugate, or a pharmaceutically acceptable salt thereof, comprising an antigen-binding protein (ABP) and at least one glucagon-like peptide-1 (GLP-1) receptor (GLP1R) and glucose-dependent insulinotropic polypeptide (GIP) receptor (GIPR) dual agonist (GLP1R-GIPR dual agonist), wherein the GLP1R and GIPR dual agonist is covalently conjugated to the ABP through a linker.

2.-40. (canceled)

41. A glucagon-like peptide-1 (GLP-1) receptor (GLP1R) and glucose-dependent insulinotropic polypeptide (GIP) receptor (GIPR) dual agonist, or a pharmaceutically acceptable salt thereof, comprising: Tyr1 Aib2 XX3 Gly4 XX5 XX6 Thr Ser Asp Tyr Ser Ser XX13 Leu Glu Glu Gln Ala Ala XX20 Glu XX22 Ile XX24 XX25 Leu Val XX28 Gly Gly Gly (SEQ ID NO: 3), wherein Aib represents 2-aminoisobutyric acid (α-aminoisobutyric acid); XX3 is Glu or Atz ((S)-2-amino-3-(2H-tetrazol-5-yl)propanoic acid); XX5 is Ser or Thr; XX6 is phenylalanine, alpha methyl phenylalanine (amF) or 2-fluoro alpha methyl phenylalanine (amF(2-fluoro)); XX13 is Tyr, amF, or alpha methyl tyrosine (amY); XX20 is Lys, sidechain-acylated or alkylated Lys, or Aib; XX22 is Phe or amF; XX24 is Ala or Glu; XX25 is tryptophan or alpha methyl tyrosine (amY); XX28 is Lys, Ala, Gln, or Glu; and wherein the C-terminal end is an acid or an amide.

42.-49. (canceled)

50. The GLP1R and GIPR dual agonist, or a pharmaceutically acceptable salt thereof, according to claim 41, wherein said GLP1R-GIPR dual agonist has a sequence selected from the group consisting of SEQ ID NOs: 3, 14-17, and 19-40.

51. The GLP1R and GIPR dual agonist, or a pharmaceutically acceptable salt thereof, according to claim 41, wherein the GLP1R and GIPR dual agonist binds to and activates the signaling cascade of GLP1R and GIPR.

52. (canceled)

53. A conjugate, or a pharmaceutically acceptable salt thereof, comprising a GLP1R and GIPR dual agonist according to claim 41, an antigen binding protein (ABP), and a linker covalently linking said dual agonist and ABP, wherein said ABP is a GDF8 antibody, or the antigen-binding fragment thereof.

54. The GLP1R and GIPR dual agonist, or a pharmaceutically acceptable salt thereof, according to claim 41, further comprising a linker with a reactive moiety for conjugation with a target antibody or antigen binding fragment thereof.

55. The GLP1R and GIPR dual agonist, or a pharmaceutically acceptable salt thereof, according to claim 54, wherein the linker with a reactive moiety for conjugation with a target antibody or antigen binding fragment thereof, has the following structure: -(Gly3Ser)ₙ-Lys(X)- (SEQ ID NO: 97) or -(Gly₄Ser)ₙ-Lys(X)- (SEQ ID NO: 98), wherein n is an integer from one to six; and wherein X is a reactive moiety attached to the side chain of Lys for conjugation with an antigen binding protein (ABP).

56. The GLP1R and GIPR dual agonist, or a pharmaceutically acceptable salt thereof, according to claim 55, wherein X is a reactive moiety comprising an azido, 2-bromo-acetyl, 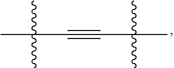 [SMILES: *C#C*]
2-(cyclooct-2-yn-lyloxy) acetyl, 2′-maleimido-acetyl, and 3′-maleimido-propanoyl, —N₃, 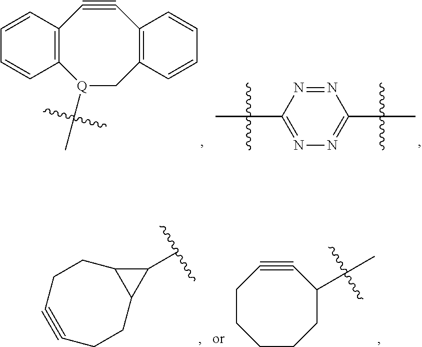
where Q is CH or N.

57. The GLP1R and GIPR dual agonist, or a pharmaceutically acceptable salt thereof, according to claim 55, wherein Lys(X) has a structure of

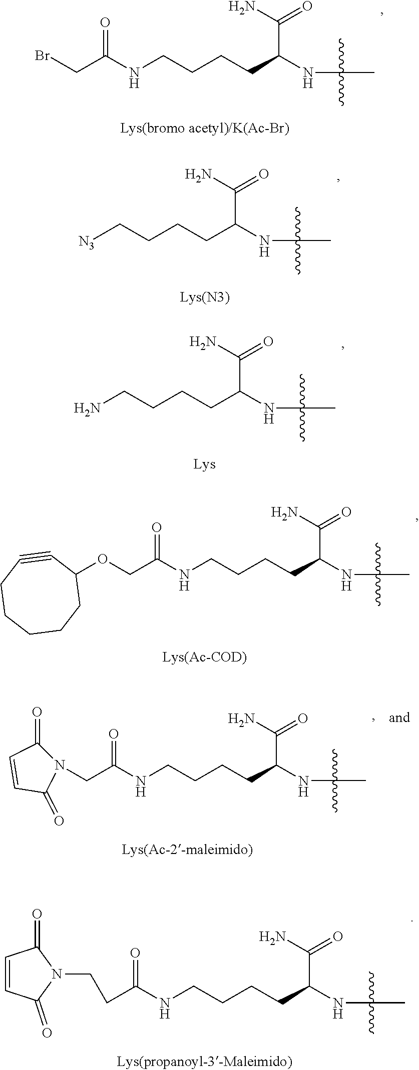 [SMILES: *N[C@@H](CCCCNC(=O)CBr)C(N)=O ; *NC(CCCCN=[N+]=[N-])C(N)=O ; *NC(CCCCN)C(N)=O ; *N[C@@H](CCCCNC(=O)COC1C#CCCCCC1)C(N)=O ; *N[C@@H](CCCCNC(=O)CN1C(=O)C=CC1=O)C(N)=O ; *N[C@@H](CCCCNC(=O)CCN1C(=O)C=CC1=O)C(N)=O]

58. The GLP1R and GIPR dual agonist, or a pharmaceutically acceptable salt thereof, according to claim 54, wherein the linker comprises 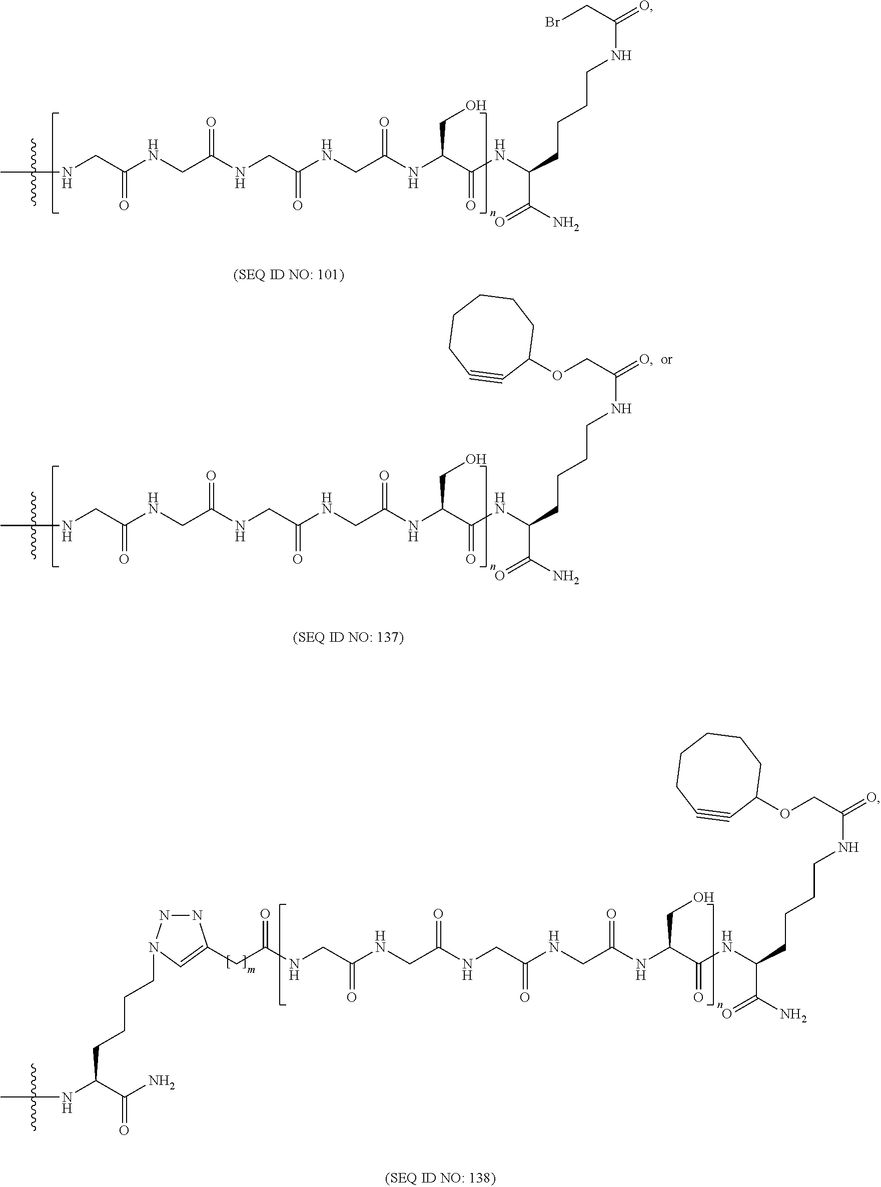
wherein m is an integer of one to three; and n is an integer of one to four.

59. A pharmaceutical composition comprising the GLP1R and GIPR dual agonist of claim 41, or a pharmaceutically acceptable salt thereof.

60. (canceled)

61. A GLP1R-GIPR dual agonist and an antigen binding protein (ABP) conjugate, or a pharmaceutically acceptable salt thereof, having a Formula (I): 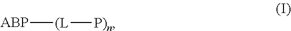
wherein
ABP is an antibody, or an antigen-binding fragment thereof, that specifically binds to and/or blocks the biological activity of human Growth and Differentiation Factor-8 (GDF8);
P is a glucagon-like peptide-1 (GLP-1) receptor (GLP1R) and glucose-dependent insulinotropic polypeptide (GIP) receptor (GIPR) dual agonist;
L is a linker that covalently links P and ABP; and
n ranges from about 1 to about 6.

62.-80. (canceled)

81. A method for treating obesity by reducing body weight while maintaining or increasing lean body mass in a subject, comprising administering a therapeutically effective amount of a GLP1R-GIPR dual agonist according to claim 41, to the subject in need thereof.

82. A method for treating obesity by reducing fat mass while maintaining or increasing lean body mass in a subject, comprising administering a therapeutically effective amount of a GLP1R-GIPR dual agonist according to claim 41, to the subject in need thereof.

83. A method for treating Type 2 diabetes by improving glycemic control and maintaining or increasing lean body mass while reducing fat mass in a subject, comprising administering a therapeutically effective amount of a GLP1R-GIPR dual agonist according to claim 41, to the subject in need thereof.

84. A method for treating obesity and Type 2 diabetes by improving glycemic control and maintaining or increasing lean body mass while reducing fat mass in a subject, comprising administering a therapeutically effective amount of a GLP1R-GIPR dual agonist according to claim 41, to the subject in need thereof.

85. A method for treating obesity, diabetes, and/or liver diseases associated with increased fat mass in a subject, comprising administering a therapeutically effective amount of a GLP1R-GIPR dual agonist according to claim 41, to the subject in need thereof.

86. A method for treating obesity, diabetes, and/or liver diseases associated with increased fat mass in a subject, comprising administering a therapeutically effective amount of a GLP1R-GIPR dual agonist according to claim 41, together with one or more other therapeutic agents, to the subject in need thereof.

87. A method for treating a subject of metabolic syndrome by improving glycemic control, maintaining or increasing lean body mass while reducing fat mass in a subject, comprising administering a therapeutically effective amount of a GLP1R-GIPR dual agonist according to claim 41, to the subject in need thereof.

88. A method for treating a subject of metabolic syndrome by improving glycemic control, maintaining or increasing lean body mass while reducing fat mass in a subject, comprising administering a therapeutically effective amount of a GLP1R-GIPR dual agonist according to claim 41, together with one or more other therapeutic agents, to the subject in need thereof.

89. A process for manufacturing GLP1R-GIPR dual agonist and an antigen binding protein (ABP) conjugate, wherein ABP is a GDF8 antibody or antigen binding fragment thereof, comprising a) optionally attaching a handle comprising a first reactive moiety; b) exposing a GLP1R-GIPR dual agonist comprising a second reactive moiety; c) initiating conjugation reaction between the first and second reactive moieties; and d) isolating/purifying said conjugate of GLP1R-GIPR dual agonist and ABP.

90.-94. (canceled)

95. A product of the process according to claim 89.

96. A pharmaceutical composition comprising a GLP1R-GIPR dual agonist and an antigen binding protein (ABP) conjugate manufactured according to the process of claim 89, together with one or more pharmaceutically acceptable excipients.

97.-103. (canceled)

104. The GLP1R and GIPR dual agonist, or a pharmaceutically acceptable salt thereof, according to claim 41, wherein XX3 is Glu, Gln, or Atz ((S)-2-amino-3-(2H-tetrazol-5-yl)propanoic acid).

105. (canceled)

106. The GLP1R and GIPR dual agonist, or a pharmaceutically acceptable salt thereof, according to claim 41, wherein XX24 is Ala, Gln, or Glu.

107.-109. (canceled)
